(19) United States
(12) Patent Application Publication
(10) Pub. No.: US 2026/0209208 A1
(43) Pub. Date: Jul. 23, 2026

(54) PHENYL-PYRAZOLE CARBOXAMIDE COMPOUNDS

(71) Applicant: Acrivon Therapeutics, Inc., Watertown, MA (US)

(72) Inventors: Magnus Munck af Rosenschöld, Lund (SE); Lars Thomas Brimert, Blentarp (SE); Hans Mattias Jönsson, Lund (SE); Filip Alexander Paulsen, Malmö (SE); Bo Roger Svensson, Lund (SE)

(21) Appl. No.: 19/142,148

(22) PCT Filed: Dec. 22, 2023
(86) PCT No.: PCT/US23/85642

§ 371 Date: Jun. 20, 2025

Related U.S. Application Data
(60) Provisional application No. 63/435,042, filed on Dec. 23, 2022.

(51) Int. Cl. C07D 405/04; A61K 31/4155; A61K 31/416; A61K 31/4439; A61K 45/06; A61P 35/00; C07D 405/14

(52) U.S. Cl. CPC C07D 405/04; A61K 31/4155; A61K 31/416; A61K 31/4439; A61K 45/06; A61P 35/00; C07D 405/14

(57) ABSTRACT

Phenyl-pyrazole carboxamide compounds of formula I (I) for use in methods of inhibiting Myt1 kinase.

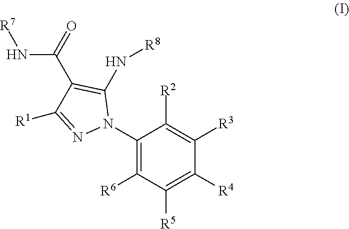

### BACKGROUND

Cells are continuously challenged with endogenous and exogenous agents that influence DNA integrity. To maintain genomic stability and prevent unwanted propagation of damaged DNA, cells have established an organized signaling network that recognizes DNA lesions and halts the cell cycle to allow the DNA to be correctly repaired before resuming DNA replication or cell division. The DNA damage response and the cell cycle are tightly linked via several cell cycle checkpoints that are important control steps for maintaining genomic integrity.

Cancer cells frequently have a defective G1/S checkpoint, often via disrupted p53 activity due to mutations or deletion, or inactivation by viral oncoproteins. Therefore, cancer cells rely heavily on other cell cycle checkpoints, including the G2/M checkpoint, to avoid accumulation of deleterious DNA damage and cell death. As such, cancer cells are hypothesized to be particularly vulnerable to inhibition of proteins that safeguard the entry into mitosis. Matheson, C. J., Backos, D. S. & Reigan, P. Targeting WEE1 Kinase in Cancer. Trends Pharmacol Sci 37, 872-881 (2016).

The gene PKMYT1 encodes the membrane-associated tyrosine/threonine-specific cdc2-inhibitory protein kinase PMyt1 (also known as Myt1 kinase), a member of the WEE family of serine/threonine kinases. While Wee1A kinase phosphorylates both Cdk1 and Cdk2 on tyrosine 15 (Tyr15), PMyt1 only phosphorylates cyclin-dependent kinase 1 (Cdk1), primarily at threonine 14 (Thr14) and to some extent at Tyr15. Phosphorylation of these sites inhibits Cdk1's kinase activity and its ability, in complex with cyclin B1, to trigger mitosis. In addition to inhibiting Cdk1-cyclin B1 complex by phosphorylation, it is proposed that Myt1 kinase also binds and sequesters Cdk1 from the nucleus, thereby further preventing the Cdk1-cyclinB1 complex from inducing mitosis. In addition to regulating mitotic entry, Myt1 kinase has been shown to have an important role in reassembly of the Golgi and ER during mitotic exit (Nakajima, H. et al. Myt1 protein kinase is essential for Golgi and ER assembly during mitotic exit. J Cell Biology 181, 89 103 (2008)). Thus, the main biological function of Myt1 kinase is to prevent replication of cells with high levels of damaged DNA or perturbed checkpoints. Cancers with amplification of CCNE1, a cyclin that drives entry and progression of S phase, have been shown to be highly sensitive to Myt1 kinase inhibition (Gallo, D. et al. CCNE1 amplification is synthetic lethal with PKMYT1 kinase inhibition. Nature 604, 749-756 (2022)). Similarly, mutations in FBXW7 that deactivate the ubiquitin ligase responsible for cyclin E1 degradation, may also result in abnormally high cyclin E1 levels.

Many cancer cell types overexpress Myt1 kinase, which may play a role in cancer cell survival by sustaining a high replication rate, replicative stress and genetic instability caused by oncogene expression. It is overexpressed in many cancer types including breast cancer (Liu, Y. et al. Systematic expression analysis of WEE family kinases reveals the importance of PKMYT1 in breast carcinogenesis. Cell Proliferat 53, e12741 (2020)), clear cell renal carcinoma (Chen, P., Zhang, Z. & Chen, X. Overexpression of PKMYT1 Facilitates Tumor Development and Is Correlated with Poor Prognosis in Clear Cell Renal Cell Carcinoma. Medical Sci Monit Int Medical J Exp Clin Res 26, e₉₂₆₇₅₅-1-e₉₂₆₇₅₅₋₂₂ (2020); Chen, J. et al. PKMYT1, exacerbating the progression of clear cell renal cell carcinoma, is implied as a biomarker for the diagnosis and prognosis. Aging Albany Ny 13, 25778-25798 (2021)), hepatocellular carcinoma (Liu, L. et al. PKMYT1 promoted the growth and motility of hepatocellular carcinoma cells by activating beta-catenin/TCF signaling. Exp Cell Res 358, 209-216 (2017)), non-small cell lung cancer (Sun, Q.-S., Luo, M., Zhao, H.-M. & Sun, H. Overexpression of PKMYT1 indicates the poor prognosis and enhances proliferation and tumorigenesis in non-small cell lung cancer via activation of Notch signal pathway. Eur Rev Med Pharmaco 23, 4210-4219 (2019)), and colorectal cancer (Jeong, D. et al. Protein kinase, membrane-associated tyrosine/threonine 1 is associated with the progression of colorectal cancer. Oncol Rep 39, 2829-2836 (2018)). By inhibiting Myt1 kinase, cancer cells lose their cell cycle checkpoints, accumulate DNA damage, increase genetic instability and eventually die of apoptosis. Moreover, when inhibition of Myt1 kinase is combined with administration of DNA damaging agents, such as chemo/radiotherapy, and other cell cycle checkpoint inhibitors, the DNA damaging agents become more cytotoxic because cell cycle progression is promoted before DNA repair can be achieved. Thus, the cells accumulate large amounts of DNA damage and eventually die of apoptosis.

Recently it has been discovered that resistance to the selective Wee1A kinase inhibitor, Adavosertib, is driven at least in part by upregulation of Myt1 kinase (C W Lewis et al, Cancer Res 2019; 79:5971-85).

Very few Myt1 kinase inhibitors have been described in the art and only one, RP-6306, is currently in human clinical trials (J Szychowski et al, J. Med. Chem. 2022, 65, 15, 10251-10284). Thus, there is an urgent need for new Myt1 kinase inhibitors for anti-cancer therapies.

### SUMMARY

In some embodiments, the present disclosure provides a compound of formula I:

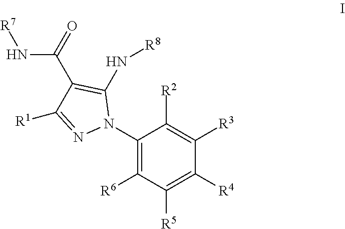

or a pharmaceutically acceptable salt thereof, wherein each of R¹, R², R³, R⁴, R⁵, R⁶, R⁷, and R⁸ are as defined below and described herein.

In some embodiments, the present disclosure provides a pharmaceutical composition comprising a compound of formula I, or a pharmaceutically acceptable salt thereof, and a pharmaceutically acceptable carrier.

In some embodiments, the present disclosure provides a method of inhibiting Myt1 kinase in a patient or in a biological sample, the method comprising administering to the patient or contacting the biological sample with a compound of formula I, or a pharmaceutically acceptable salt thereof.

In some embodiments, the present disclosure provides a method of treating a disease or disorder associated with Myt1 kinase, the method comprising administering to a patient in need thereof a compound of formula I, or a pharmaceutically acceptable salt thereof.

In some embodiments, the present disclosure provides a method of treating a disease or disorder associated with Myt1 kinase, the method comprising administering to a patient in need thereof a compound of formula I, or a pharmaceutically acceptable salt thereof.

In some such embodiments, the disease or disorder associated with Myt1 kinase is a cancer. In some embodiments, a cancer is selected from breast cancer, clear cell renal carcinoma, hepatocellular carcinoma, uterine cancer, ovarian cancer, stomach cancer, esophageal cancer, lung cancer, and colorectal cancer.

### DETAILED DESCRIPTION

### 1. General Description of Compounds of the Disclosure

In some embodiments, the present disclosure provides inhibitors of Myt1 kinase. In some embodiments, such compounds include those of the formulae described herein, or a pharmaceutically acceptable salt thereof, wherein each variable is as defined and described herein.

In some embodiments, the present disclosure provides a compound of formula I:

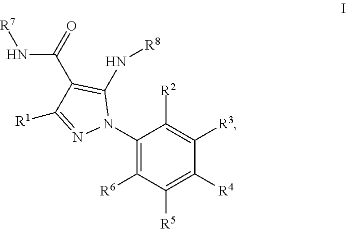

or a solvate, enantiomer, tautomer, or diastereomer thereof, or a pharmaceutically acceptable salt of any of the foregoing wherein:
R¹ is aryl, carbocyclyl, heteroaryl or heterocyclyl, each of which is optionally substituted;
each of R², R³, R⁴ and R⁶ is independently hydrogen, halo, —C₁-C₄ alkyl, —O—C₁-C₄ alkyl, —C₂-C₄ alkynyl, —CN, cyclopropyl, or phenyl; and
R⁵ is —OH; or
R² is halo, —C₁-C₄ alkyl, —O—C₁-C₄ alkyl, —C₂-C₄ alkynyl, —CN, or cyclopropyl;
each of R³ and R⁴ is independently hydrogen, halo, —C₁-C₄ alkyl, —O—C₁-C₄ alkyl, —CN, cyclopropyl, or phenyl; and
R⁵ and R⁶ are taken together with carbon atoms to which they are bound to form Ring A;
Ring A is a 4-7 membered heterocyclic or heteroaromatic ring comprising a ring nitrogen atom and optionally substituted with one or more R^{a} groups;
each R^{a} is independently selected from halo, ═O, —C₁-C₄ alkyl, —O—C₁-C₄ alkyl, —CN, and cyclopropyl;
each of R⁷ and R⁸ is independently hydrogen or optionally substituted C₁-C₄ alkyl; and
wherein one or more hydrogen atoms in the compound is optionally replaced with a deuterium atom.

In some embodiments, the compound of formula I may be a compound, or a pharmaceutically acceptable salt thereof, selected from Table 1:

TABLE 1
Exemplary Compounds of Formula I

| # | Structure |
|---|---|
| 1 | 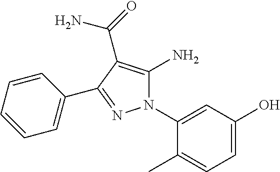 [SMILES: Cc1ccc(O)cc1-n1nc(-c2ccccc2)c(C(N)=O)c1N] |
| 2 | 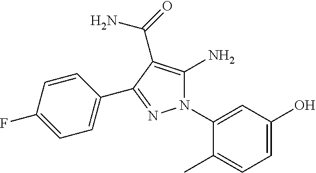 [SMILES: Cc1ccc(O)cc1-n1nc(-c2ccc(F)cc2)c(C(N)=O)c1N] |
| 3 | 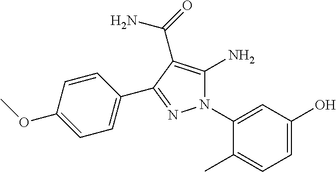 [SMILES: COc1ccc(-c2nn(-c3cc(O)ccc3C)c(N)c2C(N)=O)cc1] |
| 4 | 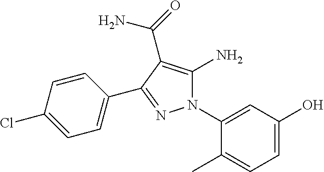 [SMILES: Cc1ccc(O)cc1-n1nc(-c2ccc(Cl)cc2)c(C(N)=O)c1N] |
| 5 | 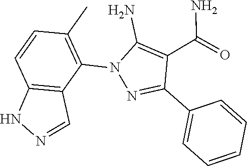 [SMILES: Cc1ccc2[nH]ncc2c1-n1nc(-c2ccccc2)c(C(N)=O)c1N] |
| 6 | 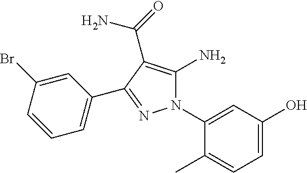 [SMILES: Cc1ccc(O)cc1-n1nc(-c2cccc(Br)c2)c(C(N)=O)c1N] |
| 7 | 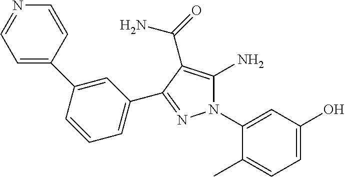 [SMILES: Cc1ccc(O)cc1-n1nc(-c2cccc(-c3ccncc3)c2)c(C(N)=O)c1N] |
| 8 | 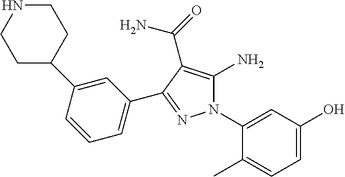 [SMILES: Cc1ccc(O)cc1-n1nc(-c2cccc(C3CCNCC3)c2)c(C(N)=O)c1N] |
| 9 | 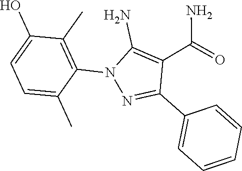 [SMILES: Cc1ccc(O)c(C)c1-n1nc(-c2ccccc2)c(C(N)=O)c1N] |
| 10 | 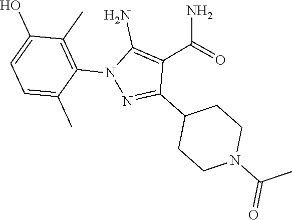 [SMILES: CC(=O)N1CCC(c2nn(-c3c(C)ccc(O)c3C)c(N)c2C(N)=O)CC1] |
| 11 | 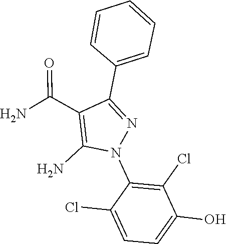 [SMILES: NC(=O)c1c(-c2ccccc2)nn(-c2c(Cl)ccc(O)c2Cl)c1N] |
| 12 | 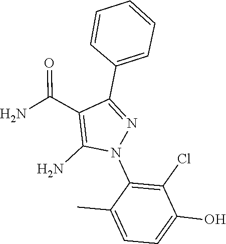 [SMILES: Cc1ccc(O)c(Cl)c1-n1nc(-c2ccccc2)c(C(N)=O)c1N] |
| 13 | 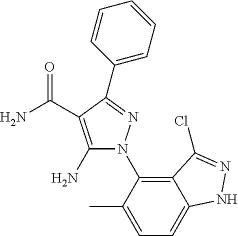 [SMILES: Cc1ccc2[nH]nc(Cl)c2c1-n1nc(-c2ccccc2)c(C(N)=O)c1N] |
| 14 | 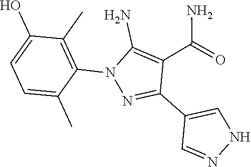 [SMILES: Cc1ccc(O)c(C)c1-n1nc(-c2cn[nH]c2)c(C(N)=O)c1N] |
| 15 | 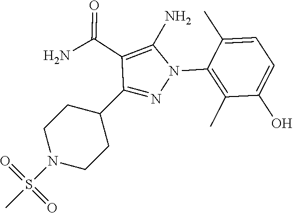 [SMILES: Cc1ccc(O)c(C)c1-n1nc(C2CCN(S(C)(=O)=O)CC2)c(C(N)=O)c1N] |
| 16 | 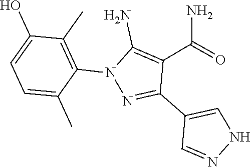 [SMILES: Cc1ccc(O)c(C)c1-n1nc(-c2cn[nH]c2)c(C(N)=O)c1N] |
| 17 | 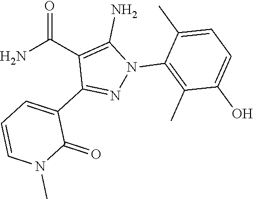 [SMILES: Cc1ccc(O)c(C)c1-n1nc(-c2cccn(C)c2=O)c(C(N)=O)c1N] |
| 18 | 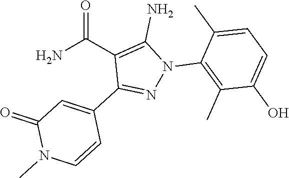 [SMILES: Cc1ccc(O)c(C)c1-n1nc(-c2ccn(C)c(=O)c2)c(C(N)=O)c1N] |
| 19 | 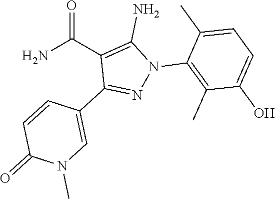 [SMILES: Cc1ccc(O)c(C)c1-n1nc(-c2ccc(=O)n(C)c2)c(C(N)=O)c1N] |
| 20 | 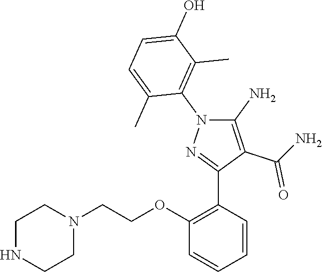 [SMILES: Cc1ccc(O)c(C)c1-n1nc(-c2ccccc2OCCN2CCNCC2)c(C(N)=O)c1N] |
| 21 | 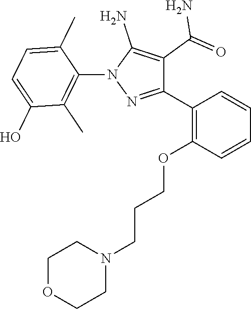 [SMILES: Cc1ccc(O)c(C)c1-n1nc(-c2ccccc2OCCCN2CCOCC2)c(C(N)=O)c1N] |
| 22 | 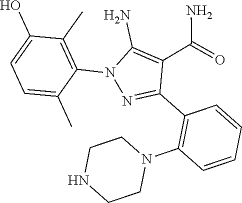 [SMILES: Cc1ccc(O)c(C)c1-n1nc(-c2ccccc2N2CCNCC2)c(C(N)=O)c1N] |
| 23 | 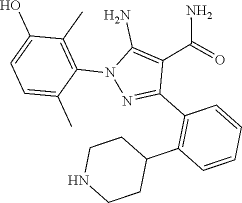 [SMILES: Cc1ccc(O)c(C)c1-n1nc(-c2ccccc2C2CCNCC2)c(C(N)=O)c1N] |
| 24 | 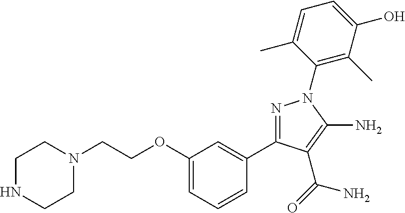 [SMILES: Cc1ccc(O)c(C)c1-n1nc(-c2cccc(OCCN3CCNCC3)c2)c(C(N)=O)c1N] |
| 25 | 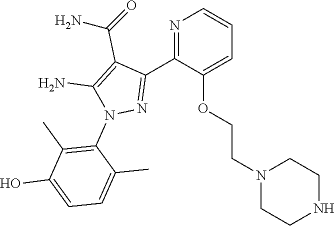 [SMILES: Cc1ccc(O)c(C)c1-n1nc(-c2ncccc2OCCN2CCNCC2)c(C(N)=O)c1N] |
| 26 | 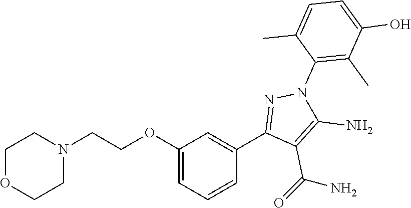 [SMILES: Cc1ccc(O)c(C)c1-n1nc(-c2cccc(OCCN3CCOCC3)c2)c(C(N)=O)c1N] |
| 27 | 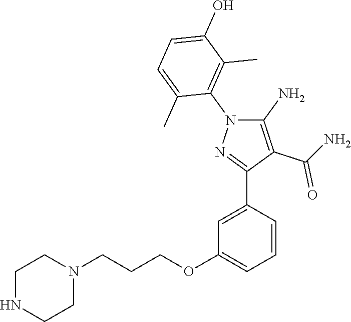 [SMILES: Cc1ccc(O)c(C)c1-n1nc(-c2cccc(OCCCN3CCNCC3)c2)c(C(N)=O)c1N] |
| 28 | 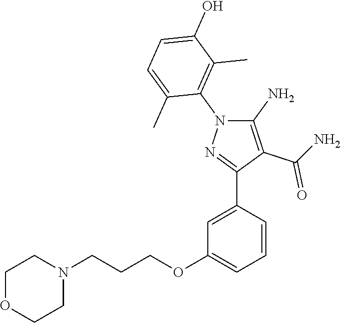 [SMILES: Cc1ccc(O)c(C)c1-n1nc(-c2cccc(OCCCN3CCOCC3)c2)c(C(N)=O)c1N] |
| 29 | 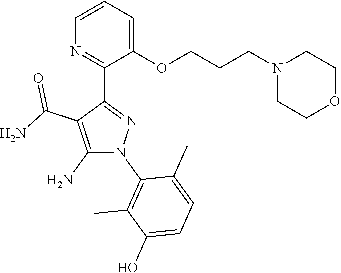 [SMILES: Cc1ccc(O)c(C)c1-n1nc(-c2ncccc2OCCCN2CCOCC2)c(C(N)=O)c1N] |
| 30 | 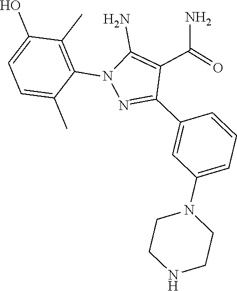 [SMILES: Cc1ccc(O)c(C)c1-n1nc(-c2cccc(N3CCNCC3)c2)c(C(N)=O)c1N] |
| 31 | 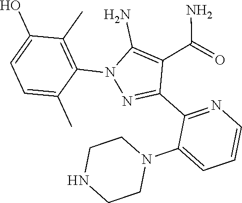 [SMILES: Cc1ccc(O)c(C)c1-n1nc(-c2ncccc2N2CCNCC2)c(C(N)=O)c1N] |
| 32 | 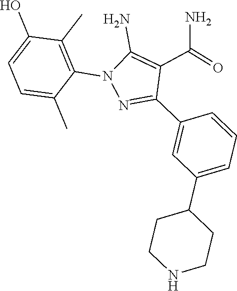 [SMILES: Cc1ccc(O)c(C)c1-n1nc(-c2cccc(C3CCNCC3)c2)c(C(N)=O)c1N] |
| 33 | 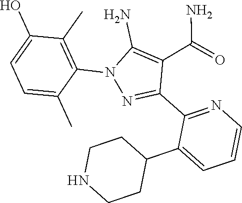 [SMILES: Cc1ccc(O)c(C)c1-n1nc(-c2ncccc2C2CCNCC2)c(C(N)=O)c1N] |
| 34 | 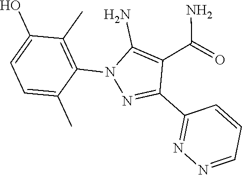 [SMILES: Cc1ccc(O)c(C)c1-n1nc(-c2cccnn2)c(C(N)=O)c1N] |
| 35 | 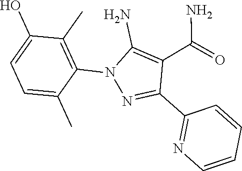 [SMILES: Cc1ccc(O)c(C)c1-n1nc(-c2ccccn2)c(C(N)=O)c1N] |
| 36 | 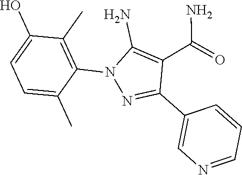 [SMILES: Cc1ccc(O)c(C)c1-n1nc(-c2cccnc2)c(C(N)=O)c1N] |
| 37 | 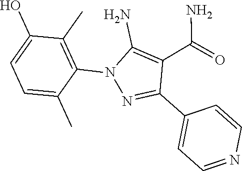 [SMILES: Cc1ccc(O)c(C)c1-n1nc(-c2ccncc2)c(C(N)=O)c1N] |
| 38 | 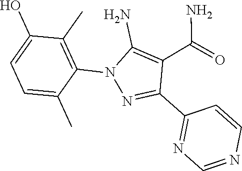 [SMILES: Cc1ccc(O)c(C)c1-n1nc(-c2ccncn2)c(C(N)=O)c1N] |
| 39 | 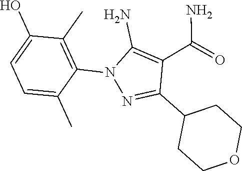 [SMILES: Cc1ccc(O)c(C)c1-n1nc(C2CCOCC2)c(C(N)=O)c1N] |
| 40 | 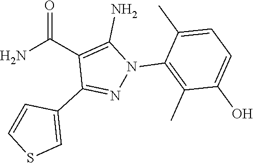 [SMILES: Cc1ccc(O)c(C)c1-n1nc(-c2ccsc2)c(C(N)=O)c1N] |
| 41 | 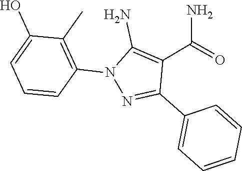 [SMILES: Cc1c(O)cccc1-n1nc(-c2ccccc2)c(C(N)=O)c1N] |
| 42 | 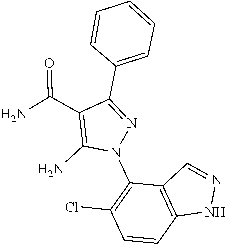 [SMILES: NC(=O)c1c(-c2ccccc2)nn(-c2c(Cl)ccc3[nH]ncc23)c1N] |
| 43 | 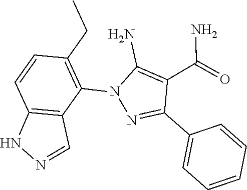 [SMILES: CCc1ccc2[nH]ncc2c1-n1nc(-c2ccccc2)c(C(N)=O)c1N] |
| 44 | 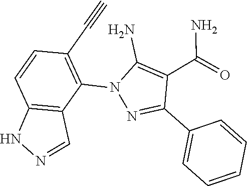 [SMILES: C#Cc1ccc2[nH]ncc2c1-n1nc(-c2ccccc2)c(C(N)=O)c1N] |
| 45 | 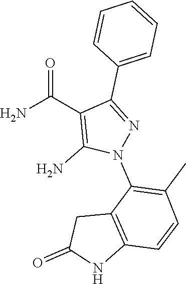 [SMILES: Cc1ccc2c(c1-n1nc(-c3ccccc3)c(C(N)=O)c1N)CC(=O)N2] |
| 46 | 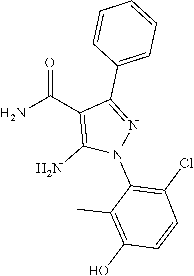 [SMILES: Cc1c(O)ccc(Cl)c1-n1nc(-c2ccccc2)c(C(N)=O)c1N] |
| 47 | 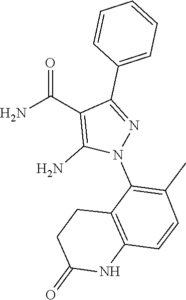 [SMILES: Cc1ccc2c(c1-n1nc(-c3ccccc3)c(C(N)=O)c1N)CCC(=O)N2] |
| 48 | 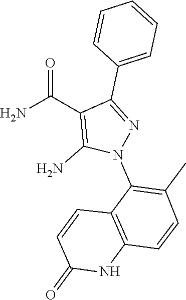 [SMILES: Cc1ccc2[nH]c(=O)ccc2c1-n1nc(-c2ccccc2)c(C(N)=O)c1N] |
| 49 | 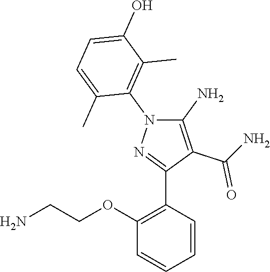 [SMILES: Cc1ccc(O)c(C)c1-n1nc(-c2ccccc2OCCN)c(C(N)=O)c1N] |
| 50 | 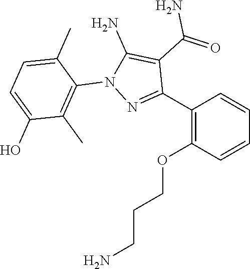 [SMILES: Cc1ccc(O)c(C)c1-n1nc(-c2ccccc2OCCCN)c(C(N)=O)c1N] |
| 51 | 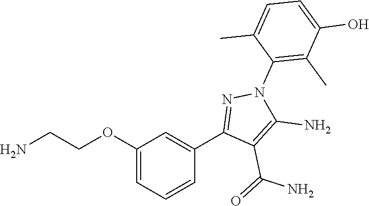 [SMILES: Cc1ccc(O)c(C)c1-n1nc(-c2cccc(OCCN)c2)c(C(N)=O)c1N] |
| 52 | 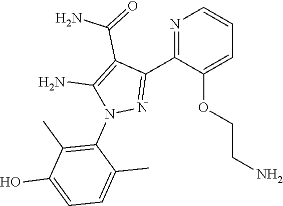 [SMILES: Cc1ccc(O)c(C)c1-n1nc(-c2ncccc2OCCN)c(C(N)=O)c1N] |
| 53 | 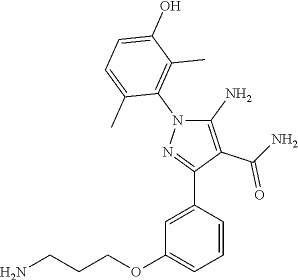 [SMILES: Cc1ccc(O)c(C)c1-n1nc(-c2cccc(OCCCN)c2)c(C(N)=O)c1N] |
| 54 | 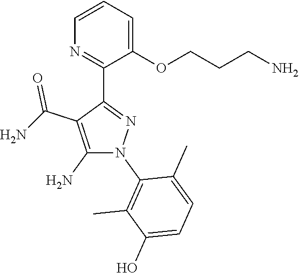 [SMILES: Cc1ccc(O)c(C)c1-n1nc(-c2ncccc2OCCCN)c(C(N)=O)c1N] |
| 55 | 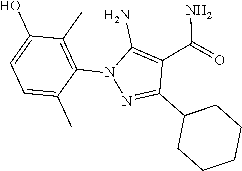 [SMILES: Cc1ccc(O)c(C)c1-n1nc(C2CCCCC2)c(C(N)=O)c1N] |
| 56 | 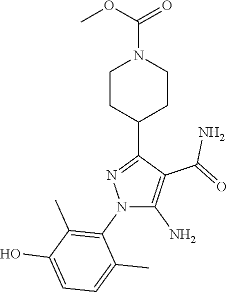 [SMILES: COC(=O)N1CCC(c2nn(-c3c(C)ccc(O)c3C)c(N)c2C(N)=O)CC1] |
| 57 | 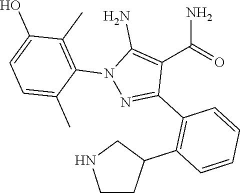 [SMILES: Cc1ccc(O)c(C)c1-n1nc(-c2ccccc2C2CCNC2)c(C(N)=O)c1N] |
| 58 | 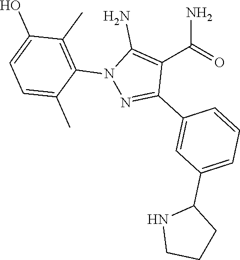 [SMILES: Cc1ccc(O)c(C)c1-n1nc(-c2cccc(C3CCCN3)c2)c(C(N)=O)c1N] |
| 59 | 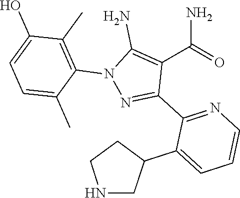 [SMILES: Cc1ccc(O)c(C)c1-n1nc(-c2ncccc2C2CCNC2)c(C(N)=O)c1N] |
| 60 | 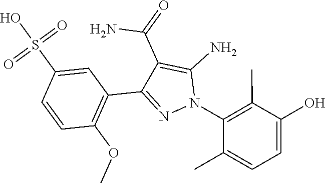 [SMILES: COc1ccc(S(=O)(=O)O)cc1-c1nn(-c2c(C)ccc(O)c2C)c(N)c1C(N)=O] |
| 61 | 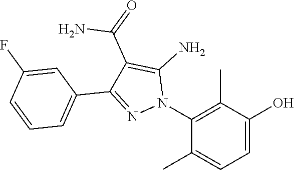 [SMILES: Cc1ccc(O)c(C)c1-n1nc(-c2cccc(F)c2)c(C(N)=O)c1N] |
| 62 | 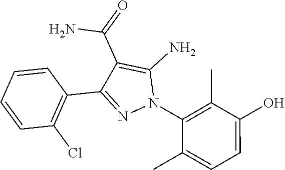 [SMILES: Cc1ccc(O)c(C)c1-n1nc(-c2ccccc2Cl)c(C(N)=O)c1N] |
| 63 | 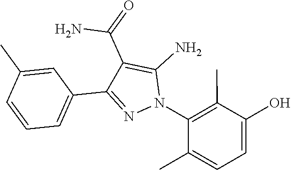 [SMILES: Cc1cccc(-c2nn(-c3c(C)ccc(O)c3C)c(N)c2C(N)=O)c1] |
| 64 | 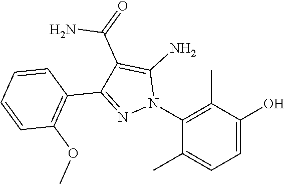 [SMILES: COc1ccccc1-c1nn(-c2c(C)ccc(O)c2C)c(N)c1C(N)=O] |
| 65 | 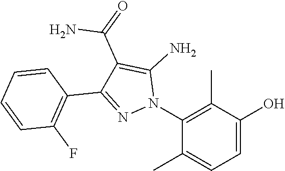 [SMILES: Cc1ccc(O)c(C)c1-n1nc(-c2ccccc2F)c(C(N)=O)c1N] |
| 66 | 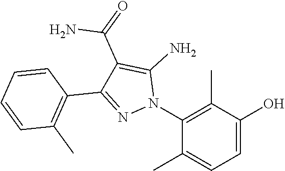 [SMILES: Cc1ccccc1-c1nn(-c2c(C)ccc(O)c2C)c(N)c1C(N)=O] |
| 67 | 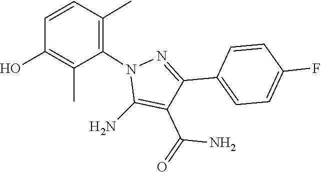 [SMILES: Cc1ccc(O)c(C)c1-n1nc(-c2ccc(F)cc2)c(C(N)=O)c1N] |
| 68 | 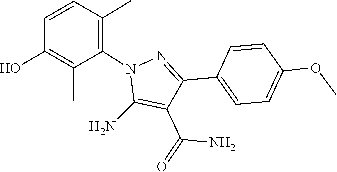 [SMILES: COc1ccc(-c2nn(-c3c(C)ccc(O)c3C)c(N)c2C(N)=O)cc1] |
| 69 | 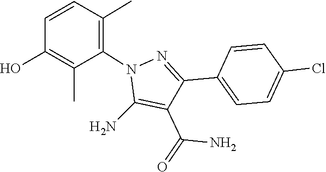 [SMILES: Cc1ccc(O)c(C)c1-n1nc(-c2ccc(Cl)cc2)c(C(N)=O)c1N] |
| 70 | 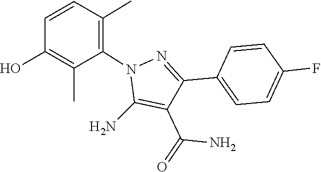 [SMILES: Cc1ccc(O)c(C)c1-n1nc(-c2ccc(F)cc2)c(C(N)=O)c1N] |
| 71 | 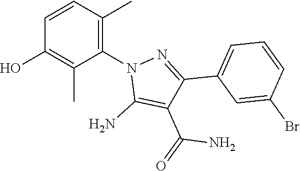 [SMILES: Cc1ccc(O)c(C)c1-n1nc(-c2cccc(Br)c2)c(C(N)=O)c1N] |
| 72 | 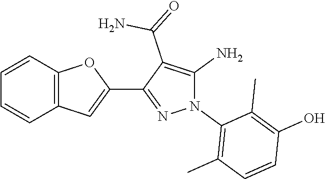 [SMILES: Cc1ccc(O)c(C)c1-n1nc(-c2cc3ccccc3o2)c(C(N)=O)c1N] |
| 73 | 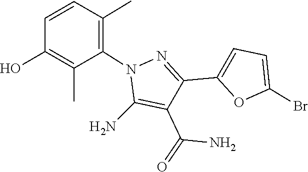 [SMILES: Cc1ccc(O)c(C)c1-n1nc(-c2ccc(Br)o2)c(C(N)=O)c1N] |
| 74 | 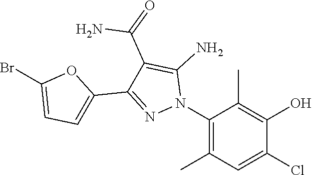 [SMILES: Cc1cc(Cl)c(O)c(C)c1-n1nc(-c2ccc(Br)o2)c(C(N)=O)c1N] |
| 75 | 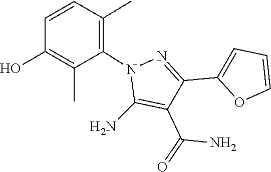 [SMILES: Cc1ccc(O)c(C)c1-n1nc(-c2ccco2)c(C(N)=O)c1N] |
| 76 | 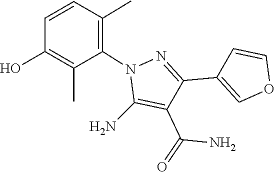 [SMILES: Cc1ccc(O)c(C)c1-n1nc(-c2ccoc2)c(C(N)=O)c1N] |
| 77 | 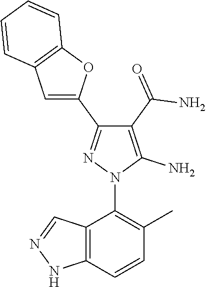 [SMILES: Cc1ccc2[nH]ncc2c1-n1nc(-c2cc3ccccc3o2)c(C(N)=O)c1N] |
| 78 | 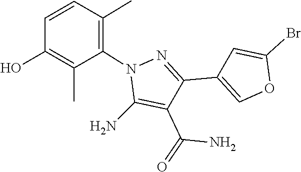 [SMILES: Cc1ccc(O)c(C)c1-n1nc(-c2coc(Br)c2)c(C(N)=O)c1N] |
| 79 | 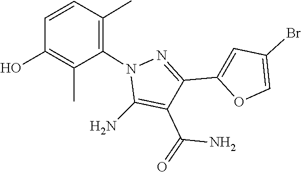 [SMILES: Cc1ccc(O)c(C)c1-n1nc(-c2cc(Br)co2)c(C(N)=O)c1N] |
| 80 | 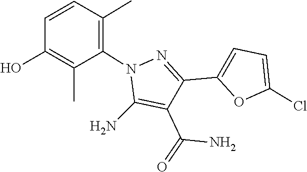 [SMILES: Cc1ccc(O)c(C)c1-n1nc(-c2ccc(Cl)o2)c(C(N)=O)c1N] |
| 81 | 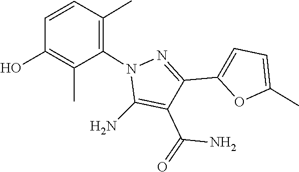 [SMILES: Cc1ccc(-c2nn(-c3c(C)ccc(O)c3C)c(N)c2C(N)=O)o1] |
| 82 | 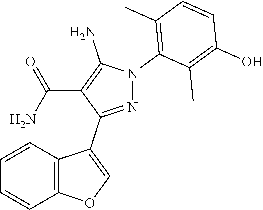 [SMILES: Cc1ccc(O)c(C)c1-n1nc(-c2coc3ccccc23)c(C(N)=O)c1N] |
| 83 | 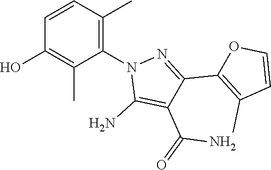 [SMILES: Cc1ccoc1-c1nn(-c2c(C)ccc(O)c2C)c(N)c1C(N)=O] |
| 84 | 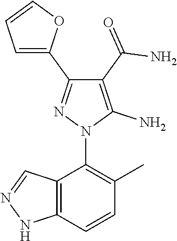 [SMILES: Cc1ccc2[nH]ncc2c1-n1nc(-c2ccco2)c(C(N)=O)c1N] |
| 85 | 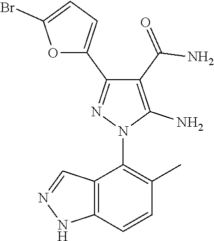 [SMILES: Cc1ccc2[nH]ncc2c1-n1nc(-c2ccc(Br)o2)c(C(N)=O)c1N] |
| 86 | 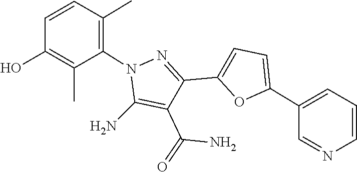 [SMILES: Cc1ccc(O)c(C)c1-n1nc(-c2ccc(-c3cccnc3)o2)c(C(N)=O)c1N] |
| 87 | 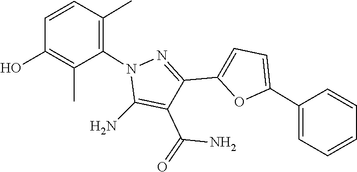 [SMILES: Cc1ccc(O)c(C)c1-n1nc(-c2ccc(-c3ccccc3)o2)c(C(N)=O)c1N] |
| 88 | 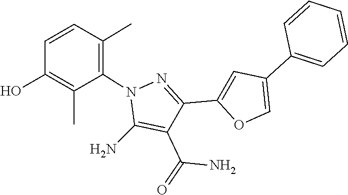 [SMILES: Cc1ccc(O)c(C)c1-n1nc(-c2cc(-c3ccccc3)co2)c(C(N)=O)c1N] |
| 89 | 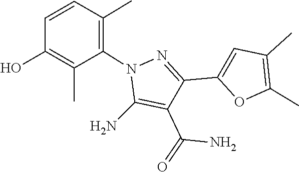 [SMILES: Cc1cc(-c2nn(-c3c(C)ccc(O)c3C)c(N)c2C(N)=O)oc1C] |
| 90 | 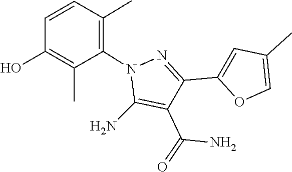 [SMILES: Cc1coc(-c2nn(-c3c(C)ccc(O)c3C)c(N)c2C(N)=O)c1] |
| 91 | 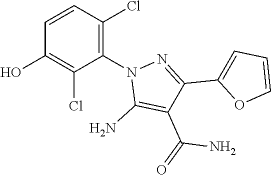 [SMILES: NC(=O)c1c(-c2ccco2)nn(-c2c(Cl)ccc(O)c2Cl)c1N] |
| 92 | 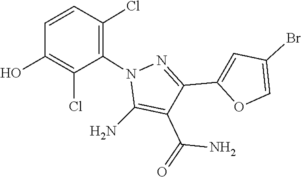 [SMILES: NC(=O)c1c(-c2cc(Br)co2)nn(-c2c(Cl)ccc(O)c2Cl)c1N] |
| 93 | 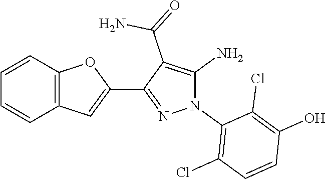 [SMILES: NC(=O)c1c(-c2cc3ccccc3o2)nn(-c2c(Cl)ccc(O)c2Cl)c1N] |
| 94 | 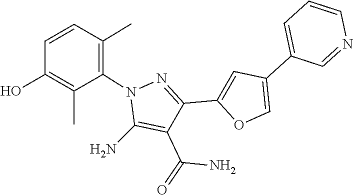 [SMILES: Cc1ccc(O)c(C)c1-n1nc(-c2cc(-c3cccnc3)co2)c(C(N)=O)c1N] |
| 95 | 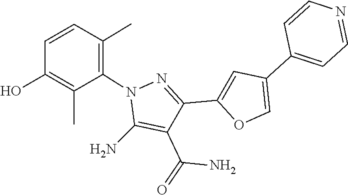 [SMILES: Cc1ccc(O)c(C)c1-n1nc(-c2cc(-c3ccncc3)co2)c(C(N)=O)c1N] |
| 96 | 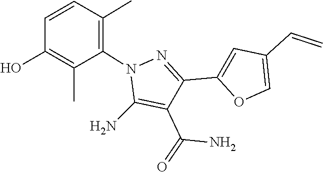 [SMILES: C=Cc1coc(-c2nn(-c3c(C)ccc(O)c3C)c(N)c2C(N)=O)c1] |
| 97 | 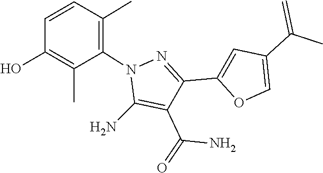 [SMILES: C=C(C)c1coc(-c2nn(-c3c(C)ccc(O)c3C)c(N)c2C(N)=O)c1] |
| 98 | 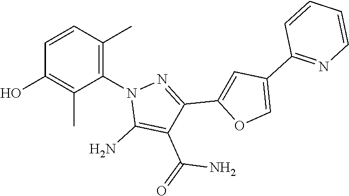 [SMILES: Cc1ccc(O)c(C)c1-n1nc(-c2cc(-c3ccccn3)co2)c(C(N)=O)c1N] |
| 99 | 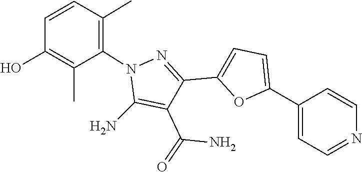 [SMILES: Cc1ccc(O)c(C)c1-n1nc(-c2ccc(-c3ccncc3)o2)c(C(N)=O)c1N] |
| 100 | 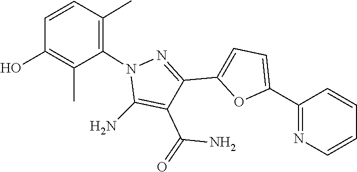 [SMILES: Cc1ccc(O)c(C)c1-n1nc(-c2ccc(-c3ccccn3)o2)c(C(N)=O)c1N] |
| 101 | 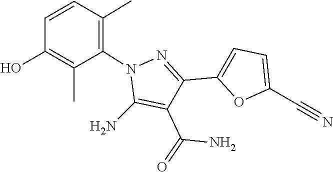 [SMILES: Cc1ccc(O)c(C)c1-n1nc(-c2ccc(C#N)o2)c(C(N)=O)c1N] |
| 102 | 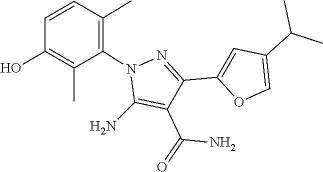 [SMILES: Cc1ccc(O)c(C)c1-n1nc(-c2cc(C(C)C)co2)c(C(N)=O)c1N] |
| 103 | 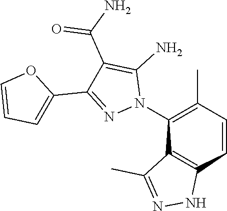 |
| 104 | 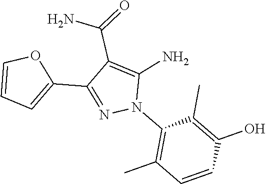 |
| 105 | 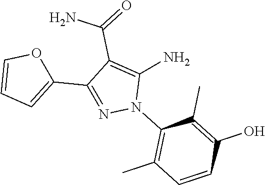 |
| 106 | 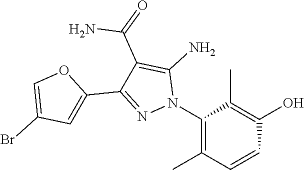 |
| 107 | 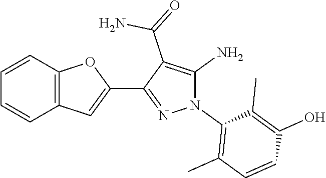 |
| 108 | 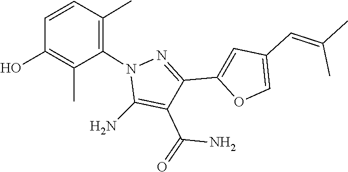 [SMILES: CC(C)=Cc1coc(-c2nn(-c3c(C)ccc(O)c3C)c(N)c2C(N)=O)c1] |
| 109 | 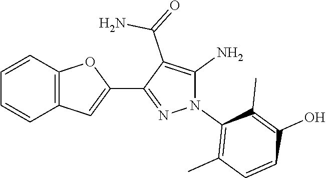 |
| 110 | 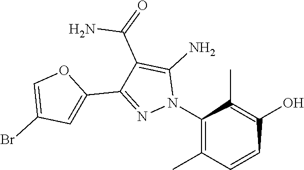 |
| 111 | 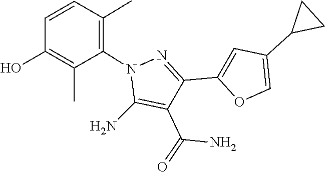 [SMILES: Cc1ccc(O)c(C)c1-n1nc(-c2cc(C3CC3)co2)c(C(N)=O)c1N] |
| 112 | 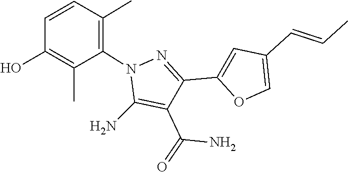 [SMILES: C/C=C/c1coc(-c2nn(-c3c(C)ccc(O)c3C)c(N)c2C(N)=O)c1] |
| 113 | 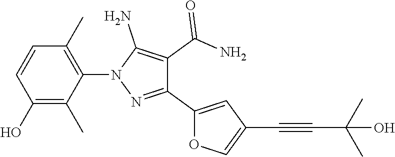 [SMILES: Cc1ccc(O)c(C)c1-n1nc(-c2cc(C#CC(C)(C)O)co2)c(C(N)=O)c1N] |
| 114 | 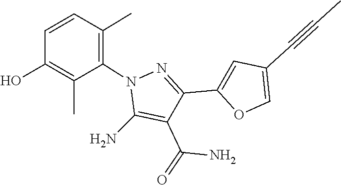 [SMILES: CC#Cc1coc(-c2nn(-c3c(C)ccc(O)c3C)c(N)c2C(N)=O)c1] |
| 115 | 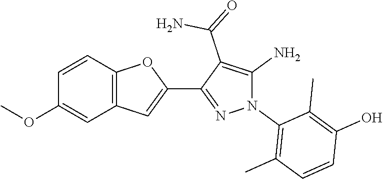 [SMILES: COc1ccc2oc(-c3nn(-c4c(C)ccc(O)c4C)c(N)c3C(N)=O)cc2c1] |
| 116 | 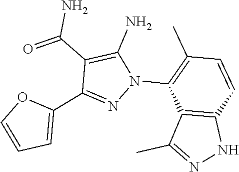 |
| 117 | 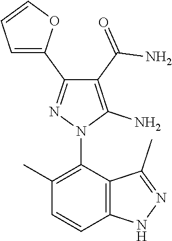 [SMILES: Cc1ccc2[nH]nc(C)c2c1-n1nc(-c2ccco2)c(C(N)=O)c1N] |
| 118 | 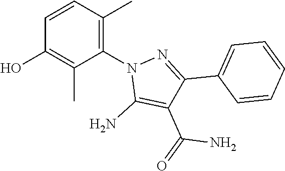 [SMILES: Cc1ccc(O)c(C)c1-n1nc(-c2ccccc2)c(C(N)=O)c1N] |
| 119 | 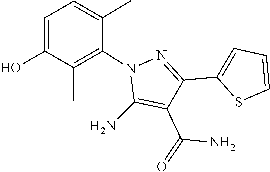 [SMILES: Cc1ccc(O)c(C)c1-n1nc(-c2cccs2)c(C(N)=O)c1N] |
| 120 | 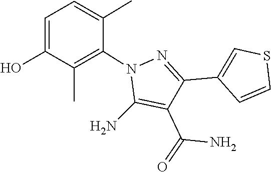 [SMILES: Cc1ccc(O)c(C)c1-n1nc(-c2ccsc2)c(C(N)=O)c1N] |
| 121 | 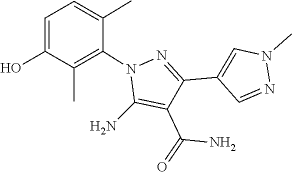 [SMILES: Cc1ccc(O)c(C)c1-n1nc(-c2cnn(C)c2)c(C(N)=O)c1N] |
| 122 | 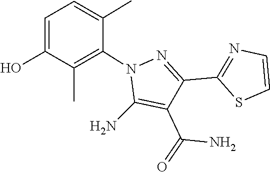 [SMILES: Cc1ccc(O)c(C)c1-n1nc(-c2nccs2)c(C(N)=O)c1N] |
| 123 | 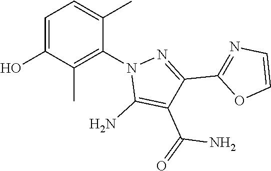 [SMILES: Cc1ccc(O)c(C)c1-n1nc(-c2ncco2)c(C(N)=O)c1N] |
| 124 | 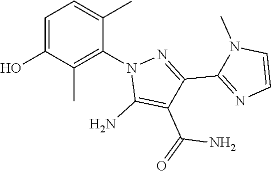 [SMILES: Cc1ccc(O)c(C)c1-n1nc(-c2nccn2C)c(C(N)=O)c1N] |
| 125 | 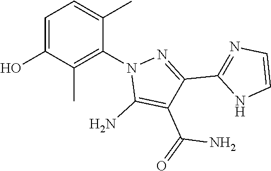 [SMILES: Cc1ccc(O)c(C)c1-n1nc(-c2ncc[nH]2)c(C(N)=O)c1N] |
| 126 | 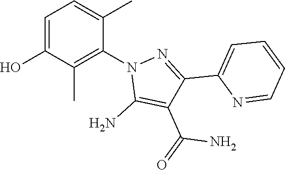 [SMILES: Cc1ccc(O)c(C)c1-n1nc(-c2ccccn2)c(C(N)=O)c1N] |

or a pharmaceutically acceptable salt thereof.

### 2. Compounds and Definitions

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocyclyl” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁻ (as in N-substituted pyrrolidinyl)).

The term “unsaturated”, as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic carbocyclic rings.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridinyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl or heteroaryl rings such that the resulting bi- or multicyclic ring system as a whole is fully aromatic. Nonlimiting examples include indolyl, isoindolyl, benzothienyl, benzofuranyl, dibenzofuranyl, indazolyl, benzimidazolyl, benzothiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, and phenoxazinyl. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene) O—N(R^{∘}₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{∘}, —(CH₂)₀₋₂SR^{∘}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{*}₂, —NNHC(O)R^{*}, ═NNHC(O)OR^{*}, ═NNHS(O)₂R^{*}, ═NR^{*}, ═NOR^{*}, —O(C(R*₂))₂₋₃O—, or —S(C(R*₂))₂₋₃S—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{*}₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{*} include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, rotational isomers (atropisomers) and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Inhibition of activity of a protein kinase, for example, Myt1 kinase or a mutant thereof, in a biological sample is useful for a variety of purposes that are known to one of skill in the art. Examples of such purposes include, but are not limited to, blood transfusion, organ transplantation, biological specimen storage, and biological assays.

As used herein, a “disease or disorder associated with Myt1 kinase” or, alternatively, “a Myt1 kinase-mediated disease or disorder” means any disease or other deleterious condition in which Myt1 kinase, or a mutant thereof, is known or suspected to play a role.

The term “subject”, as used herein, means a mammal and includes human and animal subjects, such as domestic animals (e.g., horses, dogs, cats, etc.). The terms “subject” and “patient” are used interchangeably. In some embodiments, the “patient” or “subject” means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The amount of compounds of the present disclosure that may be combined with the carrier materials to produce a composition in a single dosage form will vary depending upon the host treated, the particular mode of administration, etc. Preferably, provided compositions are formulated so that a dosage of between 0.01 to about 100 mg/kg, or about 0.1 mg/kg to about 50 mg/kg, and preferably from about 1 mg/kg to about 25 mg/kg, of subject body weight/day of the inhibitor can be administered to a patient receiving these compositions to obtain the desired therapeutic effect. The amount of a compound of the present disclosure in the composition will also depend upon the particular compound in the composition.

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

As used herein, the term “inhibitor” is defined as a compound that binds to and/or inhibits the target protein kinase with measurable affinity. In certain embodiments, an inhibitor has an IC₅₀ and/or binding constant of less about 50 μM, less than about 1 μM, less than about 500 nM, less than about 100 nM, less than about 50 nM, or less than about 10 nM.

The terms “measurable affinity” and “measurably inhibit,” as used herein, means a measurable change in Myt1 kinase activity between a sample comprising a compound of the present disclosure, or composition thereof, and an equivalent sample comprising Myt1 kinase, in the absence of said compound, or composition thereof.

### 3. Description of Exemplary Compounds

In some embodiments, the present disclosure provides a compound of formula I:

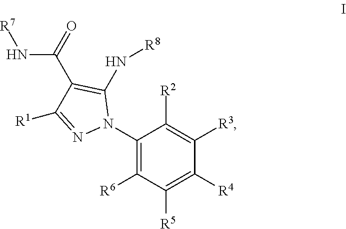

or a solvate, enantiomer, tautomer, atropisomer, or diastereomer thereof, or a pharmaceutically acceptable salt of any of the foregoing wherein:
R¹ is aryl, carbocyclyl, heteroaryl or heterocyclyl, each of which is optionally substituted;
each of R², R³, R⁴ and R⁶ is independently hydrogen, halo, —C₁-C₄ alkyl, —O—C₁-C₄ alkyl, —C₂-C₄ alkynyl, —CN, cyclopropyl, or phenyl; and
R⁵ is —OH; or
R² is hydrogen, halo, —C₁-C₄ alkyl, —O—C₁-C₄ alkyl, —C₂-C₄ alkynyl, —CN, or cyclopropyl;
each of R³ and R⁴ is independently hydrogen, halo, —C₁-C₄ alkyl, —O—C₁-C₄ alkyl, —CN, cyclopropyl, or phenyl; and
R⁵ and R⁶ are taken together with carbon atoms to which they are bound to form Ring A;
Ring A is a 4-7 membered heterocyclic or heteroaromatic ring comprising a ring nitrogen atom and optionally substituted with one or more R^{a} groups;
each R^{a} is independently selected from halo, ═O, —C₁-C₄ alkyl, —O—C₁-C₄ alkyl, —CN, and cyclopropyl;
each of R⁷ and R⁸ is independently hydrogen or optionally substituted C₁-C₄ alkyl; and
wherein one or more hydrogen atoms in the compound is optionally replaced with a deuterium atom.

In some embodiments, the present disclosure provides a compound of formula I-a:

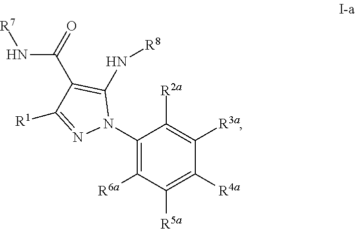

or a solvate, enantiomer, tautomer, or diastereomer thereof, or a pharmaceutically acceptable salt of any of the foregoing wherein:
R¹ is aryl, carbocyclyl, heteroaryl or heterocyclyl, each of which is optionally substituted;
each of R^{2a}, R^{3a}, R^{4a} and R^{6a} is independently hydrogen, halo, —C₁-C₄ alkyl, —O—C₁-C₄ alkyl, —C₂-C₄ alkynyl, —CN, cyclopropyl, or phenyl; and
R^{5a} is —OH;
each of R⁷ and R⁸ is independently hydrogen or optionally substituted C₁-C₄ alkyl; and
wherein one or more hydrogen atoms in the compound is optionally replaced with a deuterium atom.

In some embodiments, the present disclosure provides a compound of formula I-b:

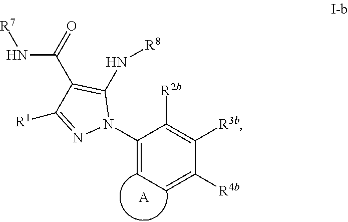

or a solvate, enantiomer, tautomer, or diastereomer thereof, or a pharmaceutically acceptable salt of any of the foregoing wherein:
R¹ is aryl, carbocyclyl, heteroaryl or heterocyclyl, each of which is optionally substituted;
R^{2b} is hydrogen, halo, —C₁-C₄ alkyl, —O—C₁-C₄ alkyl, —C₂-C₄ alkynyl, —CN, or cyclopropyl;
each of R^{3b} and R^{4b} is independently hydrogen, halo, —C₁-C₄ alkyl, —O—C₁-C₄ alkyl, —CN, cyclopropyl, or phenyl;
Ring A is a 4-7 membered heterocyclic or heteroaromatic ring comprising a ring nitrogen atom and optionally substituted with one or more R^{a} groups;
each R^{a} is independently selected from halo, ═O, —C₁-C₄ alkyl, —O—C₁-C₄ alkyl, —CN, and cyclopropyl;
each of R⁷ and R⁸ is independently hydrogen or optionally substituted C₁-C₄ alkyl; and
wherein one or more hydrogen atoms in the compound is optionally replaced with a deuterium atom.

As defined generally above and discussed throughout, R¹ is selected from aryl, carbocyclyl, heteroaryl and heterocyclyl, each of which is optionally substituted. In some embodiments, R¹ is optionally substituted aryl. In some such embodiments, R¹ is optionally substituted phenyl. In some embodiments, R¹ is phenyl optionally substituted with one or more groups selected from halogen, —SO₃H, —(CH₂)₀₋₄R^{∘}, and —(CH₂)₀₋₄OR^{∘}. In some embodiments, R¹ is phenyl optionally substituted with one or more groups selected from halogen, —SO₃H, —R^{∘}, and —OR^{∘}. In some such embodiments, R^{∘} is selected from C₁₋₆ aliphatic or a 5-6-membered saturated, partially unsaturated, or aryl ring having 0-4 heteroatoms independently selected from nitrogen, oxygen, or sulfur, wherein R^{∘} is optionally substituted with a group selected from —(CH₂)₀₋₂NH₂ and —(CH₂)₀₋₂R^{∘}, wherein R^{∘} is a 5-6-membered saturated, partially unsaturated, or aryl ring having 0-4 heteroatoms independently selected from nitrogen, oxygen, or sulfur. In some embodiments, R^{∘} is selected from C₁₋₃ aliphatic or a 5-6-membered saturated ring having 1-2 heteroatoms independently selected from nitrogen and oxygen, wherein R^{∘} is optionally substituted with a group selected from —NH₂ and —R^{•}, wherein R^{•} is a 6-membered saturated ring having 1-2 heteroatoms independently selected from nitrogen and oxygen. In some embodiments, R^{∘} is C₂₋₃ aliphatic optionally substituted with a group selected from —NH₂ and —R^{•}, wherein R^{•} is a 6-membered saturated ring having 1-2 heteroatoms independently selected from nitrogen and oxygen. In some embodiments, R^{∘} is a 5-6-membered saturated ring having 1-2 heteroatoms independently selected from nitrogen and oxygen. In some embodiments, R¹ is phenyl having 1 to 3 substituents independently selected from those set forth above.

In some embodiments, R¹ is selected from:

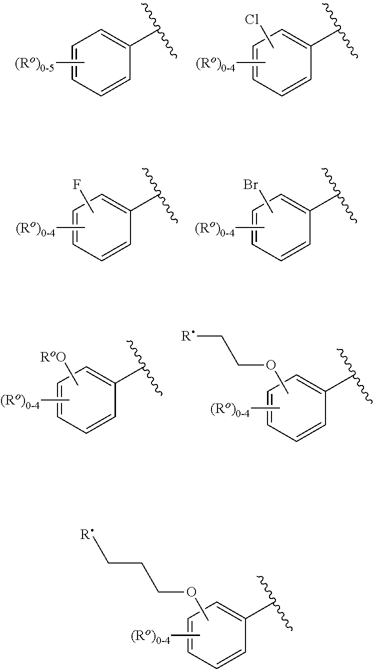

In some embodiments, R¹ is selected from:

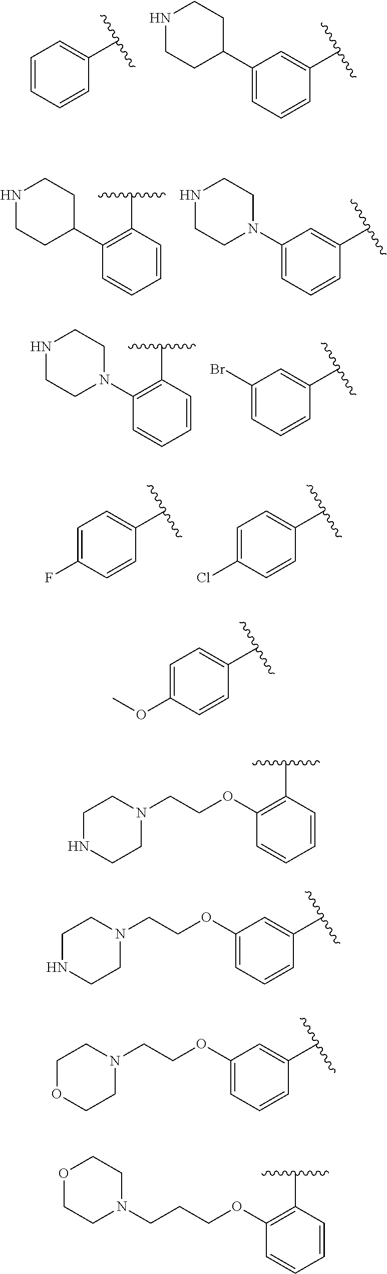

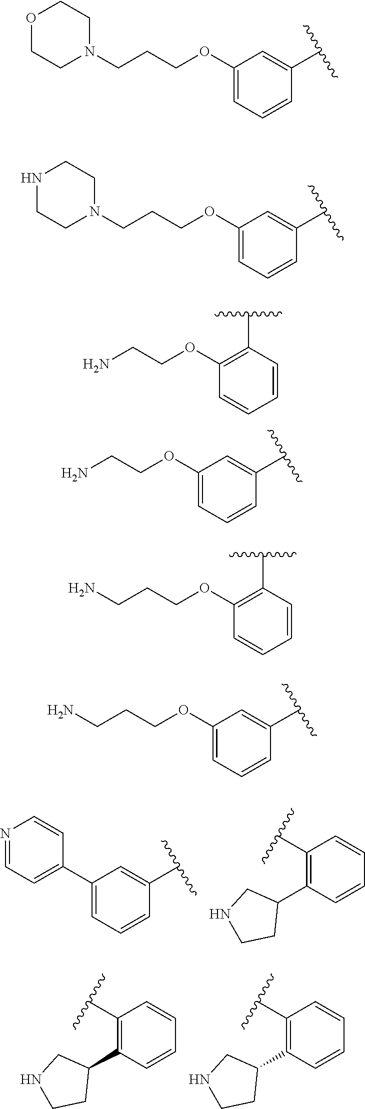

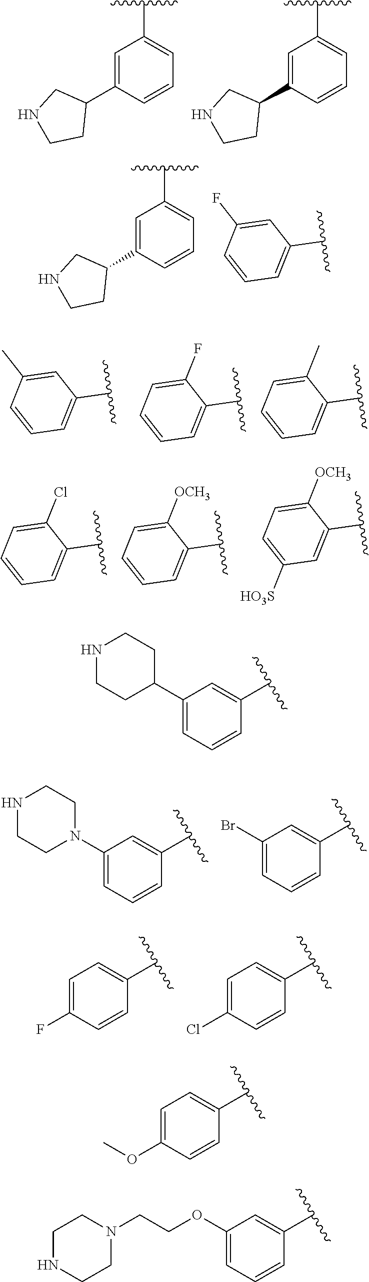

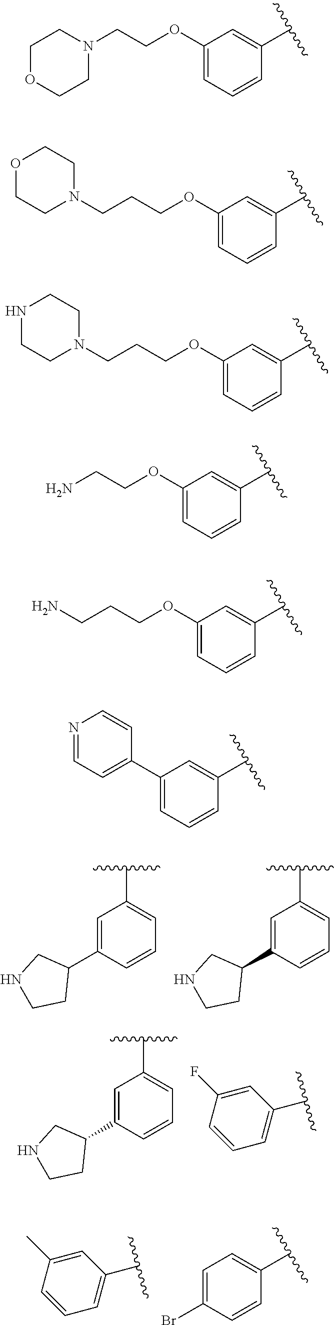

In some embodiments, R¹ is selected from:

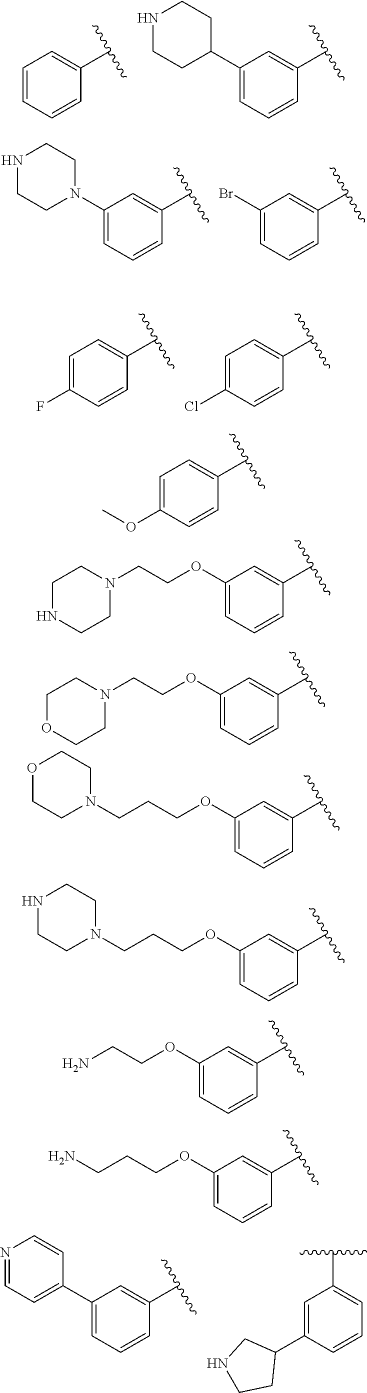

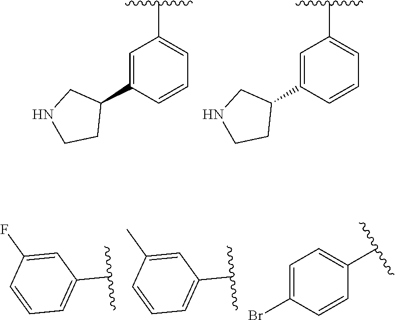

In some embodiments, R¹ is phenyl having 1-3 independently selected substituents, wherein each of the 1-3 substituents is in a meta or para position on R¹.

In some embodiments, R¹ is optionally substituted carbocyclyl. In some such embodiments, R¹ is optionally substituted 3- to 7-membered carbocyclyl. In some embodiments, R¹ is optionally substituted 3-membered carbocyclyl. In some embodiments, R¹ is optionally substituted 4-membered carbocyclyl. In some embodiments, R¹ is optionally substituted 5-membered carbocyclyl. In some embodiments, R¹ is optionally substituted 6-membered carbocyclyl. In some embodiments, R¹ is optionally substituted 7-membered carbocyclyl. In some embodiments, R¹ is optionally substituted 3- to 4-membered carbocyclyl. In some embodiments, R¹ is optionally substituted 5- to 6-membered carbocyclyl.

In some embodiments, R¹ is optionally substituted saturated 3- to 7-membered carbocyclyl. In some embodiments, R¹ is optionally substituted saturated 3- to 5-membered carbocyclyl. In some embodiments, R¹ is optionally substituted saturated 5- to 6-membered carbocyclyl.

In some embodiments, R¹ is optionally substituted cyclohexyl. In some embodiments, R¹ is cyclohexyl.

In some embodiments, R¹ is

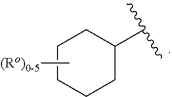

In some embodiments, R¹ is

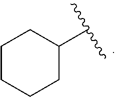 [SMILES: *C1CCCCC1]

In some embodiments, R¹ is optionally substituted partially unsaturated 5- to 7-membered carbocyclyl.

In some embodiments, R¹ is optionally substituted heteroaryl. In some embodiments, R¹ is optionally substituted heteroaryl. In some embodiments, R¹ is optionally substituted 5- and 6-membered heteroaryl having 1-4 heteroatoms independently selected from nitrogen, oxygen, and sulfur.

In some embodiments, R¹ is optionally substituted 5-membered heteroaryl having 1-4 heteroatoms independently selected from nitrogen, oxygen, and sulfur. In some embodiments, R¹ is optionally substituted thiophenyl.

In some embodiments, R¹ is

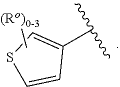

In some embodiments, R¹ is

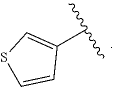 [SMILES: *c1ccsc1]

In some embodiments, R¹ is optionally substituted 5-membered heteroaryl having 1 nitrogen atom. In some embodiments, R¹ is optionally substituted pyrrolyl.

In some embodiments, R¹ is selected from

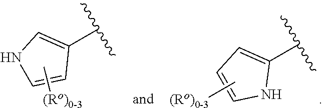

In some embodiments, R¹ is selected from

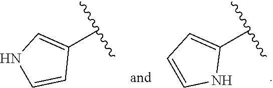 [SMILES: *c1cc[nH]c1 ; *c1ccc[nH]1]

In some embodiments, R¹ is optionally substituted 5-membered heteroaryl having 1-2 nitrogen atoms and/or 1 sulfur atom. In some such embodiments, R¹ is optionally substituted thiazolyl. In some embodiments, R¹ is thiazolyl.

In some embodiments, R¹ is selected from

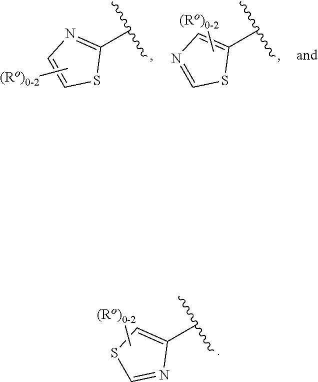

In some embodiments, R¹ is selected from

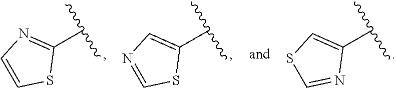 [SMILES: *c1nccs1 ; *c1cncs1 ; *c1cscn1]

In some embodiments, R¹ is optionally substituted 5-membered heteroaryl having 1-2 nitrogen atoms.

In some embodiments, R¹ is optionally substituted 5-membered heteroaryl having 2 nitrogen atoms. In some embodiments, R¹ is optionally substituted pyrazolyl or imidazolyl.

In some embodiments R¹ is

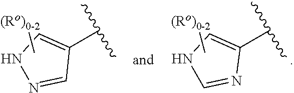

In some embodiments, R¹ is

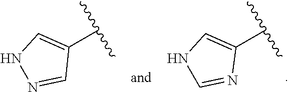 [SMILES: *c1cn[nH]c1 ; *c1c[nH]cn1]

In some embodiments, R¹ is optionally substituted 6-membered heteroaryl having 1-3 nitrogen atoms. In some embodiments, R¹ is optionally substituted 6-membered heteroaryl having 1-2 nitrogen atoms. In some embodiments, R¹ is optionally substituted pyrimidinyl. In some embodiments, R¹ is pyrimidinyl.

In some embodiments, R¹ is

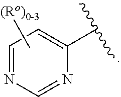

In some embodiments, R¹ is

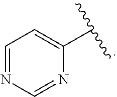 [SMILES: *c1ccncn1]

In some embodiments, R¹ is optionally substituted pyridazinyl. In some embodiments, R¹ is pyridazinyl.

In some embodiments, R¹ is

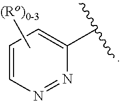

In some embodiments, R¹ is

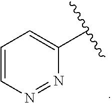 [SMILES: *c1cccnn1]

In some embodiments, R¹ is optionally substituted pyrazinyl. In some embodiments, R¹ is pyrazinyl.

In some embodiments, R¹ is

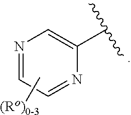

In some embodiments, R¹ is

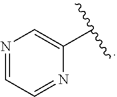 [SMILES: *c1cnccn1]

In some embodiments, R¹ is optionally substituted pyridinyl. In some embodiments, R¹ is pyridinyl. In some embodiments, R¹ is pyridinyl optionally substituted with one or more groups selected from —(CH₂)₀₋₄R^{∘} and —(CH₂)₀₋₄OR^{∘}. In some embodiments, R¹ is pyridinyl optionally substituted with one or more groups selected from —R^{∘} and —OR^{∘}. In some such embodiments, R^{∘} is selected from C₁₋₆ aliphatic or a 5-6-membered saturated, partially unsaturated, or aryl ring having 0-4 heteroatoms independently selected from nitrogen, oxygen, or sulfur, wherein R^{∘} is optionally substituted with a group selected from —(CH₂)₀₋₂NH₂ and —(CH₂)₀₋₂R^{•}. In some such embodiments, R^{•} is a 5-6-membered saturated, partially unsaturated, or aryl ring having 0-4 heteroatoms independently selected from nitrogen, oxygen, or sulfur. In some embodiments, R^{∘} is selected from C₁₋₃ aliphatic or a 5-6-membered saturated ring having 1-2 heteroatoms independently selected from nitrogen and oxygen, wherein R^{∘} is optionally substituted with a group selected from —NH₂ and —R^{•}, wherein R^{•} is a 6-membered saturated ring having 1-2 heteroatoms independently selected from nitrogen and oxygen. In some embodiments, R^{∘} is C₂-3 aliphatic, wherein R^{∘} is optionally substituted with a group selected from —NH₂ and —R^{•}, wherein R^{•} is a 6-membered saturated ring having 1-2 heteroatoms independently selected from nitrogen and oxygen. In some embodiments, R^{∘} is a 5-6-membered saturated ring having 1-2 heteroatoms independently selected from nitrogen and oxygen. In some embodiments, R¹ is pyridin-2-yl having 1 to 3 substituents independently selected from those set forth above.

In some embodiments, R¹ is selected from:

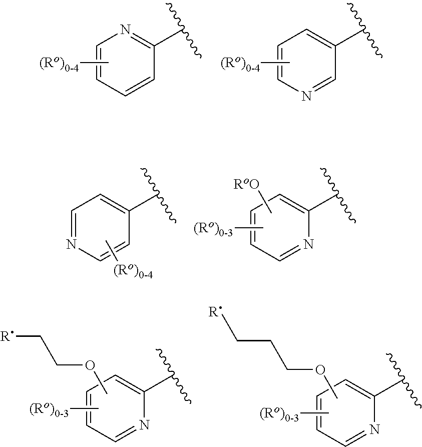

In some embodiments, R¹ is selected from:

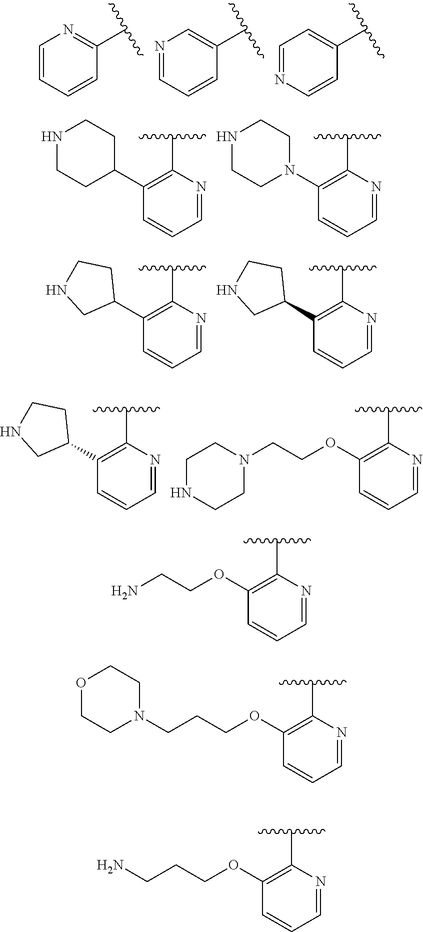

In some embodiments, R¹ is pyridin-2-yl having 1-3 independently selected substituents, wherein each of the 1-3 substituents is in a meta or para position on R¹ (relative to the point of attachment of R¹ to the rest of the molecule).

In some embodiments, R¹ is an optionally substituted saturated or partially unsaturated 6-membered heterocyclyl ring having 1-3 heteroatoms independently selected from nitrogen, oxygen, and sulfur. In some embodiments, R¹ is an optionally substituted partially unsaturated 6-membered heterocyclyl ring having 1-3 heteroatoms independently selected from nitrogen, oxygen, and sulfur. In some embodiments, R¹ is optionally substituted oxo-dihydropyridinyl. In some embodiments, R¹ is oxo-dihydropyridinyl optionally substituted with —R^{†}. In some such embodiments, —R^{†} is C₁₋₆ aliphatic. In some embodiments, —R^{†} is C₁₋₃ aliphatic. In some such embodiments, —R^{†} is methyl.

In some embodiments, R¹ is selected from

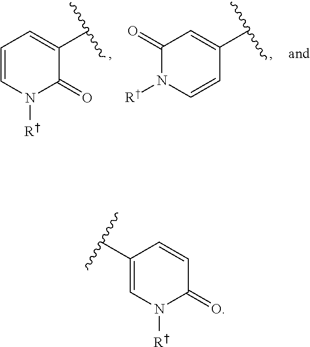

In some embodiments, R¹ is selected from

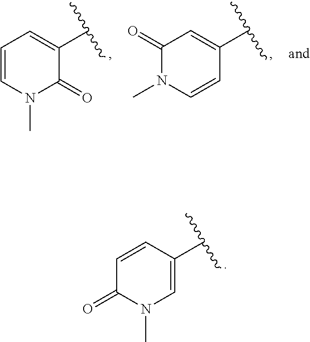

In some embodiments, R¹ is an optionally substituted heterocyclyl. In some embodiments, R¹ is an optionally substituted 5- to 7-membered monocyclic or 7- to 10-membered bicyclic heterocyclyl.

In some embodiments, R¹ is an optionally substituted 5- to 7-membered monocyclic or 7- to 10-membered bicyclic heterocyclyl having 1-4 heteroatoms independently selected from nitrogen, oxygen, and sulfur.

In some embodiments, R¹ is an optionally substituted 5- to 7-membered monocyclic saturated heterocyclyl.

In some embodiments, R¹ is an optionally substituted 6-membered monocyclic saturated heterocyclyl. In some embodiments, R¹ is an optionally substituted 6-membered monocyclic saturated heterocyclyl having 1-2 heteroatoms independently selected from nitrogen, oxygen, and sulfur. In some embodiments, R¹ is optionally substituted piperidinyl. In some embodiments, R¹ is piperidinyl optionally substituted with a group selected from —C(O)R^{†}, —S(O)₂R^{†} and —C(O)OR^{\}, wherein —R^{†} is C₁₋₆ aliphatic. In some embodiments, R¹ is piperidinyl optionally substituted with a group selected from —C(O)R^{†}, —S(O)₂R^{†} and —C(O)OR^{†}, wherein —R^{†} is C₁₋₃ aliphatic. In some embodiments, R¹ is piperidinyl optionally substituted with a group selected from —C(O)R^{†}, —S(O)₂R^{†} and —C(O)OR^{†}, wherein —R^{†} is methyl.

In some embodiments, R¹ is selected from

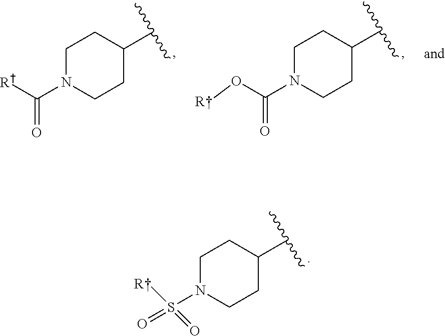

In some embodiments, R¹ is selected from

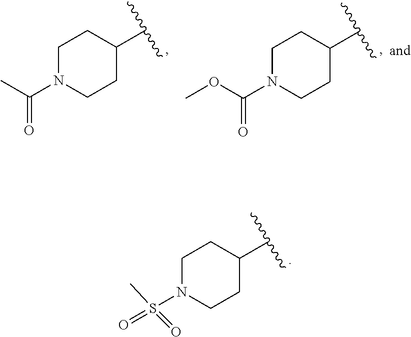

In some embodiments, R¹ is optionally substituted tetrahydropyranyl. In some embodiments, R¹ is tetrahydropyranyl.

In some embodiments, R¹ is

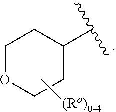

In some embodiments, R¹ is

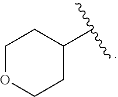 [SMILES: *C1CCOCC1]

In some embodiments, R¹ is optionally substituted 5- to 7-membered monocyclic partially saturated heterocyclyl.

In some embodiments, R¹ is an optionally substituted group selected from aryl and carbocyclyl. In some embodiments, R¹ is an optionally substituted group selected from aryl and heteroaryl. In some embodiments, R¹ is an optionally substituted group selected from heteroaryl and heterocyclyl. In some embodiments, R¹ is an optionally substituted group selected from heterocyclyl and carbocyclyl.

As defined above and generally throughout, R⁷ is selected from hydrogen and optionally substituted C₁-C₄ alkyl. In some embodiments, R⁷ is hydrogen. In some embodiments, R⁷ is optionally substituted C₁-C₄ alkyl.

In some embodiments, R⁷ is optionally substituted C₁-C₄ alkyl. In some embodiments, R⁷ is selected from methyl, ethyl, propyl, isopropyl, n-butyl, and t-butyl. In some embodiments, R⁷ is methyl. In some embodiments, R⁷ is ethyl. In some embodiments, R⁷ is propyl. In some embodiments, R⁷ is isopropyl. In some embodiments, R⁷ is n-butyl. In some embodiments, R⁷ is t-butyl.

As defined above and generally throughout, R⁸ is selected from hydrogen and optionally substituted C₁-C₄ alkyl. In some embodiments, R⁸ is hydrogen. In some embodiments, R⁸ is optionally substituted C₁-C₄ alkyl. In some embodiments, R⁸ is selected from methyl, ethyl, propyl, isopropyl, n-butyl, and t-butyl. In some embodiments, R⁸ is methyl. In some embodiments, R⁸ is ethyl. In some embodiments, R⁸ is propyl. In some embodiments, R⁸ is isopropyl. In some embodiments, R⁸ is n-butyl. In some embodiments, R⁸ is t-butyl.

As defined above and generally throughout, R^{2a} is selected from hydrogen, halogen, —C₁-C₄ alkyl, —O—C₁-C₄ alkyl, —C₂-C₄ alkynyl, —CN, cyclopropyl, and phenyl.

In some embodiments, R^{2a} is hydrogen. In some embodiments, R^{2a} is selected from halo, —C₁-C₄ alkyl, —O—C₁-C₄ alkyl, —C₂-C₄ alkynyl, —CN, cyclopropyl, and phenyl.

In some embodiments, R^{2a} is halogen. In some embodiments, R^{2a} is selected from fluoro, chloro, bromo, and iodo. In some embodiments, R^{2a} is selected from fluoro and chloro. In some embodiments, R^{2a} is selected from bromo and iodo. In some embodiments, R^{2a} is fluoro. In some embodiments, R^{2a} is chloro.

In some embodiments, R^{2a} is —C₁-C₄ alkyl. In some embodiments, R^{2a} is selected from methyl and ethyl. In some embodiments, R^{2a} is methyl. In some embodiments, R^{2a} is ethyl.

In some embodiments, R^{2a} is —C₂-C₄ alkynyl. In some embodiments, R^{2a} is ethynyl.

As defined above and generally throughout, R^{2b} is selected from hydrogen, halogen, —C₁-C₄ alkyl, —O—C₁-C₄ alkyl, —C₂-C₄ alkynyl, —CN, and cyclopropyl. In some embodiments, R^{2b} is hydrogen. In some embodiments, R^{2b} is selected from halogen, —C₁-C₄ alkyl, —O—C₁-C₄ alkyl, —C₂-C₄ alkynyl, —CN, and cyclopropyl.

In some embodiments, R^{2b} is halogen. In some embodiments, R^{2b} is selected from fluoro, chloro, bromo, and iodo. In some embodiments, R^{2b} is selected from fluoro and chloro. In some embodiments, R^{2b} is selected from bromo and iodo. In some embodiments, R^{2b} is fluoro. In some embodiments, R^{2b} is chloro.

In some embodiments, R^{2b} is —C₁-C₄ alkyl. In some embodiments, R^{2b} is selected from methyl and ethyl. In some embodiments, R^{2b} is methyl. In some embodiments, R^{2b} is ethyl.

In some embodiments, R^{2b} is —O—C₁-C₄ alkyl. In some embodiments, R^{2b} is —O—C₁-C₄ alkyl, wherein C₁-C₄ alkyl is selected from methyl, ethyl, propyl, isopropyl, n-butyl, and t-butyl. In some embodiments, R^{2b} is —O—C₁-C₄ alkyl, wherein C₁-C₄ alkyl is methyl. In some embodiments, R^{2b} is —O—C₁-C₄ alkyl, wherein C₁-C₄ alkyl is ethyl. In some embodiments, R^{2b} is —O—C₁-C₄ alkyl, wherein C₁-C₄ alkyl is propyl. In some embodiments, R^{2b} is —O—C₁-C₄ alkyl, wherein C₁-C₄ alkyl is isopropyl. In some embodiments, R^{2b} is —O—C₁-C₄ alkyl, wherein C₁-C₄ alkyl is n-butyl. In some embodiments, R^{2b} is —O—C₁-C₄ alkyl, wherein C₁-C₄ alkyl is t-butyl.

In some embodiments, R^{2b} is —C₂-C₄ alkynyl. In some embodiments, R^{2b} is ethynyl.

In some embodiments, R^{2b} is —CN.

In some embodiments, R^{2b} is cyclopropyl.

As defined above and generally throughout, R^{3a} is independently selected from hydrogen, halogen, —C₁-C₄ alkyl, —O—C₁-C₄ alkyl, —C₂-C₄ alkynyl, —CN, cyclopropyl, and phenyl.

In some embodiments, R^{3a} is selected from hydrogen, halogen, —C₁-C₄ alkyl, and —CN.

In some embodiments, R^{3a} is hydrogen. In some embodiments, R^{3a} is selected from halogen, —C₁-C₄ alkyl, —O—C₁-C₄ alkyl, —C₂-C₄ alkynyl, —CN, cyclopropyl, and phenyl.

In some embodiments, R^{3a} is halogen. In some embodiments, R^{3a} is selected from fluoro, chloro, bromo, and iodo. In some embodiments, R^{3a} is selected from fluoro and chloro. In some embodiments, R^{3a} is selected from bromo and iodo. In some embodiments, R^{3a} is fluoro. In some embodiments, R^{3a} is chloro.

In some embodiments, R^{3a} is —C₁-C₄ alkyl. In some embodiments, R^{3a} is selected from methyl, ethyl, isopropyl, t-butyl, and n-butyl. In some embodiments, R^{3a} is methyl. In some embodiments, R^{3a} is ethyl. In some embodiments, R^{3a} isopropyl. In some embodiments, R^{3a} is t-butyl. In some embodiments, R^{3a} is n-butyl.

In some embodiments, R^{3a} is —O—C₁-C₄ alkyl. In some embodiments, R^{3a} is —O—C₁-C₄ alkyl, wherein C₁-C₄ alkyl is selected from methyl, ethyl, propyl, isopropyl, n-butyl, and t-butyl. In some embodiments, R^{3a} is —O—C₁-C₄ alkyl, wherein C₁-C₄ alkyl is methyl. In some embodiments, R^{3a} is —O—C₁-C₄ alkyl, wherein C₁-C₄ alkyl is ethyl. In some embodiments, R^{3a} is —O—C₁-C₄ alkyl, wherein C₁-C₄ alkyl is propyl. In some embodiments, R^{3a} is —O—C₁-C₄ alkyl, wherein C₁-C₄ alkyl is isopropyl. In some embodiments, R^{3a} is —O—C₁-C₄ alkyl, wherein C₁-C₄ alkyl is n-butyl. In some embodiments, R^{3a} is —O—C₁-C₄ alkyl, wherein C₁-C₄ alkyl is t-butyl.

In some embodiments, R^{3a} is —C₂-C₄ alkynyl. In some embodiments, R^{3a} is ethynyl.

In some embodiments, R^{3a} is —CN.

In some embodiments, R^{3a} is cyclopropyl.

In some embodiments, R^{3a} is phenyl.

As defined above and generally throughout, R^{3b} is independently selected from hydrogen, halogen, —C₁-C₄ alkyl, —O—C₁-C₄ alkyl, —CN, cyclopropyl, and phenyl.

In some embodiments, R^{3b} is selected from hydrogen, halogen, —C₁-C₄ alkyl, and —CN.

In some embodiments, R^{3b} is hydrogen. In some embodiments, R^{3b} is selected from halogen, —C₁-C₄ alkyl, —O—C₁-C₄ alkyl, —CN, cyclopropyl, and phenyl.

In some embodiments, R^{3b} is halogen. In some embodiments, R^{3b} is selected from fluoro, chloro, bromo, and iodo. In some embodiments, R^{3b} is selected from fluoro and chloro. In some embodiments, R^{3b} is selected from bromo and iodo. In some embodiments, R^{3b} is fluoro. In some embodiments, R^{3b} is chloro.

In some embodiments, R^{3b} is —C₁-C₄ alkyl. In some embodiments, R^{3b} is selected from methyl, ethyl, isopropyl, t-butyl, and n-butyl. In some embodiments, R^{3b} is methyl. In some embodiments, R^{3b} is ethyl. In some embodiments, R^{3b} isopropyl. In some embodiments, R^{3b} is t-butyl. In some embodiments, R^{3b} is n-butyl.

In some embodiments, R^{3b} is —O—C₁-C₄ alkyl. In some embodiments, R^{3b} is —O—C₁-C₄ alkyl, wherein C₁-C₄ alkyl is selected from methyl, ethyl, propyl, isopropyl, n-butyl, and t-butyl. In some embodiments, R^{3b} is —O—C₁-C₄ alkyl, wherein C₁-C₄ alkyl is methyl. In some embodiments, R^{3b} is —O—C₁-C₄ alkyl, wherein C₁-C₄ alkyl is ethyl. In some embodiments, R^{3b} is —O—C₁-C₄ alkyl, wherein C₁-C₄ alkyl is propyl. In some embodiments, R^{3b} is —O—C₁-C₄ alkyl, wherein C₁-C₄ alkyl is isopropyl. In some embodiments, R^{3b} is —O—C₁-C₄ alkyl, wherein C₁-C₄ alkyl is n-butyl. In some embodiments, R^{3b} is —O—C₁-C₄ alkyl, wherein C₁-C₄ alkyl is t-butyl.

In some embodiments, R^{3b} is —CN.

In some embodiments, R^{3b} is cyclopropyl.

In some embodiments, R^{3b} is phenyl.

As defined above and generally throughout, R^{4a} is independently selected from hydrogen, halogen, —C₁-C₄ alkyl, —O—C₁-C₄ alkyl, —C₂-C₄ alkynyl, —CN, cyclopropyl, and phenyl.

In some embodiments, R^{4a} is selected from hydrogen, halogen, —C₁-C₄ alkyl, and —CN.

In some embodiments, R^{4a} is hydrogen. In some embodiments, R^{4a} is selected from halogen, —C₁-C₄ alkyl, —O—C₁-C₄ alkyl, —C₂-C₄ alkynyl, —CN, cyclopropyl, and phenyl.

In some embodiments, R^{4a} is halogen. In some embodiments, R^{4a} is selected from fluoro, chloro, bromo, and iodo. In some embodiments, R^{4a} is selected from fluoro and chloro. In some embodiments, R^{4a} is selected from bromo and iodo. In some embodiments, R^{4a} is fluoro. In some embodiments, R^{4a} is chloro.

In some embodiments, R^{4a} is —C₁-C₄ alkyl. In some embodiments, R^{4a} is selected from methyl, ethyl, isopropyl, t-butyl, and n-butyl. In some embodiments, R^{4a} is methyl. In some embodiments, R^{4a} is ethyl. In some embodiments, R^{4a} isopropyl. In some embodiments, R^{4a} is t-butyl. In some embodiments, R^{4a} is n-butyl.

In some embodiments, R^{4a} is —O—C₁-C₄ alkyl. In some embodiments, R^{4a} is —O—C₁-C₄ alkyl, wherein C₁-C₄ alkyl is selected from methyl, ethyl, propyl, isopropyl, n-butyl, and t-butyl. In some embodiments, R^{4a} is —O—C₁-C₄ alkyl, wherein C₁-C₄ alkyl is methyl. In some embodiments, R^{4a} is —O—C₁-C₄ alkyl, wherein C₁-C₄ alkyl is ethyl. In some embodiments, R^{4a} is —O—C₁-C₄ alkyl, wherein C₁-C₄ alkyl is propyl. In some embodiments, R^{4a} is —O—C₁-C₄ alkyl, wherein C₁-C₄ alkyl is isopropyl. In some embodiments, R^{4a} is —O—C₁-C₄ alkyl, wherein C₁-C₄ alkyl is n-butyl. In some embodiments, R^{4a} is —O—C₁-C₄ alkyl, wherein C₁-C₄ alkyl is t-butyl.

In some embodiments, R^{4a} is —C₂-C₄ alkynyl. In some embodiments, R^{4a} is ethynyl.

In some embodiments, R^{4a} is —CN.

In some embodiments, R^{4a} is cyclopropyl.

In some embodiments, R^{4a} is phenyl.

As defined above and generally throughout, R^{4b} is independently selected from hydrogen, halogen, —C₁-C₄ alkyl, —O—C₁-C₄ alkyl, —CN, cyclopropyl, and phenyl.

In some embodiments, R^{4b} is selected from hydrogen, halogen, —C₁-C₄ alkyl, and —CN.

In some embodiments, R^{4b} is hydrogen. In some embodiments, R^{4b} is selected from halogen, —C₁-C₄ alkyl, —O—C₁-C₄ alkyl, —CN, cyclopropyl, and phenyl.

In some embodiments, R^{4b} is halogen. In some embodiments, R^{4b} is selected from fluoro, chloro, bromo, and iodo. In some embodiments, R^{4b} is selected from fluoro and chloro. In some embodiments, R^{4b} is selected from bromo and iodo. In some embodiments, R^{4b} is fluoro. In some embodiments, R^{4b} is chloro.

In some embodiments, R^{4b} is —C₁-C₄ alkyl. In some embodiments, R^{4b} is selected from methyl, ethyl, isopropyl, t-butyl, and n-butyl. In some embodiments, R^{4b} is methyl. In some embodiments, R^{4b} is ethyl. In some embodiments, R^{4b} isopropyl. In some embodiments, R^{4b} is t-butyl. In some embodiments, R^{4b} is n-butyl.

In some embodiments, R^{4b} is —O—C₁-C₄ alkyl. In some embodiments, R^{4b} is —O—C₁-C₄ alkyl, wherein C₁-C₄ alkyl is selected from methyl, ethyl, propyl, isopropyl, n-butyl, and t-butyl. In some embodiments, R^{4b} is —O—C₁-C₄ alkyl, wherein C₁-C₄ alkyl is methyl. In some embodiments, R^{4b} is —O—C₁-C₄ alkyl, wherein C₁-C₄ alkyl is ethyl. In some embodiments, R^{4b} is —O—C₁-C₄ alkyl, wherein C₁-C₄ alkyl is propyl. In some embodiments, R^{4b} is —O—C₁-C₄ alkyl, wherein C₁-C₄ alkyl is isopropyl. In some embodiments, R^{4b} is —O—C₁-C₄ alkyl, wherein C₁-C₄ alkyl is n-butyl. In some embodiments, R^{4b} is —O—C₁-C₄ alkyl, wherein C₁-C₄ alkyl is t-butyl.

In some embodiments, R^{4b} is —CN.

In some embodiments, R^{4b} is cyclopropyl.

In some embodiments, R^{4b} is phenyl.

As defined above and generally throughout, R^{6a} is independently selected from hydrogen, halogen, —C₁-C₄ alkyl, —O—C₁-C₄ alkyl, —C₂-C₄ alkynyl, —CN, cyclopropyl, and phenyl.

In some embodiments, R^{6a} is selected from hydrogen, halogen, —C₁-C₄ alkyl, and —CN.

In some embodiments, R^{6a} is hydrogen. In some embodiments, R^{6a} is selected from halogen, —C₁-C₄ alkyl, —O—C₁-C₄ alkyl, —C₂-C₄ alkynyl, —CN, cyclopropyl, and phenyl.

In some embodiments, R^{6a} is halogen. In some embodiments, R^{6a} is selected from fluoro, chloro, bromo, and iodo. In some embodiments, R^{6a} is selected from fluoro and chloro. In some embodiments, R^{6a} is selected from bromo and iodo. In some embodiments, R^{6a} is fluoro. In some embodiments, R^{6a} is chloro.

In some embodiments, R^{6a} is —C₁-C₄ alkyl. In some embodiments, R^{6a} is selected from methyl, ethyl, isopropyl, t-butyl, and n-butyl. In some embodiments, R^{6a} is methyl. In some embodiments, R^{6a} is ethyl. In some embodiments, R^{6a} isopropyl. In some embodiments, R^{6a} is t-butyl. In some embodiments, R^{6a} is n-butyl.

In some embodiments, R^{6a} is —O—C₁-C₄ alkyl. In some embodiments, R^{6a} is —O—C₁-C₄ alkyl, wherein C₁-C₄ alkyl is selected from methyl, ethyl, propyl, isopropyl, n-butyl, and t-butyl. In some embodiments, R^{6a} is —O—C₁-C₄ alkyl, wherein C₁-C₄ alkyl is methyl. In some embodiments, R^{6a} is —O—C₁-C₄ alkyl, wherein C₁-C₄ alkyl is ethyl. In some embodiments, R^{6a} is —O—C₁-C₄ alkyl, wherein C₁-C₄ alkyl is propyl. In some embodiments, R^{6a} is —O—C₁-C₄ alkyl, wherein C₁-C₄ alkyl is isopropyl. In some embodiments, R^{6a} is —O—C₁-C₄ alkyl, wherein C₁-C₄ alkyl is n-butyl. In some embodiments, R^{6a} is —O—C₁-C₄ alkyl, wherein C₁-C₄ alkyl is t-butyl.

In some embodiments, R^{6a} is —C₂-C₄ alkynyl. In some embodiments, R^{6a} is ethynyl.

In some embodiments, R^{6a} is —CN.

In some embodiments, R^{6a} is cyclopropyl.

In some embodiments, R^{6a} is phenyl.

As defined above and generally throughout, Ring A is a 4-7 membered heterocyclic or heteroaromatic ring comprising a ring nitrogen atom and optionally substituted with one or more R^{a} groups.

In some embodiments, Ring A is a 5-6 membered heterocyclic or heteroaromatic ring comprising a ring nitrogen atom and optionally substituted with one or more R^{a} group.

In some embodiments, Ring A is a 5-membered heterocyclic ring comprising a ring nitrogen atom and optionally substituted with one R^{a} group. In some such embodiments, Ring A is selected from pyrazolyl, pyrrolidinyl, piperidinyl, and tetrahydropyridinyl, wherein each is optionally substituted with one R^{a} group.

In some embodiments, Ring A is selected from

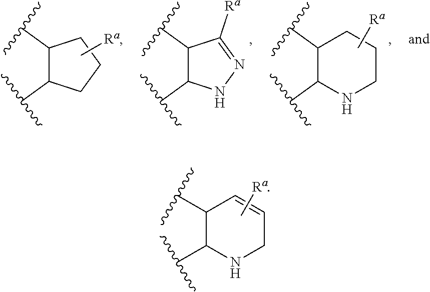

In some embodiments, Ring A is selected from

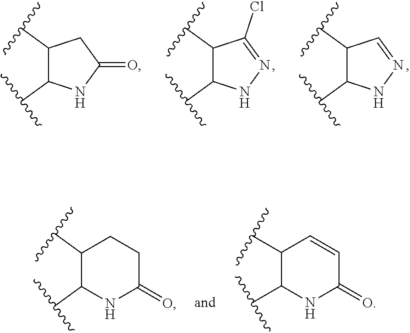 [SMILES: *C1CC(=O)NC1* ; *C1NN=C(Cl)C1* ; *C1C=NNC1* ; *C1CCC(=O)NC1* ; *C1C=CC(=O)NC1*]

As defined above and generally throughout, R^{a} is selected from halogen, ═O, —C₁-C₄ alkyl, —O—C₁-C₄ alkyl, —CN, and cyclopropyl.

In some embodiments, R^{a} is halogen. In some embodiments, R^{a} is selected from fluoro, chloro, bromo, and iodo. In some embodiments, R^{a} is selected from fluoro and chloro. In some embodiments, R^{a} is selected from bromo and iodo. In some embodiments, R^{a} is fluoro. In some embodiments, R^{a} is chloro.

In some embodiments, R^{a} is ═O.

In some embodiments, R^{a} is —C₁-C₄ alkyl. In some embodiments, R^{a} is selected from methyl, ethyl, isopropyl, t-butyl, and n-butyl. In some embodiments, R^{a} is methyl. In some embodiments, R^{a} is ethyl. In some embodiments, R^{a} isopropyl. In some embodiments, R^{a} is t-butyl. In some embodiments, R^{a} is n-butyl.

In some embodiments, R^{a} is —O—C₁-C₄ alkyl. In some embodiments, R^{a} is —O—C₁-C₄ alkyl, wherein C₁-C₄ alkyl is selected from methyl, ethyl, propyl, isopropyl, n-butyl, and t-butyl. In some embodiments, R^{a} is —O—C₁-C₄ alkyl, wherein C₁-C₄ alkyl is methyl. In some embodiments, R^{a} is —O—C₁-C₄ alkyl, wherein C₁-C₄ alkyl is ethyl. In some embodiments, R^{a} is —O—C₁-C₄ alkyl, wherein C₁-C₄ alkyl is propyl. In some embodiments, R^{a} is —O—C₁-C₄ alkyl, wherein C₁-C₄ alkyl is isopropyl. In some embodiments, R^{a} is —O—C₁-C₄ alkyl, wherein C₁-C₄ alkyl is n-butyl. In some embodiments, R^{a} is —O—C₁-C₄ alkyl, wherein C₁-C₄ alkyl is t-butyl.

In some embodiments, R^{a} is —CN.

In some embodiments, R^{a} is cyclopropyl.

In some embodiments, the present disclosure provides a compound of any of formulae I-a-i, I-a-ii, I-a-iii, I-a-iv, I-a-v, I-a-vi, I-a-vii, I-a-viii, I-a-ix, I-b-i, or I-b-ii:

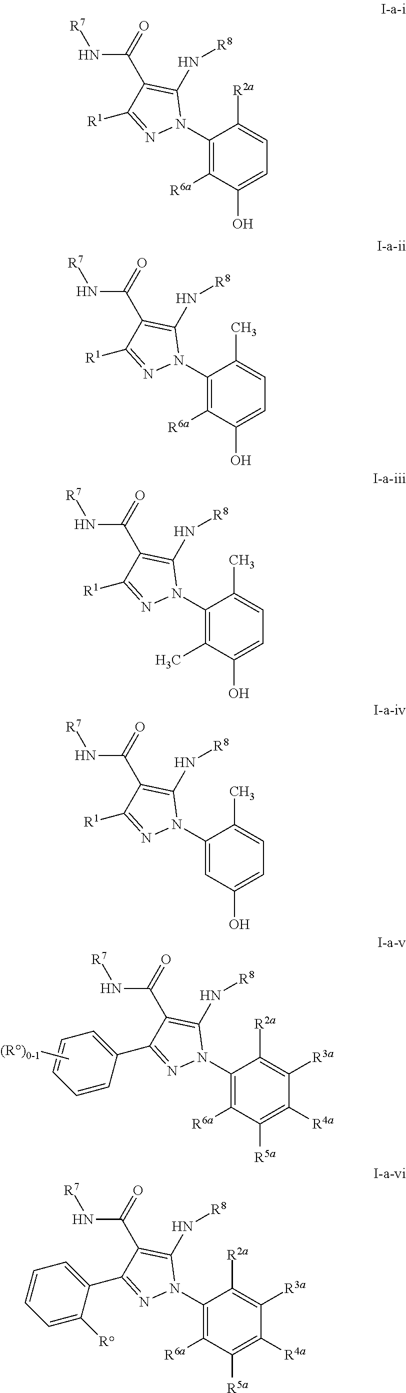

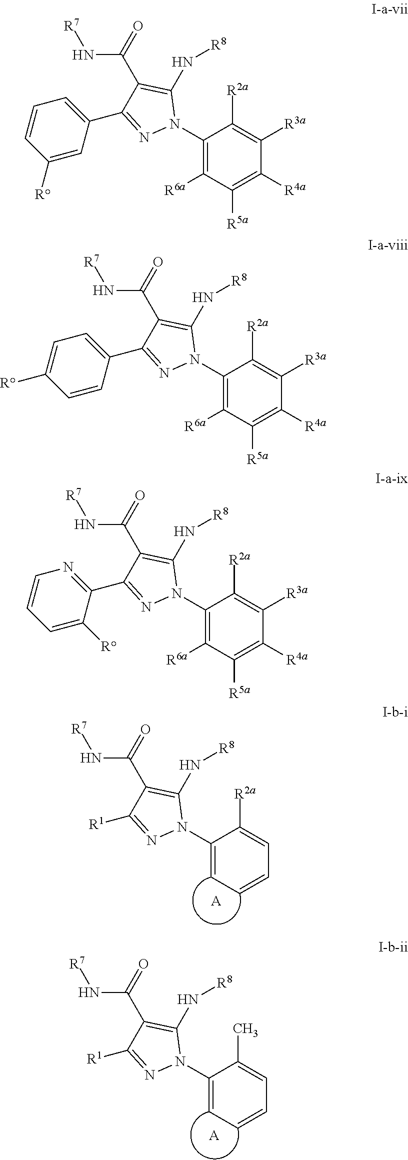

or a pharmaceutically acceptable salt thereof.

In some embodiments of any of formulae I-a-i, I-a-ii, I-a-iii, I-a-iv, I-a-v, I-a-vi, I-a-vii, I-a-viii, I-a-ix, I-b-i, or I-b-ii, R⁷ is hydrogen. In some embodiments of any of formulae I-a-i, I-a-ii, I-a-iii, I-a-iv, I-a-v, I-a-vi, I-a-vii, I-a-viii, I-a-ix, I-b-i, or I-b-ii, R⁸ is hydrogen. In some embodiments of any of formulae I-a-i, I-a-ii, I-a-iii, I-a-iv, I-a-v, I-a-vi, I-a-vii, I-a-viii, I-a-ix, I-b-i, or I-b-ii, each of R⁷ and R⁸ is hydrogen.

In some embodiments, the present disclosure provides a compound of formula II:

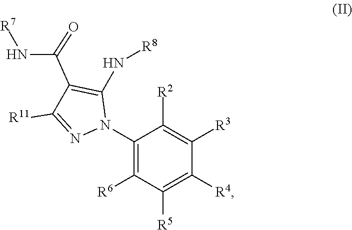

or a solvate, enantiomer, tautomer, or diastereomer thereof, or a pharmaceutically acceptable salt of any of the foregoing wherein:
R¹¹ is

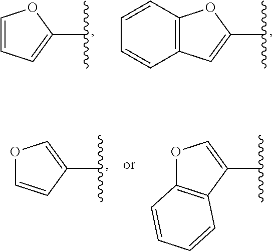

each of which is optionally substituted;
each of R², R³, R⁴ and R⁶ is independently hydrogen, halo, —C₁-C₄ alkyl, —O—C₁-C₄ alkyl, —C₂-C₄ alkynyl, —CN, cyclopropyl, or phenyl; and
R⁵ is —OH; or
R² is halo, —C₁-C₄ alkyl, —O—C₁-C₄ alkyl, —C₂-C₄ alkynyl, —CN, or cyclopropyl;
each of R³ and R⁴ is independently hydrogen, halo, —C₁-C₄ alkyl, —O—C₁-C₄ alkyl, —CN, cyclopropyl, or phenyl; and
R⁵ and R⁶ are taken together with carbon atoms to which they are bound to form Ring A;
Ring A is a 4-7 membered heterocyclic or heteroaromatic ring comprising a ring nitrogen atom and optionally substituted with one or more R^{a} groups;
each R^{a} is independently selected from halo, ═O, —C₁-C₄ alkyl, —O—C₁-C₄ alkyl, —CN, and cyclopropyl;
each of R⁷ and R⁸ is independently hydrogen or optionally substituted C₁-C₄ alkyl; and
wherein one or more hydrogen atoms in the compound is optionally replaced with a deuterium atom.

In some embodiments, the present disclosure provides a compound of formula IIa:

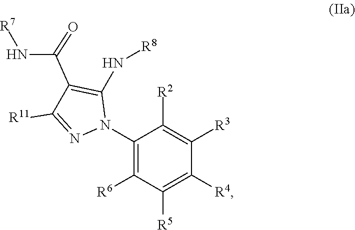

or a solvate, enantiomer, tautomer, or diastereomer thereof, or a pharmaceutically acceptable salt of any of the foregoing wherein:
R¹¹ is

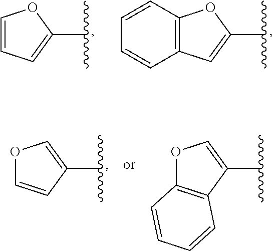

each of which is optionally substituted;
each of R², R³, R⁴ and R⁶ is independently hydrogen, halo, —C₁-C₄ alkyl, —O—C₁-C₄ alkyl, —C₂-C₄ alkynyl, —CN, cyclopropyl, or phenyl;
R⁵ is —OH;
each of R⁷ and R⁸ is independently hydrogen or optionally substituted C₁-C₄ alkyl; and
wherein one or more hydrogen atoms in the compound is optionally replaced with a deuterium atom.

In some embodiments, the present disclosure provides a compound of formula IIb:

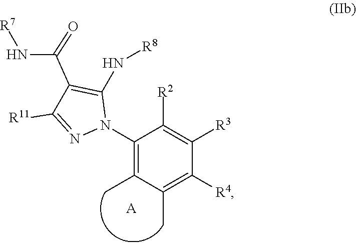

or a solvate, enantiomer, tautomer, or diastereomer thereof, or a pharmaceutically acceptable salt of any of the foregoing wherein:
R¹¹ is

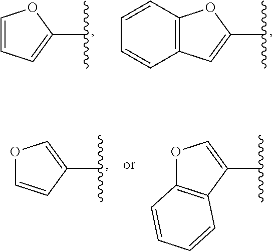

each of which is optionally substituted;
R² is halo, —C₁-C₄ alkyl, —O—C₁-C₄ alkyl, —C₂-C₄ alkynyl, —CN, or cyclopropyl;
each of R³ and R⁴ is independently hydrogen, halo, —C₁-C₄ alkyl, —O—C₁-C₄ alkyl, —CN, cyclopropyl, or phenyl; and
R⁵ and R⁶ are taken together with carbon atoms to which they are bound to form Ring A;
Ring A is a 4-7 membered heterocyclic or heteroaromatic ring comprising a ring nitrogen atom and optionally substituted with one or more R^{a} groups;
each R^{a} is independently selected from halo, ═O, —C₁-C₄ alkyl, —O—C₁-C₄ alkyl, —CN, and cyclopropyl;
each of R⁷ and R⁸ is independently hydrogen or optionally substituted C₁-C₄ alkyl; and
wherein one or more hydrogen atoms in the compound is optionally replaced with a deuterium atom.

In some embodiments of a compound of any one of formulae II, IIa, or IIb, R¹¹ is optionally substituted with one or more substituent independently selected from halo, —CN, C₁-C₄ alkyl, C₂-C₅ alkenyl, C₂-C₅ alkynyl, C₃-C₆ cycloalkyl, —O—C₁-C₄ alkyl, optionally substituted aryl, and optionally substituted heteroaryl, wherein any alkyl, alkenyl, alkynyl, aryl or heteroaryl substituent on R¹¹ is optionally further substituted with —OH.

In some specific embodiments of a compound of any one of formulae II, IIa, or IIb, R¹¹ is optionally substituted with one or more substituent independently selected from bromo, chloro, —CN, —CH₃, —CH(CH₃)₂, —CH₂═CH₂, —C(CH₃)═CH₂, —CH═CH(CH₃)₂, —CH═CH—CH₃, —C≡C—C(CH₃)₂—OH, —C≡C—CH₃, —OCH₃, cyclopropyl, phenyl, or pyridinyl.

In some embodiments of a compound of any one of formulae II, IIa, or IIb, R² is —CH₃ or —Cl.

In some embodiments of a compound of any one of formulae II, IIa, or IIb, R³ is hydrogen.

In some embodiments of a compound of any one of formulae II, IIa, or IIb, R⁴ is hydrogen or chloro.

In some embodiment of a compound of formula II, or IIa, R⁶ is —CH₃ or —Cl.

In some embodiment of a compound of formula II, or IIa, each of R² and R⁶ are simultaneously —CH₃ or —Cl.

In some embodiments of a compound of any one of formulae II, IIa, or IIb, R⁷ is hydrogen.

In some embodiments of a compound of any one of formulae II, IIa, or IIb, R⁸ is hydrogen.

In some embodiments of a compound of formula II, or IIa, the portion of the compound represented by

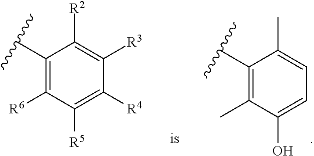

In some embodiments of a compound of formula II, or IIa, the portion of the compound represented by

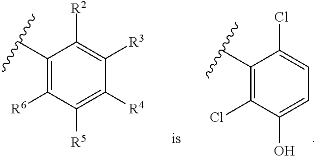

In some embodiments of a compound of formula II, or IIa, the portion of the compound represented by

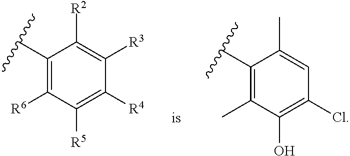

In some embodiment of a compound of formula II, or IIb, Ring A is a 5-membered heterocyclic ring comprising a ring nitrogen atom and optionally substituted with one R^{a} group. In some such embodiments, Ring A is pyrazolyl optionally substituted with one R^{a} group.

In some embodiments, Ring A is

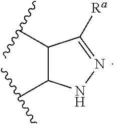

In more specific embodiments, Ring A is

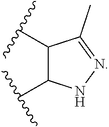 [SMILES: *C1NN=C(C)C1*]

In some embodiments, a compound of formula II may be a compound, or a pharmaceutically acceptable salt thereof, selected from Table 1A:

TABLE 1A
Exemplary Compounds of Formula II

| Compound | Structure |
|---|---|
| 72 | 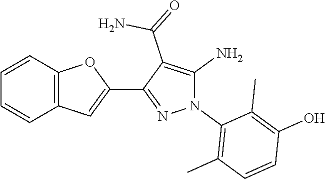 [SMILES: Cc1ccc(O)c(C)c1-n1nc(-c2cc3ccccc3o2)c(C(N)=O)c1N] |
| 73 | 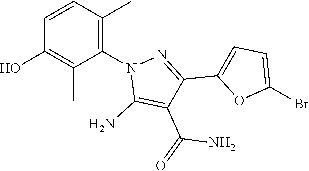 [SMILES: Cc1ccc(O)c(C)c1-n1nc(-c2ccc(Br)o2)c(C(N)=O)c1N] |
| 74 | 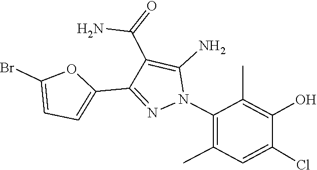 [SMILES: Cc1cc(Cl)c(O)c(C)c1-n1nc(-c2ccc(Br)o2)c(C(N)=O)c1N] |
| 75 | 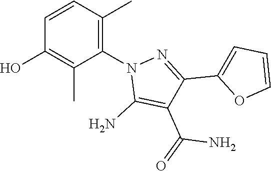 [SMILES: Cc1ccc(O)c(C)c1-n1nc(-c2ccco2)c(C(N)=O)c1N] |
| 76 | 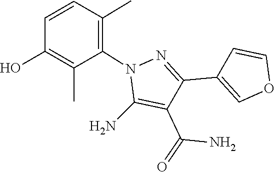 [SMILES: Cc1ccc(O)c(C)c1-n1nc(-c2ccoc2)c(C(N)=O)c1N] |
| 77 | 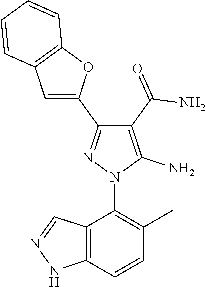 [SMILES: Cc1ccc2[nH]ncc2c1-n1nc(-c2cc3ccccc3o2)c(C(N)=O)c1N] |
| 78 | 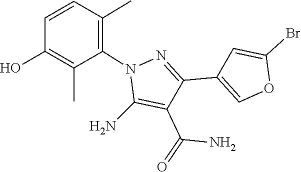 [SMILES: Cc1ccc(O)c(C)c1-n1nc(-c2coc(Br)c2)c(C(N)=O)c1N] |
| 79 | 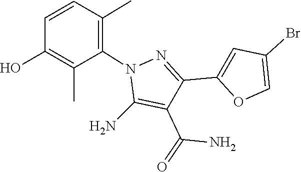 [SMILES: Cc1ccc(O)c(C)c1-n1nc(-c2cc(Br)co2)c(C(N)=O)c1N] |
| 80 | 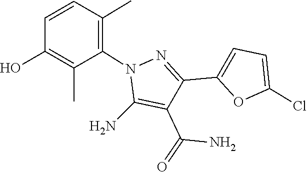 [SMILES: Cc1ccc(O)c(C)c1-n1nc(-c2ccc(Cl)o2)c(C(N)=O)c1N] |
| 81 | 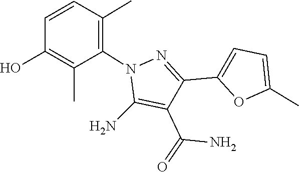 [SMILES: Cc1ccc(-c2nn(-c3c(C)ccc(O)c3C)c(N)c2C(N)=O)o1] |
| 82 | 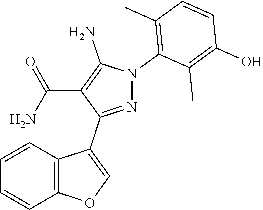 [SMILES: Cc1ccc(O)c(C)c1-n1nc(-c2coc3ccccc23)c(C(N)=O)c1N] |
| 83 | 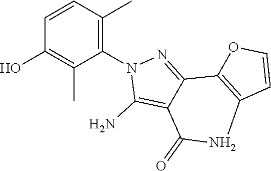 [SMILES: Cc1ccc(O)c(C)c1-n1nc2c(c1N)c(=O)[nH]c1ccoc12] |
| 84 | 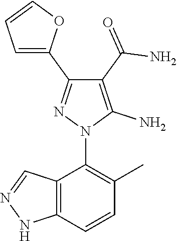 [SMILES: Cc1ccc2[nH]ncc2c1-n1nc(-c2ccco2)c(C(N)=O)c1N] |
| 85 | 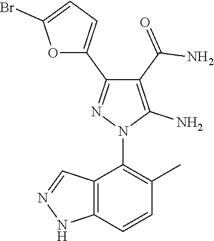 [SMILES: Cc1ccc2[nH]ncc2c1-n1nc(-c2ccc(Br)o2)c(C(N)=O)c1N] |
| 86 | 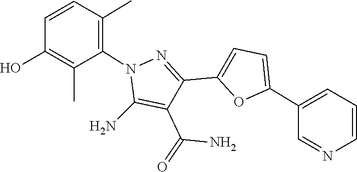 [SMILES: Cc1ccc(O)c(C)c1-n1nc(-c2ccc(-c3cccnc3)o2)c(C(N)=O)c1N] |
| 87 | 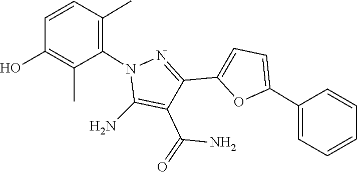 [SMILES: Cc1ccc(O)c(C)c1-n1nc(-c2ccc(-c3ccccc3)o2)c(C(N)=O)c1N] |
| 88 | 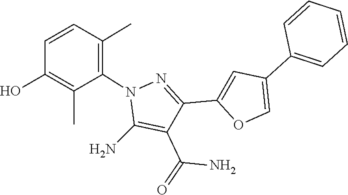 [SMILES: Cc1ccc(O)c(C)c1-n1nc(-c2cc(-c3ccccc3)co2)c(C(N)=O)c1N] |
| 89 | 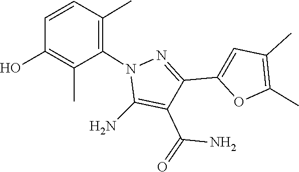 [SMILES: Cc1cc(-c2nn(-c3c(C)ccc(O)c3C)c(N)c2C(N)=O)oc1C] |
| 90 | 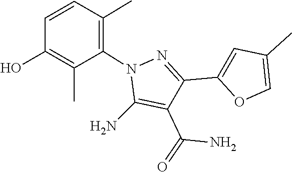 [SMILES: Cc1coc(-c2nn(-c3c(C)ccc(O)c3C)c(N)c2C(N)=O)c1] |
| 91 | 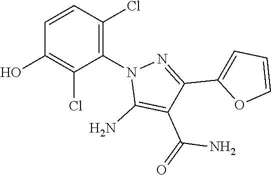 [SMILES: NC(=O)c1c(-c2ccco2)nn(-c2c(Cl)ccc(O)c2Cl)c1N] |
| 92 | 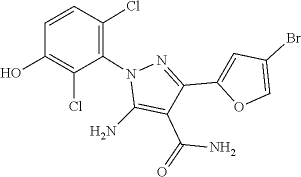 [SMILES: NC(=O)c1c(-c2cc(Br)co2)nn(-c2c(Cl)ccc(O)c2Cl)c1N] |
| 93 | 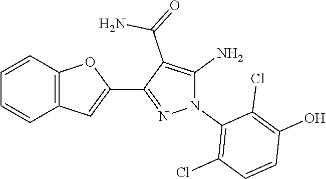 [SMILES: NC(=O)c1c(-c2cc3ccccc3o2)nn(-c2c(Cl)ccc(O)c2Cl)c1N] |
| 94 | 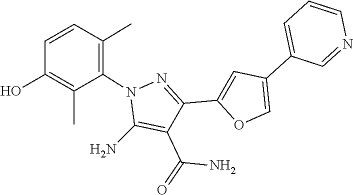 [SMILES: Cc1ccc(O)c(C)c1-n1nc(-c2cc(-c3cccnc3)co2)c(C(N)=O)c1N] |
| 95 | 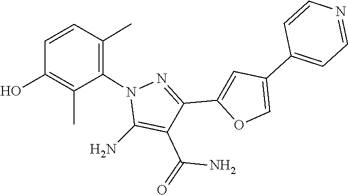 [SMILES: Cc1ccc(O)c(C)c1-n1nc(-c2cc(-c3ccncc3)co2)c(C(N)=O)c1N] |
| 96 | 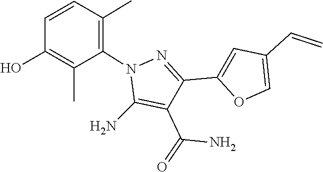 [SMILES: C=Cc1coc(-c2nn(-c3c(C)ccc(O)c3C)c(N)c2C(N)=O)c1] |
| 97 | 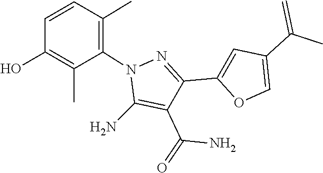 [SMILES: C=C(C)c1coc(-c2nn(-c3c(C)ccc(O)c3C)c(N)c2C(N)=O)c1] |
| 98 | 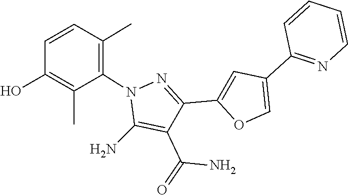 [SMILES: Cc1ccc(O)c(C)c1-n1nc(-c2cc(-c3ccccn3)co2)c(C(N)=O)c1N] |
| 99 | 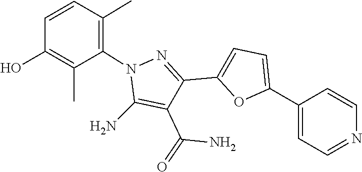 [SMILES: Cc1ccc(O)c(C)c1-n1nc(-c2ccc(-c3ccncc3)o2)c(C(N)=O)c1N] |
| 100 | 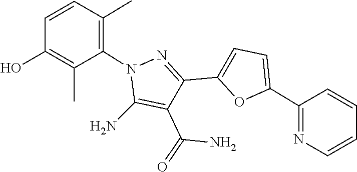 [SMILES: Cc1ccc(O)c(C)c1-n1nc(-c2ccc(-c3ccccn3)o2)c(C(N)=O)c1N] |
| 101 | 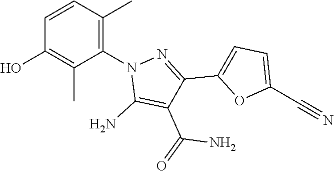 [SMILES: Cc1ccc(O)c(C)c1-n1nc(-c2ccc(C#N)o2)c(C(N)=O)c1N] |
| 102 | 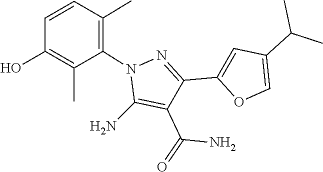 [SMILES: Cc1ccc(O)c(C)c1-n1nc(-c2cc(C(C)C)co2)c(C(N)=O)c1N] |
| 103 | 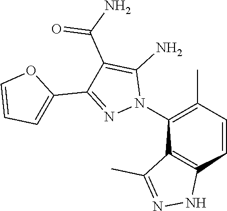 |
| 104 | 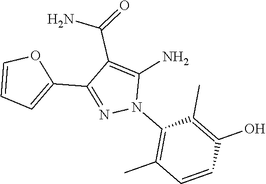 |
| 105 | 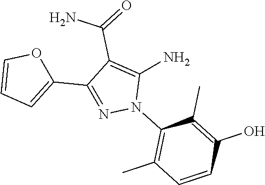 |
| 106 | 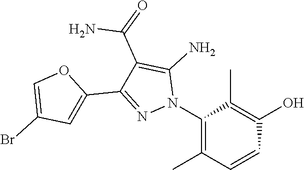 |
| 107 | 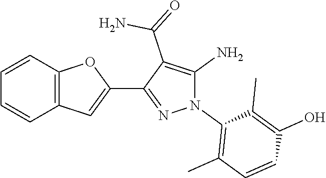 |
| 108 | 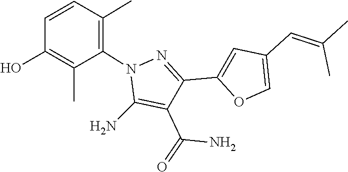 [SMILES: CC(C)=Cc1coc(-c2nn(-c3c(C)ccc(O)c3C)c(N)c2C(N)=O)c1] |
| 109 | 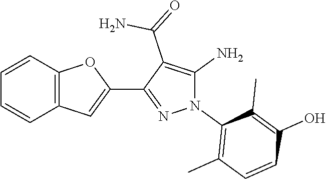 |
| 110 | 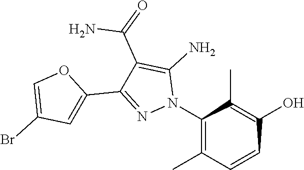 |
| 111 | 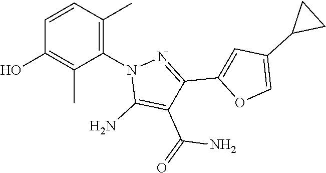 [SMILES: Cc1ccc(O)c(C)c1-n1nc(-c2cc(C3CC3)co2)c(C(N)=O)c1N] |
| 112 | 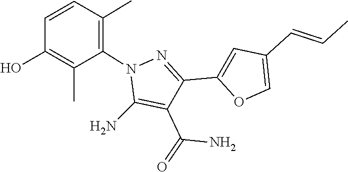 [SMILES: C/C=C/c1coc(-c2nn(-c3c(C)ccc(O)c3C)c(N)c2C(N)=O)c1] |
| 113 | 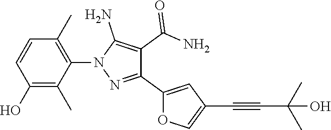 [SMILES: Cc1ccc(O)c(C)c1-n1nc(-c2cc(C#CC(C)(C)O)co2)c(C(N)=O)c1N] |
| 114 | 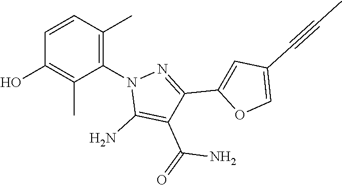 [SMILES: CC#Cc1coc(-c2nn(-c3c(C)ccc(O)c3C)c(N)c2C(N)=O)c1] |
| 115 | 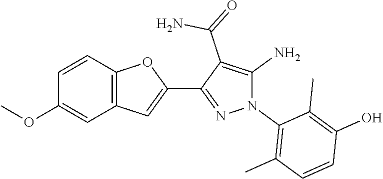 [SMILES: COc1ccc2oc(-c3nn(-c4c(C)ccc(O)c4C)c(N)c3C(N)=O)cc2c1] |
| 116 | 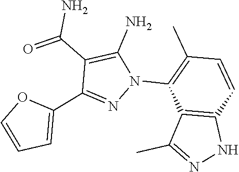 |
| 117 | 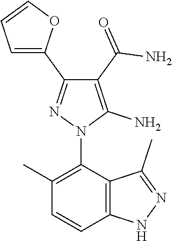 [SMILES: Cc1ccc2[nH]nc(C)c2c1-n1nc(-c2ccco2)c(C(N)=O)c1N] |

### ENUMERATED EMBODIMENTS

Embodiment 1. A compound of formula I:

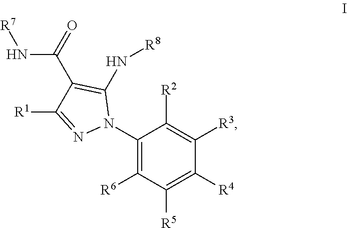

or a solvate, enantiomer, tautomer, atropisomer, or diastereomer thereof, or a pharmaceutically acceptable salt of any of the foregoing wherein:
R¹ is aryl, carbocyclyl, heteroaryl or heterocyclyl, each of which is optionally substituted;
each of R², R³, R⁴ and R⁶ is independently hydrogen, halo, —C₁-C₄ alkyl, —O—C₁-C₄ alkyl, —C₂-C₄ alkynyl, —CN, cyclopropyl, or phenyl; and
R⁵ is —OH; or
R² is hydrogen, halo, —C₁-C₄ alkyl, —O—C₁-C₄ alkyl, —C₂-C₄ alkynyl, —CN, or cyclopropyl;
each of R³ and R⁴ is independently hydrogen, halo, —C₁-C₄ alkyl, —O—C₁-C₄ alkyl, —CN, cyclopropyl, or phenyl; and
R⁵ and R⁶ are taken together with carbon atoms to which they are bound to form Ring A;
Ring A is a 4-7 membered heterocyclic or heteroaromatic ring comprising a ring nitrogen atom and optionally substituted with one or more R^{a} groups;
each R^{a} is independently selected from halo, ═O, —C₁-C₄ alkyl, —O—C₁-C₄ alkyl, —CN, and cyclopropyl;
each of R⁷ and R⁸ is independently hydrogen or optionally substituted C₁-C₄ alkyl; and
wherein one or more hydrogen atoms in the compound is optionally replaced with a deuterium atom.

Embodiment 2. The compound of embodiment 1, wherein:
R¹ is aryl, carbocyclyl, heteroaryl or heterocyclyl, each of which is optionally substituted;
each of R², R³, R⁴ and R⁶ is independently hydrogen, halo, —C₁-C₄ alkyl, —O—C₁-C₄ alkyl, —C₂-C₄ alkynyl, —CN, cyclopropyl, or phenyl; and
R⁵ is —OH; and
each of R⁷ and R⁸ is independently hydrogen or optionally substituted C₁-C₄ alkyl.

Embodiment 3. The compound of embodiment 2, wherein R¹ is aryl or heteroaryl.

Embodiment 4. The compound of embodiment 2, wherein R¹ is carbocyclyl or heterocyclyl.

Embodiment 5. The compound of embodiment 2, wherein R¹ is aryl or carbocyclyl.

Embodiment 6. The compound of embodiment 2, wherein R¹ is heteroaryl or heterocyclyl.

Embodiment 7. The compound of any one of embodiments 2-6, wherein R¹ is selected from the group consisting of:

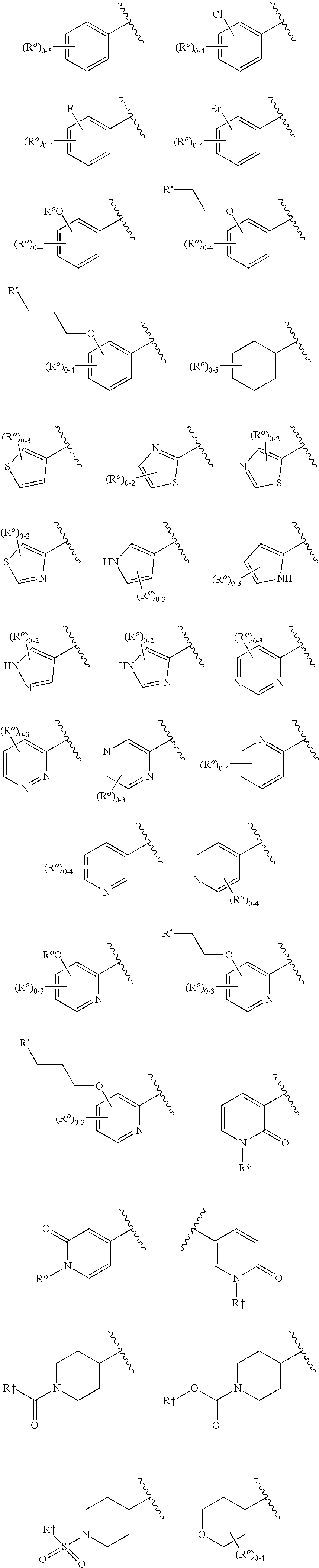

Embodiment 8. The compound of any one of embodiments 2-7, wherein R¹ is selected from the group consisting of:

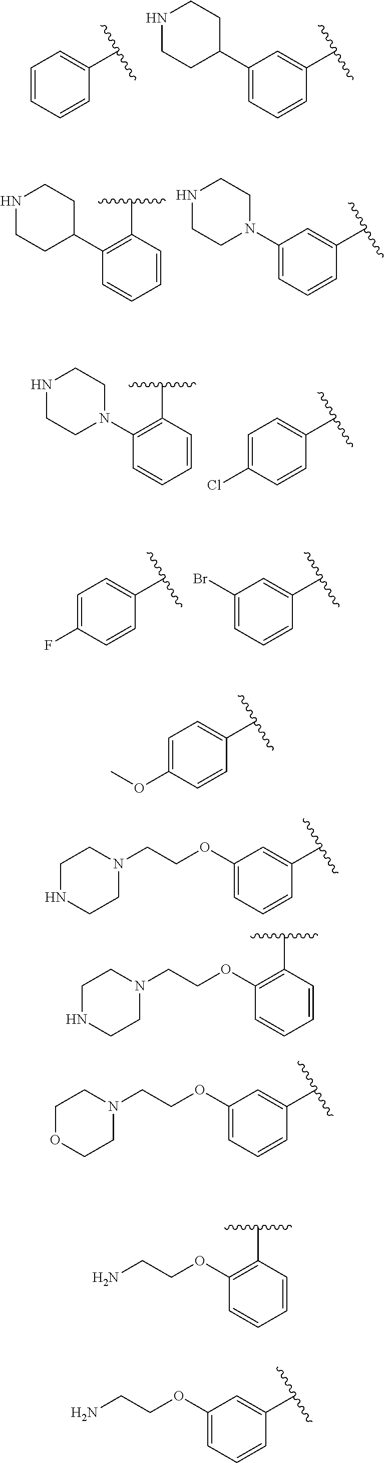

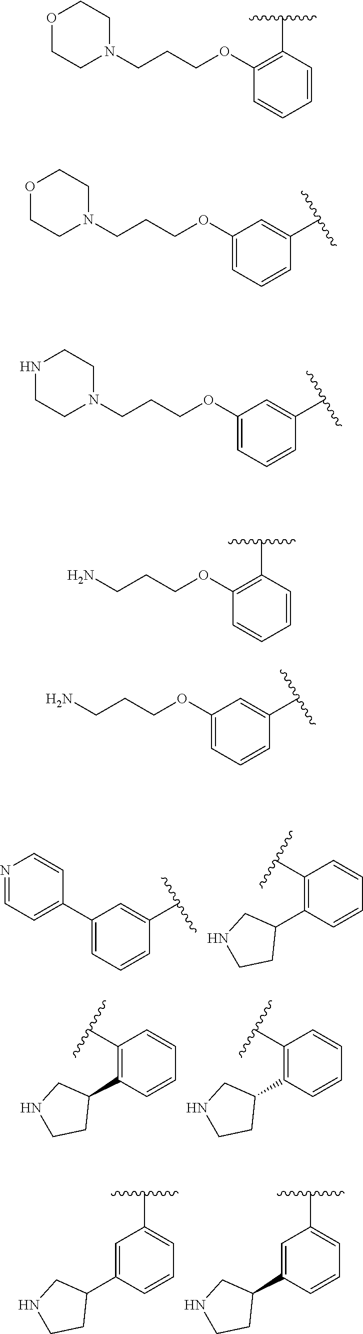

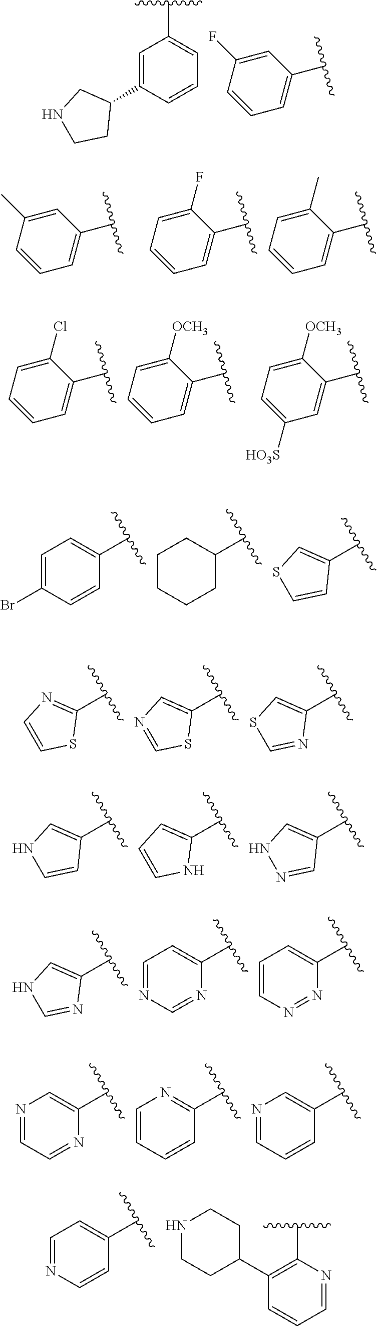

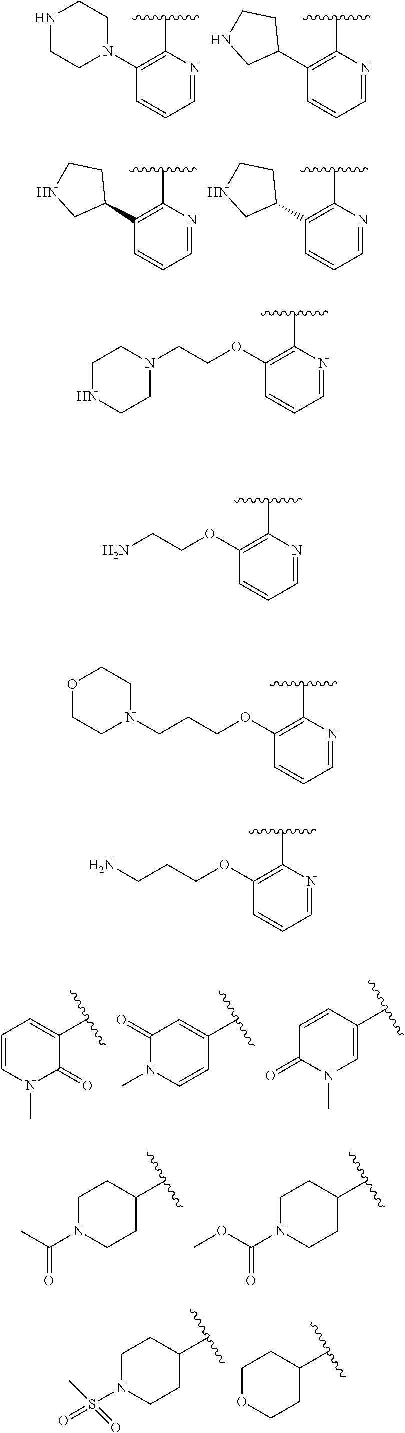

Embodiment 9. The compound of embodiment 8, wherein R¹ is selected from the group consisting of:

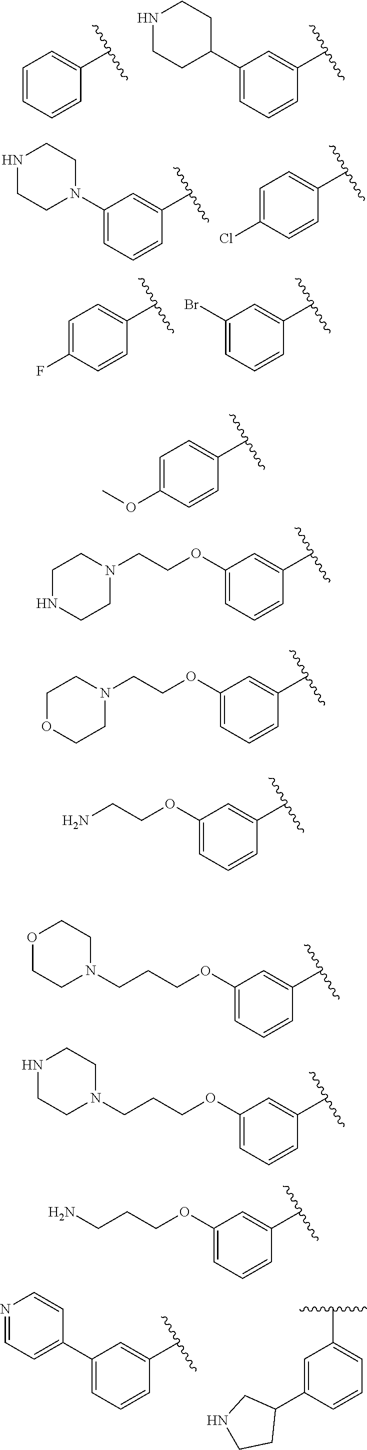

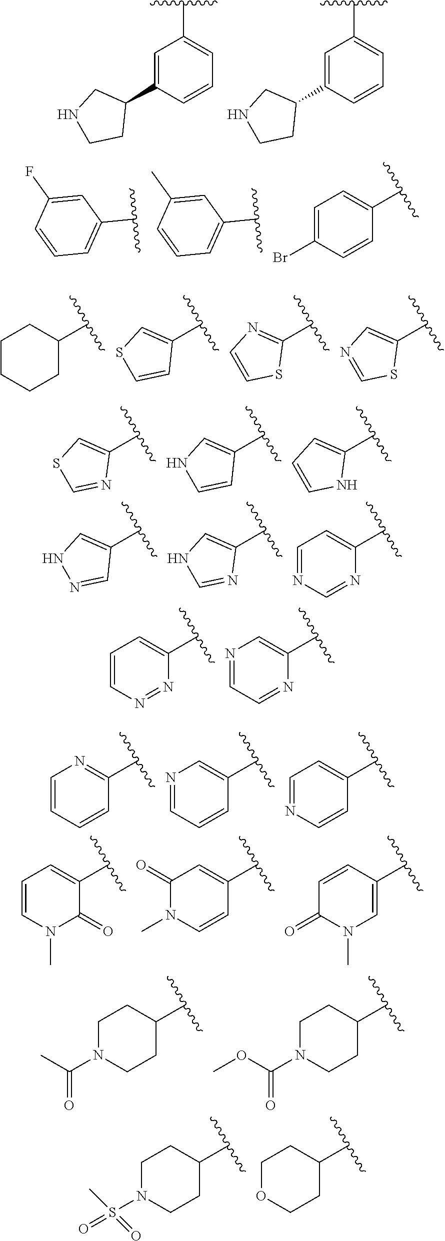

Embodiment 10. The compound of any one of embodiments 2-6, wherein R¹ is phenyl, pyridinyl, oxo-dihydropyridinyl, pyrimidinyl, pyridazinyl, pyrazinyl, thiophenyl, thiazolyl, pyrazolyl, imidazolyl, pyrrolyl, cyclohexyl, piperidinyl, or tetrahydropyranyl, each of which is optionally substituted.

Embodiment 11. The compound of embodiment 10, wherein R¹ is phenyl, pyridin-2-yl, pyridin-3-yl, pyridin-4-yl, pyrimidin-4-yl, pyridazin-3-yl, thiophen-3-yl, cyclohexyl, 6-oxo-1,6-dihydropyridin-3-yl, 2-oxo-1,2-dihydropyridin-4-yl, 2-oxo-1,2-dihydropyridin-3-yl, or piperidin-4-yl, each of which is optionally substituted.

Embodiment 12. The compound of embodiment 11, wherein R¹ is phenyl, pyridin-2-yl, pyridin-3-yl, pyridin-4-yl, pyrimidin-4-yl, pyridazin-3-yl, thiophen-3-yl, cyclohexyl, 6-oxo-1,6-dihydropyridin-3-yl, 2-oxo-1,2-dihydropyridin-4-yl, 2-oxo-1,2-dihydropyridin-3-yl, or piperidin-4-yl, each of which is optionally substituted.

Embodiment 13. The compound of embodiment 12, wherein R¹ is phenyl or pyridin-2-yl, each of which is optionally substituted.

Embodiment 14. The compound of any one of embodiments 11-13, wherein R¹ is optionally substituted with 1-4 substituents independently selected from halo, —CN, C₁-C₄ alkyl, C₁-C₄ haloalkyl, —O—C₁-C₄ alkyl, —O—C₁-C₄ haloalkyl, —C(O)—C₁-C₄ alkyl, —C(O)—O—C₁-C₄ alkyl, —S(O)₂—C₁-C₄ alkyl, —O—C₁-C₄ alkylene-C₃-C₆ cycloalkyl, C₃-C₆ cycloalkyl, —O—C₃-C₆ cycloalkyl, a 5-6 membered saturated heterocycle comprising at least one nitrogen ring atom, a 5-6 membered heteroaryl comprising at least one nitrogen ring atom, or —X—(C₂-C₄ alkylene)-NR⁹R¹⁰, wherein:
X is —O—, —CR¹¹R¹²—, —NR¹³—, —S—, —SO₂—, —NR¹³SO₂—, —C(O)NR¹³—, —NR¹³C(O)—, or —OC(O)NR¹³—;
each of R⁹, R¹⁰, and R¹³ is independently hydrogen, optionally substituted C₁-C₄ alkyl, or optionally substituted —O—C₁-C₄ alkyl, or R⁹ and R¹⁰ are taken together with the nitrogen atom to which they are bound to form an optionally substituted 5-7 membered saturated heterocycle optionally comprising one additional heteroatom selected from N, O or S; and
each of R¹¹ and R¹² is independently hydrogen, halo or C₁-C₄ alkyl.

Embodiment 15. The compound of any one of embodiments 10-14, wherein R¹ is substituted with 1-3 substituents and independently selected from fluoro, chloro, bromo, cyano, methyl, —OCH₃, —OCF₃, —OCH₂CF₃, —S(O)₂CH₃, —SO₃H, —C(O) OCH₃, —C(O)CH₃, —O(CH₂)₂NR⁹R¹⁰, —O(CH₂)₃NR⁹R¹⁰, cyclopropylmethylenoxy, piperazin-1-yl, piperidin-4-yl, pyrrolidin-3-yl, or pyridin-4-yl, wherein each of R⁹ and R¹⁰ is independently selected from hydrogen and methyl, or R⁹ and R¹⁰ are taken together with the nitrogen atom to which they are bound to form piperidin-1-yl, piperazin-1-yl, or morpholin-4-yl.

Embodiment 16. The compound of any one of embodiments 10-14, wherein R¹ is substituted with 1-3 substituents and independently selected from fluoro, chloro, bromo, methyl, —OCH₃, —S(O)₂CH₃, —SO₃H, —C(O) OCH₃, —C(O)CH₃, —O(CH₂)₂NR⁹R¹⁰, —O(CH₂)₃NR⁹R¹⁰, piperazin-1-yl, piperidin-4-yl, pyrrolidin-3-yl, or pyridin-4-yl, wherein each of R⁹ and R¹⁰ is hydrogen, or R⁹ and R¹⁰ are taken together with the nitrogen atom to which they are bound to form piperazin-1-yl or morpholin-4-yl.

Embodiment 17. The compound of any one of embodiments 14-16, wherein R¹ is phenyl or pyridin-2-yl and each of the 1-3 substituents is in a meta or para position on R¹.

Embodiment 18. The compound of any one of embodiments 2-6, wherein R¹ is phenyl, 2-fluorophenyl, 2-chlorophenyl, 2-methoxyphenyl, 2-methylphenyl, 3-bromophenyl, 4-bromophenyl, 2-methoxy-5-sulfonylphenyl, 3-pyridin-4-ylphenyl, 3-piperidin-4-ylphenyl, 3-fluorophenyl, 3-methylphenyl, 4-fluorophenyl, 4-chlorophenyl, 4-methoxyphenyl, 2-(2-aminoethan-1-yl)oxyphenyl, 3-(2-aminoethan-1-yl)oxyphenyl, 2-(3-aminopropan-1-yl)oxyphenyl, 3-(3-aminopropan-1-yl)oxyphenyl, 2-pyrrolidin-3-ylphenyl, 3-pyrrolidin-3-ylphenyl, 2-piperidin-4-ylphenyl, 3-piperidin-4-ylphenyl, 2-piperazin-1-ylphenyl, 3-piperazin-1-ylphenyl, 2-(2-piperazin-1-ylethan-1-yl)oxyphenyl, 3-(2-piperazin-1-ylethan-1-yl)oxyphenyl, 3-(2-morpholin-4-ylethan-1-yl)oxyphenyl, 3-(3-piperazin-1-ylpropan-1-yl)oxyphenyl, 2-(3-mopholin-4-ylpropan-1-yl)oxyphenyl, 3-(3-mopholin-4-ylpropan-1-yl)oxyphenyl, 1H-pyrazol-4-yl, 1H-imidazol-4-yl, pyridin-2-yl, pyrindin-3-yl, pyridin-4-yl, pyrimidin-4-yl, pyrazindin-3-yl, thiophen-3-yl, cyclohexyl, tetrahydropyran-4-yl, 1-methyl-6-oxo-1,6-dihydropyridin-3-yl, 1-methyl-2-oxo-1,2-dihydropyridin-4-yl, 1-methyl-2-oxo-1,2-dihydropyridin-3-yl, 1-acetylpiperidin-4-yl, 3-(2-aminoethan-1-yl)oxypyridin-2-yl, 1-methoxycarbonylpiperidin-4-yl, 3-pyrrolidin-3-ylpyridin-2-yl, 3-(3-aminopropan-1-yl)oxypyridin-2-yl, 3-piperidin-4-ylpyridin-2-yl, 3-piperiazn-1-ylpyridin-2-yl, 1-methylsulfonylpiperidin-4-yl, 3-(2-piperazin-1-ylethan-1-yl)oxypyridin-2-yl, or 3-(3-morpholin-4-ylpropan-1-yl)oxypyridin-2-yl.

Embodiment 19. The compound of any one of embodiments 2-18, wherein R² is hydrogen.

Embodiment 20. The compound of any one of embodiments 2-18, wherein R² is selected from halo, —C₁-C₄ alkyl, —O—C₁-C₄ alkyl, —C₂-C₄ alkynyl, —CN, cyclopropyl, and phenyl.

Embodiment 21. The compound of embodiment 20, wherein R² is halo.

Embodiment 22. The compound of embodiment 21, wherein R² is fluoro or chloro.

Embodiment 23. The compound of embodiment 20, wherein R² is —C₁-C₄ alkyl.

Embodiment 24. The compound of embodiment 23, wherein R² is —C₁-C₃ alkyl.

Embodiment 25. The compound of embodiment 24, wherein R² is methyl or ethyl.

Embodiment 26. The compound of embodiment 20, wherein R² is —O—C₁-C₄ alkyl.

Embodiment 27. The compound of embodiment 26, wherein R² is —O—C₁-C₃ alkyl.

Embodiment 28. The compound of embodiment 27, wherein R² is —OCH₃ or —OCH₂CH₃.

Embodiment 29. The compound of embodiment 20, wherein R² is —C₂-C₄ alkynyl.

Embodiment 30. The compound of embodiment 29, wherein R² is —C≡CH.

Embodiment 31. The compound of embodiment 20, wherein R² is —CN.

Embodiment 32. The compound of embodiment 20, wherein R² is cyclopropyl.

Embodiment 33. The compound of embodiment 20, wherein R² is phenyl.

Embodiment 34. The compound of embodiment 20, wherein R² is selected from —C₁-C₄ alkyl, —C₂-C₄ alkynyl, cyclopropyl, and phenyl.

Embodiment 35. The compound of embodiment 20, wherein R² is selected from halo, —O—C₁-C₄ alkyl, and —CN.

Embodiment 36. The compound of embodiment 20, wherein R² is selected from chloro, —CH₃, —CH₂CH₃, and —C≡CH.

Embodiment 37. The compound of any one of embodiments 2-36, wherein R³ is hydrogen.

Embodiment 38. The compound of any one of embodiments 2-36, wherein R³ is selected from halo, —C₁-C₄ alkyl, —O—C₁-C₄ alkyl, —C₂-C₄ alkynyl, —CN, cyclopropyl, and phenyl.

Embodiment 39. The compound of embodiment 38, wherein R³ is halo.

Embodiment 40. The compound of embodiment 39, wherein R³ is fluoro or chloro.

Embodiment 41. The compound of embodiment 38, wherein R³ is —C₁-C₄ alkyl.

Embodiment 42. The compound of embodiment 41, wherein R³ is —C₁-C₃ alkyl.

Embodiment 43. The compound of embodiment 41, wherein R³ is methyl or ethyl.

Embodiment 44. The compound of embodiment 38, wherein R³ is —O—C₁-C₄ alkyl.

Embodiment 45. The compound of embodiment 44, wherein R³ is —O—C₁-C₃ alkyl.

Embodiment 46. The compound of embodiment 44, wherein R³ is —OCH₃ or —OCH₂CH₃.

Embodiment 47. The compound of embodiment 38, wherein R³ is —C₂-C₄ alkynyl.

Embodiment 48. The compound of embodiment 47, wherein R³ is —C≡CH.

Embodiment 49. The compound of embodiment 38, wherein R³ is —CN.

Embodiment 50. The compound of embodiment 38, wherein R³ is cyclopropyl.

Embodiment 51. The compound of embodiment 38, wherein R³ is phenyl.

Embodiment 52. The compound of embodiment 38, wherein R³ is selected from —C₁-C₄ alkyl, —C₂-C₄ alkynyl, cyclopropyl, and phenyl.

Embodiment 53. The compound of embodiment 38, wherein R³ is selected from halo, —O—C₁-C₄ alkyl, and —CN.

Embodiment 54. The compound of any one of embodiments 2-53, wherein R⁴ is hydrogen.

Embodiment 55. The compound of any one of embodiments 2-53, wherein R⁴ is selected from halo, —C₁-C₄ alkyl, —O—C₁-C₄ alkyl, —C₂-C₄ alkynyl, —CN, cyclopropyl, and phenyl.

Embodiment 56. The compound of embodiment 55, wherein R⁴ is halo.

Embodiment 57. The compound of embodiment 56, wherein R⁴ is fluoro or chloro.

Embodiment 58. The compound of embodiment 55, wherein R⁴ is —C₁-C₄ alkyl.

Embodiment 59. The compound of embodiment 58, wherein R⁴ is —C₁-C₃ alkyl.

Embodiment 60. The compound of embodiment 58, wherein R⁴ is methyl or ethyl.

Embodiment 61. The compound of embodiment 55, wherein R⁴ is —O—C₁-C₄ alkyl.

Embodiment 62. The compound of embodiment 61, wherein R⁴ is —O—C₁-C₃ alkyl.

Embodiment 63. The compound of embodiment 61, wherein R⁴ is —OCH₃ or —OCH₂CH₃.

Embodiment 64. The compound of embodiment 55, wherein R⁴ is —C₂-C₄ alkynyl.

Embodiment 65. The compound of embodiment 64, wherein R⁴ is —C═CH.

Embodiment 66. The compound of embodiment 55, wherein R⁴ is —CN.

Embodiment 67. The compound of embodiment 55, wherein R⁴ is cyclopropyl.

Embodiment 68. The compound of embodiment 55, wherein R⁴ is phenyl.

Embodiment 69. The compound of embodiment 55, wherein R⁴ is selected from —C₁-C₄ alkyl, —C₂-C₄ alkynyl, cyclopropyl, and phenyl.

Embodiment 70. The compound of embodiment 55, wherein R⁴ is selected from halo, —O—C₁-C₄ alkyl, and —CN.

Embodiment 71. The compound of any one of embodiments 2-70, wherein R⁶ is hydrogen.

Embodiment 72. The compound of any one of embodiments 2-70, wherein R⁶ is selected from halo, —C₁-C₄ alkyl, —O—C₁-C₄ alkyl, —C₂-C₄ alkynyl, —CN, cyclopropyl, and phenyl.

Embodiment 73. The compound of embodiment 72, wherein R⁶ is halo.

Embodiment 74. The compound of embodiment 73, wherein R⁶ is fluoro or chloro.

Embodiment 75. The compound of embodiment 72, wherein R⁶ is —C₁-C₄ alkyl.

Embodiment 76. The compound of embodiment 75, wherein R⁶ is —C₁-C₃ alkyl.

Embodiment 77. The compound of embodiment 75, wherein R⁶ is methyl or ethyl.

Embodiment 78. The compound of embodiment 72, wherein R⁶ is —O—C₁-C₄ alkyl.

Embodiment 79. The compound of embodiment 78, wherein R⁶ is —O—C₁-C₃ alkyl.

Embodiment 80. The compound of embodiment 78, wherein R⁶ is —OCH₃ or —OCH₂CH₃.

Embodiment 81. The compound of embodiment 72, wherein R⁶ is —C₂-C₄ alkynyl.

Embodiment 82. The compound of embodiment 81, wherein R⁶ is —C═CH.

Embodiment 83. The compound of embodiment 72, wherein R⁶ is —CN.

Embodiment 84. The compound of embodiment 72, wherein R⁶ is cyclopropyl.

Embodiment 85. The compound of embodiment 72, wherein R⁶ is phenyl.

Embodiment 86. The compound of embodiment 72, wherein R⁶ is selected from —C₁-C₄ alkyl, —C₂-C₄ alkynyl, cyclopropyl, and phenyl.

Embodiment 87. The compound of embodiment 72, wherein R⁶ is selected from halo, —O—C₁-C₄ alkyl, and —CN.

Embodiment 88. The compound of any one of embodiments 2-87, wherein R⁷ is hydrogen.

Embodiment 89. The compound of any one of embodiments 2-87, wherein R⁷ is optionally substituted C₁-C₄ alkyl or optionally substituted —O—C₁-C₄ alkyl.

Embodiment 90. The compound of embodiment 89, wherein R⁷ is optionally substituted C₁-C₄ alkyl.

Embodiment 91. The compound of embodiment 90, wherein R⁷ is optionally substituted C₁-C₃ alkyl.

Embodiment 92. The compound of embodiment 91, wherein R⁷ is optionally substituted methyl or ethyl.

Embodiment 93. The compound of any one of embodiments 2-92, wherein R⁸ is hydrogen.

Embodiment 94. The compound of any one of embodiments 2-92, wherein R⁸ is optionally substituted C₁-C₄ alkyl or optionally substituted —O—C₁-C₄ alkyl.

Embodiment 95. The compound of embodiment 94, wherein R⁸ is optionally substituted C₁-C₄ alkyl.

Embodiment 96. The compound of embodiment 95, wherein R⁸ is optionally substituted C₁-C₃ alkyl.

Embodiment 97. The compound of embodiment 96, wherein R⁸ is optionally substituted methyl or ethyl.

Embodiment 98. The compound of any one of embodiments 2-6, wherein
R² is hydrogen, fluoro, chloro, bromo, iodo, methyl, ethyl, or ethynyl;
R³ is hydrogen, fluoro, methyl or —CN;
R⁴ is hydrogen, fluoro, methyl or —CN;
R⁵ is —OH; and
R⁶ is methyl, ethyl, propyl, isopropyl, —CN, —OCH₃, cyclopropyl or phenyl.

Embodiment 99. The compound of embodiment 98, wherein
R² is hydrogen, chloro, methyl, ethyl or ethynyl;
each of R³ and R⁴ is hydrogen;
R⁵ is —OH; and
R⁶ is hydrogen, chloro, or methyl.

Embodiment 100. The compound of any one of embodiments 2-99, wherein at least one of R², R³, R⁴, and R⁶ is other than hydrogen.

Embodiment 101. The compound of embodiment 100, wherein at least one of R² and R⁶ is other than hydrogen.

Embodiment 102. The compound of embodiment 1, wherein:
R¹ is aryl, carbocyclyl, heteroaryl or heterocyclyl, each of which is optionally substituted;
R² is hydrogen, halo, —C₁-C₄ alkyl, —O—C₁-C₄ alkyl, —C₂-C₄ alkynyl, —CN, or cyclopropyl;
each of R³ and R⁴ is independently hydrogen, halo, —C₁-C₄ alkyl, —O—C₁-C₄ alkyl, —CN, cyclopropyl, or phenyl;
R⁵ and R⁶ are taken together with carbon atoms to which they are bound to form Ring A;
Ring A is a 4-7 membered heterocyclic or heteroaromatic ring comprising a ring nitrogen atom and optionally substituted with one or more R^{a} groups;
each R^{a} is independently selected from halo, ═O, —C₁-C₄ alkyl, —O—C₁-C₄ alkyl, —CN, and cyclopropyl; and
each of R⁷ and R⁸ is independently hydrogen or optionally substituted C₁-C₄ alkyl.

Embodiment 103. The compound of embodiment 102, wherein R¹ is aryl or heteroaryl.

Embodiment 104. The compound of embodiment 102, wherein R¹ is carbocyclyl or heterocyclyl.

Embodiment 105. The compound of embodiment 102, wherein R¹ is aryl or carbocyclyl.

Embodiment 106. The compound of embodiment 102, wherein R¹ is heteroaryl or heterocyclyl.

Embodiment 107. The compound of any one of embodiments 102-106, wherein R¹ is selected from the group consisting of:

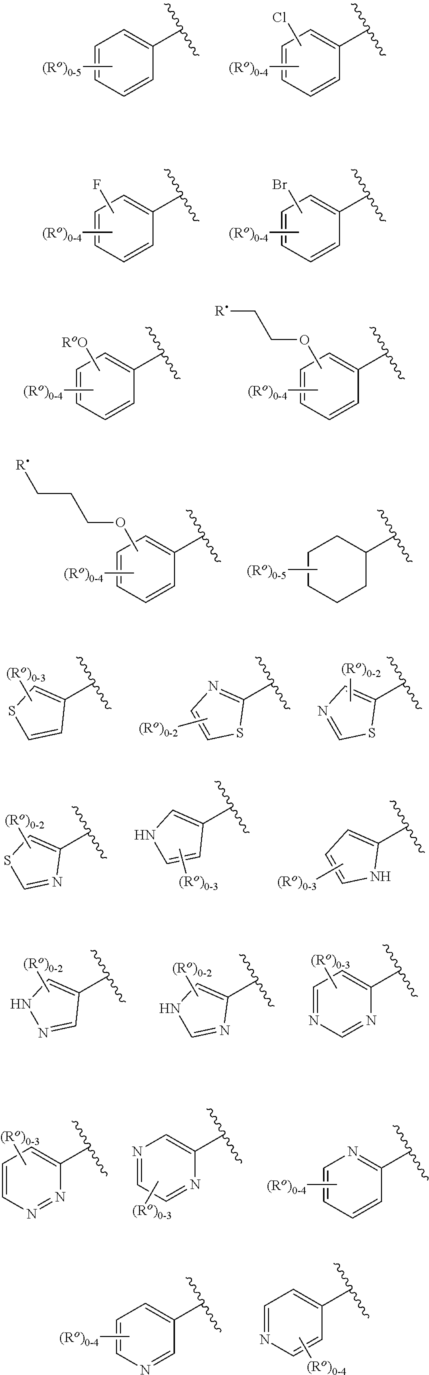

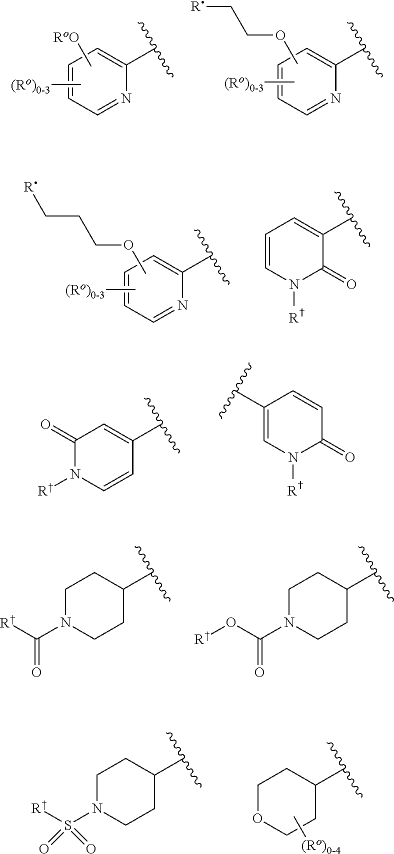

Embodiment 108. The compound of any one of embodiments 102-107, wherein R¹ is selected from the group consisting of:

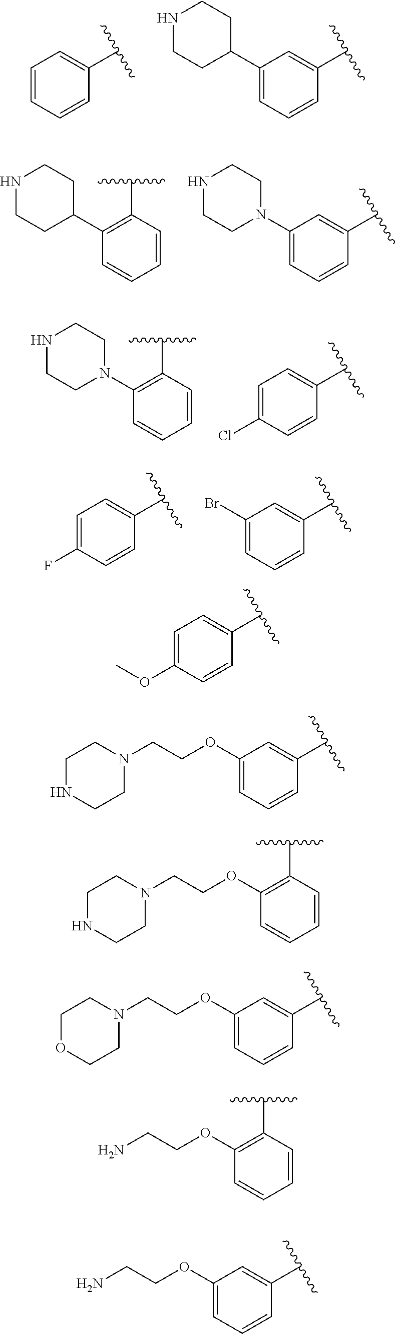

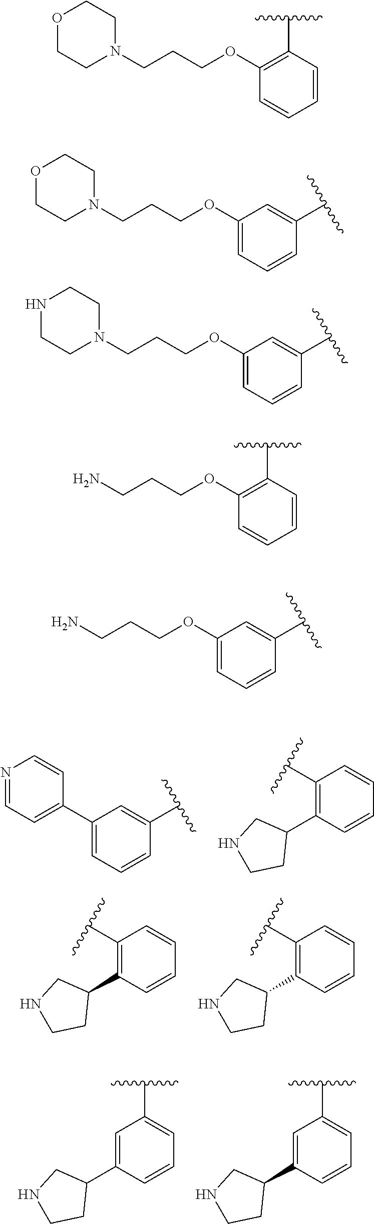

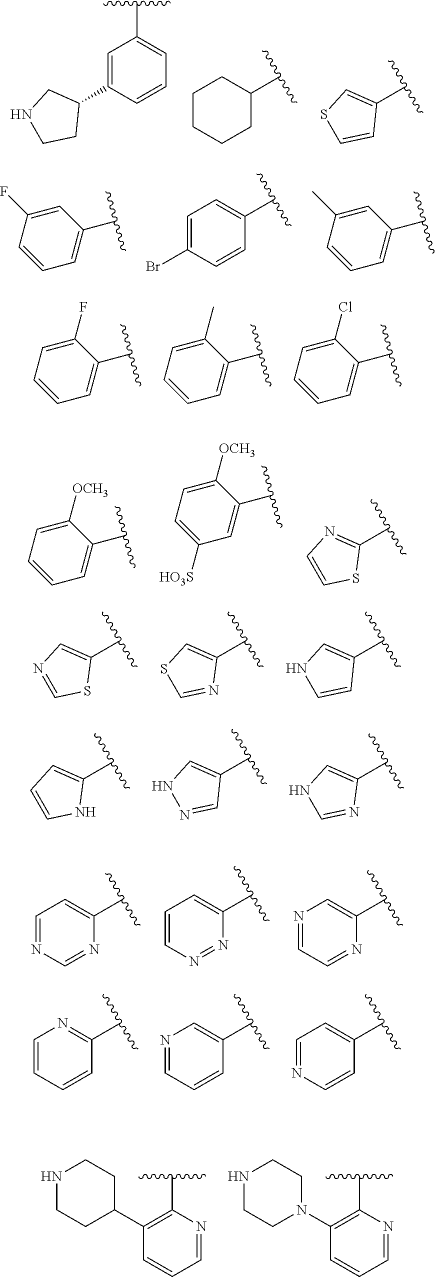

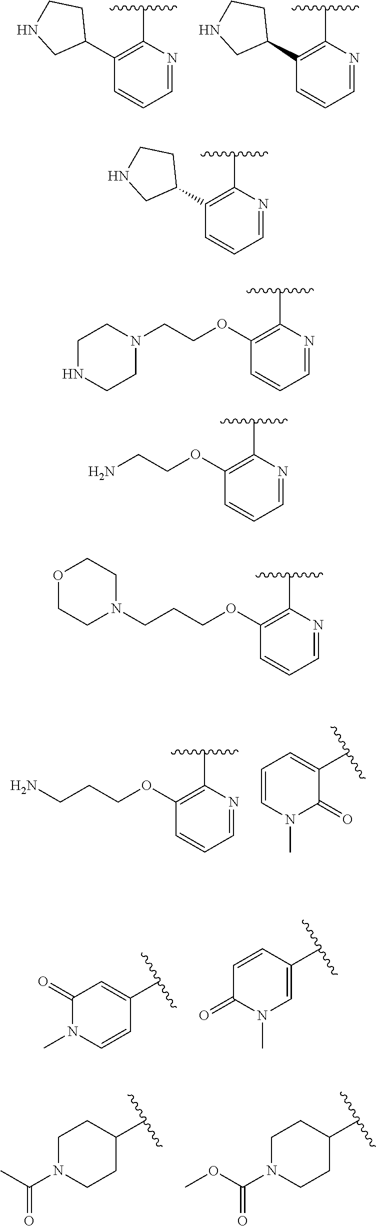

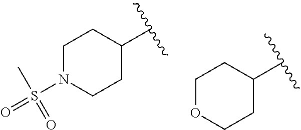 [SMILES: *C1CCN(S(C)(=O)=O)CC1 ; *C1CCOCC1]

Embodiment 109. The compound of embodiment 108, wherein R¹ is selected from the group consisting of:

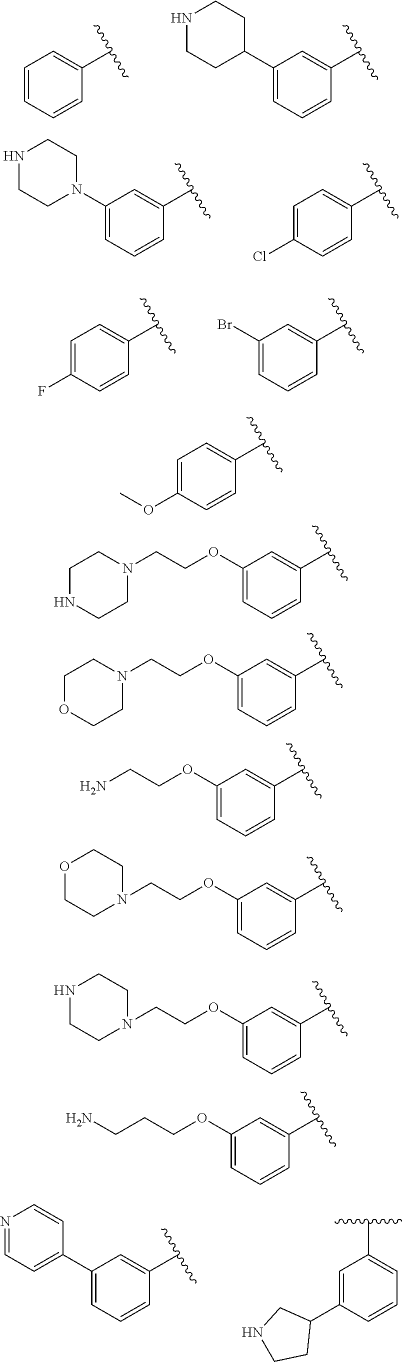

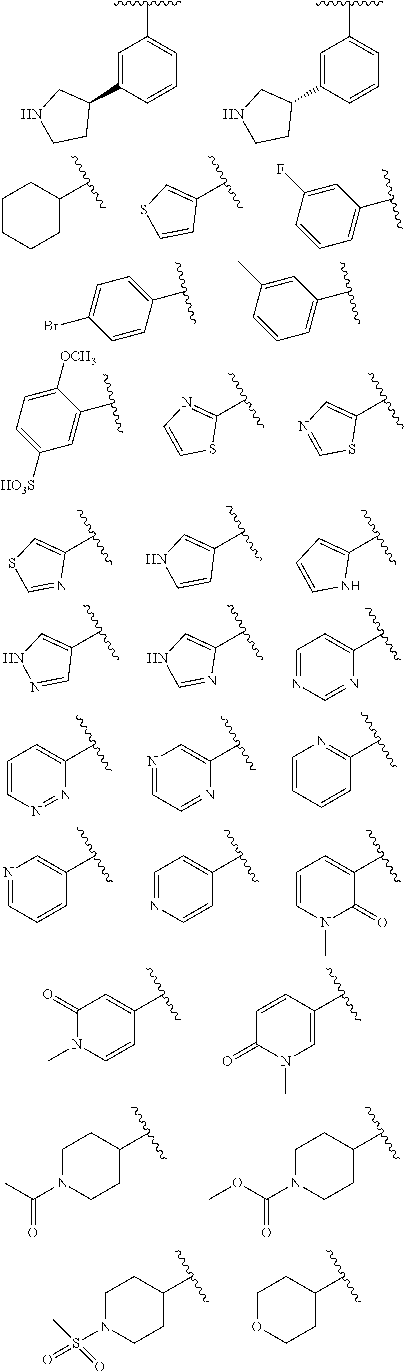

Embodiment 110. The compound of any one of embodiments 102-106, wherein R¹ is phenyl, pyridinyl, oxo-dihydropyridinyl, pyrimidinyl, pyridazinyl, pyrazinyl, thiophenyl, thiazolyl, pyrazolyl, imidazolyl, pyrrolyl, cyclohexyl, piperidinyl, or tetrahydropyranyl, each of which is optionally substituted.

Embodiment 111. The compound of embodiment 110, wherein R¹ is phenyl, pyridin-2-yl, pyridin-3-yl, pyridin-4-yl, pyrimidin-4-yl, pyridazin-3-yl, thiophen-3-yl, cyclohexyl, 6-oxo-1,6-dihydropyridin-3-yl, 2-oxo-1,2-dihydropyridin-4-yl, 2-oxo-1,2-dihydropyridin-3-yl, or piperidin-4-yl, each of which is optionally substituted.

Embodiment 112. The compound of embodiment 111, wherein R¹ is phenyl, pyridin-2-yl, pyridin-3-yl, pyridin-4-yl, pyrimidin-4-yl, pyridazin-3-yl, thiophen-3-yl, cyclohexyl, 6-oxo-1,6-dihydropyridin-3-yl, 2-oxo-1,2-dihydropyridin-4-yl, 2-oxo-1,2-dihydropyridin-3-yl, or piperidin-4-yl, each of which is optionally substituted.

Embodiment 113. The compound of embodiment 112, wherein R¹ is phenyl or pyridin-2-yl, each of which is optionally substituted.

Embodiment 114. The compound of any one of embodiments 110-113, wherein R¹ is optionally substituted with 1-4 substituents independently selected from halo, —CN, C₁-C₄ alkyl, C₁-C₄ haloalkyl, —O—C₁-C₄ alkyl, —O—C₁-C₄ haloalkyl, —C(O)—C₁-C₄ alkyl, —C(O)—O—C₁-C₄ alkyl, —S(O)₂—C₁-C₄ alkyl, —O—C₁-C₄ alkylene-C₃-C₆ cycloalkyl, C₃-C₆ cycloalkyl, —O—C₃-C₆ cycloalkyl, a 5-6 membered saturated heterocycle comprising at least one nitrogen ring atom, a 5-6 membered heteroaryl comprising at least one nitrogen ring atom, or —X—(C₂-C₄ alkylene)-NR⁹R¹⁰, wherein:
X is —O—, —CR¹¹R¹²—, —NR¹³—, —S—, —SO₂—, —NR¹³SO₂—, —C(O)NR¹³—, —NR¹³C(O)—, or —OC(O)NR¹³—;
each of R⁹, R¹⁰, and R¹³ is independently hydrogen, optionally substituted C₁-C₄ alkyl, or optionally substituted —O—C₁-C₄ alkyl, or R⁹ and R¹⁰ are taken together with the nitrogen atom to which they are bound to form an optionally substituted 5-7 membered saturated heterocycle optionally comprising one additional heteroatom selected from N, O or S; and
each of R¹¹ and R¹² is independently hydrogen, halo or C₁-C₄ alkyl.

Embodiment 115. The compound of any one of embodiments 110-114, wherein R¹ is substituted with 1-3 substituents and independently selected from fluoro, chloro, bromo, cyano, methyl, —OCH₃, —OCF₃, —OCH₂CF₃, —S(O)₂CH₃, —SO₃H, —C(O) OCH₃, —C(O)CH₃, —O(CH₂)₂NR⁹R¹⁰, —O(CH₂)₃NR⁹R¹⁰, cyclopropylmethylenoxy, piperazin-1-yl, piperidin-4-yl, pyrrolidin-3-yl, or pyridin-4-yl, wherein each of R⁹ and R¹⁰ is independently selected from hydrogen and methyl, or R⁹ and R¹⁰ are taken together with the nitrogen atom to which they are bound to form piperidin-1-yl, piperazin-1-yl, or morpholin-4-yl.

Embodiment 116. The compound of any one of embodiments 110-114, wherein R¹ is substituted with 1-3 substituents and independently selected from fluoro, chloro, bromo, methyl, —OCH₃, —S(O)₂CH₃, —SO₃H, —C(O) OCH₃, —C(O)CH₃, —O(CH₂)₂NR⁹R¹⁰, —O(CH₂)₃NR⁹R¹⁰, piperazin-1-yl, piperidin-4-yl, pyrrolidin-3-yl, or pyridin-4-yl, wherein each of R⁹ and R¹⁰ is hydrogen, or R⁹ and R¹⁰ are taken together with the nitrogen atom to which they are bound to form piperazin-1-yl or morpholin-4-yl.

Embodiment 117. The compound of any one of embodiments 114-116, wherein R¹ is phenyl or pyridin-2-yl and each of the 1-3 substituents is in a meta or para position on R¹.

Embodiment 118. The compound of any one of embodiments 102-106, wherein R¹ is phenyl, 2-fluorophenyl, 2-chlorophenyl, 2-methoxyphenyl, 2-methylphenyl, 3-bromophenyl, 4-bromophenyl, 2-methoxy-5-sulfonylphenyl, 3-pyridin-4-ylphenyl, 3-piperidin-4-ylphenyl, 3-fluorophenyl, 3-methylphenyl, 4-fluorophenyl, 4-chlorophenyl, 4-methoxyphenyl, 2-(2-aminoethan-1-yl)oxyphenyl, 3-(2-aminoethan-1-yl)oxyphenyl, 2-(3-aminopropan-1-yl)oxyphenyl, 3-(3-aminopropan-1-yl)oxyphenyl, 2-pyrrolidin-3-ylphenyl, 3-pyrrolidin-3-ylphenyl, 2-piperidin-4-ylphenyl, 3-piperidin-4-ylphenyl, 2-piperazin-1-ylphenyl, 3-piperazin-1-ylphenyl, 2-(2-piperazin-1-ylethan-1-yl)oxyphenyl, 3-(2-piperazin-1-ylethan-1-yl)oxyphenyl, 3-(2-morpholin-4-ylethan-1-yl)oxyphenyl, 3-(3-piperazin-1-ylpropan-1-yl)oxyphenyl, 2-(3-mopholin-4-ylpropan-1-yl)oxyphenyl, 3-(3-mopholin-4-ylpropan-1-yl)oxyphenyl, 1H-pyrazol-4-yl, 1H-imidazol-4-yl, pyridin-2-yl, pyrindin-3-yl, pyridin-4-yl, pyrimidin-4-yl, pyrazindin-3-yl, thiophen-3-yl, cyclohexyl, tetrahydropyran-4-yl, 1-methyl-6-oxo-1,6-dihydropyridin-3-yl, 1-methyl-2-oxo-1,2-dihydropyridin-4-yl, 1-methyl-2-oxo-1,2-dihydropyridin-3-yl, 1-acetylpiperidin-4-yl, 3-(2-aminoethan-1-yl)oxypyridin-2-yl, 1-methoxycarbonylpiperidin-4-yl, 3-pyrrolidin-3-ylpyridin-2-yl, 3-(3-aminopropan-1-yl)oxypyridin-2-yl, 3-piperidin-4-ylpyridin-2-yl, 3-piperiazn-1-ylpyridin-2-yl, 1-methylsulfonylpiperidin-4-yl, 3-(2-piperazin-1-ylethan-1-yl)oxypyridin-2-yl, or 3-(3-morpholin-4-ylpropan-1-yl)oxypyridin-2-yl.

Embodiment 119. The compound of any one of embodiments 102-118, wherein R² is hydrogen.

Embodiment 120. The compound of any one of embodiments 102-118, wherein R² is halo, —C₁-C₄ alkyl, —O—C₁-C₄ alkyl, —C₂-C₄ alkynyl, —CN, or cyclopropyl.

Embodiment 121. The compound of embodiment 120, wherein R² is halo.

Embodiment 122. The compound of embodiment 121, wherein R² is fluoro or chloro.

Embodiment 123. The compound of embodiment 121, wherein R² is —C₁-C₄ alkyl.

Embodiment 124. The compound of embodiment 123, wherein R² is —C₁-C₃ alkyl.

Embodiment 125. The compound of embodiment 124, wherein R² is methyl or ethyl.

Embodiment 126. The compound of embodiment 120, wherein R² is —O—C₁-C₄ alkyl.

Embodiment 127. The compound of embodiment 126, wherein R² is —O—C₁-C₃ alkyl.

Embodiment 128. The compound of embodiment 127, wherein R² is —OCH₃ or —OCH₂CH₃.

Embodiment 129. The compound of embodiment 120, wherein R² is —C₂-C₄ alkynyl.

Embodiment 130. The compound of embodiment 129, wherein R² is —C≡CH.

Embodiment 131. The compound of embodiment 120, wherein R² is —CN.

Embodiment 132. The compound of embodiment 120, wherein R² is cyclopropyl.

Embodiment 133. The compound of embodiment 120, wherein R² is selected from —C₁-C₄ alkyl, —C₂-C₄ alkynyl, and cyclopropyl.

Embodiment 134. The compound of embodiment 120, wherein R² is selected from halo, —O—C₁-C₄ alkyl, and —CN.

Embodiment 135. The compound of embodiment 120, wherein R² is selected from chloro, —CH₃, —CH₂CH₃, and —C≡CH.

Embodiment 136. The compound of any one of embodiments 102-135, wherein R³ is hydrogen.

Embodiment 137. The compound of any one of embodiments 102-135, wherein R³ is selected from halo, —C₁-C₄ alkyl, —O—C₁-C₄ alkyl, —CN, cyclopropyl, and phenyl.

Embodiment 138. The compound of embodiment 137, wherein R³ is halo.

Embodiment 139. The compound of embodiment 138, wherein R³ is fluoro or chloro.

Embodiment 140. The compound of embodiment 137, wherein R³ is —C₁-C₄ alkyl.

Embodiment 141. The compound of embodiment 140, wherein R³ is —C₁-C₃ alkyl.

Embodiment 142. The compound of embodiment 141, wherein R³ is methyl or ethyl.

Embodiment 143. The compound of embodiment 137, wherein R³ is —O—C₁-C₄ alkyl.

Embodiment 144. The compound of embodiment 143, wherein R³ is —O—C₁-C₃ alkyl.

Embodiment 145. The compound of embodiment 144, wherein R³ is —OCH₃ or —OCH₂CH₃.

Embodiment 146. The compound of embodiment 137, wherein R³ is —CN.

Embodiment 147. The compound of embodiment 137, wherein R³ is cyclopropyl.

Embodiment 148. The compound of embodiment 137, wherein R³ is phenyl.

Embodiment 149. The compound of embodiment 137, wherein R³ is selected from —C₁-C₄ alkyl, —C₂-C₄ alkynyl, cyclopropyl, and phenyl.

Embodiment 150. The compound of embodiment 137, wherein R³ is selected from halo, —O—C₁-C₄ alkyl, and —CN.

Embodiment 151. The compound of any one of embodiments 102-150, wherein R⁴ is hydrogen.

Embodiment 152. The compound of any one of embodiments 102-150, wherein R⁴ is selected from halo, —C₁-C₄ alkyl, —O—C₁-C₄ alkyl, —C₂-C₄ alkynyl, —CN, cyclopropyl, and phenyl.

Embodiment 153. The compound of embodiment 152, wherein R⁴ is halo.

Embodiment 154. The compound of embodiment 153, wherein R⁴ is fluoro or chloro.

Embodiment 155. The compound of embodiment 152, wherein R⁴ is —C₁-C₄ alkyl.

Embodiment 156. The compound of embodiment 155, wherein R⁴ is —C₁-C₃ alkyl.

Embodiment 157. The compound of embodiment 156, wherein R⁴ is methyl or ethyl.

Embodiment 158. The compound of embodiment 152, wherein R⁴ is —O—C₁-C₄ alkyl.

Embodiment 159. The compound of embodiment 158, wherein R⁴ is —O—C₁-C₃ alkyl.

Embodiment 160. The compound of embodiment 159, wherein R⁴ is —OCH₃ or —OCH₂CH₃.

Embodiment 161. The compound of embodiment 152, wherein R⁴ is —CN.

Embodiment 162. The compound of embodiment 152, wherein R⁴ is cyclopropyl.

Embodiment 163. The compound of embodiment 152, wherein R⁴ is phenyl.

Embodiment 164. The compound of embodiment 152, wherein R⁴ is selected from —C₁-C₄ alkyl, cyclopropyl, and phenyl.

Embodiment 165. The compound of embodiment 152, wherein R⁴ is selected from halo, —O—C₁-C₄ alkyl, and —CN.

Embodiment 166. The compound of any one of embodiments 102-165, wherein R⁷ is hydrogen.

Embodiment 167. The compound of any one of embodiments 102-165, wherein R⁷ is optionally substituted C₁-C₄ alkyl or optionally substituted —O—C₁-C₄ alkyl.

Embodiment 168. The compound of embodiment 167, wherein R⁷ is optionally substituted C₁-C₄ alkyl.

Embodiment 169. The compound of embodiment 168, wherein R⁷ is optionally substituted C₁-C₃ alkyl.

Embodiment 170. The compound of embodiment 169, wherein R⁷ is optionally substituted methyl or ethyl.

Embodiment 171. The compound of any one of embodiments 102-170, wherein R⁸ is hydrogen.

Embodiment 172. The compound of any one of embodiments 102-170, wherein R⁸ is optionally substituted C₁-C₄ alkyl.

Embodiment 173. The compound of embodiment 172, wherein R⁸ is optionally substituted C₁-C₄ alkyl.

Embodiment 174. The compound of embodiment 173, wherein R⁸ is optionally substituted C₁-C₃ alkyl.

Embodiment 175. The compound of embodiment 174, wherein R⁸ is optionally substituted methyl or ethyl.

Embodiment 176. The compound of any one of embodiments 102-175, wherein:
R² is hydrogen, fluoro, chloro, bromo, iodo, methyl, ethyl, or ethynyl;
R³ is hydrogen, fluoro, methyl or —CN;
R⁴ is hydrogen, fluoro, methyl or —CN;
R⁵ is —OH; and
R⁶ is methyl, ethyl, propyl, isopropyl, —CN, —OCH₃, cyclopropyl or phenyl.

Embodiment 177. The compound of embodiment 176, wherein:
R² is hydrogen, chloro, methyl, ethyl or ethynyl;
each of R³ and R⁴ is hydrogen;
R⁵ is —OH; and
R⁶ is hydrogen, chloro, or methyl.

The compound of any one of embodiments 102-177, wherein at least one of R², R³, R⁴, and R⁶ is other than hydrogen.

Embodiment 178. The compound of embodiment 177, wherein at least one of R² and R⁶ is other than hydrogen.

Embodiment 179. The compound of any one of embodiments 102-178, wherein R⁵ and R⁶ are taken together with carbon atoms to which they are bound to form Ring A, wherein Ring A is a 4-7 membered heterocyclic or heteroaromatic ring comprising a ring nitrogen atom such that the portion of the compound represented by:

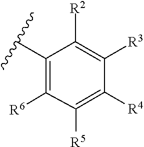

has a bicyclic ring structure selected from:

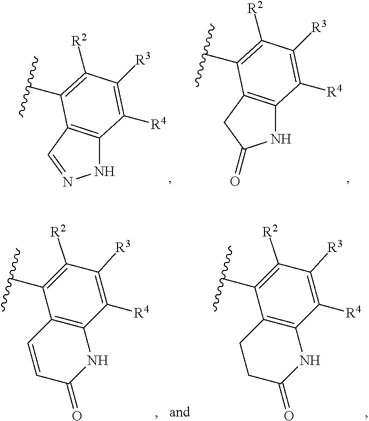

wherein the heterocyclic or heteroaromatic ring portion of the bicyclic ring is optionally further substituted with 1-2 substituents independently selected from halo, C₁-C₄ alkyl, C₁-C₄ haloalkyl, —O—C₁-C₄ alkyl, or —O—C₁-C₄ haloalkyl.

Embodiment 180. The compound of embodiment 179, wherein R² is chloro, methyl, ethyl, or ethynyl; and each of R³ and R⁴ is hydrogen.

Embodiment 181. The compound of embodiment 180, wherein the heterocyclic or heteroaromatic ring portion of the bicyclic ring is optionally further substituted with chloro.

Embodiment 182. A pharmaceutical composition comprising an effective amount of the compound of any one of embodiments 1-181; and a pharmaceutically acceptable carrier.

Embodiment 183. A method of inhibiting Myt1 kinase activity in a subject comprising the step of administering to the subject an effective amount of a compound of any one of embodiments 1-181, or a composition of embodiment 182.

Embodiment 184. A method of treating a subject suffering from a cancer characterized by aberrant Myt1 kinase activity comprising the step of administering to the subject an effective amount of a compound of any one of embodiments 1-181, or a composition of embodiment 181.

Embodiment 185. A method of treating a subject suffering from a cancer characterized by amplification and/or overexpression of CCNE1 comprising the step of administering to the subject an effective amount of a compound of any one of embodiments 1-181, or a composition of embodiment 182.

Embodiment 186. The method of embodiment 184 or 185, wherein the cancer is uterine cancer, ovarian cancer, breast cancer, stomach cancer, esophageal cancer, lung cancer, or endometrial cancer.

Embodiment 187. A method of treating a subject suffering from a cancer characterized by an inactivating mutation in a FBXW7 gene, comprising the step of administering to the subject an effective amount of a compound of any one of embodiments 1-181, or a composition of embodiment 182.

Embodiment 188. The method of embodiment 184 or 187, wherein the cancer is uterine cancer, colorectal cancer, breast cancer, lung cancer, or esophageal cancer.

Embodiment 189. The method of any one of embodiments 183-188, wherein the subject is treated only if it has been determined that the subject is resistant to a WeelA kinase inhibitor.

Embodiment 190. The method of any one of embodiments 183-188, wherein the subject is co-administered a pharmaceutically acceptable Wee1A kinase inhibitor.

Embodiment 191. The method of any one of embodiments 183-190, wherein the subject is co-administered a pharmaceutically acceptable DNA damaging agent.

In some embodiments, the present disclosure provides a compound selected from Table 1.

| # | Structure |
|---|---|
| 1 | 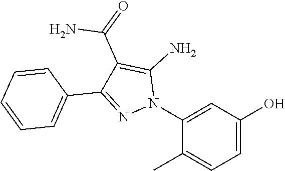 [SMILES: Cc1ccc(O)cc1-n1nc(-c2ccccc2)c(C(N)=O)c1N] |
| 2 | 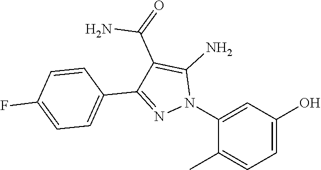 [SMILES: Cc1ccc(O)cc1-n1nc(-c2ccc(F)cc2)c(C(N)=O)c1N] |
| 3 | 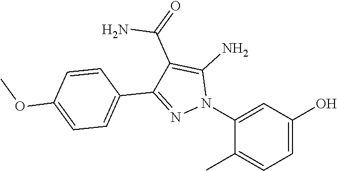 [SMILES: COc1ccc(-c2nn(-c3cc(O)ccc3C)c(N)c2C(N)=O)cc1] |
| 4 | 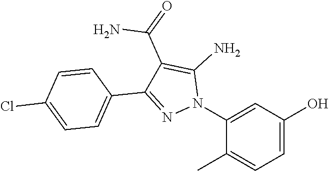 [SMILES: Cc1ccc(O)cc1-n1nc(-c2ccc(Cl)cc2)c(C(N)=O)c1N] |
| 5 | 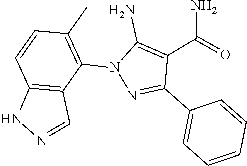 [SMILES: Cc1ccc2[nH]ncc2c1-n1nc(-c2ccccc2)c(C(N)=O)c1N] |
| 6 | 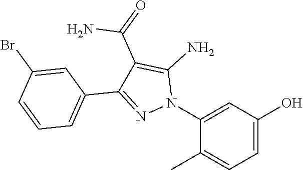 [SMILES: Cc1ccc(O)cc1-n1nc(-c2cccc(Br)c2)c(C(N)=O)c1N] |
| 7 | 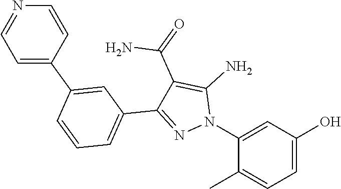 [SMILES: Cc1ccc(O)cc1-n1nc(-c2cccc(-c3ccncc3)c2)c(C(N)=O)c1N] |
| 8 | 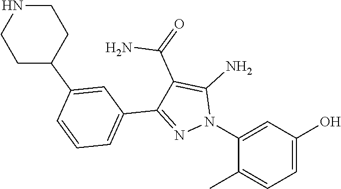 [SMILES: Cc1ccc(O)cc1-n1nc(-c2cccc(C3CCNCC3)c2)c(C(N)=O)c1N] |
| 9 | 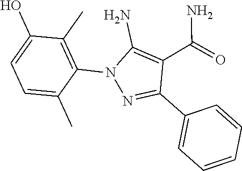 [SMILES: Cc1ccc(O)c(C)c1-n1nc(-c2ccccc2)c(C(N)=O)c1N] |
| 10 | 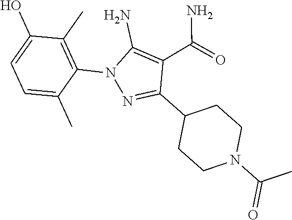 [SMILES: CC(=O)N1CCC(c2nn(-c3c(C)ccc(O)c3C)c(N)c2C(N)=O)CC1] |
| 11 | 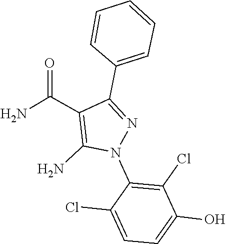 [SMILES: NC(=O)c1c(-c2ccccc2)nn(-c2c(Cl)ccc(O)c2Cl)c1N] |
| 12 | 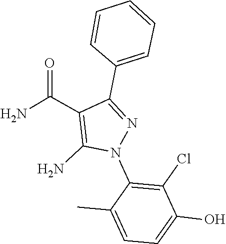 [SMILES: Cc1ccc(O)c(Cl)c1-n1nc(-c2ccccc2)c(C(N)=O)c1N] |
| 13 | 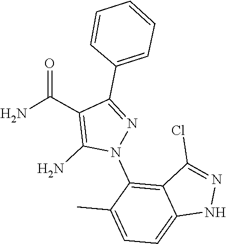 [SMILES: Cc1ccc2[nH]nc(Cl)c2c1-n1nc(-c2ccccc2)c(C(N)=O)c1N] |
| 14 | 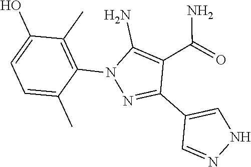 [SMILES: Cc1ccc(O)c(C)c1-n1nc(-c2cn[nH]c2)c(C(N)=O)c1N] |
| 15 | 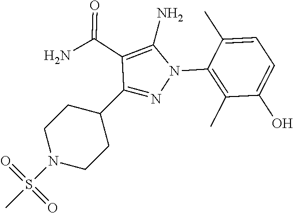 [SMILES: Cc1ccc(O)c(C)c1-n1nc(C2CCN(S(C)(=O)=O)CC2)c(C(N)=O)c1N] |
| 16 | 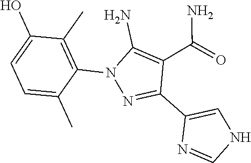 [SMILES: Cc1ccc(O)c(C)c1-n1nc(-c2c[nH]cn2)c(C(N)=O)c1N] |
| 17 | 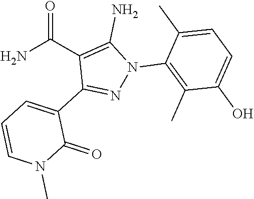 [SMILES: Cc1ccc(O)c(C)c1-n1nc(-c2cccn(C)c2=O)c(C(N)=O)c1N] |
| 18 | 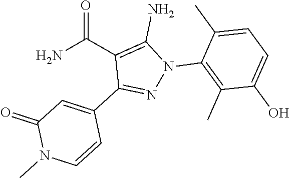 [SMILES: Cc1ccc(O)c(C)c1-n1nc(-c2ccn(C)c(=O)c2)c(C(N)=O)c1N] |
| 19 | 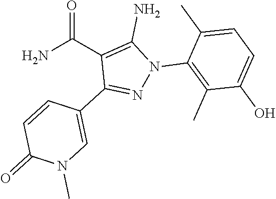 [SMILES: Cc1ccc(O)c(C)c1-n1nc(-c2ccc(=O)n(C)c2)c(C(N)=O)c1N] |
| 20 | 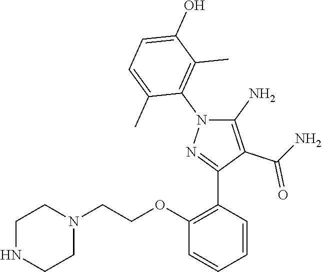 [SMILES: Cc1ccc(O)c(C)c1-n1nc(-c2ccccc2OCCN2CCNCC2)c(C(N)=O)c1N] |
| 21 | 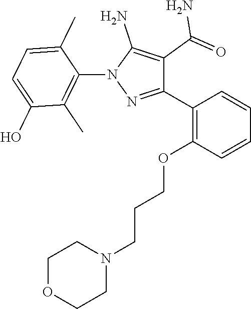 [SMILES: Cc1ccc(O)c(C)c1-n1nc(-c2ccccc2OCCCN2CCOCC2)c(C(N)=O)c1N] |
| 22 | 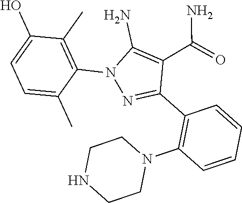 [SMILES: Cc1ccc(O)c(C)c1-n1nc(-c2ccccc2N2CCNCC2)c(C(N)=O)c1N] |
| 23 | 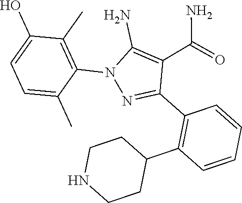 [SMILES: Cc1ccc(O)c(C)c1-n1nc(-c2ccccc2C2CCNCC2)c(C(N)=O)c1N] |
| 24 | 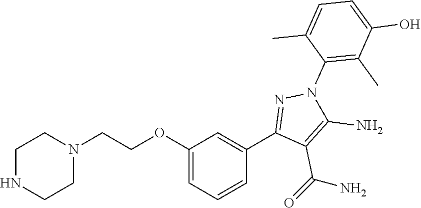 [SMILES: Cc1ccc(O)c(C)c1-n1nc(-c2cccc(OCCN3CCNCC3)c2)c(C(N)=O)c1N] |
| 25 | 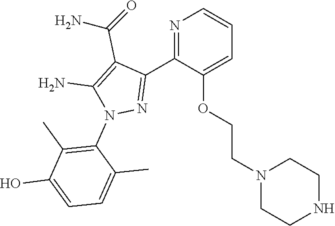 [SMILES: Cc1ccc(O)c(C)c1-n1nc(-c2ncccc2OCCN2CCNCC2)c(C(N)=O)c1N] |
| 26 | 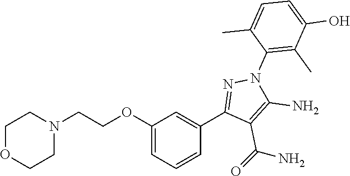 [SMILES: Cc1ccc(O)c(C)c1-n1nc(-c2cccc(OCCN3CCOCC3)c2)c(C(N)=O)c1N] |
| 27 | 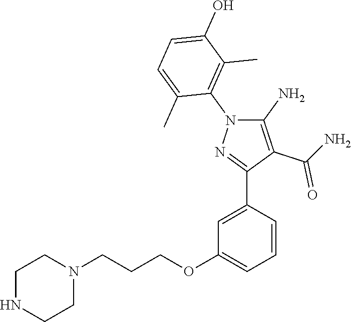 [SMILES: Cc1ccc(O)c(C)c1-n1nc(-c2cccc(OCCCN3CCNCC3)c2)c(C(N)=O)c1N] |
| 28 | 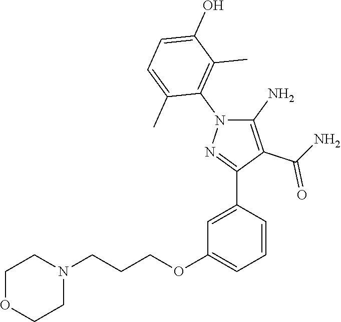 [SMILES: Cc1ccc(O)c(C)c1-n1nc(-c2cccc(OCCCN3CCOCC3)c2)c(C(N)=O)c1N] |
| 29 | 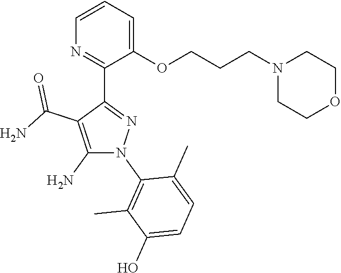 [SMILES: Cc1ccc(O)c(C)c1-n1nc(-c2ncccc2OCCCN2CCOCC2)c(C(N)=O)c1N] |
| 30 | 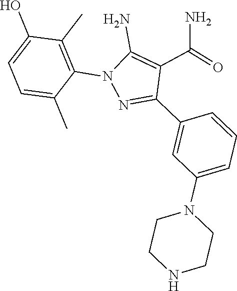 [SMILES: Cc1ccc(O)c(C)c1-n1nc(-c2cccc(N3CCNCC3)c2)c(C(N)=O)c1N] |
| 31 | 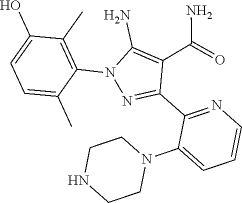 [SMILES: Cc1ccc(O)c(C)c1-n1nc(-c2ncccc2N2CCNCC2)c(C(N)=O)c1N] |
| 32 | 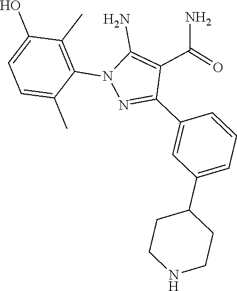 [SMILES: Cc1ccc(O)c(C)c1-n1nc(-c2cccc(C3CCNCC3)c2)c(C(N)=O)c1N] |
| 33 | 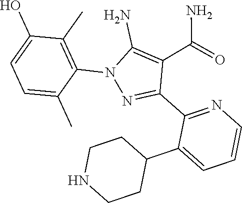 [SMILES: Cc1ccc(O)c(C)c1-n1nc(-c2ncccc2C2CCNCC2)c(C(N)=O)c1N] |
| 34 | 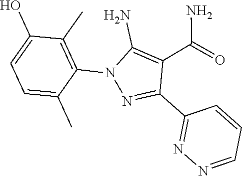 [SMILES: Cc1ccc(O)c(C)c1-n1nc(-c2cccnn2)c(C(N)=O)c1N] |
| 35 | 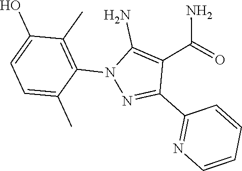 [SMILES: Cc1ccc(O)c(C)c1-n1nc(-c2ccccn2)c(C(N)=O)c1N] |
| 36 | 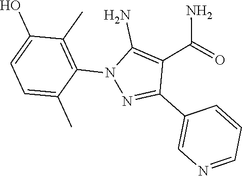 [SMILES: Cc1ccc(O)c(C)c1-n1nc(-c2cccnc2)c(C(N)=O)c1N] |
| 37 | 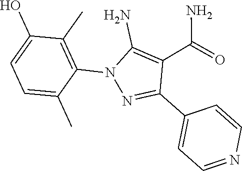 [SMILES: Cc1ccc(O)c(C)c1-n1nc(-c2ccncc2)c(C(N)=O)c1N] |
| 38 | 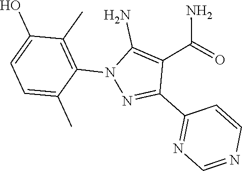 [SMILES: Cc1ccc(O)c(C)c1-n1nc(-c2ccncn2)c(C(N)=O)c1N] |
| 39 | 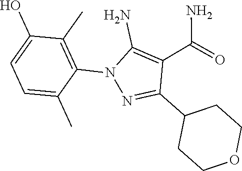 [SMILES: Cc1ccc(O)c(C)c1-n1nc(C2CCOCC2)c(C(N)=O)c1N] |
| 40 | 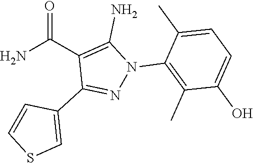 [SMILES: Cc1ccc(O)c(C)c1-n1nc(-c2ccsc2)c(C(N)=O)c1N] |
| 41 | 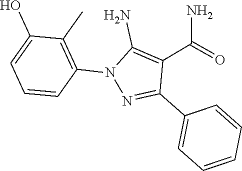 [SMILES: Cc1c(O)cccc1-n1nc(-c2ccccc2)c(C(N)=O)c1N] |
| 42 | 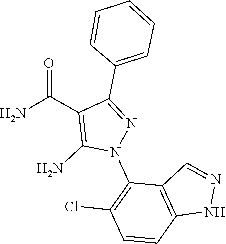 [SMILES: NC(=O)c1c(-c2ccccc2)nn(-c2c(Cl)ccc3[nH]ncc23)c1N] |
| 43 | 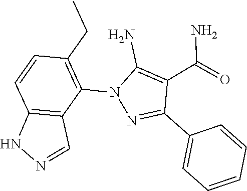 [SMILES: CCc1ccc2[nH]ncc2c1-n1nc(-c2ccccc2)c(C(N)=O)c1N] |
| 44 | 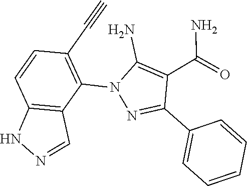 [SMILES: C#Cc1ccc2[nH]ncc2c1-n1nc(-c2ccccc2)c(C(N)=O)c1N] |
| 45 | 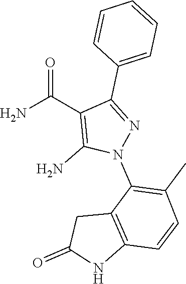 [SMILES: Cc1ccc2c(c1-n1nc(-c3ccccc3)c(C(N)=O)c1N)CC(=O)N2] |
| 46 | 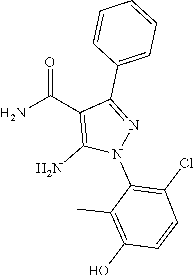 [SMILES: Cc1c(O)ccc(Cl)c1-n1nc(-c2ccccc2)c(C(N)=O)c1N] |
| 47 | 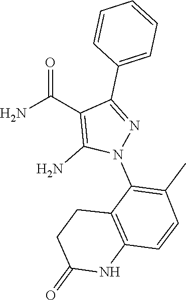 [SMILES: Cc1ccc2c(c1-n1nc(-c3ccccc3)c(C(N)=O)c1N)CCC(=O)N2] |
| 48 | 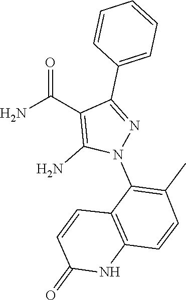 [SMILES: Cc1ccc2[nH]c(=O)ccc2c1-n1nc(-c2ccccc2)c(C(N)=O)c1N] |
| 49 | 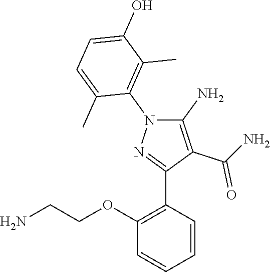 [SMILES: Cc1ccc(O)c(C)c1-n1nc(-c2ccccc2OCCN)c(C(N)=O)c1N] |
| 50 | 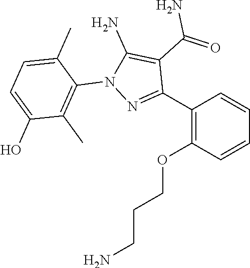 [SMILES: Cc1ccc(O)c(C)c1-n1nc(-c2ccccc2OCCCN)c(C(N)=O)c1N] |
| 51 | 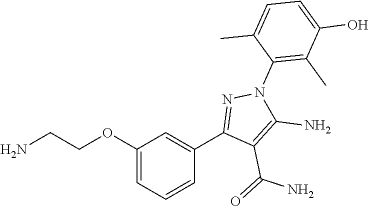 [SMILES: Cc1ccc(O)c(C)c1-n1nc(-c2cccc(OCCN)c2)c(C(N)=O)c1N] |
| 52 | 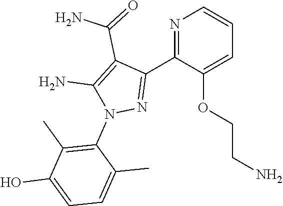 [SMILES: Cc1ccc(O)c(C)c1-n1nc(-c2ncccc2OCCN)c(C(N)=O)c1N] |
| 53 | 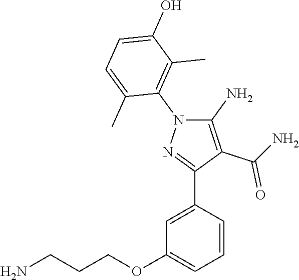 [SMILES: Cc1ccc(O)c(C)c1-n1nc(-c2cccc(OCCCN)c2)c(C(N)=O)c1N] |
| 54 | 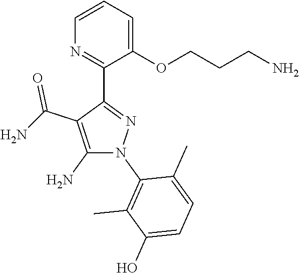 [SMILES: Cc1ccc(O)c(C)c1-n1nc(-c2ncccc2OCCCN)c(C(N)=O)c1N] |
| 55 | 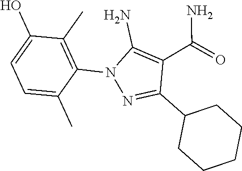 [SMILES: Cc1ccc(O)c(C)c1-n1nc(C2CCCCC2)c(C(N)=O)c1N] |
| 56 | 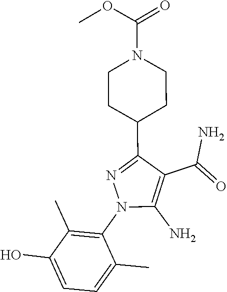 [SMILES: COC(=O)N1CCC(c2nn(-c3c(C)ccc(O)c3C)c(N)c2C(N)=O)CC1] |
| 57 | 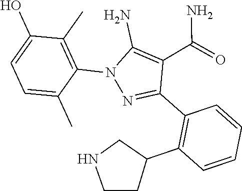 [SMILES: Cc1ccc(O)c(C)c1-n1nc(-c2ccccc2C2CCNC2)c(C(N)=O)c1N] |
| 58 | 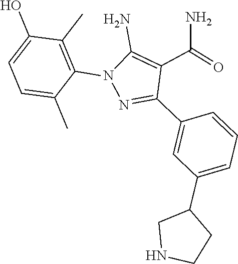 [SMILES: Cc1ccc(O)c(C)c1-n1nc(-c2cccc(C3CCNC3)c2)c(C(N)=O)c1N] |
| 59 | 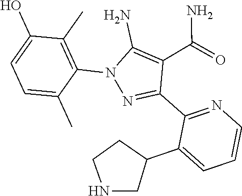 [SMILES: Cc1ccc(O)c(C)c1-n1nc(-c2ncccc2C2CCNC2)c(C(N)=O)c1N] |
| 60 | 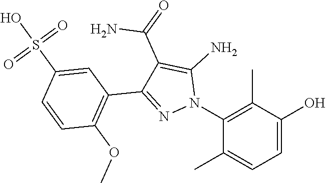 [SMILES: COc1ccc(S(=O)(=O)O)cc1-c1nn(-c2c(C)ccc(O)c2C)c(N)c1C(N)=O] |
| 61 | 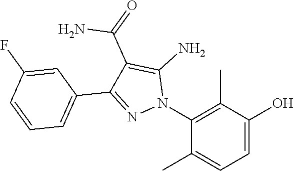 [SMILES: Cc1ccc(O)c(C)c1-n1nc(-c2cccc(F)c2)c(C(N)=O)c1N] |
| 62 | 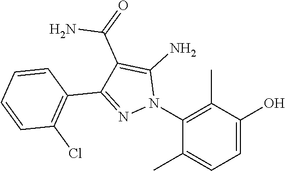 [SMILES: Cc1ccc(O)c(C)c1-n1nc(-c2ccccc2Cl)c(C(N)=O)c1N] |
| 63 | 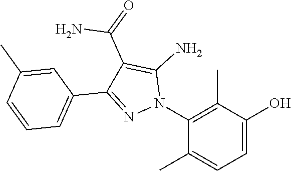 [SMILES: Cc1cccc(-c2nn(-c3c(C)ccc(O)c3C)c(N)c2C(N)=O)c1] |
| 64 | 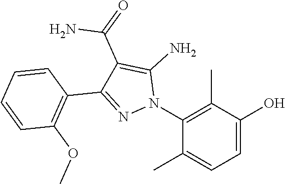 [SMILES: COc1ccccc1-c1nn(-c2c(C)ccc(O)c2C)c(N)c1C(N)=O] |
| 65 | 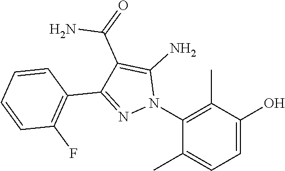 [SMILES: Cc1ccc(O)c(C)c1-n1nc(-c2ccccc2F)c(C(N)=O)c1N] |
| 66 | 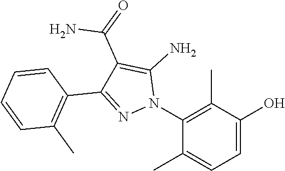 [SMILES: Cc1ccccc1-c1nn(-c2c(C)ccc(O)c2C)c(N)c1C(N)=O] |
| 67 | 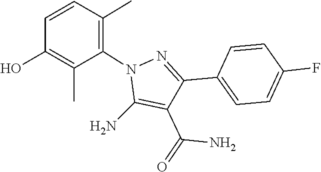 [SMILES: Cc1ccc(O)c(C)c1-n1nc(-c2ccc(F)cc2)c(C(N)=O)c1N] |
| 68 | 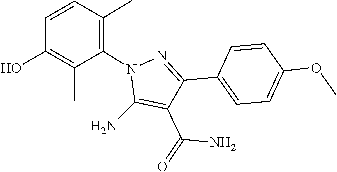 [SMILES: COc1ccc(-c2nn(-c3c(C)ccc(O)c3C)c(N)c2C(N)=O)cc1] |
| 69 | 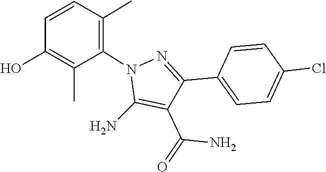 [SMILES: Cc1ccc(O)c(C)c1-n1nc(-c2ccc(Cl)cc2)c(C(N)=O)c1N] |
| 70 | 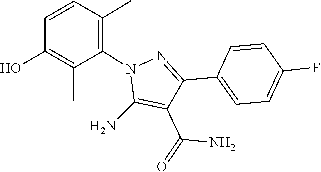 [SMILES: Cc1ccc(O)c(C)c1-n1nc(-c2ccc(F)cc2)c(C(N)=O)c1N] |
| 71 | 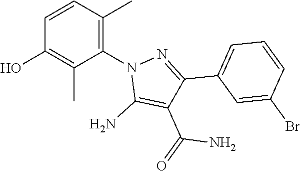 [SMILES: Cc1ccc(O)c(C)c1-n1nc(-c2cccc(Br)c2)c(C(N)=O)c1N] |
| 72 | 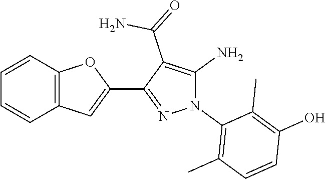 [SMILES: Cc1ccc(O)c(C)c1-n1nc(-c2cc3ccccc3o2)c(C(N)=O)c1N] |
| 73 | 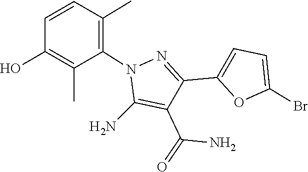 [SMILES: Cc1ccc(O)c(C)c1-n1nc(-c2ccc(Br)o2)c(C(N)=O)c1N] |
| 74 | 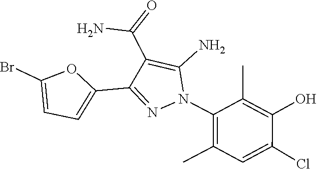 [SMILES: Cc1cc(Cl)c(O)c(C)c1-n1nc(-c2ccc(Br)o2)c(C(N)=O)c1N] |
| 75 | 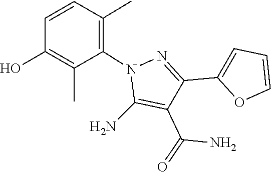 [SMILES: Cc1ccc(O)c(C)c1-n1nc(-c2ccco2)c(C(N)=O)c1N] |
| 76 | 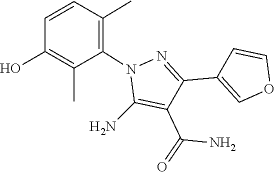 [SMILES: Cc1ccc(O)c(C)c1-n1nc(-c2ccoc2)c(C(N)=O)c1N] |
| 77 | 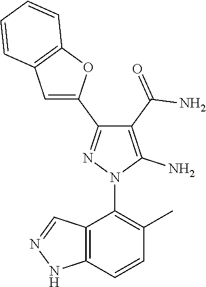 [SMILES: Cc1ccc2[nH]ncc2c1-n1nc(-c2cc3ccccc3o2)c(C(N)=O)c1N] |
| 78 | 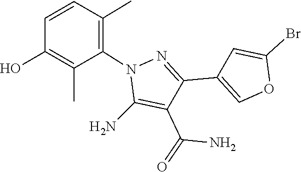 [SMILES: Cc1ccc(O)c(C)c1-n1nc(-c2coc(Br)c2)c(C(N)=O)c1N] |
| 79 | 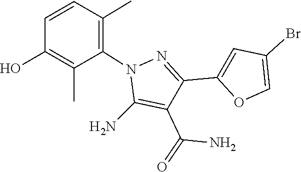 [SMILES: Cc1ccc(O)c(C)c1-n1nc(-c2cc(Br)co2)c(C(N)=O)c1N] |
| 80 | 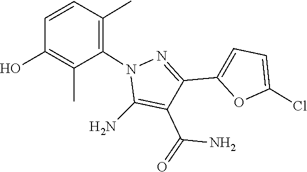 [SMILES: Cc1ccc(O)c(C)c1-n1nc(-c2ccc(Cl)o2)c(C(N)=O)c1N] |
| 81 | 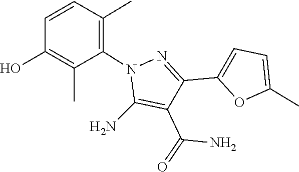 [SMILES: Cc1ccc(-c2nn(-c3c(C)ccc(O)c3C)c(N)c2C(N)=O)o1] |
| 82 | 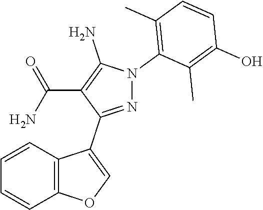 [SMILES: Cc1ccc(O)c(C)c1-n1nc(-c2coc3ccccc23)c(C(N)=O)c1N] |
| 83 | 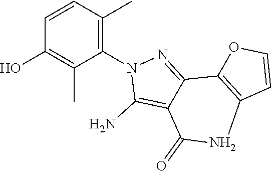 [SMILES: Cc1ccc(O)c(C)c1-n1nc2c(c1N)c(=O)[nH]c1ccoc12] |
| 84 | 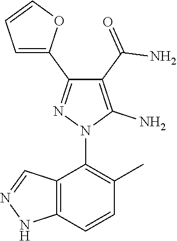 [SMILES: Cc1ccc2[nH]ncc2c1-n1nc(-c2ccco2)c(C(N)=O)c1N] |
| 85 | 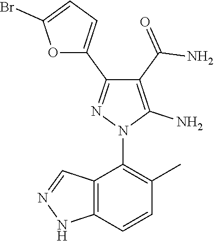 [SMILES: Cc1ccc2[nH]ncc2c1-n1nc(-c2ccc(Br)o2)c(C(N)=O)c1N] |
| 86 | 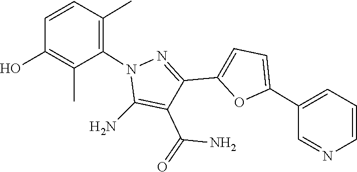 [SMILES: Cc1ccc(O)c(C)c1-n1nc(-c2ccc(-c3cccnc3)o2)c(C(N)=O)c1N] |
| 87 | 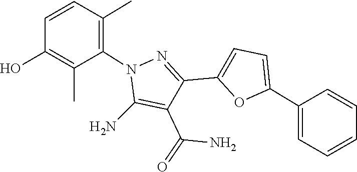 [SMILES: Cc1ccc(O)c(C)c1-n1nc(-c2ccc(-c3ccccc3)o2)c(C(N)=O)c1N] |
| 88 | 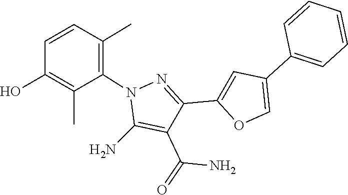 [SMILES: Cc1ccc(O)c(C)c1-n1nc(-c2cc(-c3ccccc3)co2)c(C(N)=O)c1N] |
| 89 | 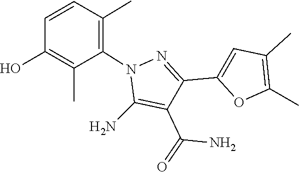 [SMILES: Cc1cc(-c2nn(-c3c(C)ccc(O)c3C)c(N)c2C(N)=O)oc1C] |
| 90 | 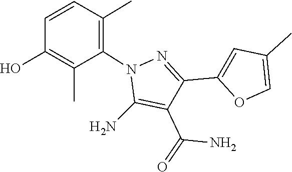 [SMILES: Cc1coc(-c2nn(-c3c(C)ccc(O)c3C)c(N)c2C(N)=O)c1] |
| 91 | 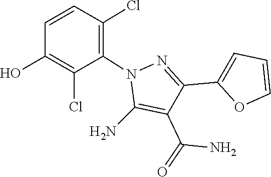 [SMILES: NC(=O)c1c(-c2ccco2)nn(-c2c(Cl)ccc(O)c2Cl)c1N] |
| 92 | 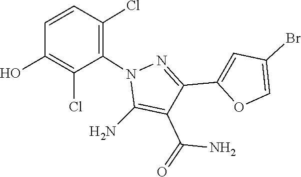 [SMILES: NC(=O)c1c(-c2cc(Br)co2)nn(-c2c(Cl)ccc(O)c2Cl)c1N] |
| 93 | 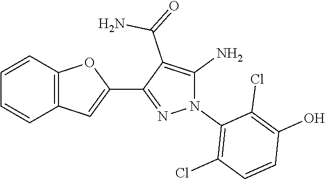 [SMILES: NC(=O)c1c(-c2cc3ccccc3o2)nn(-c2c(Cl)ccc(O)c2Cl)c1N] |
| 94 | 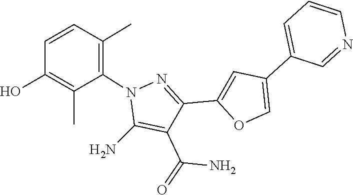 [SMILES: Cc1ccc(O)c(C)c1-n1nc(-c2cc(-c3cccnc3)co2)c(C(N)=O)c1N] |
| 95 | 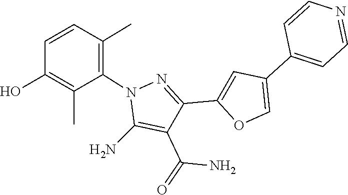 [SMILES: Cc1ccc(O)c(C)c1-n1nc(-c2cc(-c3ccncc3)co2)c(C(N)=O)c1N] |
| 96 | 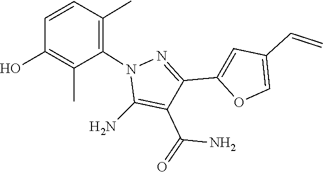 [SMILES: C=Cc1coc(-c2nn(-c3c(C)ccc(O)c3C)c(N)c2C(N)=O)c1] |
| 97 | 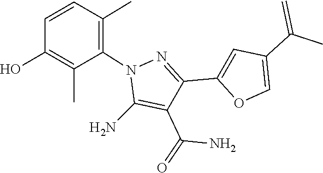 [SMILES: C=C(C)c1coc(-c2nn(-c3c(C)ccc(O)c3C)c(N)c2C(N)=O)c1] |
| 98 | 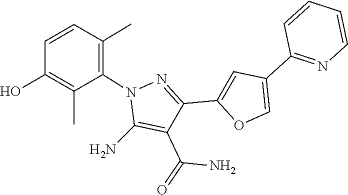 [SMILES: Cc1ccc(O)c(C)c1-n1nc(-c2cc(-c3ccccn3)co2)c(C(N)=O)c1N] |
| 99 | 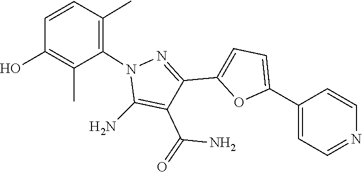 [SMILES: Cc1ccc(O)c(C)c1-n1nc(-c2ccc(-c3ccncc3)o2)c(C(N)=O)c1N] |
| 100 | 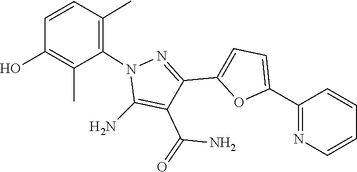 [SMILES: Cc1ccc(O)c(C)c1-n1nc(-c2ccc(-c3ccccn3)o2)c(C(N)=O)c1N] |
| 101 | 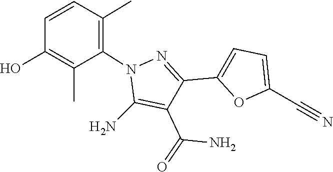 [SMILES: Cc1ccc(O)c(C)c1-n1nc(-c2ccc(C#N)o2)c(C(N)=O)c1N] |
| 102 | 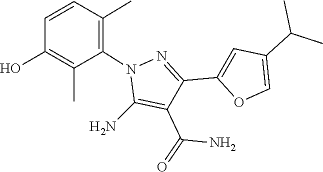 [SMILES: Cc1ccc(O)c(C)c1-n1nc(-c2cc(C(C)C)co2)c(C(N)=O)c1N] |
| 103 | 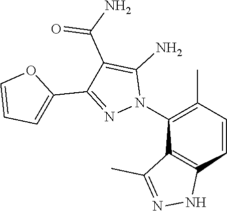 |
| 104 | 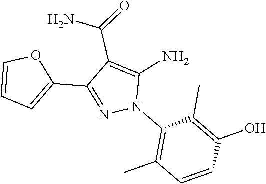 |
| 105 | 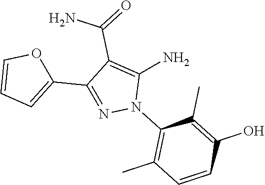 |
| 106 | 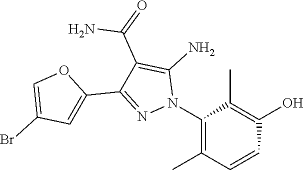 |
| 107 | 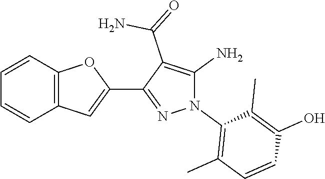 |
| 108 | 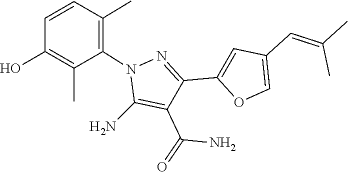 [SMILES: CC(C)=Cc1coc(-c2nn(-c3c(C)ccc(O)c3C)c(N)c2C(N)=O)c1] |
| 109 | 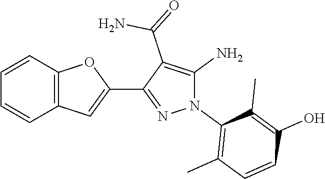 |
| 110 | 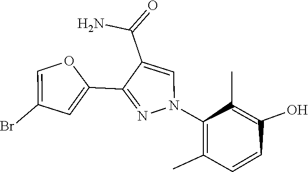 |
| 111 | 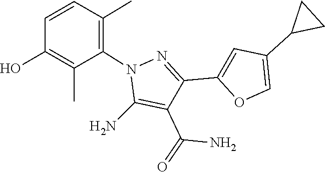 [SMILES: Cc1ccc(O)c(C)c1-n1nc(-c2cc(C3CC3)co2)c(C(N)=O)c1N] |
| 112 | 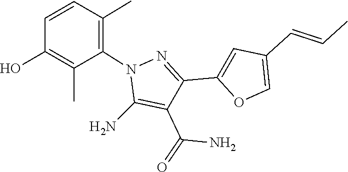 [SMILES: C/C=C/c1coc(-c2nn(-c3c(C)ccc(O)c3C)c(N)c2C(N)=O)c1] |
| 113 | 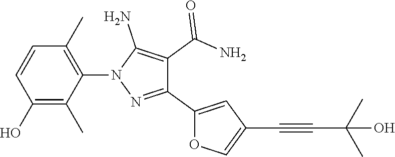 [SMILES: Cc1ccc(O)c(C)c1-n1nc(-c2cc(C#CC(C)(C)O)co2)c(C(N)=O)c1N] |
| 114 | 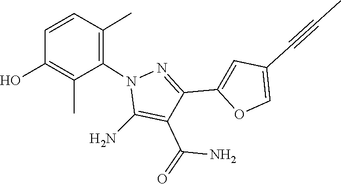 [SMILES: CC#Cc1coc(-c2nn(-c3c(C)ccc(O)c3C)c(N)c2C(N)=O)c1] |
| 115 | 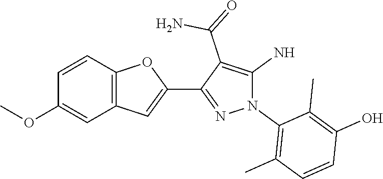 [SMILES: COc1ccc2oc(-c3nn(-c4c(C)ccc(O)c4C)c(N)c3C(N)=O)cc2c1] |
| 116 | 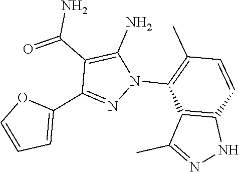 |
| 117 | 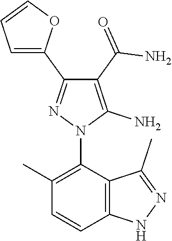 [SMILES: Cc1ccc2[nH]nc(C)c2c1-n1nc(-c2ccco2)c(C(N)=O)c1N] |
| 118 | 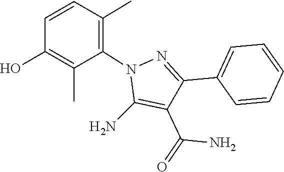 [SMILES: Cc1ccc(O)c(C)c1-n1nc(-c2ccccc2)c(C(N)=O)c1N] |
| 119 | 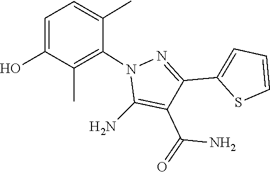 [SMILES: Cc1ccc(O)c(C)c1-n1nc(-c2cccs2)c(C(N)=O)c1N] |
| 120 | 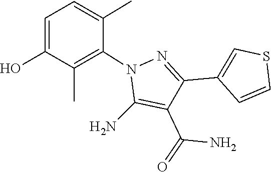 [SMILES: Cc1ccc(O)c(C)c1-n1nc(-c2ccsc2)c(C(N)=O)c1N] |
| 121 | 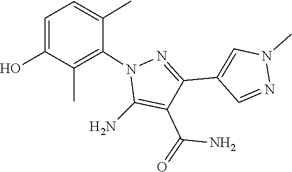 [SMILES: Cc1ccc(O)c(C)c1-n1nc(-c2cnn(C)c2)c(C(N)=O)c1N] |
| 122 | 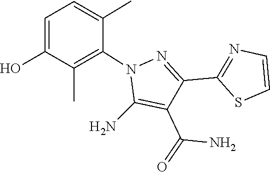 [SMILES: Cc1ccc(O)c(C)c1-n1nc(-c2nccs2)c(C(N)=O)c1N] |
| 123 | 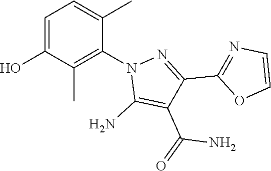 [SMILES: Cc1ccc(O)c(C)c1-n1nc(-c2ncco2)c(C(N)=O)c1N] |
| 124 | 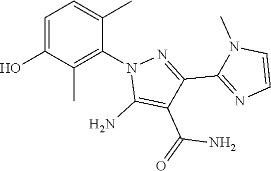 [SMILES: Cc1ccc(O)c(C)c1-n1nc(-c2nccn2C)c(C(N)=O)c1N] |
| 125 | 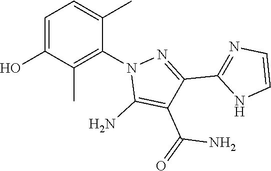 [SMILES: Cc1ccc(O)c(C)c1-n1nc(-c2ncc[nH]2)c(C(N)=O)c1N] |
| 126 | 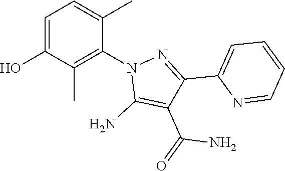 [SMILES: Cc1ccc(O)c(C)c1-n1nc(-c2ccccn2)c(C(N)=O)c1N] |

or a pharmaceutically acceptable salt thereof.

### General Synthesis of Provided Compounds

The compounds of the present disclosure may be synthesized by reacting a hydrazine intermediate having the structure (a):

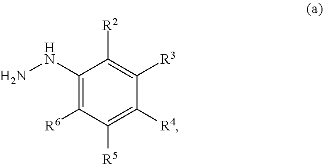

wherein R¹, R², R³, R⁴, R⁵ and R⁶ are as defined for Formula (I); with a having the structure (b):

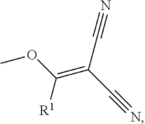

wherein R¹ is also as defined in Formula (I) to form a cyano-pyrazole intermediate having structure (c):

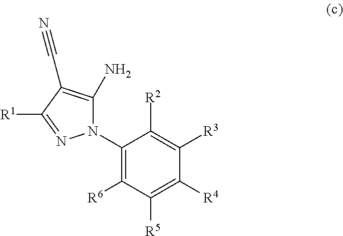

that after hydrolysis produce a carboxamide of formula (I) and optionally thereafter forming a pharmaceutically acceptable salt thereof.

The 2-(methoxymethylene) propanedinitrile derivative according to formula (III) can for example be prepared by reacting a LHS-acyl chloride with commercially available propanedinitrile followed by methylation of the intermediate 2-(hydroxymethylene) propanedinitrile derivative to form the methyl-ether compounds of formula (III).

The methodology in producing compounds of formula (I) according to the above has been previously described in the literature in at least the following references: J. Med. Chem. 2019, 62, 17, 7923-7940; Journal of Heterocyclic Chemistry, 2018, vol. 55, #4, p. 803-813; Journal of Heterocyclic Chemistry, 2018, vol. 55, #7, p. 1615-1625; Molecules 2008, 13(7), 1501-1517; and J. Med. Chem. 2004, 47, 24, 5894-5911.

It will be appreciated by those skilled in the art that in the above synthetic scheme certain potentially reactive functional groups such as hydroxyl or amino groups in the starting reagents or intermediate compounds may need to be protected by suitable protecting groups. Thus, the preparation of the compounds of the invention may involve, at various stages, the addition and removal of one or more protecting groups.

Suitable protecting groups and details of processes for adding and removing such groups are described in ‘Protective Groups in Organic Chemistry’, edited by J. W. F. McOmie, Plenum Press (1973) and ‘Protective Groups in Organic Synthesis’, 3rd edition, T.W. Greene and P.G.M. Wuts, Wiley-Interscience (1999).

The compounds of the invention and intermediates thereto may be isolated from their reaction mixtures and, if necessary, further purified by using standard techniques. The following general methods were used to confirm compound structure via NMR and mass spectroscopy.

### 4. Uses, Formulation and Administration

### Pharmaceutically Acceptable Compositions

According to another embodiment, the disclosure provides a composition comprising a compound of this disclosure or a pharmaceutically acceptable derivative thereof and a pharmaceutically acceptable carrier, adjuvant, or vehicle. The amount of compound in compositions of this disclosure is such that is effective to measurably inhibit Myt1 kinase, or a mutant thereof, in a biological sample or in a patient. In certain embodiments, a composition of this disclosure is formulated for administration to a patient in need of such composition. In some embodiments, a composition of this disclosure is formulated for oral administration to a patient.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration.

The amount of compounds of the present disclosure that may be combined with the carrier materials to produce a composition in a single dosage form will vary depending upon the host treated, the particular mode of administration. Preferably, provided compositions should be formulated so that a dosage of between 0.001-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of Compounds and Pharmaceutically Acceptable Compositions

Compounds and compositions described herein are generally useful for the inhibition of protein kinase activity of one or more enzymes.

Examples of kinases that are inhibited by the compounds and compositions described herein and against which the methods described herein are useful include Myt1 kinase.

The activity of a compound utilized in this disclosure as an inhibitor of Myt1 kinase, or a mutant thereof, may be assayed in vitro, in vivo or in a cell line. In vitro assays include assays that determine inhibition of either the phosphorylation activity and/or the subsequent functional consequences, or ATPase activity of activated Myt1 kinase, or a mutant thereof. Alternate in vitro assays quantitate the ability of the inhibitor to bind to Myt1 kinase.

The inhibition of the DNA damage response (DDR) pathway in the treatment of cancer has recently gained interest, and different DDR inhibitors have been developed. Among them, the most promising ones target the WEE1 kinase family, which has a crucial role in cell cycle regulation and DNA damage identification and repair in both nonmalignant and cancer cells.

### WEE Family

The WEE1 kinase family consists of three serine/threonine kinases sharing conserved molecular structures and encoded by the following genes: WEE1 (Wee1A or Wee1 G2 checkpoint kinase), PKMYT1 (Myt1 kinase or membrane-associated tyrosine- and threonine-specific cdc2-inhibitory kinase), and WEE2 (Wee1B kinase of WEE oocyte meiosis inhibiting kinase). In eukaryotic somatic cells, Wee1A kinase and Myt1 kinase play a key role in cell cycle regulation, in particular, in the entry into mitosis (Schmidt M, Rohe A, Platzer C, et al. Regulation of G2/M transition by inhibition of Wee1A kinase and PMyt1 Kinases. Molecules. 2017; 22:2045). Their role as regulators is crucial during normal cell cycle progression and in response to DNA damage as part of the DNA damage response (DDR) pathways. Similarly, Wee1B kinase regulates cell cycle progression and, in particular, meiosis (Solc P, Schultz R M, Motlik J. Prophase I arrest and progression to metaphase I in mouse oocytes: Comparison of resumption of meiosis and recovery from G2-arrest in somatic cells. Mol Hum Reprod. 2010; 16:654-64).

### Wee1A Kinase

Wee1A kinase regulates entry into mitosis at the G2/M transition of the S phase by phosphorylating Tyr15 of Cdk1 to inactive the Cdk1/cyclin B complex. Cells with perturbed G1 checkpoint activity (e.g., cancer cells) rely on Wee1A kinase to inhibit Cdk1 to permit a G2/M arrest for DNA repair. If Wee1A kinase activity is altered, a perturbed cell may enter mitosis prematurely without having the opportunity to fully replicate the entire DNA content or repair potential DNA that might have occurred during S phase. This characterization of Wee1A kinase's role in the cell cycle has made it an attractive target for anticancer therapeutics, especially in combination with DNA-damaging agents (JY Zhu et al., J Med Chem. 2017; 60 (18), 7863-7875).

### Wee1B Kinase

Wee1B kinase expression is germ-cell specific and inhibits meiosis by phosphorylating Tyr15 of the CDK1-cyclin B complex (JY Zhu et al., J Med Chem. 2017; 60 (18), 7863-7875). Previous and current drug discovery efforts have not been focused on Wee1B kinase due to its characterized role in cell-cycle regulation. Wee1B kinase plays a dual regulatory role in oocyte meiosis by preventing premature restart prior to ovulation and permitting metaphase II exit at fertilization (Nakanishi M, Ando H, Watanabe N, et al. Identification and characterization of human Wee1B, a new member of the Wee1 family of Cdk-inhibitory kinases. Genes Cells. 2000; 5 (10): 839-47). Despite the identification of WEE2 somatic mutations (1.9% of cases) and copy number (CN) alterations (22.5% of patients with CN loss and 22.5% with CN gain) across several cancer types (https://portal.gdc.cancer.gov), they have not yet been functionally linked to tumor development.

### Myt1 Kinase

Myt1 kinase is a multi-functional protein kinase localized to the ER-Golgi complex that is known to play a regulatory role in the cell cycle by inhibiting Cdk1/cyclin B1 mediated mitosis (JY Zhu et al., J Med Chem. 2017; 60 (18), 7863-7875). As mentioned above and throughout, Myt1 kinase inhibits the Cdk1/cyclin B1 interaction through the phosphorylation of Tyr15 and Thr14 of Cdk1 and sequestration of Cdk1 from the nucleus. Additionally, Myt1 kinase has been tied to orchestrating the ER-Golgi complex reassembly during mitotic exit.

Due to the importance of Wee1A kinase's role in regulating the cell cycle, and Myt1 kinase's perceived lack of importance in regulating the cell cycle, Myt1 kinase has garnered less attention as a viable therapeutic target. However, in perturbed cells (e.g., a cell with amplified CCNE1 and/or deregulation of cyclin E1) Myt1 kinase dysfunction can cause cells to lose major checkpoint regulation leading to hyperactive Cdk1, unscheduled mitosis and catastrophic DNA damage, ultimately resulting in cell death (JY Zhu et al., J Med Chem. 2017; 60 (18), 7863-7875).

The lack of perceived importance of Myt1 kinase as a therapeutic target has led to a gap in the development of Myt1 kinase inhibitors. In fact, there is only one Myt1 kinase inhibitor currently in human clinical trials (RP-6306) and few others in development. Our improved understanding of Myt1 kinase's role in a disease associated with its activity, and the lack of developed compounds, demonstrates the need to develop new Myt1 kinase inhibitors.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of Myt1 kinase and are therefore useful for treating one or more disorders associated with activity of Myt1 kinase. Thus, in certain embodiments, the present disclosure provides a method for treating a Myt1 kinase-mediated disorder comprising the step of administering to a patient in need thereof a compound of the present disclosure, or pharmaceutically acceptable composition thereof.

As used herein, the term “Myt1 kinase-mediated” disorder or condition as used herein means any disease or other deleterious condition in which Myt1 kinase, or a mutant thereof, is known to play a role. Accordingly, another embodiment of the present disclosure relates to treating or lessening the severity of one or more diseases in which Myt1 kinase, or a mutant thereof, is known to play a role. Specifically, the present disclosure relates to a method of treating or lessening the severity of a disease or condition selected from a proliferative disorder, wherein said method comprises administering to a patient in need thereof a compound or composition according to the present disclosure.

In some embodiments, the present disclosure provides a method for treating or lessening the severity of one or more disorders selected from a cancer comprising the step of administering to the subject an effective amount of a compound, or a pharmaceutically acceptable composition thereof, of the present disclosure. In some embodiments, the cancer is associated with a solid tumor.

In some embodiments, the present disclosure provides a method of treating a subject suffering from a cancer characterized by amplification and/or overexpression of CCNE1 comprising the step of administering to the subject an effective amount of a compound, or a pharmaceutically acceptable composition thereof, of the present disclosure. In some embodiments, the subject is treated only if it has been determined that the subject is resistant to a Wee1A kinase inhibitor. In some embodiments, the cancer is selected from uterine cancer, ovarian cancer, breast cancer, stomach cancer, esophageal cancer, lung cancer, and endometrial cancer. In some embodiments, the cancer is uterine cancer. In some embodiments, the cancer is ovarian cancer. In some embodiments, the cancer is breast cancer. In some embodiments, the cancer is stomach cancer. In some embodiments, the cancer is esophageal cancer. In some embodiments, the cancer is lung cancer. In some embodiments, the cancer is endometrial cancer.

In some embodiments, the present disclosure provides a method of treating a subject suffering from a cancer characterized by aberrant Myt1 kinase activity comprising the step of administering to the subject an effective amount of a compound, or a pharmaceutically acceptable composition thereof, of the present disclosure. In some embodiments, the subject is treated only if it has been determined that the subject is resistant to a Wee1A kinase inhibitor. In some embodiments, aberrant Myt1 kinase activity includes elevated activity, or overexpression, or undesirable activity as compared to a non-diseased state. In some such embodiments, aberrant Myt1 kinase activity includes perturbed Cdk1 activity, altered mitosis and DNA damage. In some embodiments, the cancer is selected from breast cancer, clear cell renal carcinoma, hepatocellular carcinoma, uterine cancer, ovarian cancer, stomach cancer, esophageal cancer, lung cancer, and colorectal cancer. In some such embodiments, the cancer is selected from uterine cancer, ovarian cancer, breast cancer, stomach cancer, esophageal cancer, lung cancer, and endometrial cancer. In some embodiments, the cancer is ovarian cancer. In some embodiments, the cancer is breast cancer. In some embodiments, the cancer is stomach cancer. In some embodiments, the cancer is esophageal cancer. In some embodiments, the cancer is lung cancer. In some embodiments, the cancer is endometrial cancer.

In some embodiments, the present disclosure provides a method for treating or lessening the severity of one or more disorders selected from breast cancer, clear cell renal carcinoma, hepatocellular carcinoma, uterine cancer, ovarian cancer, stomach cancer, esophageal cancer, lung cancer, and colorectal cancer. In some embodiments, the disorders are selected from uterine cancer, ovarian cancer, breast cancer, stomach cancer, esophageal cancer, lung cancer, and endometrial cancer.

In some embodiments, the breast cancer is selected from ductal carcinoma in situ (DCIS), invasive ductal carcinoma (IDC), lobular carcinoma in situ (LCIS), invasive lobular cancer (ILC), triple negative breast cancer (TNBC), inflammatory breast cancer (IBC), metastatic breast cancer (MBC), medullary carcinoma, tubular carcinoma, mucinous carcinoma (colloid), and Paget disease of the breast or nipple (commonly known as Paget disease).

In some embodiments, the uterine cancer is selected from endometrial cancer and uterine sarcoma. In some embodiments, the uterine cancer is endometrial cancer. In some embodiments, the uterine cancer is uterine sarcoma.

In some embodiments, the ovarian cancer is selected from epithelial ovarian carcinomas, germ cell tumors, and stromal cell tumors.

In some embodiments, the stomach cancer is selected from adenocarcinoma, lymphoma, gastrointestinal stromal tumors (GISTs), carcinoid tumors, and hereditary (familial) diffuse gastric cancer.

In some embodiments the esophageal cancer is selected from squamous cell carcinoma, small cell carcinoma, and adenocarcinoma. In some embodiments the esophageal cancer is selected from squamous cell carcinoma and adenocarcinoma. In some embodiments, the esophageal cancer is squamous cell carcinoma. In some embodiments, the esophageal cancer is adenocarcinoma.

In some embodiments, the lung cancer is selected from non-small cell lung cancer, lung nodules, small cell lung cancer, and mesothelioma. In some embodiments, the lung cancer is non-small cell lung cancer.

In some embodiments the colorectal cancer is selected from adenocarcinoma, gastrointestinal stromal tumors (GIST), lymphoma, carcinoids, Turcot syndrome, Peutz-Jeghers syndrome (PJS), familial colorectal cancer (FCC), and juvenile polyposis coli.

In some embodiments, the cancer is associated with deregulation of cyclin E1. In some embodiments, the cancer associated with deregulation of cyclin E1 is ovarian cancer.

In some embodiments, the present disclosure provides a method of treating a subject suffering from a cancer characterized by a mutation in a FBXW7 gene, comprising the step of administering to the subject an effective amount of a compound, or a pharmaceutically acceptable composition thereof, of the present disclosure. In some such embodiments, the mutation in a FBXW7 gene is inactivating. In some embodiments, the subject is treated only if it has been determined that the subject is resistant to a Wee1A kinase inhibitor. In some embodiments, the cancer associated with a mutation in a FBXW7 gene is selected from uterine cancer, colorectal cancer, breast cancer, lung cancer, or esophageal cancer. In some embodiments, the cancer associated with a mutation in a FBXW7 gene is uterine cancer. In some embodiments, the cancer associated with a mutation in a FBXW7 gene is colorectal cancer. In some embodiments, the cancer associated with a mutation in a FBXW7 gene is breast cancer. In some embodiments, the cancer associated with a mutation in a FBXW7 gene is lung cancer. In some embodiments, the cancer associated with a mutation in a FBXW7 gene is esophageal cancer.

In some embodiments, the cancer is associated with deregulation of Cdk1. In some embodiments, the cancer associated with deregulation of Cdk1 is selected from breast cancer, clear cell renal carcinoma, hepatocellular carcinoma, uterine cancer, ovarian cancer, stomach cancer, esophageal cancer, lung cancer, and colorectal cancer.

Depending upon the particular condition, or disease, to be treated, additional therapeutic agents, which are normally administered to treat that condition, may also be present in the compositions of this disclosure. As used herein, additional therapeutic agents that are normally administered to treat a particular disease, or condition, are known as “appropriate for the disease, or condition, being treated.”

For example, compounds of the present disclosure, or a pharmaceutically acceptable composition thereof, are administered in combination with chemotherapeutic agents to treat proliferative diseases and cancer. Examples of known chemotherapeutic agents include, but are not limited to, Adriamycin, dexamethasone, vincristine, cyclophosphamide, fluorouracil, topotecan, taxol, interferons, platinum derivatives, taxane (e.g., paclitaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, emetine, trimetrexate, metoprine, cyclosporine, daunorubicin, teniposide, amphotericin, alkylating agents (e.g., chlorambucil), 5-fluorouracil, camptothecin, cisplatin, metronidazole, and Gleevec™, among others. In other embodiments, a compound of the present disclosure is administered in combination with a biologic agent, such as Avastin or VECTIBIX.

In certain embodiments, compounds of the present disclosure, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG Live, bevacizumab, fluorouracil, bexarotene, bleomycin, bortezomib, busulfan, calusterone, capecitabine, camptothecin, carboplatin, carmustine, celecoxib, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, erlotinib, estramustine, etoposide phosphate, etoposide, exemestane, filgrastim, floxuridine fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

In certain embodiments, compounds of the present disclosure, or a pharmaceutically acceptable composition thereof, are co-administered with a pharmaceutically acceptable Wee1A kinase inhibitor. In some such embodiments, the Wee1A kinase inhibitor is selected from Adavosertib (also known as AZD1775 and MK1775), ZNL-02-096, Debio0123, SGR-3515, IMP7068, or Azenosertib (also known as ZN-c3). In some embodiments, the Wee1A kinase inhibitor is Adavosertib. In some embodiments, the Wee1A kinase inhibitor is ZNL-02-096. In some embodiments, the Wee1A kinase inhibitor is Debio0123. In some embodiments, the Wee1A kinase inhibitor is SGR-3515. In some embodiments, the Wee1A kinase inhibitor is IMP7068. In some embodiments, the Wee1A kinase inhibitor is ZN-c3.

In certain embodiments, compounds of the present disclosure, or a pharmaceutically acceptable composition thereof, are co-administered with a pharmaceutically acceptable DNA damaging agent.

In certain embodiments, compounds of the present disclosure, or a pharmaceutically acceptable composition thereof, are administered in combination with a monoclonal antibody or an siRNA therapeutic.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another, for example, within one, two, three, four, five, six, seven, eight, nine, ten, eleven, or twelve hours from one another.

In some such embodiments, compounds of the present disclosure, or a pharmaceutically acceptable composition thereof, are administered as part of a multiple dosage regimen with a pharmaceutically acceptable Wee1A kinase inhibitor. In certain embodiments, compounds of the present disclosure, or a pharmaceutically acceptable composition thereof, are administered as part of a multiple dosage regimen with a Wee1A kinase inhibitor selected from Adavosertib (also known as AZD1775 and MK1775), ZNL-02-096, and ZN-c3. In some embodiments, compounds of the present disclosure, or a pharmaceutically acceptable composition thereof, are administered as part of a multiple dosage regimen with Adavosertib. In some embodiments, compounds of the present disclosure, or a pharmaceutically acceptable composition thereof, are administered as part of a multiple dosage regimen with ZNL-02-096. In some embodiments, compounds of the present disclosure, or a pharmaceutically acceptable composition thereof, are administered as part of a multiple dosage regimen with ZN-c3.

In some embodiments, compounds of the present disclosure, or a pharmaceutically acceptable salt thereof, are administered to a subject wherein it has been determined that the subject is resistant to a Wee1A kinase inhibitor. In some embodiments, compounds of the present disclosure, or a pharmaceutically acceptable salt thereof, are administered to a subject wherein a Wee1A kinase inhibitor is used as the first or second line therapy. In some embodiments, compounds of the present disclosure, or a pharmaceutically acceptable salt thereof, are administered to a subject wherein a Wee1A kinase inhibitor is used as a first line therapy. In some embodiments, compounds of the present disclosure, or a pharmaceutically acceptable salt thereof, are administered to a subject wherein a Wee1A kinase inhibitor selected from Adavosertib (also known as AZD1775 and MK1775), ZNL-02-096, and ZN-c3, is used as a first line therapy. In some embodiments, compounds of the present disclosure, or a pharmaceutically acceptable salt thereof, are administered to a subject wherein Adavosertib is used as a first line therapy. In some embodiments, compounds of the present disclosure, or a pharmaceutically acceptable salt thereof, are administered to a subject wherein ZNL-02-096 is used as a first line therapy. In some embodiments, compounds of the present disclosure, or a pharmaceutically acceptable salt thereof, are administered to a subject wherein ZN-c3 is used as a first line therapy.

In some embodiments, compounds of the present disclosure, or a pharmaceutically acceptable salt thereof, are administered to a subject wherein a Wee1A kinase inhibitor is used as a second line therapy. In some embodiments, compounds of the present disclosure, or a pharmaceutically acceptable salt thereof, are administered to a subject wherein a Wee1A kinase inhibitor selected from Adavosertib (also known as AZD1775 and MK1775), ZNL-02-096, and ZN-c3, is used as a second line therapy. In some embodiments, compounds of the present disclosure, or a pharmaceutically acceptable salt thereof, are administered to a subject wherein Adavosertib is used as a second line therapy. In some embodiments, compounds of the present disclosure, or a pharmaceutically acceptable salt thereof, are administered to a subject wherein ZNL-02-096 is used as a second line therapy. In some embodiments, compounds of the present disclosure, or a pharmaceutically acceptable salt thereof, are administered to a subject wherein ZN-c3 is used as a second line therapy.

As used herein, the term “combination,” “combined,” “co-administered” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a provided compound, an additional therapeutic agent, and a pharmaceutically acceptable carrier, adjuvant, or vehicle.

The amount of both, an inventive compound and additional therapeutic agent (in those compositions which comprise an additional therapeutic agent as described above)) that may be combined with the carrier materials to produce a single dosage form will vary depending upon the host treated and the particular mode of administration. Preferably, compositions of this disclosure should be formulated so that a dosage of between 0.001-100 mg/kg body weight/day of an inventive can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In such compositions a dosage of between 0.001-1,000 μg/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In some embodiments, the present disclosure provides a method for inhibiting PKMyt1 in vitro. In some such embodiments, the amount of Myt1 kinase inhibition is assessed based on a competitive ATP-binding assay.

In some embodiments, the present disclosure provides a method for inhibiting Myt1 kinase in a biological sample. In some embodiments, a biological sample may comprise a DAOY medulloblastoma cell.

In some embodiments, the present disclosure provides a method for assessing Cdk1 phosphorylation in a cell, comprising contacting said cell with a compound described herein. In one embodiment, the contacting step comprises incubating a cell with a compound presented herein. In some such embodiments, the cell is incubated for at least 4 hours. In some embodiments, the cell may comprise a DAOY medulloblastoma cell.

### EXAMPLES

### General Methods

¹H NMR and ¹³C NMR spectra were recorded on a 500 MHz (¹H NMR at 500 MHz and ¹³C NMR at 126 MHz) Bruker Avance Neo spectrometer equipped with a 5 mm iProbe BBF/H/D probe or on a 400 MHz (¹H NMR at 400 MHz and ¹³C NMR at 101 MHz) Varian Inova spectrometer equipped with a 5 mm 1H/13C auto-switchable gradient-probe at 25° C. The central peaks of chloroform-d (δ_{H} 7.27 ppm), dimethylsulfoxide-d₆ (8H 2.50 ppm), acetonitrile-d₃ (δ_{H} 1.95 ppm) or methanol-d₄ (δ_{H} 3.31 ppm) were used as internal references. Flash chromatography was performed on a Biotage Isolera One system equipped with a diode array detector using prepacked silica columns (Biotage Sfär 60 μm). UV traces were recorded between 200 and 400 nm. Unless stated otherwise, starting materials were commercially available. All solvents and commercial reagents were of laboratory grade and were used as received. The organic phases from extractions were dried over anhydrous sodium sulfate if not stated otherwise. Organic phases or solutions were concentrated by rotary evaporation. Yields were not optimized.

The following methods was used for LC-MS analysis:

Method A: LC-MS analyses were performed on an Agilent 1100 system coupled with an Agilent MSD mass spectrometer operating in ES (+) ionization mode, using a Gemini NX-C18, 3.0×50 mm, 110 Å, column and eluted with solution A (water with 0.1% TFA) and B (acetonitrile) at 40° C. UV-traces were recorded between 220 and 380 nm.

Method B: LC-MS analyses were performed on an Agilent 1260 Infinity II system coupled with an Agilent MSD XT mass spectrometer operating in ES (+ or −) ionization mode, using a Phenomenex Gemini NX-C18, 3.0×50 mm, 110 Å, column and eluted with solution A (water with 0.2% NH₄OH) and B (acetonitrile). UV-traces were recorded at 220 and/or 254 nm.

The following methods were used for HPLC analysis:
Method A: Agilent 1100 system using a Kromasil Eternity-5-C18, 4.6×150 mm column and eluted with solution A (water with 0.1% TFA) and B (acetonitrile with 0.1% TFA). UV-traces were recorded at 220 and 254 nm. 220 nm was used for purity analysis.
Method B: Agilent 1100 system using a Phenomenex Gemini 3 μm NX-C18, 3.0×150 mm, 110 Å column and eluted with solution A (water with 0.1% TFA) and B (acetonitrile) at 40° C. UV-traces were recorded at 220 and 254 nm. 220 nm was used for purity analysis.

### ABBREVIATIONS

BOC-anhydride di-tert-butyl dicarbonate
n-BuLi n-butyl lithium
DCM dichloromethane
DIPEA N,N-diisopropylethylamine
DMF N,N-dimethylformamide
DMSO dimethylsulfoxide
Et ethyl
EtOAc ethyl acetate
EtOH ethanol
GC-MS gas chromatography-mass spectrometry
LDA lithium diisopropylamide
MeOH methanol
LC-MS liquid chromatography-mass spectroscopy
PdCl₂×dppf 1,1′-bis(diphenylphosphino) ferrocene palladium (II) dichloride
RT room temperature, normally 20 to 22° C.
SFC Supercritical Fluid Chromatography
TEA triethylamine
THE tetrahydrofuran
TBME tert-butyl methyl ether
TFA trifluoroacetic acid
Tᵣₑₜ retention time
Triflic anhydride trifluoromethanesulfonic anhydride (Tf₂O)

### Example 1. Synthesis of Intermediates

### Intermediate 1. 2-[methoxy(phenyl)methylidene]propanedinitrile

Malononitrile (396 mg, 6 mmol) and DIPEA (3.1 ml, 18 mmol) were mixed in 20 ml of acetonitrile cooled on ice. Benzoyl chloride (843 mg, 6 mmol) in 20 ml of acetonitrile was added dropwise over 20 minutes. The reaction mixture was allowed to room temperature over two hours. The solvent was removed under reduced pressure and the residue partitioned between water and ethyl acetate. The organic phase was washed with 1 M HCl and water and concentrated to dryness.

The residue from above was dissolved in 5 ml of trimethyl orthoformate. The reaction mixture was stirred at 90° C. for 2 hours and concentrated. The residue was partitioned between water and ethyl acetate. The organic phase wash washed with 0.1 M HCl and water followed by concentration to dryness. The residue was purified with flash chromatography (silica, 10-30% ethyl acetate in petroleum ether). The pure fractions were pooled and concentrated to a colorless oil that solidified on standing. White solid, 0.47 g (42%).

LCMS (ESI+): m/z [M+H]+ calcd.: 185, found: 185.

¹H NMR (500 MHz, DMSO-d6) δ 7.76-7.56 (m, 5H), 3.88 (s, 3H).

### Intermediate 2. 2-[(4-fluorophenyl)(methoxy)methylidene]propanedinitrile

The title compound was synthesized in a similar way as Intermediate 1 using 4-fluorobenzoyl chloride (951 mg, 6.0 mmol) in place of benzoyl chloride. 0.39 g (32%).

LCMS (ESI+): m/z [M+H]+ calcd.: 203, found: 203.

¹H NMR (400 MHz, DMSO-d 6) δ 7.83-7.76 (m, 2H), 7.54-7.45 (m, 2H), 3.89 (s, 3H).

### Intermediate 3. 2-[methoxy(4-methoxyphenyl)methylidene]propanedinitrile

The title compound was synthesized in a similar way as Intermediate 1 using 4-methoxybenzoyl chloride (1.02 g, 6.0 mmol) in place of benzoyl chloride. 0.39 g (32%).

LCMS (ESI+): m/z [M+H]+ calcd.: 215, found: 215.

¹H NMR (400 MHz, DMSO-d6) δ 7.70-7.63 (m, 2H), 7.20-7.13 (m, 2H), 3.91 (s, 3H), 3.86 (s, 3H).

### Intermediate 4. 2-[(4-chlorophenyl)(methoxy)methylidene]propanedinitrile

The title compound was synthesized in a similar way as Intermediate 1 using 4-chlorobenzoyl chloride (1.05 g, 6.0 mmol) in place of benzoyl chloride. 0.14 g (11%).

LCMS (ESI+): m/z [M+H]+ calcd.: 219, found: 219.

¹H NMR (400 MHz, DMSO-d6) δ 7.73 (s, 4H), 3.89 (s, 3H).

### Intermediate 5. 2-[(3-bromophenyl)(methoxy)methylidene]propanedinitrile

The title compound was synthesized in a similar way as Intermediate 1 using 3-bromobenzoyl chloride (1.32 g, 6.0 mmol) in place of benzoyl chloride. 0.57 g (36%).

LCMS (ESI+): m/z [M+H]+ calcd.: 263, found: no ionization.

¹H NMR (400 MHz, DMSO-d6) δ 7.98 (t, J=1.7 Hz, 1H), 7.93-7.87 (m, 1H), 7.73-7.68 (m, 1H), 7.59 (t, J=7.9 Hz, 1H), 3.89 (s, 3H).

### Intermediate 6. N′-[(tert-butoxy)carbonyl]-N-{5-[(tert-butyldimethylsilyl)oxy]-2-methylphenyl}(tert-butoxy)carbohydrazide

Butyl lithium (2.5 M in hexanes, 2.40 ml, 6.00 mmol, 1.20 equiv.) was added dropwise over 2 min to a stirred solution of (3-bromo-4-methylphenoxy) (tert-butyl)dimethylsilane (1.50 g, 4.98 mmol, 1.00 equiv.) in THF (20 ml) under N₂ at −78° C. After stirring at this temperature for 15 min, a solution of Di-tert-butyl azodicarboxylate (5.73 ml, 20% w/v in toluene, 4.98 mmol, 1.00 equiv.) was added dropwise over 1 min. The resulting pale yellow suspension was allowed to reach R^{T} and stirred for 1 h, then quenched by the addition of ammonium chloride solution (3 ml, sat. aq.). The mixture was diluted with ethyl acetate (30 ml) and washed with brine (3×30 ml), the organic phase was filtered through a phase-separator and concentrated under reduced pressure. The crude product was purified by automated flash chromatography (Biotage 25 g sfär silica column, 0-20% ethyl acetate in petroleum ether) to give the title compound. (1.459 g, 65%) as a yellow oil.

Tᵣₑₜ 3.87 min, m/z 297 (+1, −100, −56)

### Intermediate 7. 3-hydrazinyl-4-methylphenol hydrochloride

Hydrogen chloride solution (4 M in dioxane, 8 ml, 32 mmol, 10 equiv.) was added to a solution of N′-[(tert-butoxy)carbonyl]-N-{5-[(tert-butyldimethylsilyl)oxy]-2-methylphenyl}(tert-butoxy)carbohydrazide (1.45 g, 3.20 mmol, 1.00 equiv.) in methanol (5 ml) and the resulting solution was stirred at room temperature for 18 h. The solution was then concentrated under reduced pressure and the resulting solid was triturated with diethyl ether and dried in vacuo to give the title compound (534 mg, 95%) as a brown powder.

Tᵣₑₜ 0.30 min, m/z 139 (+1)

### Intermediate 8. 3-bromo-2,4-dimethylphenoxy(tert-butyl)dimethylsilane

### 3-bromo-2,4-dimethylphenol

To a solution of the 3-bromo-2,4-dimethylaniline (2.00 g, 10.0 mmol, 1.00 equiv.) in glacial acetic acid (20 ml) was added sulfuric acid (conc., 536 μl, 10.0 mmol, 1.00 equiv.) drop wise, followed by water (15 ml) and resulting yellow suspension was cooled to <0° C. A solution of sodium nitrite (759 mg, 11.0 mmol, 1.10 equiv.) in water (3 ml) was then added drop wise at a rate where the internal temperature never exceeded 5° C. After stirring the then clear yellow solution at 0-5° C. for 20 min LCMS analysis indicated complete conversion to the diazonium intermediate. Additional sulfuric acid (conc., 2.0 ml, 37 mmol, 3.7 equiv.) was added and the mixture was heated to reflux for 30 min. After cooling to room temperature, water (150 ml) was added and the mixture was extracted with ethyl acetate (3×50 ml), the combined organic phases were washed with NaHCO₃ (sat. aq.) until the aqueous phase was neutral (3×20 ml), the combined organic phases were filtered through a phase-separator, and then concentrated under reduced pressure to yield the title compound (2.005 g, quant.) as a red solid, which was used in the next step without purification.

Tᵣₑₜ 2.56 min, m/z 243/245 (+1)

### 3-bromo-2,4-dimethylphenoxy(tert-butyl)dimethylsilane

To a solution of 3-bromo-2,4-dimethylphenol (490 mg, 2.44 mmol, 1.00 equiv.) and imidazole (249 mg, 3.66 mmol, 1.50 equiv.) in dry dichloromethane (15 ml) was added tert-butyl-chloro dimethylsilylsilane (404 mg, 2.68 mmol, 1.10 equiv.). After stirring at room temperature for 2.5 h the mixture was filtered and the filtrate was washed with HCl (0.5 M aq., 2×20 ml) followed by brine (20 ml), the organic phase was filtered through a phase-separator and concentrated. The residue was purified by automated flash chromatography (Biotage 10 g sfär silica column, 0-1% ethyl acetate in petroleum ether) to give the title compound (575 mg, 75%) as an orange oil.

Tᵣₑₜ 4.22 min, m/z 315/317 (+1)

### Intermediate 9. N′-[(tert-butoxy)carbonyl]-N-{3-[(tert-butyldimethylsilyl)oxy]-2,6-dimethylphenyl}(tert-butoxy)carbohydrazide

To a solution of 3-bromo-2,4-dimethylphenoxy(tert-butyl)dimethylsilane (575 mg, 1.82 mmol, 1.00 equiv.) in dry THF (15 ml) under N₂ at −78° C. was added butyl lithium (875 μl, 2.5 M in hexanes, 2.19 mmol, 1.20 equiv.) dropwise over 2 min. After stirring at this temperature for 30 min, a solution of di-tert-butyl azodicarboxylate (420 mg, 1.82 mmol, 1.00 equiv.) in dry THF (4 ml) was added dropwise, and the resulting cloudy solution was allowed warm to room temperature over 30 min, at which point LCMS indicated full conversion to the arylhydrazide. Water (10 ml) was added and the pH was adjusted to ca. 5 with HCl (1 M, aq.). The mixture was extracted with ethyl acetate (30 ml) and the organic phase was washed with brine (2×20 ml), filtered through a phase-separator, and concentrated. The residue was purified by automated flash chromatography (Biotage 10 g sfär silica column, 0-15% ethyl acetate in petroleum ether) to give the title compound (580 mg, 68%) as an oil.

Tᵣₑₜ 3.98 min m/z 311 (+1, −100, −56)

### Intermediate 10. 3-hydrazinyl-2,4-dimethylphenol hydrochloride

HCl in dioxane (3.1 ml, 4 M, 12 mmol, 10 equiv.) was added to N′-[(tert-butoxy)carbonyl]-N-{3-[(tert-butyldimethylsilyl)oxy]-2,6-dimethylphenyl}(tert-butoxy)carbohydrazide (580 mg, 1.24 mmol, 1.00 equiv.) in methanol (5 ml). After stirring at room temperature for 7 h the mixture was concentrated under reduced pressure. The resulting yellow solid was washed with diethyl ether and dried in vacuo to give the title compound containing 4 wt % dioxane (238 mg, 94%) as a yellow powder.

Tᵣₑₜ 0.81 min, m/z 153 (+1)

¹H NMR (400 MHz, dmso) δ 9.52 (s, 3H), 9.35 (s, 1H), 6.86 (d, J=8.2 Hz, 1H), 6.69 (d, J=8.2 Hz, 1H), 6.65 (s, 1H), 2.26 (s, 3H), 2.16 (s, 3H).

### Intermediate 11. N′-[(tert-butoxy)carbonyl]-N-[5-methyl-1-(oxan-2-yl)-1H-indazol-4-yl](tert-butoxy)carbohydrazide

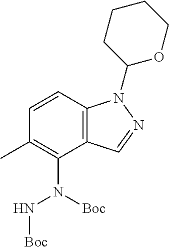 [SMILES: Cc1ccc2c(cnn2C2CCCCO2)c1N(NC(=O)OC(C)(C)C)C(=O)OC(C)(C)C]

To a stirred solution of 4-Bromo-5-methyl-1-(tetrahydro-2H-pyran-2-yl)-1H-indazole (250 mg, 847 μmol, 1.00 equiv.) in dry THF (10 ml) at −78° C. under nitrogen was added n-butyl lithium (407 μl, 2.5 M in hexanes, 1.02 mmol, 1.20 equiv.) dropwise, and the resulting yellow solution was stirred at this temperature for 30 min, when TLC indicated full conversion of the bromoindazole. At this point, a solution of di-tert-butyl azodicarboxylate (195 mg, 847 μmol, 1.00 equiv.) in THF (2 ml) was added dropwise and the resulting cloudy solution was allowed to reach room temperature and stirred for an additional 50 min. The reaction was quenched by the addition of NH₄Cl (3 ml, sat. aq.), diluted with ethyl acetate (15 ml) and washed with brine (3×15 ml). The organic phase was filtered through a phase-separator and concentrated to give a crude product, which was purified by automated flash chromatography (Biotage 10 g sfär silica column, 0-40% ethyl acetate in petroleum ether) to give the title compound (324 mg, 86%) as a colorless oil.

### Intermediate 12. 4-hydrazinyl-5-methyl-1H-indazole dihydrochloride

HCl (1.00 ml, 4M in dioxane, 4 mmol, 5.5 equiv.) was added to a stirred solution of (N′-[(tert-butoxy)carbonyl]-N-[5-methyl-1-(oxan-2-yl)-1H-indazol-4-yl](tert-butoxy)carbohydrazide (324 mg, 726 μmol, 1.00 equiv.) in dry dioxane (5 ml) at room temperature. After 17 h, additional HCl (2 ml, 11 equiv.) was added and the solution was heated at 50° C. for 6.5 h, then at 80° C. for 30 min. After cooling to room temperature, a black solid precipitate was collected by filtration and washed with diethyl ether to give the title compound (125 mg, 97%) as a brown powder, which was used without further purification.

Tᵣₑₜ 1.8 min, m/z 163 (+1)

### Intermediate 13. 2,4-dichloro-3-hydrazinylphenol hydrochloride

### tert-butyl(2,4-dichlorophenoxy)dimethylsilane

A solution of 2,4-dichlorophenol (5.00 g, 30.7 mmol, 1.0 equiv.), imidazole (4.59 g, 67.5 mmol, 2.2 equiv.), and tert-butyldimethylsilyl chloride (5.09 g, 33.7 mmol, 1.1 equiv.) in DCM (40 ml) was stirred at RT for 24 h, when full conversion of the phenol was observed by TLC. The resulting suspension was filtered, and the filtrate was diluted with DCM (50 ml) and washed with NaCl 15% aq. (30 ml) acidified with HCl aq. followed by sat. brine (30 ml). The organic phase was filtered through a phase-separator and concentrated under reduced pressure to give the title compound (8.6 g, quant.) as a clear, colorless liquid.

### N′-[(tert-butoxy)carbonyl]-N-{3-[(tert-butyldimethylsilyl)oxy]-2,6-dichlorophenyl}(tert-butoxy)carbohydrazide

To a solution of tert-butyl(2,4-dichlorophenoxy)dimethylsilane (1.00 g, 3.61 mmol, 1.0 equiv.) in dry THF (30 ml) under N₂ at −78° C. was added butyl lithium (1.59 ml, 2.5 M in hexanes, 3.97 mmol, 1.1 equiv.) dropwise. After stirring at this temperature for 30 min, a solution of di-tert-butyl azodicarboxylate (830 mg, 3.61 mmol, 1.0 equiv.) in dry THF (5 ml) was added dropwise, and the resulting yellow solution was allowed warm to room temperature over 30 min, at which point LCMS indicated full conversion to the arylhydrazide. The reaction was quenched by addition of NaH₂PO₄ (4 ml, 1 M aq.), diluted with ethyl acetate (30 ml) and washed with NaCl (3×20 ml, 15% aq.), followed by sat. brine (20 ml), filtered through a phase-separator, and concentrated. The residue was purified by automated flash chromatography (Biotage 10 g sfär silica column, 0-10% ethyl acetate in petroleum ether) to give the title compound (1.52 g, 83%) as a pale-yellow oil.

LCMS m/z [M+H, −2C₄H₈, —CO₂] found 351.0

### 2,4-dichloro-3-hydrazinylphenol hydrochloride

HCl (7 ml, 7 M in dioxane, 28 mmol, 9.5 equiv.) was added to a solution of N′-[(tert-butoxy)carbonyl]-N-{3-[(tert-butyldimethylsilyl)oxy]-2,6-dichlorophenyl}(tert-butoxy)carbohydrazide in methanol (7 ml) and the solution was stirred at RT for 3 days and then concentrated under reduced pressure. The resulting black residue was dissolved in ethanol (4 ml) to which diethyl ether (40 ml) was added. The resulting yellow precipitate was collected by filtration, washed with diethyl ether, and dried in vacuo to give 2,4-dichloro-3-hydrazinylphenol hydrochloride (598 mg, 88%) as a yellow powder.

¹H NMR (400 MHz, DMSO) § 10.87 (s, 1H), 10.14-9.72 (m, 3H), 7.31 (d, J=8.9 Hz, 1H), 7.17 (s, 1H), 6.94 (d, J=8.9 Hz, 1H).

### Intermediate 14. [(5-chloro-2-furyl)methoxymethylene]propanedinitrile

Malononitrile (0.30 g, 4.5 mmol) and Et₃N (1.7 ml, 12 mmol) were mixed in 20 ml of acetonitrile cooled on ice. 5-Chloro-2-furoyl chloride (0.49 g, 3.0 mmol) in 10 ml of acetonitrile was added dropwise over 30 minutes. The reaction mixture was allowed to room temperature over night. The solvent was removed under reduced pressure and the residue partitioned between water and ethyl acetate. The organic phase was washed with 1 M HCl and water and concentrated to dryness. The residue was dissolved in 5 ml of trimethyl orthoformate. The reaction mixture was stirred at 90° C. overnight and concentrated. The residue was partitioned between water and ethyl acetate. The organic phase was washed with water and sat. NaHCO₃, dried over MgSO₄, filtered and concentrated. The residue was purified with flash chromatography (silica, 5-30% ethyl acetate in petroleum ether). The pure fractions were pooled and concentrated giving 0.23 g (37%) of the title compound.

LCMS (ESI+): m/z 209.

¹H NMR (500 MHz, CDCl₃) δ 7.70 (d, J=3.9 Hz, 1H), 7.00 (d, J=3.9 Hz, 1H), 4.24 (s, 3H).

### Intermediate 15. [methoxy(3-methyl-2-furyl)methylene]propanedinitrile

This intermediate was synthesized in a similar way as [(5-chloro-2-furyl)methoxymethylene]propanedinitrile using 3-methyl-2-furoyl chloride (0.29 g, 2.0 mmol) giving 0.15 g (41%).

LCMS (ESI+): m/z [M+H]+ found: 189.

¹H NMR (500 MHz, DMSO-d6) δ 8.14-8.11 (m, 1H), 6.77-6.75 (m, 1H), 4.05 (s, 3H), 2.23 (s, 3H).

### Intermediate 16. [(4,5-dimethyl-2-furyl)methoxymethylene]propanedinitrile

This intermediate was synthesized in a similar way as [(5-chloro-2-furyl)methoxymethylene]propanedinitrile using 4,5-dimethyl-2-furoyl chloride (0.28 g, 1.8 mmol) giving 0.23 g (65%).

LCMS (ESI+): m/z [M+H]+ found: 203

¹H NMR (400 MHz, DMSO-d6) δ 7.51 (s, 1H), 4.21 (s, 3H), 2.35 (s, 3H), 2.02 (s, 3H).

### Intermediate 17. 2-((5-bromofuran-3-yl)(methoxy)methylene)malononitrile

### 5-bromofuran-3-carboxylic acid

To the stirred solution of pyridinium tribromide (5.54 g, 17.31 mmol) in acetic acid (3 ml) was added furan-3-carboxylic acid (2 g, 17.84 mmol) at 25° C. The reaction mixture was allowed to stir at 40° C. for 16 h. The progress of the reaction was monitored by TLC and LCMS. After completion of the reaction the reaction mixture was concentrated under reduced pressure, then 30 ml of ice-cold water was added to the reaction mass. The product solidified it was filtered and washed with water and dried over vacuum to give 5-bromofuran-3-carboxylic acid (1.2 g, 3.77 mmol, 21.13% yield) as off-white solid.

LCMS (ESI) m/z=188.8 (M−H).

### 2-((5-bromofuran-3-yl)(hydroxy)methylene)malononitrile

To a stirred solution of 5-bromofuran-3-carboxylic acid (1.2 g, 6.28 mmol) in ethyl acetate (20 ml) was added triethylamine (3.07 ml, 21.99 mmol), propylphosphonic acid anhydride (11.11 ml as a 50% solution in EtOAc, 18.85 mmol) followed by the addition of malononitrile (0.623 g, 9.43 mmol) at 0° C. under inert atmosphere, then the reaction mixture was stirred at rt for 16 h. Progress of the reaction was monitored by TLC and LCMS. After completion of the reaction, the reaction mixture was concentrated under reduced pressure, water (50 mL) was added and the mixture extracted with ethyl acetate (2×100 mL). the combined organic layer was dried over anhydrous Na₂SO₄ and concentrated under reduced pressure to give the crude compound 2-((5-bromofuran-3-yl)(hydroxy)methylene)malononitrile (1 g, 2.97 mmol, 47.3% yield) as an light brown solid. This crude compound as such taken for the next step without any further purification.

LCMS (ESI) m/z=237.0 (M−H).

### 2-((5-bromofuran-3-yl)(methoxy)methylene)malononitrile

In a flask, 2-((5-bromofuran-3-yl)(hydroxy)methylene)malononitrile (1 g, 4.18 mmol) was taken and trimethyl orthoformate (10 ml) was added at rt under inert atmosphere, then the temperature was raised to 90° C. and the mixture stirred for 48 h. Progress of the reaction was monitored by TLC and LCMS. After completion of the reaction, water (100 mL) was added and the mixture extracted with ethyl acetate (2×200 mL). The combined organic layer and washed with 10% NaHCO₃ solution (100 mL), dried over anhydrous Na₂SO₄ and concentrated under reduced pressure to give the crude compound which was purified by silica gel column chromatography (SiO₂/100-200 mesh; ethyl acetate-n-hexane, 50-100% as an eluent) to give the pure compound 2-((5-bromofuran-3-yl)(methoxy)methylene)malononitrile (1 g, 3.59 mmol, 94% yield) as an off-white solid.

GCMS (ESI) m/z=252.0 (M).

¹H NMR (400 MHz, DMSO-d6): δ 8.58 (d, J=1.20 Hz, 1H), 7.11 (d, J=0.80 Hz, 1H), 4.05 (s, 3H).

### Intermediate 18. 2-((4-bromofuran-2-yl)(methoxy)methylene)malononitrile

### 2-((4-bromofuran-2-yl)(hydroxy)methylene)malononitrile

To a stirred solution of 4-bromofuran-2-carboxylic acid (1.0 g, 5.24 m mol) in ethyl acetate (20 ml) was added TEA (1.854 g, 18.33 mmol), propylphosphonic acid anhydride (10.00 ml as a 50% solution in EtOAc, 15.71 m mol), malononitrile (0.761 g, 11.52 mmol). The resulting mixture was allowed to stir for 16 hours at rt. Progress of the reaction was monitored by TLC and LCMS. After completion of the reaction, the reaction mixture was concentrated under reduced pressure, then water (50 mL) was added and the mixture extracted with ethyl acetate (2×100 mL). The combined organic layer was dried over anhydrous Na₂SO₄. Concentrated under reduced pressure to get the crude compound 2-((4-bromofuran-2-yl)(hydroxy)methylene)malononitrile (1.5 g, 5.02 mmol, 96% yield) as a brown gummy compound.

LCMS (ESI) m/z=236.9 (M−H).

### 2-((4-bromofuran-2-yl)(methoxy)methylene)malononitrile

In a flask, 2-((4-bromofuran-2-yl)(hydroxy)methylene)malononitrile (1 g, 4.18 mmol) and trimethyl orthoformate (10 mL) were mixed at 25° C. under inert atmosphere, then the temperature was raised to 90° C. and the mixture stirred for 48 hours. Progress of the reaction was monitored by TLC and LCMS. After completion of the reaction, water (100 mL) was added and the mixture extracted with ethyl acetate (2×200 mL). The combined organic layer was washed with 10% NaHCO₃ solution (100 mL), dried over anhydrous Na₂SO₄, concentrated under reduced pressure to give the crude compound which was purified by silica gel column chromatography (SiO₂/100-200 mesh; ethyl acetate-n-hexane, 10-50% as an eluent) to give the pure compound 2-((4-bromofuran-2-yl)(methoxy)methylene)malononitrile (400 mg, 1.581 mmol, 37.8% yield) as an off-white solid.

GCMS (ESI) m/z=252.0 (M).

¹H NMR (400 MHz, DMSO-d6): δ 8.51 (d, J=0.40 Hz, 1H), 7.82 (d, J=0.40 Hz, 1H), 4.23 (s, 3H).

### Intermediate 19. Synthesis of 2-(methoxy(5-phenylisoxazol-3-yl)methylene)malononitrile

### 2-(hydroxy(5-methylfuran-2-yl)methylene)malononitrile

To a stirred solution of 5-methylfuran-2-carboxylic acid (1.5 g, 11.89 mmol) in ethyl acetate (10 ml) was added TEA (4.97 ml, 35.7 mmol) followed by the addition of propylphosphonic acid anhydride (8.76 ml as a 50% solution in EtOAc, 29.7 mmol) at 0° C. under inert atmosphere. The mixture was then stirred for 15 min. Malononitrile (1.179 g, 17.84 mmol) was added and the resulting mixture stirred at 25° C. for 16 h. Progress of the reaction was monitored by TLC and LCMS. After completion of the reaction, the reaction mixture was concentrated under reduced pressure, water (100 mL) was added and extracted with ethyl acetate (2×100 mL). The combined organic layer was washed with 1.5 N HCl (100 mL), dried over anhydrous Na₂SO₄, concentrated under reduced pressure to get 2-(hydroxy(5-methylfuran-2-yl)methylene)malononitrile (1.2 g, 3.586 mmol, 29.5% yield) as a gummy solid.

LCMS (ESI) m/z=172.9 (M−H).

### 2-(methoxy(5-methylfuran-2-yl)methylene)malononitrile

In a flask, 2-(hydroxy(5-methylfuran-2-yl)methylene)malononitrile (1 g, 5.74 mmol) and trimethyl orthoformate (10 ml) were mixed at 25° C. under inert atmosphere, then the temperature was raised to 90° C. and reaction mixture was stirred for 48 h. Progress of the reaction was monitored by TLC and LCMS. After completion of the reaction, water (100 mL) was added and the mixture extracted with ethyl acetate (2×200 mL). The combined organic layer was washed with 10% NaHCO₃ solution (100 mL), dried over anhydrous Na₂SO₄, concentrated under reduced pressure to give the crude compound which was purified by silica gel column chromatography (SiO₂/100-200 mesh; ethyl acetate-n-hexane, 50-100% as an eluent) to give 2-(methoxy(5-methylfuran-2-yl)methylene)malononitrile (350 mg, 1.804 mmol, 31.4% yield) as an off-white solid.

LCMS (ESI) m/z=189.2 (M+H).

¹H NMR (400 MHz, DMSO-d6): δ 7.58 (d, J=3.60 Hz, 1H), 6.60 (dd, J=0.80, 3.60 Hz, 1H), 4.23 (s, 3H), 2.44 (s, 3H).

### Intermediate 20. 2-(benzofuran-2-yl(methoxy)methylene)malononitrile

### 2-(benzofuran-2-yl(hydroxy)methylene)malononitrile

To a stirred solution of benzofuran-2-carboxylic acid (1 g, 6.17 mmol) in ethyl acetate (10 ml) was added DIPEA (3.21 ml, 18.50 mmol) at 0° C., followed by n-propylphosphonic acid anhydride (cyclic trimer, 6.87 g, 21.59 mmol) and the mixture was stirred for 15 mins. After 15 mins, malononitrile (0.611 g, 9.25 mmol) was added and the mixture was stirred at 25° C. for 16 h. Progress of the reaction was monitored by TLC and LCMS. The reaction mixture was concentrated and then diluted with 10 ml 10% hexane-EtOAc. It was then washed with 1.5 N HCl solution, dried over Na₂SO₄ and concentrated in vacuo to give 2-(benzofuran-2-yl(hydroxy)methylene)malononitrile.

LCMS: m/z found 209.

### 2-(benzofuran-2-yl(methoxy)methylene)malononitrile

To 2-(benzofuran-2-yl(hydroxy)methylene)malononitrile (1 g, 4.76 mmol) was added trimethylorthoformate (10 ml, 4.76 mmol) and the mixture was stirred at 90° C. for 48 h. Progress of the reaction was monitored by TLC and LCMS. After completion, the reaction mixture was concentrated under reduced pressure and the residue diluted with 10% n-heptane-EtOAc solution, washed with sodium bicarbonate solution. The organic layer was dried over Na₂SO₄ and concentrated under reduced pressure. The crude was purified by flash silica-gel (100-200 mesh; 80 g) column with 20-25% EtOAc/Hexane and fractions were evaporated under reduced pressure to obtain 2-(benzofuran-2-yl(methoxy)methylene)malononitrile (311 mg, 1.39 mmol).

¹H NMR (400 MHz, DMSO-d6): δ 8.06 (d, J=0.80 Hz, 1H), 7.93 (dd, J=0.80, 1.00 Hz, 1H), 7.91 (dd, J=0.80, 1.20 Hz, 1H), 7.62 (dt, J=1.20, 8.40 Hz, 1H), 7.45 (dt, J=1.20, 7.73 Hz, 1H), 4.30 (s, 3H).

### Intermediate 21. 2-((5-bromofuran-2-yl)(methoxy)methylene)malononitrile

### 2-((5-bromofuran-2-yl)(hydroxy)methylene)malononitrile

To a stirred solution of 5-bromofuran-2-carboxylic acid (1.0 g, 5.24 mmol) in ethyl acetate (10 ml) was added DIPEA (2.80 ml, 15.71 mmol) and propanephosphonic acid cyclic anhydride 50% in ethyl acetate (5.30 ml, 18.33 mmol) at 0° C. under inert atmosphere, and the resulting mixture was then stirred for 15 mins. To the reaction mixture was added malononitrile (0.519 g, 7.85 mmol) and the mixture was stirred at rt for 16 hours. Progress of the reaction was monitored by TLC and LCMS. After completion of the reaction, the solvent was evaporated, water (100 mL) was added and the mixture was extracted with ethyl acetate (2×100 mL). The combined organic layer was washed with 1.5 N HCl (50 mL), dried over anhydrous Na₂SO₄, evaporated under reduced pressure to give the crude compound 2-((5-bromofuran-2-yl)(hydroxy)methylene)malononitrile (1.0 g, 2.80 mmol, 53.5% yield) as an off-white solid. This crude compound taken as such for the next step without any further purification.

LCMS (ESI) m/z=237.0 (M−H).

### 2-((5-bromofuran-2-yl)(methoxy)methylene)malononitrile

In a flask, 2-((5-bromofuran-2yl)(hydroxy)methylene)malononitrile (1.0 g, 4.18 mmol) and trimethyl orthoformate (10 ml) were mixed at rt under inert atmosphere, and then the reaction mixture was stirred at 100° C. for 48 hours. Progress of the reaction was monitored by TLC and LCMS. After completion of the reaction, water (100 mL) was added and the mixture extracted with ethyl acetate (2×200 mL). The combined organic layer was washed with 10% NaHCO₃ solution (100 mL), dried over anhydrous Na₂SO₄, evaporated under reduced pressure to give the crude compound which was purified by silica gel column chromatography (SiO₂/100-200 mesh; ethyl acetate-n-hexane, 10-20% as an eluent) to afford 2-((5-bromofuran-2-yl)(methoxy)methylene)malononitrile (0.315 mg, 1.175 μmol, 0.028% yield) as a yellow solid. GCMS: m/z=252.0.

¹H NMR (400 MHz, CDCl₃): δ 7.41-7.40 (d, 1H), 6.64-6.63 (d, 1H), 4.29 (s, 3H).

### Intermediate 22. 2-(benzofuran-3-yl(methoxy)methylene)malononitrile

### Ethyl benzofuran-3-carboxylate

To a stirred solution of 2-hydroxybenzaldehyde (1 g, 8.19 mmol) in DCM (10 mL) was added tetrafluoroboric acid diethyl ether (0.133 g, 0.819 mmol) followed by ethyl 2-diazoacetate (1.9 g, 16.4 mmol) drop wise at room temperature, and stirred at 25° C. for 1 h. The progress of reaction was monitored by TLC. After completion of the reaction, the reaction mixture was concentrated under reduced pressure. Then 1.0 mL of conc. Sulphuric acid was added to the reaction mixture which was stirred for 30 min. pH was adjusted with solium sulphate to pH=6. Then the reaction mixture was filtered on celite pad, and the filtrate was concentrated under reduced pressure. The resulting crude material was purified by flash chromatography (SiO₂/230-400 mesh; 0-10% EtOAc/Hexane) to afford ethyl benzofuran-3-carboxylate (2 g, 5.05 mmol, 61.6% yield) as an off white solid.

### Benzofuran-3-carboxylic acid

To a stirred solution of ethyl benzofuran-3-carboxylate (2 g, 5.78 mmol) in ethanol (20 mL) was added sodium hydroxide (2.313 g, 5.78 mmol) at room temperature and the resulting mixture was stirred at 85° C. for 16 h. The progress of reaction was monitored by TLC. After completion of the reaction, the reaction mixture was concentrated under reduced pressure and diluted with water. The aqueous solution was acidified with 1M HCl to precipitate a solid that was collected by filtering and washed with water (5 mL), and dried to get benzofuran-3-carboxylic acid (700 mg, 4.32 mmol, 74.6% yield) as an off white solid.

LCMS (ESI) m/z=161.1 (M−H).

### 2-(benzofuran-3-yl(hydroxy)methylene)malononitrile

To a stirred solution of benzofuran-3-carboxylic acid (700 mg, 4.32 mmol) in ethyl acetate (20 ml) was added DIPEA (3.01 mL, 17.27 mmol) followed by the addition of propanephosphonic acid anhydride (50% solution in EtOAc) (5.72 mL, 19.43 mmol) at 0° C. under inert atmosphere. The resulting mixture was then stirred for 15 minutes. Then malononitrile (570 mg, 8.63 mmol) was added an the mixture stirred at 25° C. for 16 h. The progress of the reaction was monitored by TLC and LCMS. After completion of the reaction, the reaction mixture was concentrated under reduced pressure, water (100 mL) was added and the mixture extracted with ethyl acetate (2×100 mL). The combined organic layer was washed with 1.5 N HCl (100 mL), dried over anhydrous Na₂SO₄, concentrated under reduced pressure to give 2-(benzofuran-3-yl(hydroxy)methylene)malononitrile (850 mg, 3.72 mmol, 86% yield) as a gummy solid.

LCMS (ESI) m/z=209.0 (M−H).

### 2-(benzofuran-3-yl(methoxy)methylene)malononitrile

The solution of 2-(benzofuran-3-yl(hydroxy)methylene)malononitrile (1 g, 4.76 mmol) in trimethyl orthoformate (5.05 g, 47.6 mmol) was stirred at 100° C. for 48 h. The progress of the reaction was monitored by TLC and LCMS. After completion of the reaction, water (100 mL) was added to the reaction mixture and it was extracted with ethyl acetate (2×200 mL). The combined organic layer was washed with 10% NaHCO₃ solution (100 mL), dried over anhydrous Na₂SO₄, concentrated under reduced pressure to give the crude compound which was purified by silica gel column chromatography (SiO₂/230-400 mesh; ethyl acetate-n-hexane, 10-40% as an eluent) to get the pure compound 2-(benzofuran-3-yl(methoxy)methylene)malononitrile (239.3 mg, 0.971 mmol, 20.41% yield) as an pale brown solid.

¹H NMR (400 MHz, DMSO-d6): δ 8.83 (s, 1H), 7.78 (d, J=7.60 Hz, 1H), 7.78 (d, J=7.60 Hz, 1H), 7.55-7.51 (m, 1H), 7.49-7.45 (m, 1H), 4.05 (s, 3H)

### Intermediate 23. Synthesis of 2-(furan-2-yl(methoxy)methylene)malononitrile

### 2-(furan-2-yl(hydroxy)methylene)malononitrile

To the solution of furan-2-carboxylic acid (5 g, 44.6 mmol) in ethyl acetate (50 mL) were added TEA (18.62 mL, 134 mmol) and propanephosphonic acid anhydride (50% solution in EtOAc) (40.1 mL, 66.9 mmol) followed by the addition of malononitrile (4.42 g, 66.9 mmol) at 0° C. under inert atmosphere. The mixture was then stirred at rt for 16 h. The progress of the reaction was monitored by TLC and LCMS. After completion of the reaction, the reaction mixture was diluted with water (200 mL) and extracted with ethyl acetate (3×200 mL). The combined organic layers were washed with brine (100 mL), dried over anhydrous sodium sulphate, filtered and concentrated under reduced pressure to give the crude compound 2-(furan-2-yl(hydroxy)methylene)malononitrile (2.5 g, 15.30 mmol, 34.3% yield) as an brown gummy solid. This crude compound as such taken for the next step without any further purification.

LCMS (ESI) m/z=159.1 (M−H).

### 2-(furan-2-yl(methoxy)methylene)malononitrile

A solution of 2-(furan-2-yl(hydroxy)methylene)malononitrile (2.5 g, 15.61 mmol) in trimethyl orthoformate (25 mL) was stirred at 90° C. for 16 h. The progress of the reaction was monitored by TLC and LCMS. After completion of the reaction, the reaction mixture was diluted with water (200 mL) and extracted with ethyl acetate (3×200 mL). The combined organic layers were washed with brine (200 mL) followed by aq. NaHCO₃ (200 mL), dried over anhydrous sodium sulphate, filtered and concentrated under reduced pressure to afford the crude compound which was purified by flash column chromatography (silica-gel, mesh size 60-120: ethyl acetate in n-hexane 40-45% as an eluent) to give 2-(furan-2-yl(methoxy)methylene)malononitrile (910 mg, 5.17 mmol, 33.1% yield) as a yellowish solid.

GCMS (ESI) m/z=174.1 (M).

¹H NMR (400 MHz, DMSO-d6): § 7.78 (d, J=1.20 Hz, 1H), 7.44 (d, J=3.20 Hz, 1H), 6.70 (dd, J=1.60, 3.60 Hz, 1H), 4.28 (s, 3H).

### Intermediate 24. [5-methyl-1-(tetrahydro-2H-pyran-2-yl)-1H-indazol-4-yl]hydrazine

### N′-[(tert-butoxy)carbonyl]-N-[5-methyl-1-(oxan-2-yl)-1H-indazol-4-yl](tert-butoxy)carbohydrazide

To a stirred solution of 4-Bromo-5-methyl-1-(tetrahydro-2H-pyran-2-yl)-1H-indazole (250 mg, 847 μmol, 1.00 equiv.) in dry THF (10 ml) at −78° C. under nitrogen was added butyl lithium (407 μl, 2.5 M in hexanes, 1.02 mmol, 1.20 equiv.) dropwise, and the resulting yellow solution was stirred at this temperature for 30 min, when TLC indicated full conversion of the bromoindazole. At this point, a solution of di-tert-butyl azodicarboxylate (195 mg, 847 μmol, 1.00 equiv.) in THF (2 ml) was added dropwise and the resulting cloudy solution was allowed to reach room temperature and stirred for an additional 50 min. The reaction was quenched by the addition of NH₄Cl (3 ml, sat. aq.), diluted with ethyl acetate (15 ml) and washed with brine (3×15 ml). The organic phase was filtered through a phase-separator and concentrated to give a crude product, which was purified by automated flash chromatography (Biotage 10 g sfär silica column, 0-40% ethyl acetate in petroleum ether) to give N′-[(tert-butoxy)carbonyl]-N-[5-methyl-1-(oxan-2-yl)-1H-indazol-4-yl](tert-butoxy)carbohydrazide (324 mg, 86%) as a colorless oil.

LCMS (ESI+): m/z [M+H]+ found: 447

### [5-methyl-1-(tetrahydro-2H-pyran-2-yl)-1H-indazol-4-yl]hydrazine

A solution of N′-[(tert-butoxy)carbonyl]-N-[5-methyl-1-(oxan-2-yl)-1H-indazol-4-yl](tert-butoxy)carbohydrazide (566 mg, 1.27 mmol) in a mixture of CH₂Cl₂ (3 mL)/CF₃CH₂OH (1 mL) was rapidly added to a solution of TfOH (0.75 g, 5 mmol) in CF₃CH₂OH (1 mL) at −40° C. under Argon. The mixture was stirred for 1.5 min, and CH₂Cl₂ (5 mL) followed by sat. NaHCO₃ (10 mL) were added under very vigorous stirring. The organic layer was separated and the aqueous layer extracted with CH₂Cl₂. The extract was dried (Na₂SO₄) and the solvent evaporated to give [5-methyl-1-(tetrahydro-2H-pyran-2-yl)-1H-indazol-4-yl]hydrazine as a viscous, yellow to orange oil that darkened upon standing (150 mg, 48%).

LCMS (ESI+): m/z [M+H]+ calc: 247, found: 247.

¹H NMR (400 MHz, Methanol-d4) δ 8.20 (s, 1H), 7.47 (d, J=8.5 Hz, 1H), 7.33 (d, J=8.6 Hz, 1H), 5.80 (dd, J=9.9, 2.5 Hz, 1H), 4.00 (d, J=11.5 Hz, 1H), 3.81 (td, J=11.4, 3.4 Hz, 1H), 2.56-2.46 (m, 1H), 2.45 (s, 3H), 2.19-1.98 (m, 2H), 1.88-1.63 (m, 3H).

### Example 2. Synthesis of 5-amino-1-(5-hydroxy-2-methylphenyl)-3-phenyl-1H-pyrazole-4-carboxamide (Compound 1)

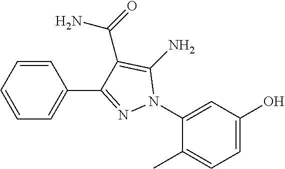 [SMILES: Cc1ccc(O)cc1-n1nc(-c2ccccc2)c(C(N)=O)c1N]

2-[Methoxy (phenyl)methylidene]propanedinitrile (Intermediate 1, 9 mg, 50 μmol), 3-hydrazinyl-4-methylphenol hydrochloride (Intermediate 7, 9 mg, 50 μmol) and Et₃N (21 μl, 150 μmol) were mixed in 1 ml of methanol. The reaction mixture was stirred over night at 60° C. Sodium hydroxide (100 μl, 5 M) and hydrogen peroxide (35%, 100 μl) were added. The reaction mixture was stirred over the weekend at 50° C. The reaction mixture was diluted with methanol/water, acidified with TFA and purified with reversed phase chromatography (Gemini NX-C18, 21*150 mm, water (0.1% TFA)/acetonitrile, gradient over 12 minutes, 25 ml/min). The pure fractions were pooled and concentrated giving 5 mg (12%) of the title compound as a TFA salt.

LCMS (ESI+): m/z [M+H]+ calcd.: 309, found: 309.

¹H NMR (500 MHz, DMSO-d6) δ 7.58-7.41 (m, 5H), 7.19 (d, J=8.6 Hz, 1H), 6.83 (dd, J=8.3, 2.6 Hz, 1H), 6.71 (d, J=2.6 Hz, 1H), 2.00 (s, 3H).

### Example 3. Synthesis of 5-amino-3-(4-fluorophenyl)-1-(5-hydroxy-2-methylphenyl)-1H-pyrazole-4-carboxamide (Compound 2)

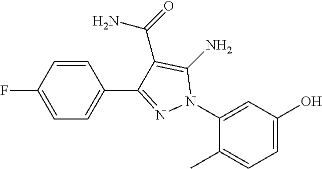 [SMILES: Cc1ccc(O)cc1-n1nc(-c2ccc(F)cc2)c(C(N)=O)c1N]

The title compound was synthesized in a similar way as Example 1 using 2-[(4-fluorophenyl)(methoxy)methylidene]propanedinitrile (Intermediate 2, 20 mg, 100 μmol) and 3-hydrazinyl-4-methylphenol hydrochloride (Intermediate 7, 18 mg, 100 μmol) giving 11 mg (25%) of the title compound as a TFA salt.

LCMS (ESI+): m/z [M+H]+ calcd.: 327, found: 327.

¹H NMR (500 MHz, DMSO-d6) δ 7.62-7.57 (m, 2H), 7.32-7.25 (m, 2H), 7.19 (d, J=8.6 Hz, 1H), 6.83 (dd, J=8.3, 2.6 Hz, 1H), 6.70 (d, J=2.6 Hz, 1H), 1.99 (s, 3H).

### Example 4. Synthesis of 5-amino-1-(5-hydroxy-2-methylphenyl)-3-(4-methoxyphenyl)-1H-pyrazole-4-carboxamide (Compound 3)

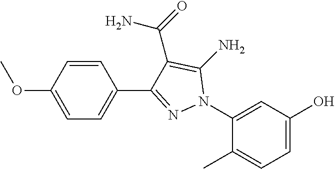 [SMILES: COc1ccc(-c2nn(-c3cc(O)ccc3C)c(N)c2C(N)=O)cc1]

The title compound was synthesized in a similar way as Example 1 using 2-[methoxy(4-methoxyphenyl)methylidene]propanedinitrile (Intermediate 3, 21 mg, 100 μmol) and 3-hydrazinyl-4-methylphenol hydrochloride (Intermediate 7, 18 mg, 100 μmol) giving 8 mg (18%) of the title compound as a TFA salt.

LCMS (ESI+): m/z [M+H]+ calcd.: 327, found: 327.

¹H NMR (500 MHz, DMSO-d6) δ 7.62-7.57 (m, 2H), 7.32-7.25 (m, 2H), 7.19 (d, J=8.6 Hz, 1H), 6.83 (dd, J=8.3, 2.6 Hz, 1H), 6.70 (d, J=2.6 Hz, 1H), 1.99 (s, 3H).

### Example 5. Synthesis of 5-amino-3-(4-chlorophenyl)-1-(5-hydroxy-2-methylphenyl)-1H-pyrazole-4-carboxamide (Compound 4)

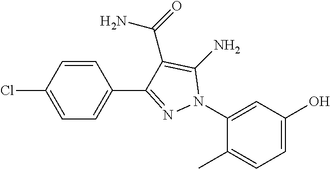 [SMILES: Cc1ccc(O)cc1-n1nc(-c2ccc(Cl)cc2)c(C(N)=O)c1N]

The title compound was synthesized in a similar way as for Compound 1 using 2-[(4-chlorophenyl)(methoxy)methylidene]propanedinitrile (Intermediate 4, 22 mg, 100 μmol) and 3-hydrazinyl-4-methylphenol hydrochloride (Intermediate 7, 18 mg, 100 μmol) giving 12 mg (26%) of the title compound as a TFA salt.

LCMS (ESI+): m/z [M+H]+ calcd.: 343, found: 343.

¹H NMR (500 MHz, DMSO-d6) δ 9.62 (s, 1H), 7.61-7.57 (m, 2H), 7.53-7.48 (m, 2H), 7.19 (d, J=8.6 Hz, 1H), 6.83 (dd, J=8.3, 2.6 Hz, 1H), 6.70 (d, J=2.6 Hz, 1H), 6.07 (s, 2H), 1.99 (s, 3H).

### Example 6. Synthesis of 5-amino-1-(5-methyl-1H-indazol-4-yl)-3-phenyl-1H-pyrazole-4-carboxamide (Compound 5)

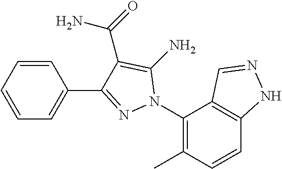 [SMILES: Cc1ccc2[nH]ncc2c1-n1nc(-c2ccccc2)c(C(N)=O)c1N]

2-[methoxy (phenyl)methylidene]propanedinitrile (intermediate 1, 30 mg, 0.16 mmol), 4-hydrazinyl-5-methyl-1H-indazole hydrochloride (Intermediate 12, 50 mg, 0.25 mmol, ~50% pure) and Et₃N (68 μl, 0.49 mmol) were mixed in 1 ml of methanol and stirred at room temperature for 3 hours. The reaction mixture was diluted with methanol/water, acidified with TFA and purified with reversed phase chromatography (Gemini NX-C18, 21*150 mm, water (0.1% TFA)/acetonitrile, gradient over 12 minutes, 25 ml/min). The pure fractions were pooled and concentrated.

The residue from above was mixed in 300 μl of conc. H₂SO₄ and stirred at 40° C. overnight.

The reaction mixture was diluted with ice cold methanol/water and purified with reversed phase chromatography (Gemini NX-C18, 21*150 mm, water (0.1% TFA)/acetonitrile, gradient over 12 minutes, 25 ml/min). The pure fractions were pooled and concentrated giving 10 mg (13%) of the title compound as a TFA salt.

LCMS (ESI+): m/z [M+H]+ calcd.: 333, found: 333.

¹H NMR (400 MHz, DMSO-d6) δ 7.72 (s, 1H), 7.65-7.58 (m, 3H), 7.53-7.43 (m, 3H), 7.39 (d, J=8.6 Hz, 1H), 2.22 (s, 3H).

### Example 7. Synthesis of 5-amino-3-(3-bromophenyl)-1-(5-hydroxy-2-methylphenyl)-1H-pyrazole-4-carboxamide (Compound 6)

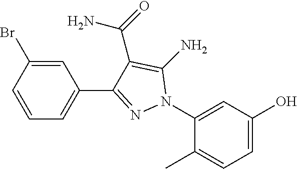 [SMILES: Cc1ccc(O)cc1-n1nc(-c2cccc(Br)c2)c(C(N)=O)c1N]

2-[(3-Bromophenyl)(methoxy)methylidene]propanedinitrile (Intermediate 5, 0.20 g, 0.76 mmol), 3-hydrazinyl-4-methylphenol hydrochloride (Intermediate 7, 0.13 g mg, 0.76 mmol) and Et₃N (316 μl, 2.3 mmol) were mixed in 20 ml of methanol and stirred at 60° C. overnight. The solvent was removed under reduced pressure. The residue was partitioned between water and ethyl acetate. The organic phase was washed with 1 M HCl and water followed by concentrating to dryness. The residue was purified with flash chromatography (silica, 20-50% EtOAc in petroleum ether). The pure fractions were pooled and concentrated.

The residue from above was mixed in 2 ml of conc. H₂SO₄ and stirred at 50° C. overnight. The reaction mixture was cooled on ice and quenched by the addition of 10 ml of ice/water slurry. The reaction mixture was stirred for 1 hour and concentrated.

The residue was purified with reversed phase chromatography (Gemini NX-C18, 21*150 mm, water (0.1% TFA)/acetonitrile, gradient over 12 minutes, 25 ml/min). The pure fractions were pooled and concentrated giving 72 mg (19%) of the title compound as a TFA salt.

LCMS (ESI+): m/z [M+H]+ calcd.: 387, found: 387.

¹H NMR (400 MHz, DMSO-d6) δ 9.65 (s, 1H), 7.73 (t, J=1.7 Hz, 1H), 7.63-7.55 (m, 2H), 7.41 (t, J=7.9 Hz, 1H), 7.20 (d, J=8.7 Hz, 1H), 6.83 (dd, J=8.3, 2.6 Hz, 1H), 6.70 (d, J=2.5 Hz, 1H), 6.05 (s, 2H), 1.98 (s, 3H).

### Example 8. Synthesis of 5-amino-1-(5-hydroxy-2-methylphenyl)-3-[3-(pyridin-4-yl)phenyl]-1H-pyrazole-4-carboxamide (Compound 7)

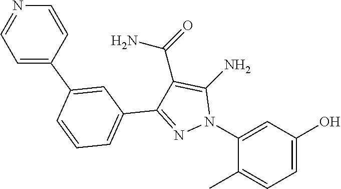 [SMILES: Cc1ccc(O)cc1-n1nc(-c2cccc(-c3ccncc3)c2)c(C(N)=O)c1N]

5-Amino-3-(3-bromophenyl)-1-(5-hydroxy-2-methylphenyl)-1H-pyrazole-4-carboxamide (Example 6, 35 mg, 70 μmol), 4-(4,4,5,5-tetramethyl-1,3,2-dioxaborolan-2-yl)pyridine (17 mg, 84 μmol), potassium carbonate (39 mg, 0.28 mmol) and Pd(dppf)Cl₂ (2.6 mg. 3.5 μmol) were mixed in 5 ml of dioxane+1 ml of water. The reaction mixture was evacuated and purged with nitrogen twice. The mixture was stirred at 90° C. in a closed flask overnight. After cooling the mixture was diluted with ethyl acetate, filtered and concentrated.

The residue was dissolved in water/methanol and purified with reversed phase chromatography (Gemini NX-C18, 30*150 mm, water (50 mM NH₄OH)/acetonitrile, gradient over 12 minutes, 50 ml/min). The pure fractions were pooled and concentrated giving 19 mg (71%) of the title compound.

LCMS (ESI+): m/z [M+H]+ calcd.: 385, found: 386.

¹H NMR (400 MHz, DMSO-d6) δ 8.67-8.63 (m, 2H), 7.96 (s, 1H), 7.86 (d, J=8.0 Hz, 1H), 7.77-7.72 (m, 2H), 7.68 (d, J=7.7 Hz, 1H), 7.62 (t, J=7.6 Hz, 1H), 7.20 (d, J=8.7 Hz, 1H), 6.83 (dd, J=8.3, 2.5 Hz, 1H), 6.72 (d, J=2.5 Hz, 1H), 6.11 (s, 2H), 2.01 (s, 3H).

### Example 9. Synthesis of 5-amino-1-(5-hydroxy-2-methylphenyl)-3-[3-(piperidin-4-yl)phenyl]-1H-pyrazole-4-carboxamide (Compound 8)

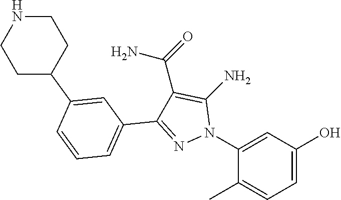 [SMILES: Cc1ccc(O)cc1-n1nc(-c2cccc(C3CCNCC3)c2)c(C(N)=O)c1N]

5-Amino-1-(5-hydroxy-2-methylphenyl)-3-[3-(pyridin-4-yl)phenyl]-1H-pyrazole-4-carboxamide (Example 8, 19 mg, 49 μmol) was dissolved in 30 ml of ethanol. 20 μl of TFA was added. The solution was pumped through an H-cube flow hydrogenation unit (1 ml/min, Pd/C, 50 bar hydrogen pressure, 50° C.). The solvent was removed under reduced pressure and the residue lyophilized from water/acetonitrile giving 23 mg (92%) of the title compound as a TFA salt.

LCMS (ESI+): m/z [M+H]+ calcd.: 392, found: 392.

¹H NMR (400 MHz, Methanol-d4) δ 7.54-7.37 (m, 4H), 7.24 (d, J=8.4 Hz, 1H), 6.90 (dd, J=8.4, 2.5 Hz, 1H), 6.80 (d, J=2.4 Hz, 1H), 3.50 (d, J=12.8 Hz, 2H), 3.15 (t, J=11.9 Hz, 2H), 3.00 (t, J=12.1 Hz, 1H), 2.12 (d, J=13.4 Hz, 2H), 2.08 (s, 3H), 2.01-1.87 (m, 2H).

### Example 10. Synthesis of 5-amino-1-(3-hydroxy-2,6-dimethylphenyl)-3-phenyl-1H-pyrazole-4-carboxamide; trifluoroacetic acid (Compound 9)

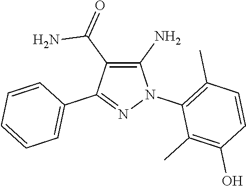 [SMILES: Cc1ccc(O)c(C)c1-n1nc(-c2ccccc2)c(C(N)=O)c1N]

### 5-amino-1-(3-hydroxy-2,6-dimethylphenyl)-3-phenyl-1H-pyrazole-4-carbonitrile

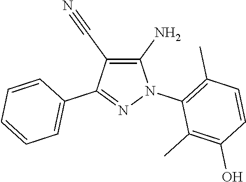 [SMILES: Cc1ccc(O)c(C)c1-n1nc(-c2ccccc2)c(C#N)c1N]

Diisopropylethylamine (59 μl, 339 μmol, 3.0 equiv.) was added to a solution of 3-hydrazinyl-2,4-dimethylphenol hydrochloride (Intermediate 10, 21 mg, 112 μmol, 1.0 equiv.) and 2-[methoxy (phenyl)methylidene]-propanedinitrile (21 mg, 112 μmol, 1.0 equiv.) in dry ethanol (1 ml) and the mixture was heated at 60° C. in a closed vial for 6 h. The mixture was diluted with ethyl acetate and washed with HCl (0.1 M aq.) followed by brine, the organic phase was filtered through a phase-separator and concentrated to give the title compound (34 mg, quant.) as a brown solid, which was used in the next step without further purification.

### 5-amino-1-(3-hydroxy-2,6-dimethylphenyl)-3-phenyl-1H-pyrazole-4-carboxamide; trifluoroacetic acid (Compound 9)

5-amino-1-(3-hydroxy-2,6-dimethylphenyl)-3-phenyl-1H-pyrazole-4-carbonitrile (25 mg, 82 μmol) was dissolved in sulfuric acid (conc., 375 μl) followed by the addition of water (300 μl) and the mixture was heated at 40-60° C. for 3 days. An additional portion of sulfuric acid (500 μl) was then added and after 2.5 h at 60° C. LCMS indicated complete conversion of the nitrile starting material. The mixture was diluted with water (30 ml), the pH adjusted to ca. 6 with sodium hydroxide and acetic acid, and then extracted with dichloromethane (3×20 ml) The organic phase was washed with brine, filtered through a phase-separator, and concentrated. The residue was dissolved in methanol/water, acidified with TFA and purified with reversed phase chromatography (Gemini NX-C18, 21*150 mm, water (0.1% TFA)/acetonitrile, 10-40% gradient over 12 minutes, 25 ml/min). The pure fractions were pooled and concentrated to give the title compound (16.5 mg, 46%) as a white solid.

¹H NMR (500 MHz, DMSO) δ 9.49 (s, 1H), 7.61-7.54 (m, 2H), 7.51-7.41 (m, 3H), 7.02 (d, J=8.2 Hz, 1H), 6.88 (d, J=8.2 Hz, 1H), 6.00 (s, 2H), 1.92 (s, 3H), 1.82 (s, 3H).

HPLC: Oklahoma method, 5-95 (0.1% TFA) 98% purity @220 nm

LCMS: calc m/z+H 323, found 323.

### Example 11. Synthesis of 5-amino-3-(4-fluorophenyl)-1-(3-hydroxy-2,6-dimethylphenyl)-1H-pyrazole-4-carboxamide (Compound 67)

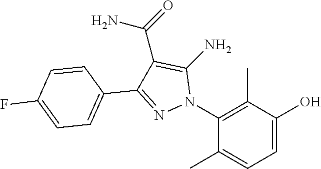 [SMILES: Cc1ccc(O)c(C)c1-n1nc(-c2ccc(F)cc2)c(C(N)=O)c1N]

The title compound was synthesized in a similar way as Example 1.2 using 2-[(4-fluorophenyl)(methoxy)methylidene]propanedinitrile (Intermediate 2, 20 mg, 100 μmol) and 3-hydrazinyl-2,4-dimethylphenol hydrochloride (Intermediate 10, 19 mg, 100 μmol) giving 13 mg (29%) of the title compound as a TFA salt.

LCMS (ESI+): m/z [M+H]+ calcd.: 325, found: 325.

¹H NMR (500 MHz, Methanol-d4) δ 7.66-7.60 (m, 2H), 7.28-7.22 (m, 2H), 7.05 (d, J=8.3 Hz, 1H), 6.88 (d, J=8.3 Hz, 1H), 2.02 (s, 3H), 1.95 (s, 3H).

### Example 12. Synthesis of 5-amino-1-(3-hydroxy-2,6-dimethylphenyl)-3-(4-methoxyphenyl)-1H-pyrazole-4-carboxamide (Compound 68)

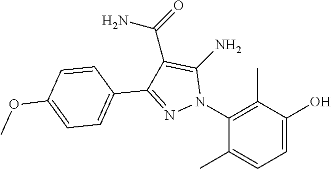 [SMILES: COc1ccc(-c2nn(-c3c(C)ccc(O)c3C)c(N)c2C(N)=O)cc1]

The title compound was synthesized in a similar way as Example 1.2 using 2-[methoxy(4-methoxyphenyl)methylidene]propanedinitrile (Intermediate 3, 20 mg, 100 μmol) and 3-hydrazinyl-2,4-dimethylphenol hydrochloride (Intermediate 10, 19 mg, 100 μmol) giving 7 mg (15%) of the title compound as a TFA salt.

LCMS (ESI+): m/z [M+H]+ calcd.: 353, found: 353.

¹H NMR (500 MHz, Methanol-d4) δ 7.54-7.50 (m, 2H), 7.09-7.04 (m, 3H), 6.88 (d, J=8.3 Hz, 1H), 3.86 (s, 3H), 2.03 (s, 3H), 1.96 (s, 3H).

### Example 13. Synthesis of 5-amino-3-(4-chlorophenyl)-1-(3-hydroxy-2,6-dimethylphenyl)-1H-pyrazole-4-carboxamide (Compound 69)

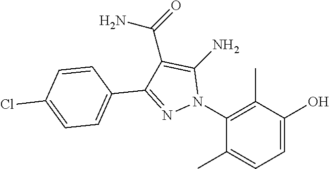 [SMILES: Cc1ccc(O)c(C)c1-n1nc(-c2ccc(Cl)cc2)c(C(N)=O)c1N]

The title compound was synthesized in a similar way as Example 1.2 using 2-[(4-chlorophenyl)(methoxy)methylidene]propanedinitrile (Intermediate 4, 22 mg, 100 μmol) and 3-hydrazinyl-2,4-dimethylphenol hydrochloride (Intermediate 10, 19 mg, 100 μmol) giving 13 mg (29%) of the title compound as a TFA salt.

LCMS (ESI+): m/z [M+H]+ calcd.: 357, found: 357.

¹H NMR (500 MHz, Methanol-d4) δ 7.62-7.57 (m, 2H), 7.54-7.49 (m, 2H), 7.05 (d, J=8.3 Hz, 1H), 6.88 (d, J=8.3 Hz, 1H), 2.02 (s, 3H), 1.95 (s, 3H).

### Example 14. Synthesis of 5-amino-3-(4-fluorophenyl)-1-(3-hydroxy-2,6-dimethylphenyl)-1H-pyrazole-4-carboxamide (Compound 70)

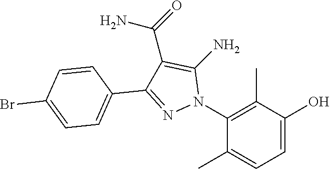 [SMILES: Cc1ccc(O)c(C)c1-n1nc(-c2ccc(Br)cc2)c(C(N)=O)c1N]

The title compound was synthesized in a similar way as Example 1.2 using 2-[(4-bromophenyl)(methoxy)methylidene]propanedinitrile (Intermediate 9, 26 mg, 100 μmol) and 3-hydrazinyl-2,4-dimethylphenol hydrochloride (Intermediate 10, 19 mg, 100 μmol) giving 10 mg (19%) of the title compound as a TFA salt.

LCMS (ESI+): m/z [M+H]+ calcd.: 401, found: 401.

¹H NMR (500 MHz, Methanol-d4) δ 7.69-7.65 (m, 2H), 7.56-7.50 (m, 2H), 7.05 (d, J=8.3 Hz, 1H), 6.88 (d, J=8.3 Hz, 1H), 2.01 (s, 3H), 1.94 (s, 3H).

### Example 15. Synthesis of 5-amino-3-(3-bromophenyl)-1-(3-hydroxy-2,6-dimethylphenyl)-1H-pyrazole-4-carboxamide (Compound 71)

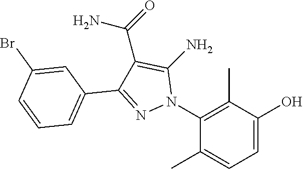 [SMILES: Cc1ccc(O)c(C)c1-n1nc(-c2cccc(Br)c2)c(C(N)=O)c1N]

The title compound was synthesized in a similar way as Example 1.2 using 2-[(3-bromophenyl)(methoxy)methylidene]propanedinitrile (Intermediate 5, 26 mg, 100 μmol) and 3-hydrazinyl-2,4-dimethylphenol hydrochloride (Intermediate 10, 19 mg, 100 μmol) giving 13 mg (25%) of the title compound as a TFA salt.

LCMS (ESI+): m/z [M+H]+ calcd.: 401, found: 401.

¹H NMR (500 MHz, Methanol-d4) δ 7.77 (t, J=1.7 Hz, 1H), 7.64 (ddd, J=8.0, 2.0, 1.0 Hz, 1H), 7.61-7.58 (m, 1H), 7.43 (t, J=7.9 Hz, 1H), 7.05 (d, J=8.3 Hz, 1H), 6.88 (d, J=8.3 Hz, 1H), 2.02 (s, 3H), 1.95 (s, 3H).

### Example 16. Synthesis of 5-amino-3-(1-benzofuran-2-yl)-1-(3-hydroxy-2,6-xylyl)-4-pyrazolecarboxamide (Compound 72) and Separation of Atropisomers Thereof (Compounds 107 and 109)

### 5-amino-3-(1-benzofuran-2-yl)-1-(3-hydroxy-2,6-xylyl)-4-pyrazolecarboxamide

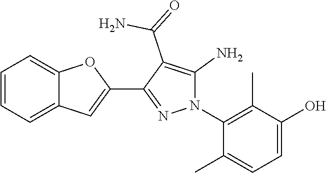 [SMILES: Cc1ccc(O)c(C)c1-n1nc(-c2cc3ccccc3o2)c(C(N)=O)c1N]

[(1-Benzofuran-2-yl)methoxymethylene]propanedinitrile (22 mg, 100 μmol), 3-hydrazino-2,4-xylenol hydrochloride (19 mg, 100 μmol) and Et₃N (41 μl, 300 μmol) were mixed in 1 ml of methanol. The reaction mixture was stirred overnight at 60° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated. The residue was dissolved on 300 μl of 75% H₂SO₄. The reaction mixture was stirred at 50° C. overnight, diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 18 mg (38%) of the title compound as a TFA salt.

LCMS (ESI+): m/z [M+H]+ found: 363.

¹H NMR (500 MHz, Methanol-d4) δ 7.69-7.66 (m, 1H), 7.62-7.59 (m, 1H), 7.40-7.35 (m, 1H), 7.30 (td, J=7.6, 0.9 Hz, 1H), 7.24 (d, J=0.9 Hz, 1H), 7.08 (d, J=8.3 Hz, 1H), 6.90 (d, J=8.3 Hz, 1H), 2.02 (s, 3H), 1.95 (s, 3H).

### Separation of Atropisomers of 5-amino-3-(4-bromofuran-2-yl)-1-(3-hydroxy-2,6-dimethylphenyl)-1H-pyrazole-4-carboxamide

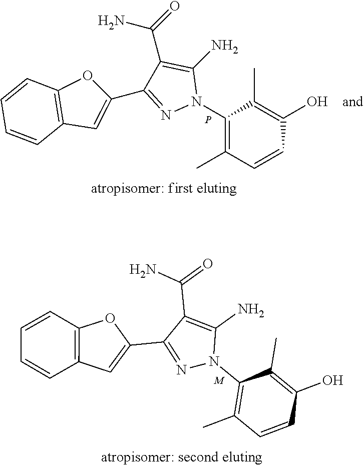

Racemic 5-amino-3-(1-benzofuran-2-yl)-1-(3-hydroxy-2,6-xylyl)-4-pyrazolecarboxamide (100 mg) was subjected to SFC chiral (I-cellulose-Z_0.5% IPAm in IPA) purification. The first fraction was lyophilized to give 5-amino-3-(benzofuran-2-yl)-1-(3-hydroxy-2,6-dimethylphenyl)-1H-pyrazole-4-carboxamide (first eluting, 20 mg, 0.054 mmol, 21.78% yield) and the second fraction likewise gave (5-amino-3-(benzofuran-2-yl)-1-(3-hydroxy-2,6-dimethylphenyl)-1H-pyrazole-4-carboxamide (second eluting, 21 mg, 0.056 mmol, 22.40% yield) as off white solids. The absolute configuration of the atropisomers were not determined but assigned arbitrarily.

Compound 107 (first eluting):

LCMS (ESI) m/z=363.1 (M+H).

¹H NMR (400 MHz, DMSO-d6): δ 9.55 (bs, 1H), 7.70-7.64 (m, 2H), 7.38-7.25 (m, 3H), 7.06-7.03 (m, 3H), 6.92-6.90 (d, J=8.4 Hz, 1H), 6.16 (bs, 2H), 1.91 (s, 3H), 1.81 (s, 3H).

Compound 109 (second eluting):

LCMS (ESI) m/z=363.1 (M+H).

¹H NMR (400 MHz, DMSO-d6): δ 9.54 (s, 1H), 7.70-7.64 (m, 2H), 7.38-7.25 (m, 3H), 7.05-7.007 (m, 3H), 6.92-6.90 (d, J=8.4 Hz, 1H), 6.16 (bs, 2H), 1.91 (s, 3H), 1.81 (s, 3H).

### Example 17. Synthesis of 5-amino-3-(5-bromo-2-furyl)-1-(3-hydroxy-2,6-xylyl)-4-pyrazolecarboxamide (Compound 73)

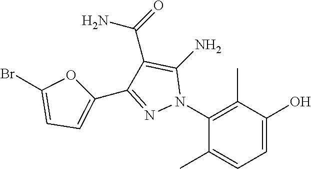 [SMILES: Cc1ccc(O)c(C)c1-n1nc(-c2ccc(Br)o2)c(C(N)=O)c1N]

[(5-Bromo-2-furyl)methoxymethylene]propanedinitrile (127 mg, 0.5 mmol), 3-hydrazino-2,4-xylenol hydrochloride (94 mg, 0.5 mmol) and triethylamine (208 μl, 1.5 mmol) were mixed in 5 ml of methanol and stirred at 60° C. overnight. The reaction mixture was concentrated and the residue partitioned between water and ethyl acetate. The organic phase was washed with sat. NaHCO₃ and brine, dried over MgSO₄, filtered and concentrated. The residue was dissolved on 1 ml of 75% H₂SO₄. The reaction mixture was stirred at 50° C. for 2 days, diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 78 mg (31%) of the title compound as a TFA salt.

LCMS (ESI+): m/z [M+H]+ found: 391.

¹H NMR (500 MHz, Methanol-d4) δ 7.05 (d, J=8.3 Hz, 1H), 6.88 (d, J=8.3 Hz, 1H), 6.84 (d, J=3.5 Hz, 1H), 6.59 (d, J=3.5 Hz, 1H), 1.98 (s, 3H), 1.92 (s, 3H).

### Example 18. Synthesis of 5-amino-3-(5-bromo-2-furyl)-1-(4-chloro-3-hydroxy-2,6-xylyl)-4-pyrazolecarboxamide (Compound 74)

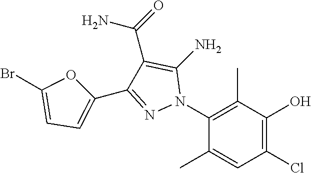 [SMILES: Cc1cc(Cl)c(O)c(C)c1-n1nc(-c2ccc(Br)o2)c(C(N)=O)c1N]

The title compound was isolated as a by-product from the reaction mixture in the synthesis of 5-amino-3-(5-bromo-2-furyl)-1-(3-hydroxy-2,6-xylyl)-4-pyrazolecarboxamide. TFA salt, 7 mg (3%).

LCMS (ESI+): m/z [M+H]+ found: 425.

¹H NMR (500 MHz, Methanol-d4) δ 7.24 (s, 1H), 6.85 (d, J=3.5 Hz, 1H), 6.60 (d, J=3.5 Hz, 1H), 1.99 (s, 3H), 1.98 (s, 3H).

### Example 19. Synthesis of 5-amino-3-(2-furyl)-1-(3-hydroxy-2,6-xylyl)-4-pyrazolecarboxamide (Compound 75) and separation of atropisomers thereof (Compounds 104 and 105)

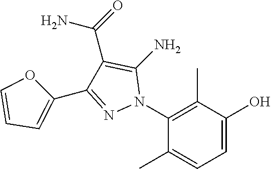 [SMILES: Cc1ccc(O)c(C)c1-n1nc(-c2ccco2)c(C(N)=O)c1N]

### 5-amino-3-(2-furyl)-1-(3-hydroxy-2,6-xylyl)-4-pyrazolecarboxamide

5-Amino-3-(5-bromo-2-furyl)-1-(3-hydroxy-2,6-xylyl)-4-pyrazolecarboxamide (50 mg, 99 μmol) was dissolved in 20 ml of ethanol and pumped through an H-cube hydrogenation unit, 1 ml/min, Pd/C cartridge at 50° C., 50 bar hydrogen pressure. The reaction mixture was concentrated, diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 33 mg (78%) of the title compound as a TFA salt.

LCMS (ESI+): m/z [M+H]+ calcd.: 313, found: 313.

¹H NMR (500 MHz, Methanol-d4) δ 7.72 (dd, J=1.9, 0.8 Hz, 1H), 7.06 (d, J=8.3 Hz, 1H), 6.89 (d, J=8.3 Hz, 1H), 6.85 (dd, J=3.4, 0.8 Hz, 1H), 6.62 (dd, J=3.4, 1.9 Hz, 1H), 1.99 (s, 3H), 1.93 (s, 3H).

### Separation of atropisomers of 5-amino-3-(furan-2-yl)-1-(3-hydroxy-2,6-dimethylphenyl)-1H-pyrazole-4-carboxamide

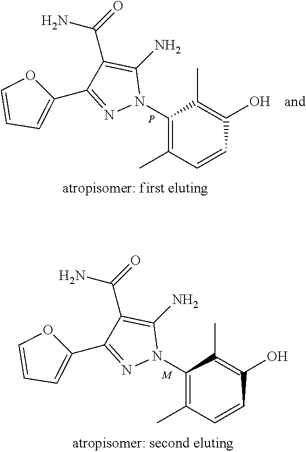

90 mg of racemic compound was separated by chiral SFC (I-cellulose-Z_0.5% IPAm in IPA). After SFC chiral purification, both isomers were concentrated under reduced pressure to give pure fractions. The fractions were lyophilized to give the first eluting isomer of 5-amino-3-(furan-2-yl)-1-(3-hydroxy-2,6-dimethylphenyl)-1H-pyrazole-4-carboxamide (first eluting, 28.41 mg, 0.090 mmol, 31.3% yield) and the second eluting isomer of 5-amino-3-(furan-2-yl)-1-(3-hydroxy-2,6-dimethylphenyl)-1H-pyrazole-4-carboxamide (second eluting, 36.47 mg, 0.116 mmol, 40.3% yield). The absolute configuration of the atropisomers were not determined but assigned arbitrarily.

Compound 104 (first eluting)

LCMS: m/z=313 [M+1].

¹H NMR (400 MHz, DMSO-d6): δ 1.79 (s, 3H), 1.88 (s, 3H), 6.20 (s, 2H), 6.64-6.63 (m, 1H), 6.78 (t, J=0.8 Hz, 1H), 7.04-6.88 (m, 4H), 7.85 (d, J=0.80 Hz, 1H), 9.54 (s, 1H).

Compound 105 (second eluting):

LCMS: m/z=313.1 [M+1].

¹H NMR (400 MHz, DMSO-d6): δ 1.79 (s, 3H), 1.88 (s, 3H), 6.20 (s, 2H), 6.64-6.63 (m, 1H), 6.78 (t, J=0.80 Hz, 1H), 6.89 (d, J=8.00 Hz, 3H), 7.03 (d, J=8.40 Hz, 1H), 7.85 (d, J=0.80 Hz, 1H), 9.54 (s, 1H).

### Example 20. Synthesis of 5-amino-3-(5-bromo-3-furyl)-1-(3-hydroxy-2,6-xylyl)-4-pyrazolecarboxamide (Compound 78) and Separation of Atropisomers Thereof (Compounds 106 and 110)

### 5-amino-3-(5-bromo-3-furyl)-1-(3-hydroxy-2,6-xylyl)-4-pyrazolecarboxamide

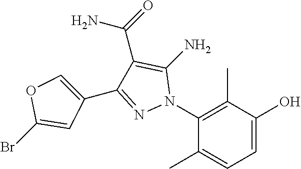 [SMILES: Cc1ccc(O)c(C)c1-n1nc(-c2coc(Br)c2)c(C(N)=O)c1N]

[(5-Bromo-3-furyl)methoxymethylene]propanedinitrile (127 mg, 0.5 mmol), 3-hydrazino-2,4-xylenol hydrochloride (94 mg, 0.5 mmol) and triethylamine (208 μl, 1.5 mmol) were mixed in 5 ml of methanol and stirred at 60° C. overnight. The reaction mixture was concentrated and the residue partitioned between water and ethyl acetate. The organic phase was washed with sat. NaHCO₃ and brine, dried over MgSO₄, filtered and concentrated. The residue was dissolved in 3 ml of methanol and treated with 500 μl of 5 M NaOH and 500 μl of 35% H₂O₂. The reaction mixture was stirred at 50° C. for overnight, acidified with TFA, diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 47 mg (15%) of the title compound as a TFA salt.

LCMS (ESI+): m/z [M+H]+ calcd.: 391, found: 391.

¹H NMR (400 MHz, Methanol-d4) δ 7.96 (d, J=0.9 Hz, 1H), 7.05 (d, J=8.2 Hz, 1H), 6.88 (d, J=8.3 Hz, 1H), 6.73 (d, J=0.9 Hz, 1H), 1.99 (s, 3H), 1.92 (s, 3H).

### Separation of the Atropisomers of 5-amino-3-(4-bromofuran-2-yl)-1-(3-hydroxy-2,6-dimethylphenyl)-1H-pyrazole-4-carboxamide

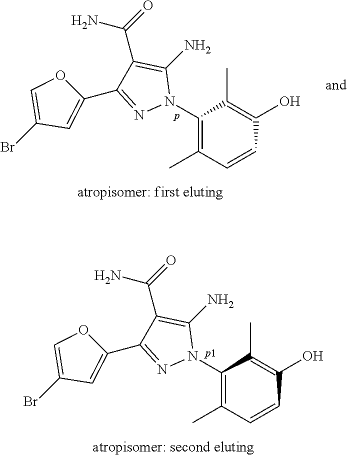

100 mg compound separated by chiral SFC (Lux Amylose-1_0.5% IPAm in MeOH). After SFC chiral purification, both isomers were concentrated under reduced pressure. The first fraction was lyophilized to give the first eluting isomer of 5-amino-3-(4-bromofuran-2-yl)-1-(3-hydroxy-2,6-dimethylphenyl)-1H-pyrazole-4-carboxamide (Compound 106; first eluting, 47 mg, 0.118 mmol, 32.9% yield) and the second eluting isomer of 5-amino-3-(4-bromofuran-2-yl)-1-(3-hydroxy-2,6-dimethylphenyl)-1H-pyrazole-4-carboxamide (Compound 110; second eluting, 34 mg, 0.086 mmol, 24.04% yield) as off white solids. The absolute configuration of the atropisomers were not determined but assigned arbitrarily.

Compound 106 (first eluting):

LCMS (ESI) m/z=393.0 (M+2).

¹H NMR (400 MHz, DMSO-d6): δ 9.53 (bs, 1H), 8.05-8.04 (bs, 1H), 7.03-7.01 (d, J=8 Hz 1H), 6.94-6.80 (m, 3H), 6.13 (bs, 2H), 1.87 (s, 3H), 1.77 (s, 3H).

Compound 110 (second eluting):

LCMS (ESI) m/z=392.7 (M+2).

¹H NMR (400 MHz, DMSO-d6): δ 9.53 (s, 1H), 8.05-8.04 (bs, 1H), 7.03-7.01 (d, J=8 Hz 1H), 6.94-6.88 (m, 3H), 6.13 (bs, 2H), 1.87 (s, 3H), 1.77 (s, 3H).

### Example 21. Synthesis of 5-amino-3-(3-furyl)-1-(3-hydroxy-2,6-xylyl)-4-pyrazolecarboxamide (Compound 76)

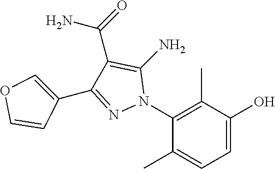 [SMILES: Cc1ccc(O)c(C)c1-n1nc(-c2ccoc2)c(C(N)=O)c1N]

5-Amino-3-(5-bromo-3-furyl)-1-(3-hydroxy-2,6-xylyl)-4-pyrazolecarboxamide (15 mg, 30 μmol) was dissolved in 10 ml of ethanol and pumped through an H-cube hydrogenation unit, 1 ml/min, Pd/C cartridge at 50° C., 50 bar hydrogen pressure. The reaction mixture was concentrated, diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 13 mg (100%) of the title compound as a TFA salt.

LCMS (ESI+): m/z [M+H]+ calcd.: 313, found: 313.

¹H NMR (400 MHz, Methanol-d4) δ 7.93-7.90 (m, 1H), 7.68 (t, J=1.7 Hz, 1H), 7.06 (d, J=8.3 Hz, 1H), 6.88 (d, J=8.2 Hz, 1H), 6.76-6.73 (m, 1H), 2.00 (s, 3H), 1.93 (s, 3H).

### Example 22. Synthesis of 5-amino-3-(4-bromo-2-furyl)-1-(3-hydroxy-2,6-xylyl)-4-pyrazolecarboxamide (Compound 79)

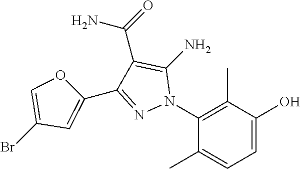 [SMILES: Cc1ccc(O)c(C)c1-n1nc(-c2cc(Br)co2)c(C(N)=O)c1N]

[(4-Bromo-2-furyl)methoxymethylene]propanedinitrile (506 mg, 2 mmol), 3-hydrazino-2,4-xylenol hydrochloride (377 mg, 2 mmol) and triethylamine (843 μl, 6 mmol) were mixed in 50 ml of methanol and stirred at room temperature for 2 hours. The reaction mixture was concentrated and the residue partitioned between water and ethyl acetate. The organic phase was washed with sat. NaHCO₃ and brine, dried over MgSO₄, filtered and concentrated. The residue was recrystallized from ethyl acetate/heptane.

The compound from above was dissolved in 5 ml of 75% H₂SO₄ and stirred at 50° C. overnight. The reaction mixture was poured over an ice/water slurry and neutralized with solid NaHCO₃ until pH~8. Ethyl acetate was added. The organic phase was washed with sat. NaHCO₃ and brine, dried over MgSO₄, filtered and concentrated. The residue was precipitated from ethyl acetate/heptane at 70° C. After cooling the title compound was collected by filtration (0.51 g, 65%).

LCMS (ESI+): m/z [M+H]+ found: 391.

¹H NMR (400 MHz, Methanol-d4) δ 7.81 (d, J=0.7 Hz, 1H), 7.06 (d, J=8.2 Hz, 1H), 6.91 (d, J=0.7 Hz, 1H), 6.89 (d, J=8.3 Hz, 1H), 1.98 (s, 3H), 1.91 (s, 3H).

### Example 23. Synthesis of 5-amino-3-(5-chloro-2-furyl)-1-(3-hydroxy-2,6-xylyl)-4-pyrazolecarboxamide (Compound 80)

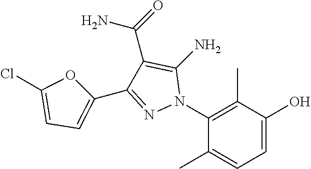 [SMILES: Cc1ccc(O)c(C)c1-n1nc(-c2ccc(Cl)o2)c(C(N)=O)c1N]

[(5-Chloro-2-furyl)methoxymethylene]propanedinitrile (133 mg, 0.64 mmol), 3-hydrazino-2,4-xylenol hydrochloride (120 mg, 0.64 mmol) and triethylamine (263 μl, 1.9 mmol) were mixed in 10 ml of methanol and stirred at 60° C. overnight. The reaction mixture was concentrated and the residue partitioned between water and ethyl acetate. The organic phase was washed with sat. NaHCO₃ and brine, dried over MgSO₄, filtered and concentrated. The residue was dissolved in 3 ml of methanol and treated with 500 μl of 5 M NaOH and 500 μl of 35% H₂O₂. The reaction mixture was stirred at 50° C. for overnight, acidified with TFA, diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 77 mg (26%) of the title compound as a TFA salt.

LCMS (ESI+): m/z [M+H]+ found: 347.

¹H NMR (400 MHz, Methanol-d4) δ 7.06 (d, J=8.3 Hz, 1H), 6.91-6.86 (m, 2H), 6.47 (d, J=3.5 Hz, 1H), 1.99 (s, 3H), 1.92 (s, 3H).

### Example 24. Synthesis of 5-amino-1-(3-hydroxy-2,6-xylyl)-3-(5-methyl-2-furyl)-4-pyrazolecarboxamide (Compound 81)

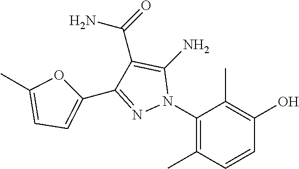 [SMILES: Cc1ccc(-c2nn(-c3c(C)ccc(O)c3C)c(N)c2C(N)=O)o1]

[Methoxy (5-methyl-2-furyl)methylene]propanedinitrile (120 mg, 0.64 mmol), 3-hydrazino-2,4-xylenol hydrochloride (120 mg, 0.64 mmol) and triethylamine (263 μl, 1.9 mmol) were mixed in 10 ml of methanol and stirred at 60° C. overnight. The reaction mixture was concentrated and the residue partitioned between water and ethyl acetate. The organic phase was washed with sat. NaHCO₃ and brine, dried over MgSO₄, filtered and concentrated. The residue was dissolved in 3 ml of methanol and treated with 500 μl of 5 M NaOH and 500 μl of 35% H₂O₂. The reaction mixture was stirred at 50° C. for overnight, acidified with TFA, diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 35 mg (12%) of the title compound as a TFA salt. LCMS (ESI+): m/z [M+H]+ found: 327.

¹H NMR (500 MHz, Methanol-d₄) δ 7.05 (d, J=8.3 Hz, 1H), 6.88 (d, J=8.3 Hz, 1H), 6.76-6.70 (m, 1H), 6.21 (m, 1H), 2.43-2.38 (m, 3H), 1.99 (s, 3H), 1.92 (s, 3H).

### Example 25. Synthesis of 5-amino-3-(1-benzofuran-3-yl)-1-(3-hydroxy-2,6-xylyl)-4-pyrazolecarboxamide (Compound 82)

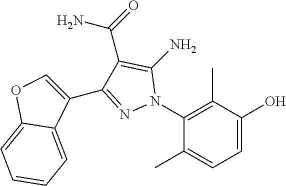 [SMILES: Cc1ccc(O)c(C)c1-n1nc(-c2coc3ccccc23)c(C(N)=O)c1N]

[(1-Benzofuran-3-yl)methoxymethylene]propanedinitrile (22 mg, 100 μmol), 3-hydrazino-2,4-xylenol hydrochloride (19 mg, 100 μmol) and Et₃N (41 μl, 300 μmol) were mixed in 1 ml of methanol. The reaction mixture was stirred overnight at 60° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated. The residue was dissolved in 0.5 ml of methanol and treated with 100 μl of 5 M NaOH and 100 μl of 35% H₂O₂. The reaction mixture was stirred at 50° C. overnight, acidified with TFA, diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 9 mg (19%) of the title compound as a TFA salt.

LCMS (ESI+): m/z [M+H]+ found: 363.

¹H NMR (400 MHz, Methanol-d4) δ 8.11 (s, 1H), 7.65 (d, J=7.7 Hz, 1H), 7.59 (d, J=8.2 Hz, 1H), 7.43-7.37 (m, 1H), 7.32 (t, J=7.4 Hz, 1H), 7.07 (d, J=8.3 Hz, 1H), 6.89 (d, J=8.3 Hz, 1H), 2.06 (s, 3H), 1.98 (s, 3H).

### Example 26. Synthesis of 5-amino-1-(3-hydroxy-2,6-xylyl)-3-(3-methyl-2-furyl)-4-pyrazolecarboxamide (Compound 83)

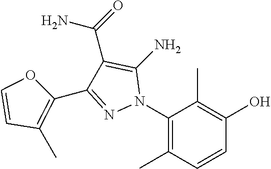 [SMILES: Cc1ccoc1-c1nn(-c2c(C)ccc(O)c2C)c(N)c1C(N)=O]

[Methoxy (3-methyl-2-furyl)methylene]propanedinitrile (19 mg, 100 μmol), 3-hydrazino-2,4-xylenol hydrochloride (19 mg, 100 μmol) and Et₃N (41 μl, 300 μmol) were mixed in 1 ml of methanol. The reaction mixture was stirred overnight at 60° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated. The residue was dissolved in 400 μl of 75% H₂SO₄ and stirred at 50° C. overnight. The reaction mixture was diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 11 mg (25%) of the title compound as a TFA salt.

LCMS (ESI+): m/z [M+H]+ found: 327.

¹H NMR (400 MHz, Methanol-d4) δ 7.61 (d, J=1.7 Hz, 1H), 7.06 (d, J=8.2 Hz, 1H), 6.88 (d, J=8.3 Hz, 1H), 6.51 (d, J=1.7 Hz, 1H), 2.16 (s, 3H), 2.01 (s, 3H), 1.93 (s, 3H).

### Example 27. Synthesis of 5-amino-3-(4,5-dimethyl-2-furyl)-1-(3-hydroxy-2,6-xylyl)-4-pyrazolecarboxamide (Compound 89)

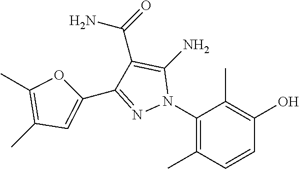 [SMILES: Cc1cc(-c2nn(-c3c(C)ccc(O)c3C)c(N)c2C(N)=O)oc1C]

[(4,5-Dimethyl-2-furyl)methoxymethylene]propanedinitrile (20 mg, 100 μmol), 3-hydrazino-2,4-xylenol hydrochloride (19 mg, 100 μmol) and Et₃N (41 μl, 300 μmol) were mixed in 1 ml of methanol. The reaction mixture was stirred overnight at 60° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated. The residue was dissolved in 300 μl of 75% H₂SO₄ and stirred at 50° C. overnight. The reaction mixture was diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 20 mg (44%) of the title compound as a TFA salt.

LCMS (ESI+): m/z [M+H]+ found: 341.

¹H NMR (400 MHz, Methanol-d4) δ 7.05 (d, J=8.3 Hz, 1H), 6.88 (d, J=8.3 Hz, 1H), 6.61 (s, 1H), 2.31 (s, 3H), 2.00 (s, 3H), 1.98 (s, 3H), 1.91 (s, 3H).

### Example 28. Synthesis of 5-amino-3-(1-benzofuran-2-yl)-1-(5-methyl-1H-indazol-4-yl)-4-pyrazolecarboxamide (Compound 77)

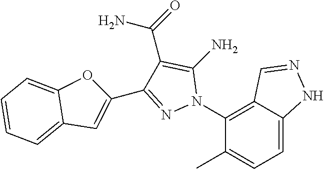 [SMILES: Cc1ccc2[nH]ncc2c1-n1nc(-c2cc3ccccc3o2)c(C(N)=O)c1N]

[(1-Benzofuran-2-yl)methoxymethylene]propanedinitrile (22 mg, 100 μmol), [5-methyl-1-(tetrahydro-2H-pyran-2-yl)-1H-indazol-4-yl]hydrazine trifluoraacetate (36 mg, 100 μmol) and Et₃N (41 μl, 300 μmol) were mixed in 1 ml of methanol. The reaction mixture was stirred overnight at 60° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated. The residue was dissolved in 300 μl of 75% H₂SO₄ and stirred at 50° C. overnight. The reaction mixture was diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 3.5 mg (7%) of the title compound as a TFA salt.

LCMS (ESI+): m/z [M+H]+ found: 373.

¹H NMR (400 MHz, Methanol-d4) δ 7.80 (s, 1H), 7.70-7.66 (m, 2H), 7.62 (d, J=8.6 Hz, 1H), 7.49 (d, J=8.6 Hz, 1H), 7.42-7.36 (m, 1H), 7.34-7.28 (m, 2H), 2.32 (s, 3H).

### Example 29. Synthesis of 5-amino-3-(2-furyl)-1-(5-methyl-1H-indazol-4-yl)-4-pyrazolecarboxamide (Compound 84)

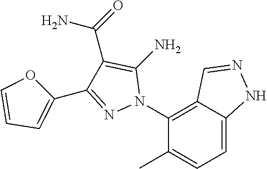 [SMILES: Cc1ccc2[nH]ncc2c1-n1nc(-c2ccco2)c(C(N)=O)c1N]

[(2-Furyl)methoxymethylene]propanedinitrile (26 mg, 150 μmol), [5-methyl-1-(tetrahydro-2H-pyran-2-yl)-1H-indazol-4-yl] hydrazine trifluoroacetate (54 mg, 150 μmol) and Et₃N (62 μl, 450 μmol) were mixed in 1 ml of methanol. The reaction mixture was stirred overnight at 60° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated. The residue was dissolved in 300 μl of 75% H₂SO₄ and stirred at 50° C. overnight. The reaction mixture was diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 1.1 mg (1.7%) of the title compound as a TFA salt.

LCMS (ESI+): m/z [M+H]+ found: 323.

¹H NMR (500 MHz, Methanol-d4) δ 7.76 (d, J=0.9 Hz, 1H), 7.74 (dd, J=1.9, 0.8 Hz, 1H), 7.68-7.64 (m, 1H), 7.46 (d, J=8.6 Hz, 1H), 6.88 (dd, J=3.4, 0.8 Hz, 1H), 6.63 (dd, J=3.4, 1.9 Hz, 1H), 2.29 (s, 3H).

### Example 30. Synthesis of 5-amino-3-(5-bromo-2-furyl)-1-(5-methyl-1H-indazol-4-yl)-4-pyrazolecarboxamide (Compound 85)

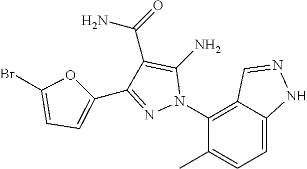 [SMILES: Cc1ccc2[nH]ncc2c1-n1nc(-c2ccc(Br)o2)c(C(N)=O)c1N]

[(5-Bromo-2-furyl)methoxymethylene]propanedinitrile (38 mg, 150 μmol), [5-methyl-1-(tetrahydro-2H-pyran-2-yl)-1H-indazol-4-yl] hydrazine trifluoroacetate (54 mg, 150 μmol) and Et₃N (62 μl, 450 μmol) were mixed in 1 ml of methanol. The reaction mixture was stirred overnight at 60° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated. The residue was dissolved in 300 μl of 75% H₂SO₄ and stirred at 50° C. overnight. The reaction mixture was diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 6 mg (8%) of the title compound as a TFA salt.

LCMS (ESI+): m/z [M+H]+ found: 401.

¹H NMR (500 MHz, Methanol-d4) δ 7.75 (d, J=0.9 Hz, 1H), 7.68-7.64 (m, 1H), 7.46 (d, J=8.6 Hz, 1H), 6.88 (d, J=3.5 Hz, 1H), 6.61 (d, J=3.5 Hz, 1H), 2.28 (s, 3H).

### Example 31. Synthesis of 5-amino-1-(2,6-dichloro-3-hydroxyphenyl)-3-(2-furyl)-4-pyrazolecarboxamide (Compound 91)

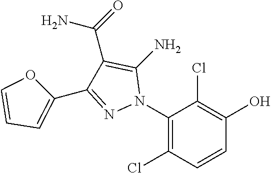 [SMILES: NC(=O)c1c(-c2ccco2)nn(-c2c(Cl)ccc(O)c2Cl)c1N]

[(2-Furyl)methoxymethylene]propanedinitrile (17 mg, 100 μmol), 2,4-dichloro-3-hydrazinophenol hydrochloride (23 mg, 100 μmol) and Et₃N (41 μl, 300 μmol) were mixed in 1 ml of methanol. The reaction mixture was stirred overnight at 60° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated. The residue was dissolved in 300 μl of 75% H₂SO₄ and stirred at 50° C. overnight. The reaction mixture was diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 24 mg (51%) of the title compound as a TFA salt.

LCMS (ESI+): m/z [M+H]+ found: 353.

¹H NMR (500 MHz, Methanol-d4) δ 7.73 (dd, J=1.9, 0.8 Hz, 1H), 7.42 (d, J=9.0 Hz, 1H), 7.14 (d, J=9.0 Hz, 1H), 6.86 (dd, J=3.4, 0.8 Hz, 1H), 6.62 (dd, J=3.4, 1.9 Hz, 1H).

### Example 32. Synthesis of 5-amino-3-(4-bromo-2-furyl)-1-(2,6-dichloro-3-hydroxyphenyl)-4-pyrazolecarboxamide (Compound 92)

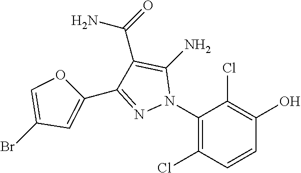 [SMILES: NC(=O)c1c(-c2cc(Br)co2)nn(-c2c(Cl)ccc(O)c2Cl)c1N]

[(4-Bromo-2-furyl)methoxymethylene]propanedinitrile (25 mg, 100 μmol), 2,4-dichloro-3-hydrazinophenol hydrochloride (23 mg, 100 μmol) and Et₃N (41 μl, 300 μmol) were mixed in 1 ml of methanol. The reaction mixture was stirred overnight at 60° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated. The residue was dissolved in 300 μl of 75% H₂SO₄ and stirred at 50° C. overnight. The reaction mixture was diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 18 mg (33%) of the title compound as a TFA salt.

LCMS (ESI+): m/z [M+H]+ found: 431.

¹H NMR (500 MHz, Methanol-d₄) δ 7.81 (d, J=0.9 Hz, 1H), 7.42 (d, J=9.0 Hz, 1H), 7.13 (d, J=9.0 Hz, 1H), 6.93 (d, J=0.9 Hz, 1H).

### Example 33. Synthesis of 5-amino-3-(1-benzofuran-2-yl)-1-(2,6-dichloro-3-hydroxyphenyl)-4-pyrazolecarboxamide (Compound 93)

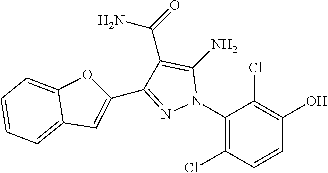 [SMILES: NC(=O)c1c(-c2cc3ccccc3o2)nn(-c2c(Cl)ccc(O)c2Cl)c1N]

[(1-Benzofuran-2-yl)methoxymethylene]propanedinitrile (22 mg, 100 μmol), 2,4-dichloro-3-hydrazinophenol hydrochloride (23 mg, 100 μmol) and Et₃N (41 μl, 300 μmol) were mixed in 1 ml of methanol. The reaction mixture was stirred overnight at 60° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated. The residue was dissolved in 300 μl of 75% H₂SO₄ and stirred at 50° C. overnight. The reaction mixture was diluted with methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 26 mg (50%) of the title compound as a TFA salt.

LCMS (ESI+): m/z [M+H]+ found: 403.

¹H NMR (500 MHz, Methanol-d4) δ 7.70-7.67 (m, 1H), 7.61 (dd, J=8.3, 0.8 Hz, 1H), 7.44 (d, J=9.0 Hz, 1H), 7.38 (ddd, J=8.4, 7.3, 1.3 Hz, 1H), 7.33-7.29 (m, 1H), 7.26 (d, J=0.9 Hz, 1H), 7.15 (d, J=9.0 Hz, 1H).

### Example 34. Synthesis of 5-amino-1-(3-hydroxy-2,6-xylyl)-3-(4-methyl-2-furyl)-4-pyrazolecarboxamide (Compound 90)

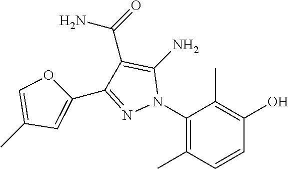 [SMILES: Cc1coc(-c2nn(-c3c(C)ccc(O)c3C)c(N)c2C(N)=O)c1]

5-Amino-3-(4-bromo-2-furyl)-1-(3-hydroxy-2,6-xylyl)-4-pyrazolecarboxamide (20 mg, 50 μmol), methylboronic acid (4.5 mg, 75 μmol), potassium carbonate (28 mg, 200 μmol) and Pd(dppf)Cl₂ (2 mg, 2.5 μmol) were mixed in 400 μl of dioxane+200 μl of water. The reaction mixture was stirred overnight at 100° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 3 mg (14%) of the title compound as a TFA salt.

LCMS (ESI+): m/z [M+H]+ found: 327.

¹H NMR (400 MHz, Methanol-d4) δ 7.48 (s, 1H), 7.06 (d, J=8.2 Hz, 1H), 6.88 (d, J=8.3 Hz, 1H), 6.71 (s, 1H), 2.09 (s, 3H), 1.99 (s, 3H), 1.92 (s, 3H).

### Example 35. Synthesis of 5-amino-3-(5-cyano-2-furyl)-1-(3-hydroxy-2,6-xylyl)-4-pyrazolecarboxamide (Compound 101)

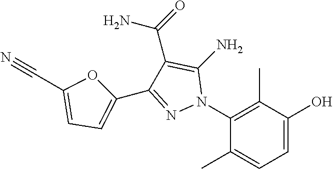 [SMILES: Cc1ccc(O)c(C)c1-n1nc(-c2ccc(C#N)o2)c(C(N)=O)c1N]

5-Amino-3-(5-bromo-2-furyl)-1-(3-hydroxy-2,6-xylyl)-4-pyrazolecarboxamide (20 mg, 50 μmol) and Cu (I) CN (9 mg, 100 μmol) were mixed in 300 μl of dimethylacetamide. The reaction mixture was stirred at 120° C. for 4 days, diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 2.4 mg (11%) of the title compound as a TFA salt.

LCMS (ESI+): m/z [M+H]+ found: 338.

¹H NMR (500 MHz, Methanol-d4) δ 7.44 (d, J=3.8 Hz, 1H), 7.06 (d, J=8.3 Hz, 1H), 7.01 (d, J=3.7 Hz, 1H), 6.89 (d, J=8.3 Hz, 1H), 1.98 (s, 3H), 1.91 (s, 3H).

### Example 36. Synthesis of 5-Amino-1-(3-hydroxy-2,6-xylyl)-3-(5-phenyl-2-furyl)-4-pyrazolecarboxamide (Compound 87)

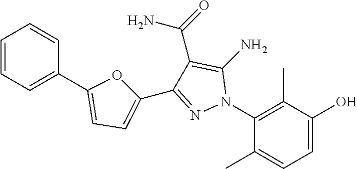 [SMILES: Cc1ccc(O)c(C)c1-n1nc(-c2ccc(-c3ccccc3)o2)c(C(N)=O)c1N]

5-Amino-3-(5-bromo-2-furyl)-1-(3-hydroxy-2,6-xylyl)-4-pyrazolecarboxamide (20 mg, 50 μmol), Et₃N (21 μl, 150 μmol), Pd(dppf)Cl₂ (2 mg, 2.5 μmol) and phenylboronic acid (6 mg, 50 μmol) were mixed in 500 μl of dioxane+100 μl of water. The reaction mixture was stirred overnight at 90° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 10 mg (40%) of the title compound as a TFA salt.

LCMS (ESI+): m/z [M+H]+ found: 389.

### Example 37. Synthesis of 5-amino-1-(3-hydroxy-2,6-xylyl)-3-[5-(3-pyridyl)-2-furyl]-4-pyrazolecarboxamide (Compound 86)

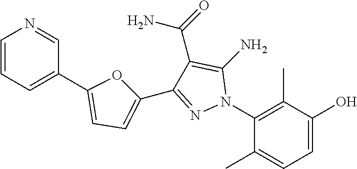 [SMILES: Cc1ccc(O)c(C)c1-n1nc(-c2ccc(-c3cccnc3)o2)c(C(N)=O)c1N]

5-Amino-3-(5-bromo-2-furyl)-1-(3-hydroxy-2,6-xylyl)-4-pyrazolecarboxamide (20 mg, 50 μmol), Et₃N (21 μl, 150 μmol), Pd(dppf)Cl₂ (2 mg, 2.5 μmol) and 3-pyridylboronic acid (6 mg, 50 μmol) were mixed in 500 μl of dioxane+100 μl of water. The reaction mixture was stirred overnight at 90° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 20 mg (79%) of the title compound as a TFA salt.

LCMS (ESI+): m/z [M+H]+ found: 390.

¹H NMR (500 MHz, Methanol-d4) δ 9.21-9.17 (m, 1H), 8.70 (dt, J=8.2, 1.7 Hz, 1H), 8.63 (d, J=5.3 Hz, 1H), 7.92 (dd, J=8.2, 5.5 Hz, 1H), 7.34 (d, J=3.6 Hz, 1H), 7.12 (d, J=3.7 Hz, 1H), 7.08 (d, J=8.3 Hz, 1H), 6.91 (d, J=8.3 Hz, 1H), 2.02 (s, 3H), 1.95 (s, 3H).

### Example 38. Synthesis of 5-amino-1-(3-hydroxy-2,6-xylyl)-3-[5-(4-pyridyl)-2-furyl]-4-pyrazolecarboxamide (Compound 99)

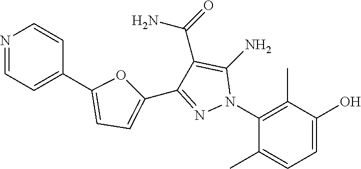 [SMILES: Cc1ccc(O)c(C)c1-n1nc(-c2ccc(-c3ccncc3)o2)c(C(N)=O)c1N]

5-Amino-3-(5-bromo-2-furyl)-1-(3-hydroxy-2,6-xylyl)-4-pyrazolecarboxamide (20 mg, 50 μmol), Et₃N (21 μl, 150 μmol), Pd(dppf)Cl₂ (2 mg, 2.5 μmol) and 4,4,5,5-tetramethyl-2-(4-pyridyl)-1,3,2-dioxaborolane (12 mg, 60 μmol) were mixed in 500 μl of dioxane+100 μl of water. The reaction mixture was stirred overnight at 95° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 15 mg (60%) of the title compound as a TFA salt.

LCMS (ESI+): m/z [M+H]+ found: 390.

¹H NMR (400 MHz, Methanol-d₄) δ 8.72 (d, J=7.0 Hz, 2H), 8.29 (d, J=7.0 Hz, 2H), 7.76 (d, J=3.8 Hz, 1H), 7.23 (d, J=3.8 Hz, 1H), 7.09 (d, J=8.4 Hz, 1H), 6.92 (d, J=8.3 Hz, 1H), 2.02 (s, 3H), 1.94 (s, 3H).

### Example 39. Synthesis of 5-amino-1-(3-hydroxy-2,6-xylyl)-3-[5-(2-pyridyl)-2-furyl]-4-pyrazolecarboxamide (Compound 100)

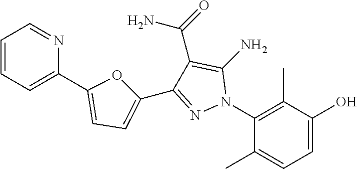 [SMILES: Cc1ccc(O)c(C)c1-n1nc(-c2ccc(-c3ccccn3)o2)c(C(N)=O)c1N]

5-Amino-3-(5-bromo-2-furyl)-1-(3-hydroxy-2,6-xylyl)-4-pyrazolecarboxamide (20 mg, 50 μmol), Pd(dppf)Cl₂ (2 mg, 2.5 μmol) and tributyl(2-pyridyl) stannane (22 mg, 60 μmol) were mixed in 500 μl of dioxane. The reaction mixture was stirred overnight at 90° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 14 mg (56%) of the title compound as a TFA salt.

LCMS (ESI+): m/z [M+H]+ found: 390.

¹H NMR (400 MHz, Methanol-d4) δ 8.64 (d, J=5.5 Hz, 1H), 8.26-8.20 (m, 1H), 8.15 (d, J=8.1 Hz, 1H), 7.61-7.56 (m, 1H), 7.50 (d, J=3.7 Hz, 1H), 7.18 (d, J=3.7 Hz, 1H), 7.09 (d, J=8.2 Hz, 1H), 6.91 (d, J=8.3 Hz, 1H), 2.02 (s, 3H), 1.95 (s, 3H).

### Example 40. Synthesis of 5-amino-1-(3-hydroxy-2,6-xylyl)-3-(4-phenyl-2-furyl)-4-pyrazolecarboxamide (Compound 88)

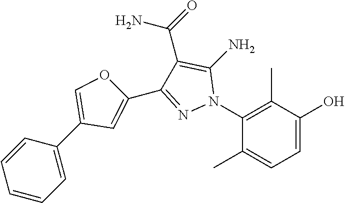 [SMILES: Cc1ccc(O)c(C)c1-n1nc(-c2cc(-c3ccccc3)co2)c(C(N)=O)c1N]

5-Amino-3-(4-bromo-2-furyl)-1-(3-hydroxy-2,6-xylyl)-4-pyrazolecarboxamide (20 mg, 50 μmol), Et₃N (21 μl, 150 μmol), Pd(dppf)Cl₂ (2 mg, 2.5 μmol) and phenylboronic acid (6 mg, 50 μmol) were mixed in 500 μl of dioxane+100 μl of water. The reaction mixture was stirred overnight at 90° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 10 mg (40%) of the title compound as a TFA salt.

LCMS (ESI+): m/z [M+H]+ found: 389.

¹H NMR (500 MHz, Methanol-d4) δ 8.09 (d, J=0.9 Hz, 1H), 7.64-7.59 (m, 2H), 7.42-7.35 (m, 2H), 7.31-7.25 (m, 1H), 7.22 (d, J=0.9 Hz, 1H), 7.07 (d, J=8.3 Hz, 1H), 6.90 (d, J=8.3 Hz, 1H), 2.01 (s, 3H), 1.94 (s, 3H).

### Example 41. Synthesis of 5-amino-1-(3-hydroxy-2,6-xylyl)-3-[4-(4-pyridyl)-2-furyl]-4-pyrazolecarboxamide (Compound 95)

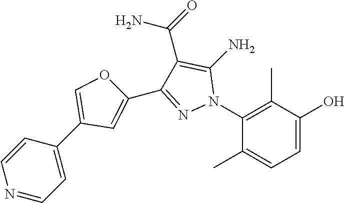 [SMILES: Cc1ccc(O)c(C)c1-n1nc(-c2cc(-c3ccncc3)co2)c(C(N)=O)c1N]

5-Amino-3-(4-bromo-2-furyl)-1-(3-hydroxy-2,6-xylyl)-4-pyrazolecarboxamide (20 mg, 50 μmol), Et₃N (21 μl, 150 μmol), Pd(dppf)Cl₂ (2 mg, 2.5 μmol) and 4,4,5,5-tetramethyl-2-(4-pyridyl)-1,3,2-dioxaborolane (12 mg, 60 μmol) were mixed in 500 μl of dioxane+100 μl of water. The reaction mixture was stirred overnight at 95° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 12 mg (48%) of the title compound as a TFA salt.

LCMS (ESI+): m/z [M+H]+ found: 390.

¹H NMR (500 MHz, Methanol-d4) δ 8.77 (d, J=6.9 Hz, 2H), 8.70 (d, J=0.9 Hz, 1H), 8.30 (d, J=6.9 Hz, 2H), 7.50 (d, J=1.0 Hz, 1H), 7.08 (d, J=8.3 Hz, 1H), 6.91 (d, J=8.3 Hz, 1H), 2.01 (s, 3H), 1.94 (s, 3H).

### Example 42. Synthesis of 5-amino-1-(3-hydroxy-2,6-xylyl)-3-[4-(3-pyridyl)-2-furyl]-4-pyrazolecarboxamide (Compound 94)

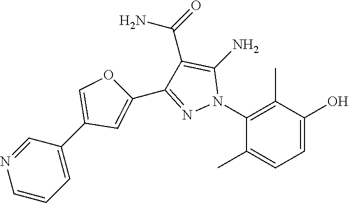 [SMILES: Cc1ccc(O)c(C)c1-n1nc(-c2cc(-c3cccnc3)co2)c(C(N)=O)c1N]

5-Amino-3-(4-bromo-2-furyl)-1-(3-hydroxy-2,6-xylyl)-4-pyrazolecarboxamide (20 mg, 50 μmol), Et₃N (21 μl, 150 μmol), Pd(dppf)Cl₂ (2 mg, 2.5 μmol) and 3-pyridylboronic acid (7 mg, 60 μmol) were mixed in 500 μl of dioxane+100 μl of water. The reaction mixture was stirred overnight at 95° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 23 mg (91%) of the title compound as a TFA salt.

LCMS (ESI+): m/z [M+H]+ found: 390.

¹H NMR (500 MHz, DMSO-d6) δ 9.54 (s, 1H), 9.05 (d, J=1.9 Hz, 1H), 8.60 (dd, J=5.1, 1.3 Hz, 1H), 8.55-8.52 (m, 1H), 8.36 (d, J=8.0 Hz, 1H), 7.67 (dd, J=7.7, 5.1 Hz, 1H), 7.44 (d, J=0.8 Hz, 1H), 7.03 (d, J=8.3 Hz, 1H), 6.90 (d, J=8.3 Hz, 1H), 1.90 (s, 3H), 1.80 (s, 3H).

### Example 43. Synthesis of 5-amino-1-(3-hydroxy-2,6-xylyl)-3-[4-(2-pyridyl)-2-furyl]-4-pyrazolecarboxamide (Compound 98)

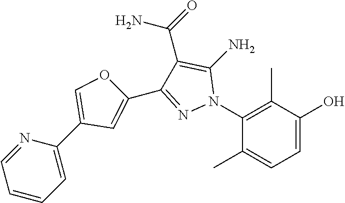 [SMILES: Cc1ccc(O)c(C)c1-n1nc(-c2cc(-c3ccccn3)co2)c(C(N)=O)c1N]

5-Amino-3-(4-bromo-2-furyl)-1-(3-hydroxy-2,6-xylyl)-4-pyrazolecarboxamide (20 mg, 50 μmol), Pd(dppf)Cl₂ (2 mg, 2.5 μmol) and tributyl(2-pyridyl) stannane (22 mg, 60 μmol) were mixed in 500 μl of dioxane. The reaction mixture was stirred overnight at 90° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 8.6 mg (34%) of the title compound as a TFA salt.

LCMS (ESI+): m/z [M+H]+ found: 390.

¹H NMR (400 MHz, Methanol-d4) δ 8.69 (d, J=5.6 Hz, 1H), 8.54 (s, 1H), 8.35 (td, J=8.1, 1.5 Hz, 1H), 8.17 (d, J=8.2 Hz, 1H), 7.76-7.70 (m, 1H), 7.45 (d, J=0.8 Hz, 1H), 7.08 (d, J=8.3 Hz, 1H), 6.90 (d, J=8.2 Hz, 1H), 2.01 (s, 3H), 1.94 (s, 3H).

### Example 44. Synthesis of 5-amino-1-(3-hydroxy-2,6-xylyl)-3-(4-vinyl-2-furyl)-4-pyrazolecarboxamide (Compound 96)

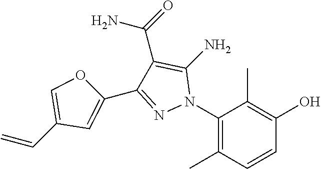 [SMILES: C=Cc1coc(-c2nn(-c3c(C)ccc(O)c3C)c(N)c2C(N)=O)c1]

5-Amino-3-(4-bromo-2-furyl)-1-(3-hydroxy-2,6-xylyl)-4-pyrazolecarboxamide (20 mg, 50 μmol), Et₃N (21 μl, 150 μmol), Pd(dppf)Cl₂ (2 mg, 2.5 μmol) and 4,4,5,5-tetramethyl-2-vinyl-1,3,2-dioxaborolane (9 mg, 60 μmol) were mixed in 500 μl of dioxane+100 μl of water. The reaction mixture was stirred overnight at 95° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 12 mg (53%) of the title compound as a TFA salt.

LCMS (ESI+): m/z [M+H]+ found: 339.

¹H NMR (500 MHz, Methanol-d4) δ 7.72 (s, 1H), 7.06 (d, J=8.3 Hz, 1H), 7.04 (s, 1H), 6.89 (d, J=8.3 Hz, 1H), 6.62 (dd, J=17.6, 10.9 Hz, 1H), 5.60 (dd, J=17.6, 1.0 Hz, 1H), 5.21 (dd, J=10.9, 1.3 Hz, 1H), 1.99 (s, 3H), 1.93 (s, 3H).

### Example 45. Synthesis of 5-amino-1-(3-hydroxy-2,6-xylyl)-3-(4-isopropenyl-2-furyl)-4-pyrazolecarboxamide (Compound 97)

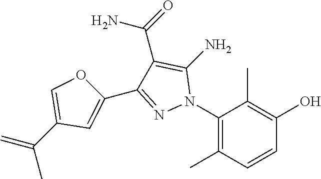 [SMILES: C=C(C)c1coc(-c2nn(-c3c(C)ccc(O)c3C)c(N)c2C(N)=O)c1]

5-Amino-3-(4-bromo-2-furyl)-1-(3-hydroxy-2,6-xylyl)-4-pyrazolecarboxamide (20 mg, 50 μmol), Et₃N (21 μl, 150 μmol), Pd(dppf)Cl₂ (2 mg, 2.5 μmol) and 2-isopropenyl-4,4,5,5-tetramethyl-1,3,2-dioxaborolane (10 mg, 60 μmol) were mixed in 500 μl of dioxane+100 μl of water. The reaction mixture was stirred overnight at 95° C., diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 1.8 mg (8%) of the title compound as a TFA salt.

LCMS (ESI+): m/z [M+H]+ found: 353.

¹H NMR (400 MHz, Methanol-d4) δ 7.75 (s, 1H), 7.06 (d, J=8.4 Hz, 1H), 7.03 (s, 1H), 6.89 (d, J=8.3 Hz, 1H), 5.34 (s, 1H), 5.00 (s, 1H), 2.06 (s, 3H), 1.99 (s, 3H), 1.93 (s, 3H).

### Example 46. Synthesis of 5-amino-1-(3-hydroxy-2,6-xylyl)-3-(4-isopropyl-2-furyl)-4-pyrazolecarboxamide (Compound 102)

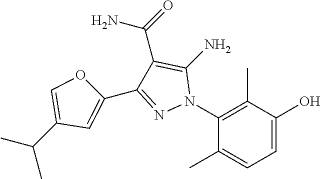 [SMILES: Cc1ccc(O)c(C)c1-n1nc(-c2cc(C(C)C)co2)c(C(N)=O)c1N]

5-Amino-1-(3-hydroxy-2,6-xylyl)-3-(4-isopropenyl-2-furyl)-4-pyrazolecarboxamide (10 mg, 21 μmol) was dissolved in 5 ml of ethanol and pumped through an H-cube hydrogenation unit, 1 ml/min, Pd/C cartridge at 50° C., 50 bar hydrogen pressure. The reaction mixture was concentrated, dissolved in methanol/water and purified with reversed phase chromatography (Gemini NX-C18, 21×150 mm, water (0.1% TFA)/acetonitrile, gradient over 12 minutes, 25 ml/min). The pure fractions were pooled and concentrated giving 6 mg (61%) of the title compound as a TFA salt.

LCMS (ESI+): m/z [M+H]+ found: 355.

¹H NMR (500 MHz, Methanol-d4) δ 7.49 (t, J=0.9 Hz, 1H), 7.06 (d, J=8.3 Hz, 1H), 6.88 (d, J=8.3 Hz, 1H), 6.79 (d, J=0.7 Hz, 1H), 2.84 (dq, J=13.5, 6.5 Hz, 1H), 1.99 (s, 3H), 1.92 (s, 3H), 1.25 (d, J=6.9 Hz, 6H).

### Example 47. Synthesis of 5-amino-1-(3-hydroxy-2,6-xylyl)-3-[4-(2-methyl-1-propenyl)-2-furyl]-4-pyrazolecarboxamide (Compound 108)

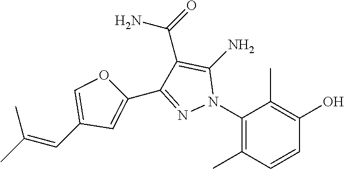 [SMILES: CC(C)=Cc1coc(-c2nn(-c3c(C)ccc(O)c3C)c(N)c2C(N)=O)c1]

5-Amino-3-(4-bromo-2-furyl)-1-(3-hydroxy-2,6-xylyl)-4-pyrazolecarboxamide (39 mg, 100 μmol), potassium carbonate (55 mg, 400 μmol), Peppsi-iPr (3 mg, 5 μmol) and 4,4,5,5-tetramethyl-2-(2-methyl-1-propenyl)-1,3,2-dioxaborolane (36 mg, 200 μmol) were mixed in 3 ml of toluene+2 ml of methanol. The reaction mixture was stirred at 120° C. for 90 minutes in a microwave reactor. After cooling the reaction mixture was filtered and concentrated. The residue was dissolved in methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 6 mg (12%) of the title compound as a TFA salt.

LCMS (ESI+): m/z [M+H]+ found: 367.

¹H NMR (500 MHz, Methanol-d4) δ 7.64 (s, 1H), 7.06 (d, J=8.3 Hz, 1H), 6.90-6.87 (m, 2H), 6.02 (s, 1H), 1.99 (s, 3H), 1.92 (s, 3H), 1.90 (s, 6H).

### Example 48. Synthesis of 5-amino-3-(4-cyclopropyl-2-furyl)-1-(3-hydroxy-2,6-xylyl)-4-pyrazolecarboxamide (Compound 111)

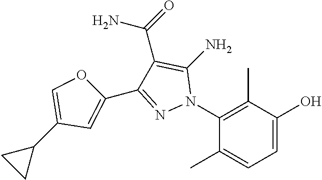 [SMILES: Cc1ccc(O)c(C)c1-n1nc(-c2cc(C3CC3)co2)c(C(N)=O)c1N]

5-Amino-3-(4-bromo-2-furyl)-1-(3-hydroxy-2,6-xylyl)-4-pyrazolecarboxamide (39 mg, 100 μmol), potassium carbonate (55 mg, 400 μmol), SPhos Pd G3 (4 mg, 5 μmol) and potassium cyclopropyltris(fluoro) boranuide (22 mg, 150 μmol) were mixed in 1 ml of dioxane/water (9:1). The reaction mixture was stirred at 95° C. for 2 days, diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 3 mg (6%) of the title compound as a TFA salt.

LCMS (ESI+): m/z [M+H]+ found: 353.

¹H NMR (400 MHz, Methanol-d4) δ 7.52 (s, 1H), 7.05 (d, J=8.4 Hz, 1H), 6.88 (d, J=8.2 Hz, 1H), 6.61 (s, 1H), 1.98 (s, 3H), 1.91 (s, 3H), 1.80-1.71 (m, 1H), 0.91-0.85 (m, 2H), 0.62-0.56 (m, 2H).

### Example 49. Synthesis of 3-{4-[(E)-1-propenyl]-2-furyl}-5-amino-1-(3-hydroxy-2,6-xylyl)-4-pyrazolecarboxamide (Compound 112)

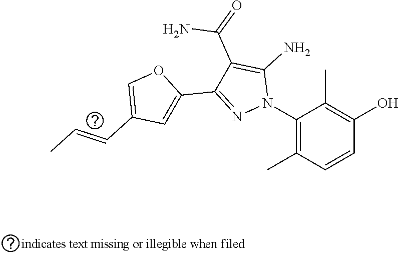 [SMILES: C/C=C/c1coc(-c2nn(-c3c(C)ccc(O)c3C)c(N)c2C(N)=O)c1]

5-Amino-3-(4-bromo-2-furyl)-1-(3-hydroxy-2,6-xylyl)-4-pyrazolecarboxamide (39 mg, 100 μmol), potassium carbonate (55 mg, 400 μmol), SPhos Pd G3 (4 mg, 5 μmol) and (E)-propenylboronic acid (17 mg, 200 μmol) were mixed in 1 ml of dioxane/water (9:1). The reaction mixture was stirred at 95° C. overnight. The reaction mixture was diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 25 mg (54%) of the title compound as a TFA salt.

LCMS (ESI+): m/z [M+H]+ found: 353.

¹H NMR (400 MHz, Methanol-d4) δ 7.62 (s, 1H), 7.06 (d, J=8.3 Hz, 1H), 6.97 (s, 1H), 6.89 (d, J=8.3 Hz, 1H), 6.29 (dd, J=15.7, 1.5 Hz, 1H), 6.15-6.05 (m, 1H), 1.99 (s, 3H), 1.92 (s, 3H), 1.83 (dd, J=6.5, 1.4 Hz, 3H).

### Example 50. Synthesis of 5-amino-3-[4-(3-hydroxy-3-methyl-1-butynyl)-2-furyl]-1-(3-hydroxy-2,6-xylyl)-4-pyrazolecarboxamide (Compound 113)

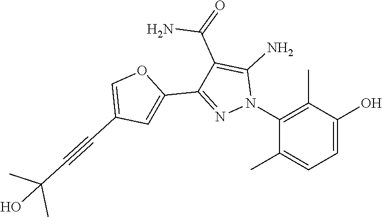 [SMILES: Cc1ccc(O)c(C)c1-n1nc(-c2cc(C#CC(C)(C)O)co2)c(C(N)=O)c1N]

5-Amino-3-(4-bromo-2-furyl)-1-(3-hydroxy-2,6-xylyl)-4-pyrazolecarboxamide (39 mg, 100 μmol), Pd(dppf)Cl₂ (7 mg, 10 μmol), copper (I) iodide (2 mg, 10 μmol) and 2-methyl-3-butyn-2-ol (13 mg, 150 μmol) were mixed in 0.5 ml of triethylamine. The reaction mixture was flushed with nitrogen and stirred at 90° C. for 2 days in a closed vial. The reaction mixture was diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 14 mg (28%) of the title compound as a TFA salt.

LCMS (ESI+): m/z [M+H]+ found: 395.

¹H NMR (400 MHz, Methanol-d4) δ 7.87 (s, 1H), 7.06 (d, J=8.4 Hz, 1H), 6.89 (d, J=8.3 Hz, 1H), 6.83 (s, 1H), 1.98 (s, 3H), 1.91 (s, 3H), 1.53 (s, 6H).

### Example 51. Synthesis of 5-amino-1-(3-hydroxy-2,6-xylyl)-3-[4-(1-propynyl)-2-furyl]-4-pyrazolecarboxamide (Compound 114)

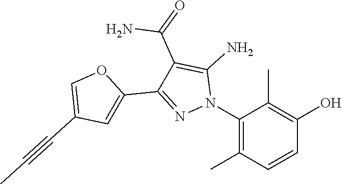 [SMILES: CC#Cc1coc(-c2nn(-c3c(C)ccc(O)c3C)c(N)c2C(N)=O)c1]

5-Amino-3-(4-bromo-2-furyl)-1-(3-hydroxy-2,6-xylyl)-4-pyrazolecarboxamide (39 mg, 100 μmol), SPhos Pd G3 (4 mg, 5 μmol) and tributyl(1-propynyl) stannane (36 mg, 110 μmol) were mixed in 1 ml of dioxane. The reaction mixture was stirred at 95° C. overnight, diluted with methanol/water, acidified with TFA and purified with reversed phase chromatography (Gemini NX-C18, 21×150 mm, water (0.1% TFA)/acetonitrile, gradient over 12 minutes, 25 ml/min). The pure fractions were pooled and concentrated giving 7 mg (15%) of the title compound as a TFA salt.

LCMS (ESI+): m/z [M+H]+ found: 351.

¹H NMR (400 MHz, Methanol-d4) δ 7.79 (s, 1H), 7.06 (d, J=8.2 Hz, 1H), 6.88 (d, J=8.3 Hz, 1H), 6.79 (s, 1H), 2.00 (s, 3H), 1.98 (s, 3H), 1.91 (s, 3H).

### Example 52. Synthesis of 5-amino-1-(3-hydroxy-2,6-dimethylphenyl)-3-(5-methoxybenzofuran-2-yl)-1H-pyrazole-4-carboxamide (Compound 115)

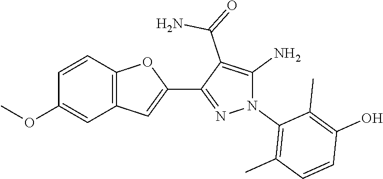 [SMILES: COc1ccc2oc(-c3nn(-c4c(C)ccc(O)c4C)c(N)c3C(N)=O)cc2c1]

### 5-amino-1-(3-hydroxy-2,6-dimethylphenyl)-3-(5-methoxybenzofuran-2-yl)-1H-pyrazole-4-carbonitrile

To a stirred solution of 2-(methoxy(5-methoxybenzofuran-2-yl)methylene)malononitrile (80 mg, 0.315 mmol) and 3-hydrazineyl-2,4-dimethylphenol (47.9 mg, 0.315 mmol) in methanol (5 ml), triethylamine (0.044 ml, 0.315 mmol) was added dropwise at 25° C. and the reaction mixture stirred at 60° C. for 2 h. The progress of the reaction was monitored by TLC. After completion of the reaction, the reaction mixture was concentrated under reduced pressure. The residue was diluted with ice-cold water and extracted with ethyl acetate. The combined organic layers were dried over anhydrous sodium sulfate and concentrated under reduced pressure to get 5-amino-1-(3-hydroxy-2,6-dimethylphenyl)-3-(5-methoxybenzofuran-2-yl)-1H-pyrazole-4-carbonitrile (70 mg, 0.125 mmol, 39.7% yield) as a brown solid.

LCMS (ESI) m/z=375.3 (M+H).

### 5-amino-1-(3-hydroxy-2,6-dimethylphenyl)-3-(5-methoxybenzofuran-2-yl)-1H-pyrazole-4-carboxamide

To a stirred solution of 5-amino-1-(3-hydroxy-2,6-dimethylphenyl)-3-(5-methoxybenzofuran-2-yl)-1H-pyrazole-4-carbonitrile (60 mg, 0.160 mmol) in methanol (6 ml) and hydrogen peroxide (4.91 μl, 0.160 mmol), 5M NaOH solution (0.641 mL, 3.21 mmol) was added dropwise at 25° C. and the reaction mixture stirred at 50° C. for 16 h. The progress of the reaction was monitored by TLC. After completion of the reaction, the reaction mixture was concentrated under reduced pressure. The crude was acidified with a dilute solution of polyphosphoric acid and extracted with ethyl acetate. The combined organic layer was washed with sodium bicarbonate solution (50 mL) followed by brine (50 mL), dried over anhydrous Na₂SO₄, filtered, and concentrated under reduced pressure. The resulting crude material was purified by prep. HPLC (Shimpak C18 (250×19 mm), Mobile phase A: 10 MM ABC in H₂O, Mobile phase B: Acetonitrile) to get 5-amino-1-(3-hydroxy-2,6-dimethylphenyl)-3-(5-methoxybenzofuran-2-yl)-1H-pyrazole-4-carboxamide (8.3 mg, 0.021 mmol, 13.07%) as an off white solid.

LCMS (ESI) m/z=393.0 (M+H).

¹H NMR (400 MHz, DMSO-d6): δ 9.56 (s, 1H), 7.55 (d, J=9.20 Hz, 1H), 7.19 (dd, J=0.80, 8.20 Hz, 2H), 7.06-7.01 (m, 2H), 6.95-6.90 (m, 2H), 6.18 (s, 2H), 3.80 (s, 3H), 1.91 (s, 3H), 1.81 (s, 3H).

### Example 53. Synthesis of 5-amino-1-(3,5-dimethyl-1H-indazol-4-yl)-3-(furan-2-yl)-1H-pyrazole-4-carboxamide (Compound 116) and Separation of Atropisomers Thereof (Compounds 103 and 117)

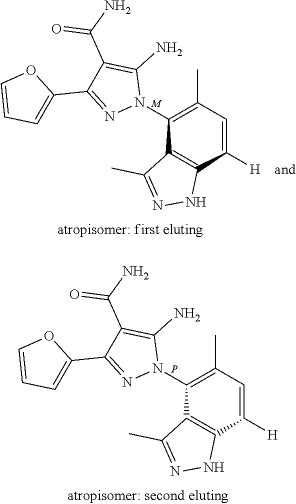

### 5-amino-1-(3,5-dimethyl-1H-indazol-4-yl)-3-(furan-2-yl)-1H-pyrazole-4-carbonitrile

To a stirred solution of 2-(furan-2-yl(methoxy)methylene)malononitrile (350 mg, 2.010 mmol) and 4-hydrazineyl-3,5-dimethyl-1H-indazole (354 mg, 2.010 mmol) in methanol (5 ml), TEA (1.099 ml, 8.04 mmol) was added dropwise at 25° C. and the resulting mixture stirred at 60° C. for 2 h. The progress of the reaction was monitored by UPLC. After the completion of the reaction, the reaction mixture was concentrated under vacuum to afford a crude compound which was poured into ice and the resulting mixture extracted with ethyl acetate. The organic layers were combined and dried over sodium sulfate and then concentrated in vacuum to afford 5-amino-1-(3,5-dimethyl-1H-indazol-4-yl)-3-(furan-2-yl)-1H-pyrazole-4-carbonitrile (620 mg, 1.305 mmol, 64.9% yield) as a brown solid.

LCMS (ESI) m/z=319.3 (M+H).

### 5-amino-1-(3,5-dimethyl-1H-indazol-4-yl)-3-(furan-2-yl)-1H-pyrazole-4-carboxamide

To a stirred solution of 5-amino-1-(3,5-dimethyl-1H-indazol-4-yl)-3-(furan-2-yl)-1H-pyrazole-4-carbonitrile (420 mg, 1.319 mmol) in methanol (15 ml) and 35% H₂O₂ in H₂O (5 ml, 1.319 mmol), NaOH 5M in H₂O (5 ml, 20.00 mmol) was added dropwise at 25° C. and the resulting mixture was stirred at 50° C. for 16 h. After the completion of the reaction, the reaction mixture was concentrated in vacuum. The crude material was acidified with a dilute solution of polyphosphoric acid and extracted with ethyl acetate. The organic layers were combined and washed with aqueous sodium bicarbonate and brine, dried over sodium sulfate and concentrated in vacuum. The residue (250 mg) was purified by Prep-HPLC (formic acid method). Pure fractions were lyophilized to give 5-amino-1-(3,5-dimethyl-1H-indazol-4-yl)-3-(furan-2-yl)-1H-pyrazole-4-carboxamide (90 mg).

LCMS (ESI) m/z=337.0 (M+H).

### Separation of the atropisomers of 5-amino-1-(3,5-dimethyl-1H-indazol-4-yl)-3-(furan-2-yl)-1H-pyrazole-4-carboxamide

Prep HPLC pure product 90 mg was separated by Chiral SFC purification (LUX-A1, 250×30 mm, 5 μm; isopropylamine/methanol). The first (35 mg) and second (54 mg) eluting isomers were separately collected and concentrated in vacuo to each afford an off white solid. The absolute configuration of the atropisomers were not determined but assigned arbitrarily.

First eluting isomer (Compound 103): LCMS (ESI) m/z=337.3 (M+H).

¹H NMR (400 MHz, DMSO-d6): δ 12.84 (s, 1H), 7.86 (s, 1H), 7.56-7.54 (d, J=8 Hz, 1H), 7.36-7.34 (d, J=8 Hz, 1H), 6.98 (bs, 3H), 6.65-6.63 (m, 2H), 6.31 (s, 1H), 2.13 (s, 3H), 1.96 (s, 3H).

Second eluting isomer (Compound 117): LCMS (ESI) m/z=337.3 (M+H).

¹H NMR (400 MHz, DMSO-d6): δ 12.8 (bs, 1H), 7.86 (s, 1H), 7.56-7.54 (d, J=8.8 Hz, 1H), 7.36-7.34 (d, J=8.0 Hz, 1H), 6.97 (bs, 2H), 6.81-6.80 (d, J=2.8 Hz, 1H), 6.65-6.63 (m, 1H), 6.31 (s, 2H), 2.13 (s, 3H), 1.96 (s, 3H).

### Example 54. Biological Assays

### Myt1 Kinase Binding Assay.

We used a LanthaScreen Europium (Eu) Kinase Binding Assay to determine the binding of provided compounds to Myt1 kinase. The assay utilizes an Alexa Fluor 647-labeled ATP competitive kinase tracer that binds to the ATP binding site of a GST-tagged Myt1 kinase, while a europium (Eu) labeled antibody binds to the GST tag. The proximity of fluorescently labeled kinase tracer and europium (Eu) donor fluorophore antibody leads to fluorescence resonance energy transfer (FRET) to the fluorescence label (acceptor) upon excitation of the Eu (donor). Displacement of tracer from the ATP-binding site by a test compound disturbs the proximity between both labels thus lowering the FRET.

This time resolved-FRET binding assay was performed in white 384-well low volume plates (Greiner, cat #784075), at room temperature in kinase buffer A (KBA; Invitrogen cat # PV3189), consisting of 50 mM HEPES-NaOH (pH 7.5), 0.01% Brij-35, 10 mM MgCl₂, and 1 mM EGTA. 5 μl of compound (diluted in reaction buffer, to 1% DMSO) were added to various wells in the plate, followed by 5 μl each of recombinant human PMYT-1 (full length PMYT-1 was expressed by baculovirus in insect cells using a N-terminal GST tag (MW: 80.8 kDa) (Invitrogen cat #A30984) and LanthaScreen Eu-anti-GST antibody (Invitrogen, cat #PV5594). After this, 5 μl of kinase tracer 178 (Invitrogen, cat #PV5593) was added to the plate and the plate was incubated for 60 minutes at room temperature. The final assay conditions in each well were: 3 nM tracer 178, 2.5 nM PMYT-1 and 1 nM Eu-labeled antibody in total assay volume of 15 μl. An Envision 2104 (Perkin-Elmer) Plate Reader with the following time-resolve fluorescence setting was used for performing LanthaScreen kinase binding assay.
Excitation 320 nm (30 nm bandpass)
Kinase Tracer Emission 665 nm (10 nm bandpass)
LanthaScreen Eu-anti-Tag Antibody Emission 615 nm (10 nm bandpass)
Dichroic Mirror Instrument dependent
Delay Time 100 μs
Integration Time 200 μs

To calculate the emission ratio, the acceptor/tracer emission (665 nM) was divided by the antibody/donor emission (615 nM) and the average of duplicate measurement was used for calculations. Data plotting, analysis of binding, and curve fitting was done using Excel add-in XLfit version 5.5.0.5 (IDBS, Guildford, United Kingdom). Results are presented in Table 2, below, where compounds having a mean IC₅₀ (since several compounds were tested more than once) less than or equal to 10 nM are represented as “A”; compounds having an IC₅₀ greater than 10 nM but less than or equal to 100 nM are represented as “B”; compounds having an IC₅₀ greater than 100 nM but less than or equal to 250 nM are represented as “C”, and compounds having an IC₅₀ greater than 250 nM are represented as “D”.

TABLE 2

| | LANTHA Kinase |
|---|---|
| Compound # | Binding for PMyt1 |
| 1 | C |
| 2 | C |
| 3 | D |
| 4 | C |
| 5 | B |
| 6 | D |
| 7 | D |
| 8 | D |
| 9 | B |
| 35 | A |
| 60 | D |
| 61 | B |
| 62 | D |
| 63 | B |
| 64 | D |
| 65 | B |
| 66 | D |
| 72 | A |
| 73 | A |
| 74 | B |
| 75 | A |
| 76 | B |
| 77 | B |
| 78 | B |
| 79 | A |
| 80 | A |
| 81 | A |
| 82 | A |
| 83 | B |
| 84 | B |
| 85 | B |
| 86 | A |
| 87 | A |
| 88 | A |
| 89 | A |
| 90 | A |
| 91 | B |
| 92 | B |
| 93 | B |
| 94 | A |
| 95 | A |
| 96 | A |
| 97 | A |
| 98 | A |
| 99 | A |
| 101 | B |
| 102 | A |
| 103 | B |
| 104 | A |
| 105 | B |
| 106 | A |
| 107 | A |
| 108 | A |
| 109 | D |
| 110 | C |
| 111 | B |
| 112 | A |
| 113 | B |
| 114 | A |
| 115 | A |
| 116 | D |
| 119 | A |
| 120 | A |
| 121 | B |
| 122 | A |
| 123 | A |
| 124 | B |
| 125 | A |

### Cellular Assay

The following cellular assay was used to determine the extent to which the compounds disclosed herein inhibit phosphorylation of CDK1 at Thr 14.

### Cell Culture

DAOY medulloblastoma cell line (ATCC) was cultured in Minimum Essential Medium Eagle supplemented with 10% fetal calf serum (Sigma), 1% Penicillin-Streptomycin and 10 mM HEPES buffer (HyClone). Cell cultures were kept in a humidified incubator at 37° C. and 5% CO₂. Cells were routinely tested for Mycoplasma contamination.

### AlphaLISA Assay

For target engagement assessment, quantification of Cdk1 phosphorylated on threonine 14 was detected using the AlphaLISA® SureFire® Ultra™ Human Phospho-CDK1 (Thr14) assay (Perkin Elmer). DAOY cells were seeded into tissue culture 96-well plates (VWR) to a density of 10,000 cells per well. Twenty-four hours post-seeding cells were incubated for 4 h with test compounds at concentrations ranging from 7 to 5000 nM. Cells were washed with PBS and lysed in 50 μl AlphaLISA® lysis buffer before freezing at −80° C. Ten μl of the lysate was transferred to 384 well plates and incubated with AlphaLISA® donor and acceptor beads according to the manufacturer's instructions. Dose response curves and EC₅₀ values were calculated and visualized using GraphPad Prism version 9.

### Cell Titer Glo Viability Assay

DAOY cells were seeded at a density of 250 cells per well in tissue culture 384 well plates (Corning Costar). After overnight incubation, cells were incubated with test compounds at concentrations ranging from 0.17 to 10,000 nM. Viability compared to untreated control was assessed at 144 hours using Cell Titer Glo® 2.0 Viability Assay (Promega).

Results for target engagement are presented in Table 3, below, where compounds having a mean EC₅₀ less than or equal to 250 nM are represented as “A”; compounds having an EC₅₀ greater than 250 nM but less than or equal to 500 nM are represented as “B”; compounds having an EC₅₀ greater than 500 nM but less than or equal to 1000 nM are represented as “C”; and compounds having an EC₅₀ greater than 1000 nM are represented as “D”.

Results for viability in the Cell Titer Glo Assay are presented in Table 4, below, where compounds having an EC₅₀ less than or equal to 500 nM are represented as “A”; compounds having an EC₅₀ greater than 500 nM but less than or equal to 1000 nM are represented as “B”; compounds having an EC₅₀ greater than 1000 nM are represented as “C”.

TABLE 3
AlphaLISA Measured Myt1 Kinase Target
Engagement (TE) of Exemplary Compounds

| | TE Myt1 Kinase |
|---|---|
| Compound # | (AlphaLISA) |
| 1 | C |
| 4 | D |
| 5 | D |
| 6 | D |
| 9 | A |
| 35 | A |
| 61 | D |
| 72 | A |
| 73 | A |
| 74 | D |
| 75 | A |
| 76 | D |
| 77 | D |
| 78 | B |
| 79 | A |
| 80 | A |
| 81 | A |
| 82 | C |
| 83 | D |
| 84 | D |
| 85 | D |
| 86 | A |
| 87 | A |
| 88 | A |
| 89 | B |
| 90 | A |
| 91 | D |
| 92 | C |
| 93 | C |
| 94 | A |
| 95 | A |
| 96 | A |
| 97 | A |
| 98 | A |
| 99 | A |
| 100 | B |
| 101 | A |
| 102 | A |
| 104 | A |
| 105 | D |
| 106 | A |
| 107 | A |
| 108 | A |
| 111 | A |
| 112 | A |
| 113 | B |
| 114 | A |
| 115 | A |
| 119 | A |
| 120 | B |
| 122 | A |
| 123 | A |
| 124 | D |
| 125 | C |

TABLE 4
Cell Titer-Glo Measured Viability Data
in DAOYCells for Exemplary Compounds

| | Viability |
|---|---|
| Compound # | (DAOY cells) |
| 9 | C |
| 35 | C |
| 72 | A |
| 73 | A |
| 75 | A |
| 79 | A |
| 80 | C |
| 81 | B |
| 82 | C |
| 86 | B |
| 87 | A |
| 88 | A |
| 90 | A |
| 94 | A |
| 95 | A |
| 96 | A |
| 97 | A |
| 98 | A |
| 99 | C |
| 100 | C |
| 101 | C |
| 102 | A |
| 104 | B |
| 106 | A |
| 107 | A |
| 108 | B |
| 111 | B |
| 112 | B |
| 113 | C |
| 114 | B |
| 115 | B |
| 119 | B |
| 122 | A |
| 123 | B |

### Example 55. Bioavailability Study

We compared the oral bioavailability of Compound 75:

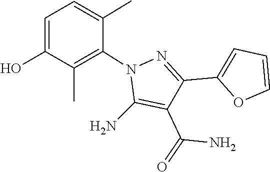 [SMILES: Cc1ccc(O)c(C)c1-n1nc(-c2ccco2)c(C(N)=O)c1N]

with that of Compound 122:

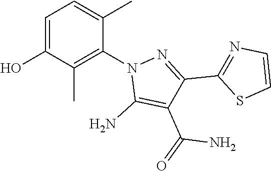 [SMILES: Cc1ccc(O)c(C)c1-n1nc(-c2nccs2)c(C(N)=O)c1N]

(Compound A), which has a thiazolyl moiety in place of the furanyl moiety in Compound 75. Female CD-1 mice treated with a single intravenous bolus (IV; 1 mg/kg) or oral gavage (PO; 3 mg/kg) of one of the compounds. The percent bioavailability (% F) was calculated by dividing the dose normalized area under the plasma concentration curve (AUC0-∞) by the dose normalized IV plasma AUC0-∞. The results are shown in the table below.

TABLE 5

| | Bioavail- |
|---|---|
| Compound | ability (% F) |
| 75 | 64 |
| A | 32 |

Compound 75 demonstrated twice the bioavailability of Compound A, leading us to conclude that compounds having a furanyl moiety at R¹ have a superior bioavailability than compounds having a thiazolyl moiety at that position.

1. A compound of formula II: 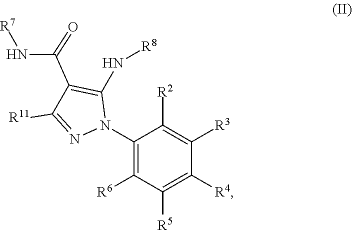
or a solvate, enantiomer, tautomer, or diastereomer thereof, or a pharmaceutically acceptable salt of any of the foregoing wherein:
R¹¹ is 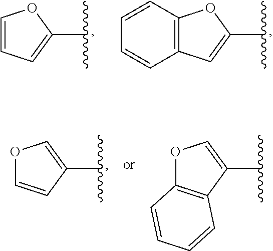
each of which is optionally substituted;
each of R², R³, R⁴ and R⁶ is independently hydrogen, halo, —C₁-C₄ alkyl, —O—C₁-C₄ alkyl, —C₂-C₄ alkynyl, —CN, cyclopropyl, or phenyl; and
R⁵ is —OH; or
R² is halo, —C₁-C₄ alkyl, —O—C₁-C₄ alkyl, —C₂-C₄ alkynyl, —CN, or cyclopropyl;
each of R³ and R⁴ is independently hydrogen, halo, —C₁-C₄ alkyl, —O—C₁-C₄ alkyl, —CN, cyclopropyl, or phenyl; and
R⁵ and R⁶ are taken together with carbon atoms to which they are bound to form Ring A;
Ring A is a 4-7 membered heterocyclic or heteroaromatic ring comprising a ring nitrogen atom and optionally substituted with one or more R^{a} groups;
each R^{a} is independently selected from halo, ═O, —C₁-C₄ alkyl, —O—C₁-C₄ alkyl, —CN, and cyclopropyl;
each of R⁷ and R⁸ is independently hydrogen or optionally substituted C₁-C₄ alkyl; and wherein one or more hydrogen atoms in the compound is optionally replaced with a deuterium atom.

2. The compound of claim 1, wherein the compound has structural formula Ila: 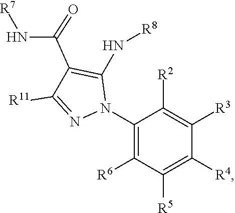
or a solvate, enantiomer, tautomer, or diastereomer thereof, or a pharmaceutically acceptable salt of any of the foregoing wherein:
R¹¹ is 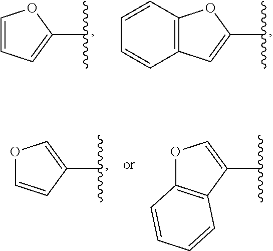
each of which is optionally substituted;
each of R², R³, R⁴ and R⁶ is independently hydrogen, halo, —C₁-C₄ alkyl, —O—C₁-C₄ alkyl, —C₂-C₄ alkynyl, —CN, cyclopropyl, or phenyl;
R⁵ is —OH;
each of R⁷ and R⁸ is independently hydrogen or optionally substituted C₁-C₄ alkyl; and wherein one or more hydrogen atoms in the compound is optionally replaced with a deuterium atom.

3. The compound of claim 1, wherein the compound has structural formula Ilb: 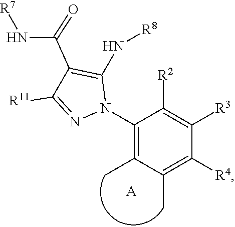
or a solvate, enantiomer, tautomer, or diastereomer thereof, or a pharmaceutically acceptable salt of any of the foregoing wherein:
R¹¹ is 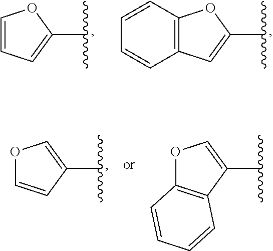
each of which is optionally substituted;
R² is halo, —C₁-C₄ alkyl, —O—C₁-C₄ alkyl, —C₂-C₄ alkynyl, —CN, or cyclopropyl;
each of R³ and R⁴ is independently hydrogen, halo, —C₁-C₄ alkyl, —O—C₁-C₄ alkyl, —CN, cyclopropyl, or phenyl; and
R⁵ and R⁶ are taken together with carbon atoms to which they are bound to form Ring A;
Ring A is a 4-7 membered heterocyclic or heteroaromatic ring comprising a ring nitrogen atom and optionally substituted with one or more R^{a} groups;
each R^{a} is independently selected from halo, ═O, —C₁-C₄ alkyl, —O—C₁-C₄ alkyl, —CN, and cyclopropyl;
each of R⁷ and R⁸ is independently hydrogen or optionally substituted C₁-C₄ alkyl; and wherein one or more hydrogen atoms in the compound is optionally replaced with a deuterium atom.

4. The compound of any one of claims 1-3, wherein R¹¹ is optionally substituted with one or more substituent independently selected from halo, —CN, C₁-C₄ alkyl, C₂-C₅ alkenyl, C₂-C₅ alkynyl, C₃-C₆ cycloalkyl, —O—C₁-C₄ alkyl, optionally substituted aryl, and optionally substituted heteroaryl, wherein any alkyl, alkenyl, alkynyl, aryl or heteroaryl substituent on R¹¹ is optionally further substituted with —OH.

5. The compound of claim 4, wherein R¹¹ is optionally substituted with one or more substituent independently selected from bromo, chloro, —CN, —CH₃, —CH(CH₃)₂, —CH₂═CH₂, —C(CH₃)═CH₂, —CH═CH(CH₃)₂, —CH═CH—CH₃, —C≡C—C(CH₃)₂—OH, —C≡C—CH₃, —OCH₃, cyclopropyl, phenyl, or pyridinyl.

6. The compound on any one of claims 1-5, wherein R² is —CH₃ or —Cl.

7. The compound on any one of claims 1-6, wherein R³ is hydrogen.

8. The compound on any one of claims 1-7, wherein R⁴ is hydrogen or chloro.

9. The compound on any one of claim 1-2 or 4-8, wherein R⁶ is —CH₃ or —Cl.

10. The compound of claim 9, wherein each of R² and R⁶ are simultaneously —CH₃ or —Cl.

11. The compound on any one of claim 1-2 or 4-5, wherein the portion of the compound represented by

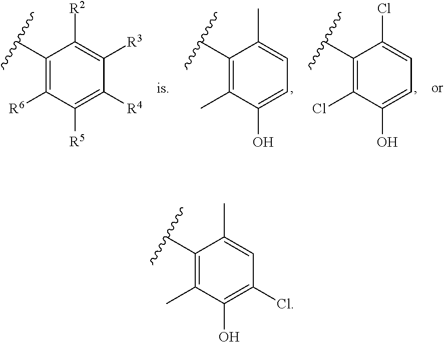

12. The compound on any one of claim 1 or 3-8, wherein Ring A is a 5-membered heterocyclic ring comprising a ring nitrogen atom and optionally substituted with one R^{a} group.

13. The compound of claim 12, wherein Ring A is pyrazolyl optionally substituted with one R^{a} group.

14. The compound of claim Ring A is

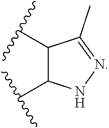 [SMILES: *C1NN=C(C)C1*]

15. The compound on any one of claims 1-14 wherein R⁷ is hydrogen.

16. The compound on any one of claims 1-15, wherein R⁸ is hydrogen.

17. The compound of any one of claims 1-16, selected from any compound set forth in the following table:

| Com- | |
|---|---|
| pound # | Structure |
| 72 | 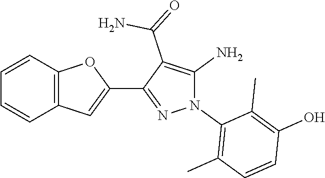 [SMILES: Cc1ccc(O)c(C)c1-n1nc(-c2cc3ccccc3o2)c(C(N)=O)c1N] |
| 73 | 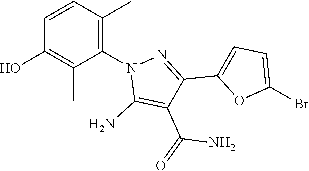 [SMILES: Cc1ccc(O)c(C)c1-n1nc(-c2ccc(Br)o2)c(C(N)=O)c1N] |
| 74 | 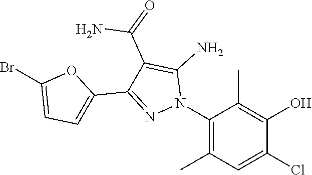 [SMILES: Cc1cc(Cl)c(O)c(C)c1-n1nc(-c2ccc(Br)o2)c(C(N)=O)c1N] |
| 75 | 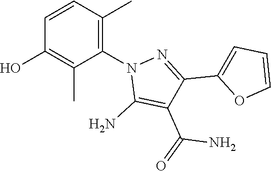 [SMILES: Cc1ccc(O)c(C)c1-n1nc(-c2ccco2)c(C(N)=O)c1N] |
| 76 | 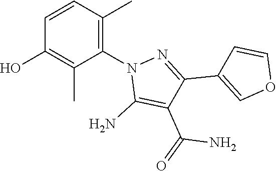 [SMILES: Cc1ccc(O)c(C)c1-n1nc(-c2ccoc2)c(C(N)=O)c1N] |
| 77 | 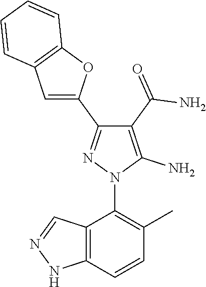 [SMILES: Cc1ccc2[nH]ncc2c1-n1nc(-c2cc3ccccc3o2)c(C(N)=O)c1N] |
| 78 | 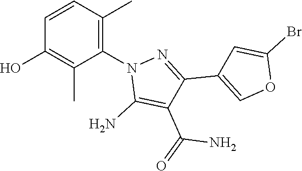 [SMILES: Cc1ccc(O)c(C)c1-n1nc(-c2coc(Br)c2)c(C(N)=O)c1N] |
| 79 | 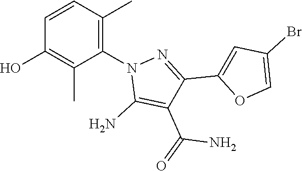 [SMILES: Cc1ccc(O)c(C)c1-n1nc(-c2cc(Br)co2)c(C(N)=O)c1N] |
| 80 | 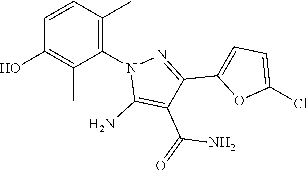 [SMILES: Cc1ccc(O)c(C)c1-n1nc(-c2ccc(Cl)o2)c(C(N)=O)c1N] |
| 81 | 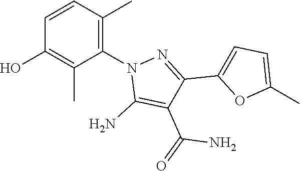 [SMILES: Cc1ccc(-c2nn(-c3c(C)ccc(O)c3C)c(N)c2C(N)=O)o1] |
| 82 | 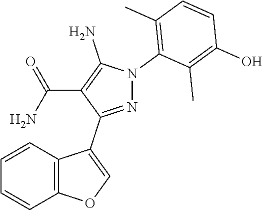 [SMILES: Cc1ccc(O)c(C)c1-n1nc(-c2coc3ccccc23)c(C(N)=O)c1N] |
| 83 | 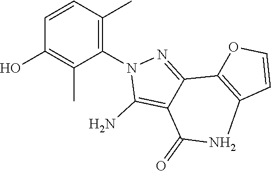 [SMILES: Cc1ccc(O)c(C)c1-n1nc2c(c1N)c(=O)[nH]c1ccoc12] |
| 84 | 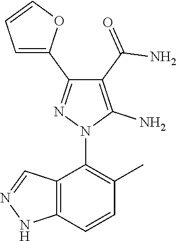 [SMILES: Cc1ccc2[nH]ncc2c1-n1nc(-c2ccco2)c(C(N)=O)c1N] |
| 85 | 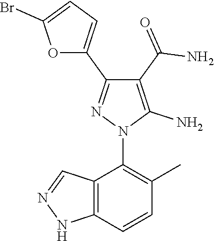 [SMILES: Cc1ccc2[nH]ncc2c1-n1nc(-c2ccc(Br)o2)c(C(N)=O)c1N] |
| 86 | 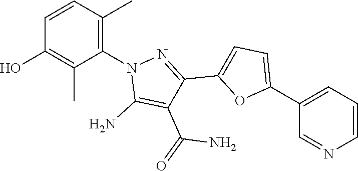 [SMILES: Cc1ccc(O)c(C)c1-n1nc(-c2ccc(-c3cccnc3)o2)c(C(N)=O)c1N] |
| 87 | 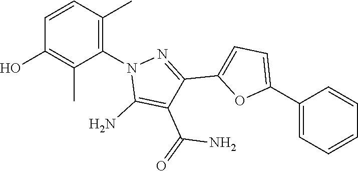 [SMILES: Cc1ccc(O)c(C)c1-n1nc(-c2ccc(-c3ccccc3)o2)c(C(N)=O)c1N] |
| 88 | 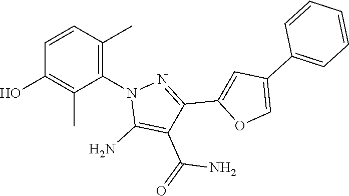 [SMILES: Cc1ccc(O)c(C)c1-n1nc(-c2cc(-c3ccccc3)co2)c(C(N)=O)c1N] |
| 89 | 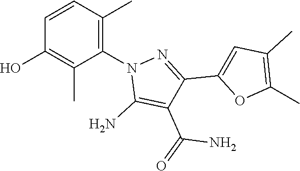 [SMILES: Cc1cc(-c2nn(-c3c(C)ccc(O)c3C)c(N)c2C(N)=O)oc1C] |
| 90 | 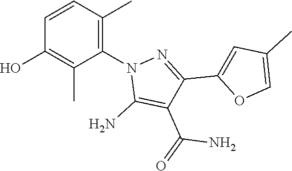 [SMILES: Cc1coc(-c2nn(-c3c(C)ccc(O)c3C)c(N)c2C(N)=O)c1] |
| 91 | 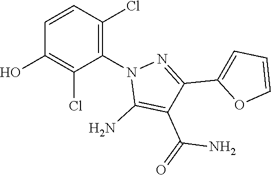 [SMILES: NC(=O)c1c(-c2ccco2)nn(-c2c(Cl)ccc(O)c2Cl)c1N] |
| 92 | 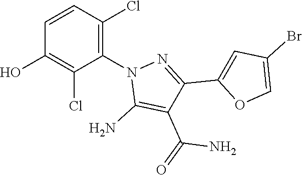 [SMILES: NC(=O)c1c(-c2cc(Br)co2)nn(-c2c(Cl)ccc(O)c2Cl)c1N] |
| 93 | 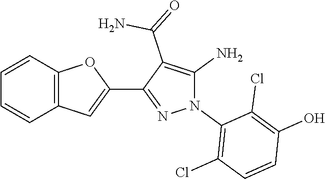 [SMILES: NC(=O)c1c(-c2cc3ccccc3o2)nn(-c2c(Cl)ccc(O)c2Cl)c1N] |
| 94 | 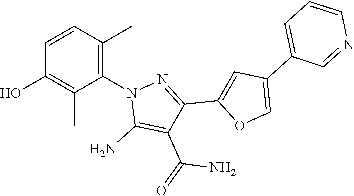 [SMILES: Cc1ccc(O)c(C)c1-n1nc(-c2cc(-c3cccnc3)co2)c(C(N)=O)c1N] |
| 95 | 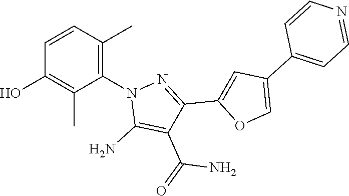 [SMILES: Cc1ccc(O)c(C)c1-n1nc(-c2cc(-c3ccncc3)co2)c(C(N)=O)c1N] |
| 96 | 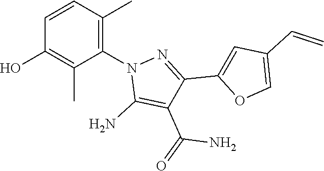 [SMILES: C=Cc1coc(-c2nn(-c3c(C)ccc(O)c3C)c(N)c2C(N)=O)c1] |
| 97 | 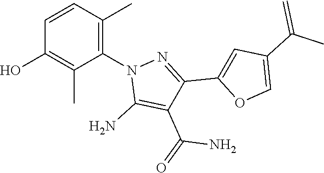 [SMILES: C=C(C)c1coc(-c2nn(-c3c(C)ccc(O)c3C)c(N)c2C(N)=O)c1] |
| 98 | 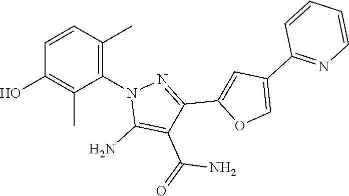 [SMILES: Cc1ccc(O)c(C)c1-n1nc(-c2cc(-c3ccccn3)co2)c(C(N)=O)c1N] |
| 99 | 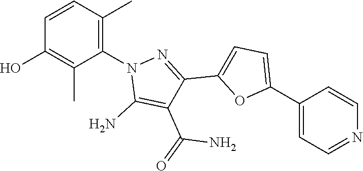 [SMILES: Cc1ccc(O)c(C)c1-n1nc(-c2ccc(-c3ccncc3)o2)c(C(N)=O)c1N] |
| 100 | 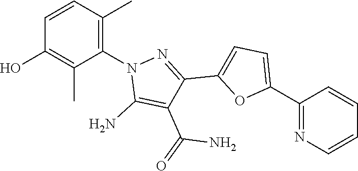 [SMILES: Cc1ccc(O)c(C)c1-n1nc(-c2ccc(-c3ccccn3)o2)c(C(N)=O)c1N] |
| 101 | 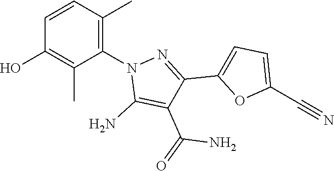 [SMILES: Cc1ccc(O)c(C)c1-n1nc(-c2ccc(C#N)o2)c(C(N)=O)c1N] |
| 102 | 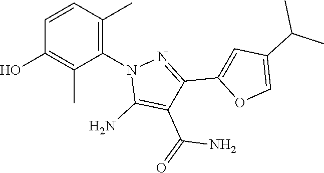 [SMILES: Cc1ccc(O)c(C)c1-n1nc(-c2cc(C(C)C)co2)c(C(N)=O)c1N] |
| 103 | 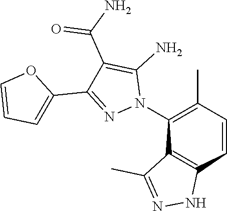 |
| 104 | 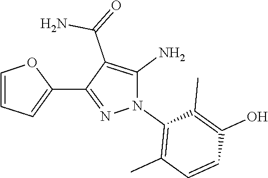 |
| 105 | 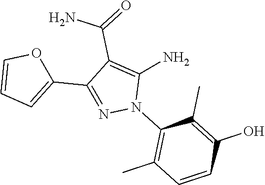 |
| 106 | 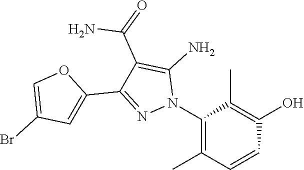 |
| 107 | 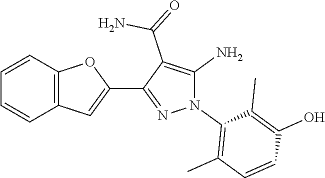 |
| 108 | 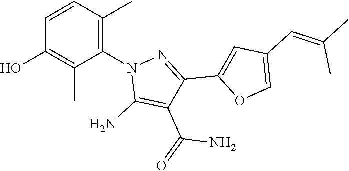 [SMILES: CC(C)=Cc1coc(-c2nn(-c3c(C)ccc(O)c3C)c(N)c2C(N)=O)c1] |
| 109 | 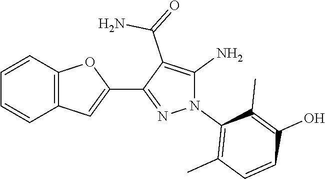 |
| 110 | 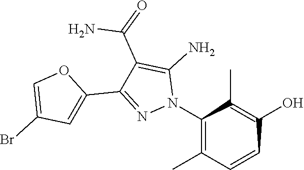 |
| 111 | 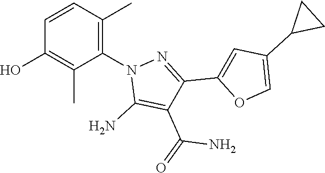 [SMILES: Cc1ccc(O)c(C)c1-n1nc(-c2cc(C3CC3)co2)c(C(N)=O)c1N] |
| 112 | 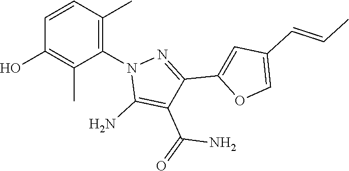 [SMILES: C/C=C/c1coc(-c2nn(-c3c(C)ccc(O)c3C)c(N)c2C(N)=O)c1] |
| 113 | 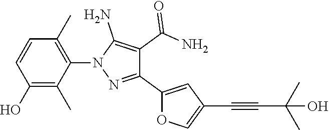 [SMILES: Cc1ccc(O)c(C)c1-n1nc(-c2cc(C#CC(C)(C)O)co2)c(C(N)=O)c1N] |
| 114 | 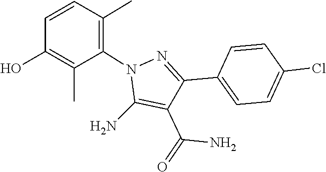 [SMILES: Cc1ccc(O)c(C)c1-n1nc(-c2ccc(Cl)cc2)c(C(N)=O)c1N] |
| 115 | 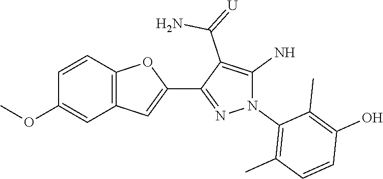 |
| 116 | 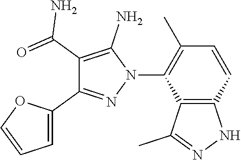 |
| 117 | 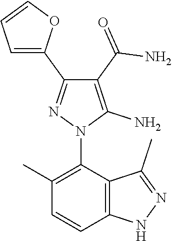 [SMILES: Cc1ccc2[nH]nc(C)c2c1-n1nc(-c2ccco2)c(C(N)=O)c1N] |

or a pharmaceutically acceptable salt thereof.

18. A pharmaceutical composition comprising an effective amount of the compound of any one of claims 1-17; and a pharmaceutically acceptable carrier.

19. A method of inhibiting Myt1 kinase activity in a subject comprising the step of administering to the subject an effective amount of a compound of any one of claims 1-17, or a composition of claim 18.

20. A method of treating a subject suffering from a cancer characterized by aberrant Myt1 kinase activity comprising the step of administering to the subject an effective amount of a compound of any one of claims 1-17, or a composition of claim 18.

21. A method of treating a subject suffering from a cancer characterized by amplification and/or overexpression of CCNE1 comprising the step of administering to the subject an effective amount of a compound of any one of claims 1-17, or a composition of claim 18.

22. The method of claim 20 or 21, wherein the cancer is uterine cancer, ovarian cancer, breast cancer, stomach cancer, esophageal cancer, lung cancer, or endometrial cancer.

23. A method of treating a subject suffering from a cancer characterized by an inactivating mutation in a FBXW7 gene, comprising the step of administering to the subject an effective amount of a compound of any one of claims 1-17, or a composition of claim 18.

24. The method of claim 20 or 23, wherein the cancer is uterine cancer, colorectal cancer, breast cancer, lung cancer, or esophageal cancer.

25. The method of any one of claims 19-24, wherein the subject is treated only if it has been determined that the subject is resistant to a Wee1A kinase inhibitor.

26. The method of any one of claims 19-24, wherein the subject is co-administered a pharmaceutically acceptable Wee1A kinase inhibitor.

27. The method of any one of claims 19-26, wherein the subject is co-administered a pharmaceutically acceptable DNA damaging agent.
